(19) United States
(12) Patent Application Publication
(10) Pub. No.: US 2024/0059658 A1
(43) Pub. Date: Feb. 22, 2024

(54) QUINOLINE DERIVATIVES AND USES IN MANAGING CANCER

(71) Applicants: Emory University, Atlanta, GA (US); University of Tennessee Research Foundation, Memphis, TN (US); Children's Healthcare of Atlanta, Inc., Atlanta, GA (US)

(72) Inventors: Muxiang Zhou, Atlanta, GA (US); Lubing Gu, Atlanta, GA (US); We Li, Memphis, TN (US); Zhongzhi Wu, Memphis, TN (US)

(21) Appl. No.: 17/642,032

(22) PCT Filed: Sep. 10, 2020
(86) PCT No.: PCT/US2020/050193

§ 371 Date: Mar. 10, 2022

Related U.S. Application Data
(60) Provisional application No. 62/898,180, filed on Sep. 10, 2019.

(51) Int. Cl. C07D 215/36; A61P 35/00; C07D 221/16; C07D 221/18; C07D 405/12; C07D 471/04; C07D 491/048; C07D 491/052

(52) U.S. Cl. CPC C07D 215/36; A61P 35/00; C07D 221/16; C07D 221/18; C07D 405/12; C07D 471/04; C07D 491/048; C07D 491/052

(57) ABSTRACT

Provided herein are compounds, pharmaceutical compositions including such compounds, and methods of using such compounds to treat diseases or disorders associated with MDM2 activity.

### CROSS-REFERENCE TO RELATED APPLICATION

The present application claims priority to U.S. Application No 62/898,180, filed Sep. 10, 2019, the disclosure of which is incorporated herein by reference in its entirety.

### FIELD OF THE INVENTION

The invention is generally directed to quinoline derivatives and methods of use thereof.

### BACKGROUND

MDM2 and XIAP are cell-survival proteins in tumor cells. MDM2 acts as an oncoprotein, promoting cancer progression mainly through inhibition of the tumor suppressor p53, while the anti-apoptotic protein XIAP plays an important role in development of resistance to treatment via inhibition of therapy-induced apoptosis. MDM2 overexpression and upregulated XIAP have been detected in various human cancers, and elevated MDM2 and XIAP expression in tumor cells is associated with disease progression and poor treatment outcome. Gu et al. report duel inhibitors of MDM2 and XIAP for cancer treatment. Cancer Cell. 2016, 30(4):623-636.

Current chemotherapy treatments are typically not universally effective, and cancers sometimes recur after treatment. Thus, there is a need to identify improved cancer therapies.

### SUMMARY OF THE INVENTION

It has been discovered that certain compounds have properties of MDM2 inhibition, p53 induction, and anti-cancer properties. This disclosure relates to such compounds, pharmaceutical compositions comprising such compounds, and uses related thereto. In an aspect, provided herein are compounds of Formula I,

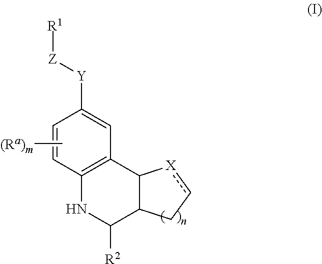

including salts and prodrugs thereof, wherein the substituents are reported herein.

In another aspect, provided herein are compounds of Formula IV:

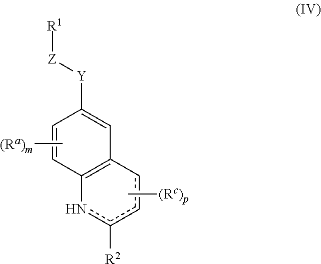

including salts and prodrugs thereof, wherein the substituents are reported herein.

In certain embodiments, this disclosure relates to methods of treating cancer comprising administering a therapeutically effective amount of a compound disclosed herein to a subject in need thereof.

### BRIEF DESCRIPTION OF THE DRAWINGS

FIG. 1 illustrates embodiments of this disclosure. A, E ring=aryl, hetero-aryl or aliphatic ring; R1, R2=H, halogens or alkyl; R3=H, halogens or alkyl; R4=H, alkyl, halogens, —COR, —COOR, —CONHR, —SO₂R or —NHCOR; X=CH, O, S or NH; n=1-3.

FIG. 2 illustrates the preparation of embodiments of this disclosure. A, E ring=aryl, hetero-aryl or aliphatic ring; R1, R2=H, halogens or alkyl; R3=H, halogens or alkyl; R4=H, alkyl, halogens, —COR, —COOR, —CONHR, —SO₂R or —NHCOR; X=CH, O, S or NH; n=1-3.

FIGS. 3A-3C show thermodynamic measurement of the binding of MX69 (FIG. 3A) and its analogs MX69-52 (69L52; FIG. 3B) and MX69-53 (69L53; FIG. 3C) to MDM2 RING protein using ITC. The upper box is the raw heating power over time and the lower box is a fit of the integrated energy values, normalized for each injection.

FIG. 4A-4D show the effects of 69L52 on cancer cell viability and growth as well as on normal human hematopoiesis. FIG. 4A shows WST assay for cytotoxic effects of 14 on two ALL cell lines (EU-1 and EU-3) and three NB cell lines (NB-1643, SHEP1 and LA1-55N). Cells were treated at the doses indicated for 48 h. Data represent mean±SD of three independent experiments. FIG. 4B shows representative colony formation of NB cell lines as indicated treated with or without 14 for two weeks. FIGS. 4C and 4D shows the comparison of inhibitory effects of 14 and Dox on CFU-GM and BFU-E in NBMM cells, using in vitro colony formation analysis. NBMM cells (1×105) were incubated with GM-CSF or Epo, in the absence or presence of 1 μM either 14 or Dox. Colonies were counted after 14 days of culture. Comparison of colony numbers, *p<0.01.

FIGS. 5A-5E show the effects of 69L52 on expression of MDM2 and XIAP and activation of p53. FIG. 5A shows the Western blot assays showed the dose-response and time-course of MDM2 and XIAP inhibition as well as p53 induction by 69L52 in EU-1 cell line treated with doses and times as indicated. FIG. 5B shows the EU-1 cells with or without 69L52 treatment (1 μM for 8 h) were treated with 10 μM MG132 for additional 4 h and then Western blots performed for expression of proteins as indicated. FIG. 5C shows the CHX chase assay for detection of protein turnover in EU-1 cells treated with or without (control) 1 μM of 69L52 for 4 h. Numerical labels under each band of Western blots represent the expression levels after normalization for GAPDH, compared with untreated (0) samples (defined as 1 unit). FIG. 5D shows the IP and Western blot assay using anti-MDM2 and anti-ubiquitin antibodies respectively, to detect effects of 69L52 (1 μM) on ubiquitination of endogenous MDM2 in EU-1 cells. FIG. 5E shows the Western blot for expression of p53 and its targets p21 and PUMA in EU-1 cells treated with 69L52.

FIG. 6A shows the EU-1 cells treated with or without 1 μM of 69L52 for 4 h and their cytoplasmic lysates were fractionated on a sucrose gradient. RNA was extracted from each of the fractions and subjected to qRT-PCR for analysis of the distribution of XIAP and Actin mRNAs. Data show the percentage of the total amount of corresponding mRNA in each fraction and represent mean±SD of three independent experiments. FIG. 6B shows the Western blot that shows the activation of caspase-3 and -9 as well as cleavage of death substrate PARP in EU-1 cells following treatment with 5 μM of MX69 and 1 μM of 69L52 for times indicated.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, particular methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

With regard to chemical structure, it is understood that claiming compounds that are racemic encompasses all of the isomers, tautomers, enantiomers, or diastereomers unless otherwise specified to be a composition of excess of a specific isomer. For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the amount of one enantiomer is greater than the amount of that one enantiomer in a control mixture of the racemic composition (e.g., greater than 1:1 by weight). For example, an enantiomerically enriched preparation of the S enantiomer, means a preparation of the compound having greater than about 50% by weight of the S enantiomer relative to the total weight of the preparation (e.g., total weight of S and R isomers) such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to the total weight of the preparation, such as at least about 90% by weight, and further such as at least about 95% by weight. In certain embodiments, the compound provided herein is made up of at least about 90% by weight of one enantiomer. In other embodiments, the compound is made up of at least about 95%, about 98%, or about 99% by weight of one enantiomer.

In certain embodiments, the pharmaceutically acceptable form is a tautomer. As used herein, the term “tautomer” is a type of isomer that includes two or more interconvertable compounds resulting from at least one formal migration of a hydrogen atom and at least one change in valency (e.g., a single bond to a double bond, a triple bond to a double bond, or a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-enol; amide-imide; lactam-lactim; enamine-imine; and enamine-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. Certain isotopically-labeled disclosed compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

The term “prodrug” refers any covalently bonded carriers, which release the active compound in vivo when such prodrug is administered to a subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a carboxyl, hydroxy, amino or mercapto group is bonded to any group that, when the prodrug of the active compound is administered to a subject, cleaves to form a free hydroxy, free carboxyl, free amino or free mercapto group, respectively. Examples of prodrugs include, but are not limited to, carboxyl esters, acetate, formate and benzoate derivatives of an alcohol or acetamide, formamide and benzamide derivatives of an amine functional group in the active compound and the like.

The term “ester” refers to esters of acidic groups, including, but not limited to, carboxylic acids, phosphoric acids, phosphinic acids, sulfonic acids, sulfinic acids, and boronic acids, e.g., a radical of formula —COOR, where R is selected from alkyl, alkenyl, alkynyl, haloalkyl, heteroalkyl (bonded through a chain carbon), cycloalkyl, cycloalkylalkyl, aryl, aralkyl, heterocycloalkyl (bonded through a ring carbon), heterocycloalkylalkyl, heteroaryl (bonded through a ring carbon), and heteroarylalkyl. Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 4th Ed., John Wiley & Sons, New York, N.Y., 2006, which is incorporated herein by reference in its entirety. Unless stated otherwise in the specification, an ester group can be optionally substituted by one or more substituents.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, “alkyl” means a noncyclic straight chain or branched, unsaturated or saturated hydrocarbon such as those containing from 1 to 10 carbon atoms (C₁-C₁₀)alkyl. In certain embodiments, any alkyl is a (C₁-C₆)alkyl, or any group containing an alkyl reported herein, e.g., a (C₁-C₆)alkoxy. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or “heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. In an embodiment, aryl is phenyl. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, and, the like, where optionally one or more pair of substituents together with the atoms to which they are bonded form a 3 to 7 member ring.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above with the indicated number of carbon atoms attached through a sulfur bridge. An example of an alkylthio is methylthio, (i.e., —S—CH3).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. In an embodiment, alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bride (i.e., —(C═O)alkyl).

“Alkylsulfonyl” refers to an alkyl as defined above with the indicated number of carbon atoms attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “Arylsulfamoyl” refers to an alkyl attached through a sulfamoyl bridge (i.e., (i.e., —NHS(═O)₂aryl).

“Alkylsulfinyl” refers to an alkyl as defined above with the indicated number of carbon atoms attached through a sulfinyl bridge (i.e. —S(═O)alkyl).

The terms “cycloalkyl” and “cycloalkenyl” refer to mono-, bi-, or tri homocyclic ring groups of 3 to 15 carbon atoms which are, respectively, fully saturated and partially unsaturated. The term “cycloalkenyl” includes bi- and tricyclic ring systems that are not aromatic as a whole, but contain aromatic portions (e.g., fluorene, tetrahydronapthalene, dihydroindene, and the like). The rings of multi-ring cycloalkyl groups may be either fused, bridged and/or joined through one or more spiro unions. The terms “substituted cycloalkyl” and “substituted cycloalkenyl” refer, respectively, to cycloalkyl and cycloalkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, and the like.

The terms “halogen” and “halo” refer to fluorine, chlorine, bromine, and iodine.

The term “carbamoyl” refers to a functional group having the formula —OC(O)NH₂ or, alternatively, —NHC(O)OH.

The term “boronic acid” refers to a functional group having the formula —B(OH)₂.

The term “boronic ester” refers to a functional group having the formula —B(Oalkyl)₂ wherein alkyl is defined above and the two alkyl groups may be connected to form a cyclic boronic ester.

The term “carboxy” refers to the functional group —C(O)—.

The term “hydroxy” refers to an alcohol functional group having the formula —OH.

The term “nitro” refers to a functional group having the formula —NO₂, wherein the nitrogen atom is positively charged and singly bound to a negatively charged oxygen atom and doubly bound to a second oxygen atom.

The term “mercapto” is synonymous with the term “thio,” which refers to a functional group having the formula —SH.

The term “cyano” refers to a functional group having the formula —CN, wherein carbon is triply bound to nitrogen.

The term “sulfamoyl” refers to a functional group having the formula —SO₂NH₂, wherein the sulfur atom is doubly bound to two oxygen atoms and singly bound to nitrogen.

An unspecified “R” group is a hydrogen, lower alkyl, or aryl all of which may be optionally substituted with one or more substituents.

Throughout the specification, groups and substituents thereof may be chosen to provide stable moieties and compounds.

As used herein, “subject” refers to any animal, preferably a human patient, livestock, or domestic pet.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression. The term “effective amount” or “therapeutically effective amount” refers to that amount of a compound or pharmaceutical composition described herein that is sufficient to affect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the term “combination with” when used to describe administration with an additional treatment means that the agent may be administered prior to, together with, or after the additional treatment, or a combination thereof.

### I. Methods of Treatment

Methods of using the disclosed compounds for treating a cancer, reducing a cancer, or treating or ameliorating one or more symptoms associated with a cancer in a subject are disclosed. Generally, the method includes (i) administering the subject an effective amount of the compound(s) to treat the cancer, reduce the cancer, or treat or ameliorate one or more symptoms associated with the cancer in the subject. The subject can be a mammal. In an embodiment, the subject is at risk of, exhibiting symptoms of, or diagnosed with cancer. The compound(s) can be administered by a medical professional or the subject being treated (e.g. self-administration).

For example, the disclosed method for treating a cancer, reducing a cancer, or treating or ameliorating one or more symptoms associated with a cancer includes administering the subject an effective amount of a compound of Formula (IV), (IVa), or (IVb).

As used herein, “cancer” refers to any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area.

In an aspect, provided herein is a method of treating cancer in a subject in need thereof, including administering to the subject a therapeutically effective amount of a compound or composition disclosed herein. In an aspect, provided herein is a method of reducing cancer in a subject in need thereof, including administering to the subject a therapeutically effective amount of a compound or composition disclosed herein. In another aspect, provided herein is a method of treating or ameliorating one or more symptoms associated with a cancer in a subject in need thereof, including administering to the subject a therapeutically effective amount of a compound or composition disclosed herein.

Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The effective amount of the present compounds depend on many factors, including the indication being treated, the route of administration, co-administration of other therapeutic compositions, and the overall condition of the patient.

In general, treatment regimens utilizing compounds include administration of from about 0.1 mg to about 300 mg of the compounds per kilogram body weight of the recipient per day in multiple doses or in a single dose. In some embodiments, a suitable dose may be in the range of 0.1 to 300 mg per kilogram body weight of the recipient per day, optionally in the range of 6 to 150 mg per kilogram body weight per day, optionally in the range of 15 to 100 mg per kilogram body weight per day, optionally in the range of 15 to 80 mg per kilogram body weight per day, optionally in the range of 15 to 50 mg per kilogram body weight per day, and optionally in the range of 15 to 30 mg per kilogram body weight per day. The desired dose may be presented as two, three, four, five or six or more sub-doses administered at appropriate intervals throughout the day. These sub-doses may be administered in unit dosage forms, for example, containing 10 to 2000 mg, optionally 10 to 1500 mg, optionally 20 to 1000 mg, and optionally 50 to 700 mg of the compounds per unit dosage form.

In certain embodiments, a compound or composition as disclosed herein is used in the production of a medicament for use in treating a cancer, reducing a cancer, or treating or ameliorating one or more symptoms associated with a cancer. For example, the compounds and/or their pharmaceutically acceptable salts can be administered in the form of a pharmaceutical composition in association with one or more pharmaceutically acceptable excipients, such as the pharmaceutical compositions described below. The choice of the pharmaceutically acceptable excipients will to a large extent depend on factors such as the particular mode of administration, the effect of the excipient on solubility and stability, and the nature of the dosage form. The cancer to be treated or reduced or the symptoms associated with the cancer to be treated or ameliorated in the context of the present disclosure may be any type of cancer or tumor.

In an embodiment, the tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

In another embodiment, the tumor is located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genito-urinary apparatus.

In yet another embodiment, the cancer is selected from the group consisting of childhood acute lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, and AIDS-related lymphoma.

In certain embodiments, the cancer is leukemia, such as childhood acute lymphoblastic leukemia, acute lymphoblastic leukemia, acute lymphocytic leukemia, acute myeloid leukemia, adult acute lymphocytic leukemia, adult acute myeloid leukemia, and adult lymphocytic leukemia.

In yet another embodiment, the cancer is selected from the group consisting of AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the oesophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, pheochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

In certain embodiments, compounds disclosed herein may be administered in combination with an additional anti-cancer agent. A “chemotherapy agent,” “chemotherapeutic,” “anti-cancer agent” or the like, refer to molecules that are recognized to aid in the treatment of a cancer. The additional anti-cancer agent in addition to the disclosed compounds may be administered to the subject throughout the method or at different intervals during the method. For example, the additional anti-cancer agent is administered to the subject prior to, during, and/or subsequent to step (i). In some embodiments, the additional anti-cancer agent is included in a pharmaceutical composition containing the compound(s) and is administered to the subject simultaneously with the compound(s) in the pharmaceutical composition in association with one or more pharmaceutically acceptable excipients.

The additional anti-cancer agent is known in the art. The amount of the additional anti-cancer agent required will vary from subject to subject according to their need.

In an embodiment, the additional anti-cancer agent is selected from the group consisting of abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

In certain embodiments, the additional anti-cancer agent is an anti-PD-1, anti-CTLA4 antibody or combinations thereof, such as an anti-CTLA4 (e.g., ipilimumab, tremelimumab) and anti-PD1 (e.g., nivolumab, pembrolizumab, atezolizumab, avelumab, durvalumab).

In another aspect, provided herein is a method of in inhibiting a protein in a subject in need thereof, including administering to the subject a therapeutically effective amount of a compound or composition disclosed herein. For example, the method of inhibiting a protein in a subject in need thereof, including administering to the subject a therapeutically effective amount of a compound of Formula (IV), (IVa), or (IVb).

In an embodiment, the protein is MDM2. In another embodiment, the protein is XIAP.

In some other embodiments, provided herein is a method for reducing the number of cancer cells in a subject in need thereof, including administering to the subject a therapeutically effective amount of a compound or composition disclosed herein. The cancer cells being treated are preferably acute lymphoblastic leukemia (ALL) cells. In some embodiments, the compound or composition used in the method for treating cancer cell has a cytotoxicity against ALL EU-1 cell line higher than MX69, tested under the same conditions and it more potent than MX69, ageist ALL EU-1 cells. The term “same conditions” means test is performed using the same assay, such as water-soluble tetrazolium salt (WST) assay, using the same protocol, such as same amount of cells and enzymes, same dye and dye concentration, and same incubation time and temperature, etc. In some embodiments, the compound or composition used in the method has an IC₅₀ below about 1 μM, against the ALL EU-1 cell line.

For example, the disclosed method for treating cancer cells includes administering the subject an effective amount of a compound of Formula (IV), (IVa), or (IVb), wherein the compound of Formula (IV), (IVa), or (IVb) has a higher cytotoxicity against ALL EU-1 cell line compared to MX69, tested under the same conditions, and optionally wherein the compound of Formula (IV), (IVa), or (IVb) has an IC₅₀ below about 1 μM, against an ALL EU-1 cell line.

### II. Compositions

The disclosed compositions include quinoline derivatives, pharmaceutically acceptable salts thereof, and pharmaceutical formulations including the quinoline derivatives and pharmaceutically acceptable salts thereof.

A. Compounds

In certain embodiments, this disclosure relates to therapeutically beneficial quinoline derivatives as compounds of this disclosure. In some embodiments, the compounds disclosed herein inhibit a protein, such as MDM2. In some embodiments, the compounds disclosed herein have a cytotoxicity against ALL EU-1 cell line higher than MX69 when tested under the same conditions. In some embodiments, the compounds disclosed herein an IC₅₀ below about 1 μM, against an ALL EU-1 cell line.

In certain embodiments, the quinoline compounds are compounds disclosed herein optionally substituted with one or more substituents, or derivatives thereof. In an aspect, the quinoline compounds have Formula I

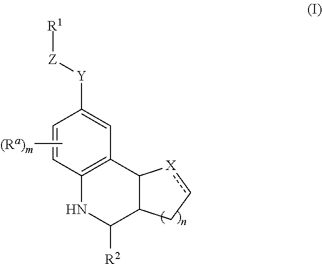

or pharmaceutically acceptable salts and prodrugs thereof wherein,
[symbol] is an optional double bond;
n is 1 or 2;
m is 0, 1, 2, or 3;
X is O, S, CH, CH₂, NR^{b}, or NH;
Y is absent, SO, SO₂, CO or NH;
Z is absent, O, S, SO₂, CO, NH, or N-alkyl;
R^{a} is independently, at each occurrence, halogen, hydroxy, nitro, cyano, or alkoxy;
R^{b} is alkyl or C(O)O-alkyl;
R¹ is absent, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵, and wherein two R¹⁵, together with the atoms to which they are attached, may form a heterocyclic ring;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), alkenyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, n is 1, X is O, Y is SO₂, Z is NH, R¹ is aryl, and R² is carbocyclyl, aryl, or heterocyclyl.

In another embodiment,
n is 1;
m is 0;
Y is SO₂;
Z is NH;
X is O, CH, CH₂, or NH;
R¹ is aryl substituted with one or two R¹⁵;
R² is aryl, carbocyclyl, or heterocyclyl, wherein R² is optionally substituted with one R¹⁵; and
R¹⁵ is independently, at each occurrence, alkyl, halogen, C(O)alkyl, C(O)carbocyclyl, C(O)aryl, CO₂alkyl, or alkylsulfonyl.

In another embodiment, n is 1; m is 0; X is O; Y is absent; Z is NH;
R¹ is arylsulfonyl optionally substituted with one or two R¹⁵; R² is aryl, carbocyclyl, or heterocyclyl, wherein R² is optionally substituted with one R¹⁵; and R¹⁵ is independently, at each occurrence, alkyl, halogen, C(O)alkyl, C(O)carbocyclyl, C(O)aryl, CO₂alkyl, or alkylsulfonyl.

In certain embodiments, a Formula I is Formula Ia or Formula Ib,

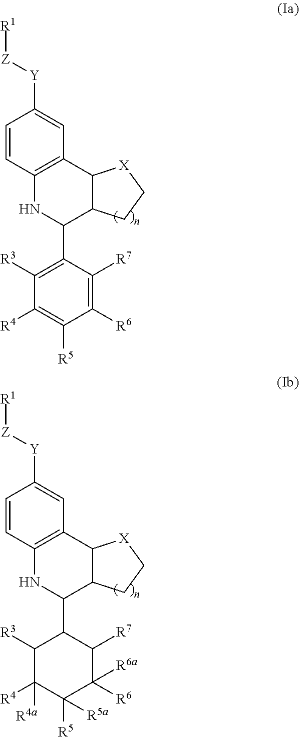

or pharmaceutically acceptable salts and prodrugs thereof wherein,
n is 1 or 2;
X is O, S, or NH;
Y is absent, SO, or SO₂;
Z is O, S, or NH;
R¹ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵;
R³ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R³ is optionally substituted with one or more, the same or different, R¹⁵;
R⁴ and R^{4a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁴ and R^{4a} are optionally substituted with one or more, the same or different, R¹⁵;
R⁵ and R^{5a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁵ and R^{5a} are optionally substituted with one or more, the same or different, R¹⁵;
R⁶ and R^{6a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁶ and R^{6a} are optionally substituted with one or more, the same or different, R¹⁵;
R⁷ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁷ is optionally substituted with one or more, the same or different, R¹⁵;
R¹⁵ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, n is 1, X is O, Y is SO₂, Z is NH, R¹ is aryl.

In an embodiment of Formula Ia or Ib,
n is 1 or 2;
X is O or NH;
Y is SO₂;
Z is NH;
R¹ is carbocyclyl, aryl, or heteroaryl, wherein R¹ is optionally substituted with one or two R¹⁵;
R³, R⁴, R^{5a}, and R^{6a} are each hydrogen;
R⁵ is carboxy or aryl; wherein R⁵ is optionally substituted with one or two R¹⁵;
R⁶ is hydrogen or alkyl;
R⁷ is hydrogen or alkyl; and
R¹⁵ is independently, at each occurrence, alkyl, halogen, cyano, hydroxy, C(O)alkyl, C(O)aryl, CO₂H, alkylsulfonyl, or alkoxy.

In an embodiment of Formula Ia or Ib,
n is 1; X is O; Y is absent; Z is NH; R¹ is arylsulfonyl optionally substituted with one or two R¹⁵; R³, R⁴, R^{5a}, and R^{6a} are each hydrogen; R⁵ is carboxy or aryl, wherein R⁵ is optionally substituted with one or two R¹⁵; R⁶ is hydrogen or alkyl; R⁷ is hydrogen or alkyl; and R¹⁵ is independently, at each occurrence, alkyl, halogen, cyano, hydroxy, C(O)alkyl, C(O)aryl, CO₂H, alkylsulfonyl, or alkoxy.

In another embodiment of Formula Ia or Ib,
n is 1; X is O; Y is absent; Z is NH; R¹ is arylsulfonyl optionally substituted with one or two R¹⁵; R³, R⁴, R^{5a}, and R^{6a} are each hydrogen; R⁵ is C(O)alkyl, CO₂alkyl, C(O)carbocyclyl, C(O)heterocyclyl, halogen, or alkylsulfonyl; R⁶ is hydrogen or alkyl; R⁷ is hydrogen or alkyl; and R¹⁵ is independently, at each occurrence, alkyl, halogen, cyano, hydroxy, C(O)alkyl, C(O)aryl, CO₂H, alkylsulfonyl, or alkoxy.

In certain embodiments, a Formula I is Formula Ic,

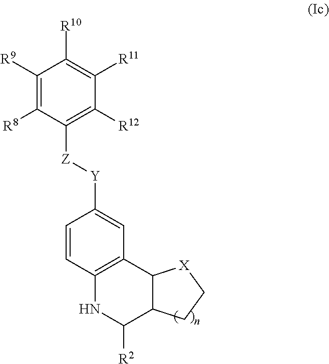

or pharmaceutically acceptable salts and prodrugs thereof wherein,
n is 1 or 2;
X is O, S, or NH;
Y is SO or SO₂;
Z is O, S, or NH;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R⁸ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁸ is optionally substituted with one or more, the same or different, R¹⁵;
R⁹ is hydrogen alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁹ is optionally substituted with one or more, the same or different, R¹⁵;
R¹⁰ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹⁵;
R¹¹ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹⁵;
or alternatively, R¹⁰ and R¹¹, together with the atoms to which they are attached, form a heterocyclic ring;
R¹² is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹² is optionally substituted with one or more, the same or different, R¹⁵;
R¹⁵ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is individually and independently at each occurrence alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, n is 1, X is O, Y is SO₂, Z is NH, R² is carbocyclyl, aryl, or heterocyclyl.

In an embodiment of Formula Ic, n is 1, Y is SO₂, Z is NH, and R⁸, R⁹, R¹¹, and R¹² are each hydrogen. In an embodiment Formula Ic,
n is 1;
X is O or NH;
Y is SO₂;
Z is NH;
R² is aryl, carbocyclyl, or carboxy wherein R² is optionally substituted with one or two R¹⁵;
R¹⁰ is hydrogen. methyl, CF₃, or halogen;
R¹¹ is hydrogen. methyl, CF₃, or halogen;
R⁸, R⁹, and R¹² are each hydrogen; and
R¹⁵ is independently, at each occurrence, C(O)alkyl, CO₂alkyl, C(O)carbocyclyl, C(O)heterocyclyl, halogen, or alkylsulfonyl.

In certain embodiments, a Formula I is Formula Ic′,

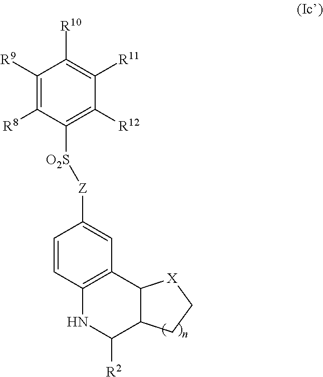

or pharmaceutically acceptable salts and prodrugs thereof, wherein
n is 1 or 2; X and Z are independently O, S, or NH; and R² and R⁸-R¹² are as defined above for Formula Ic.

In certain embodiments of Formula Ic′, n is 1, X is O, Z is NH, R² is carbocyclyl, aryl, or heterocyclyl, and R⁸-R¹² are as defined above for Formula Ic.

In certain embodiments of Formula Ic′, n is 1, X is O, Z is NH, and R⁸, R⁹, and R¹² are each hydrogen, and R¹⁰ and R¹¹ are independently hydrogen, alkyl, C(O)alkyl, CO₂alkyl, C(O)carbocyclyl, C(O)heterocyclyl, halogen, or alkylsulfonyl.

In an embodiment of Formula Ic′, n is 1; X is O or NH; Z is NH; R² is aryl, carbocyclyl, or carboxy wherein R² is optionally substituted with one or two R¹⁵; R¹⁰ and R¹¹ are independently a hydrogen. methyl, C(O)alkyl, CF₃, or halogen; R⁸, R⁹, and R¹² are each hydrogen; and R¹⁵ is independently, at each occurrence, C(O)alkyl, CO₂alkyl, C(O)carbocyclyl, C(O)heterocyclyl, halogen, or alkylsulfonyl.

In certain embodiments, compound of Formula I is Formula Id or Formula Ie,

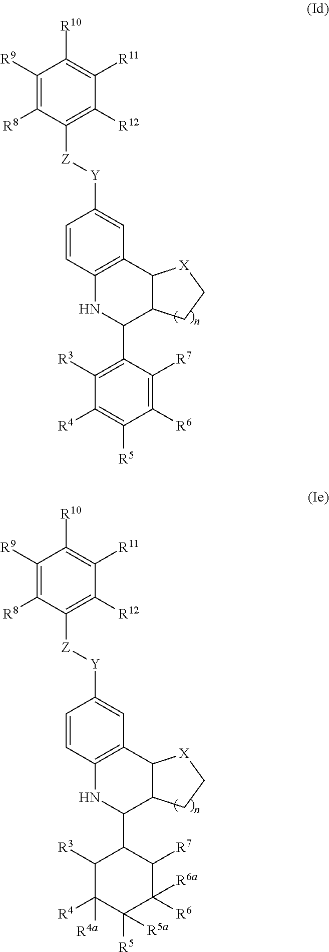

or pharmaceutically acceptable salts and prodrugs thereof;
wherein,
n is 1 or 2;
X is O, S, or NH;
Y is SO or SO₂;
Z is O, S, or NH;
R³ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R³ is optionally substituted with one or more, the same or different, R¹⁵;
R⁴ and R^{4a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁴ and R^{4a} are optionally substituted with one or more, the same or different, R¹⁵;
R⁵ and R^{5a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁵ and R^{5a} are optionally substituted with one or more, the same or different, R¹⁵;
R⁶ and R^{6a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁶ and R^{6a} are optionally substituted with one or more, the same or different, R¹⁵;
R⁷ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁷ is optionally substituted with one or more, the same or different, R¹⁵;
R⁸ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁸ is optionally substituted with one or more, the same or different, R¹⁵;
R⁹ is hydrogen alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁹ is optionally substituted with one or more, the same or different, R¹⁵;
R¹⁰ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹⁵;
R¹¹ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹⁵;
R¹² is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹² is optionally substituted with one or more, the same or different, R¹⁵;
R¹⁵ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In another embodiment of Formula Id or Formula Ie, n is 1, X is 0 or NH, Y is SO₂, Z is NH, and R³, R⁴, R^{4a}, R^{5a}, R^{6a}, R⁷, R⁸, R⁹, and R¹² are each hydrogen.

In another embodiment,
n is 1;
Y is SO₂;
Z is NH,
X is O or NH;
R³, R⁴, R^{4a}, R^{5a}, R^{6a}, R⁷, R⁸, R⁹, and R¹² are each hydrogen;
R¹⁰ is hydrogen. methyl, CF₃, or halogen;
R¹¹ is hydrogen, methyl, CF₃, or halogen;
R⁵ is C(O)alkyl, CO₂alkyl, C(O)carbocyclyl, C(O)heterocyclyl, halogen, or alkylsulfonyl; and
R⁶ is hydrogen, C(O)alkyl, or CO₂alkyl.

In certain embodiments, compound of Formula I is Formula Id′ or Formula Ie′,

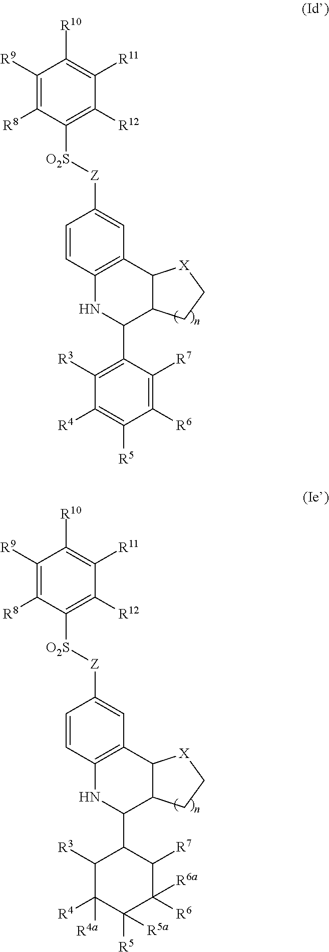

or pharmaceutically acceptable salts and prodrugs thereof; wherein,
n is 1 or 2; X and Z are independently O, S, or NH; and R³-R⁷, R^{4a}-R^{6a}, and R⁸-R¹² are as defined above for Formula Id and Formula Ie.

In certain embodiments of Formula Id′ or Formula Ie′,
n is 1; X is O or NH; Z is NH; R³, R⁴, R^{4a}, R^{1a}, R^{6a}, R⁷, R⁸, R⁹, and R¹² are each hydrogen; R⁵ is C(O)alkyl, CO₂alkyl, C(O)carbocyclyl, C(O)heterocyclyl, hydrogen, halogen, or alkylsulfonyl; R⁶ is hydrogen, C(O)alkyl, or CO₂alkyl; and R¹⁰ and R¹¹ are independently a hydrogen. methyl, C(O)alkyl, CF₃, or halogen. In an embodiment, the compound of Formula I is a compound of Formula If:

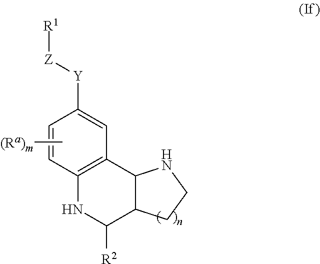

or pharmaceutically acceptable salts and prodrugs thereof, where Y, Z, R¹, R², R^{a}, and m are as defined above for Formula I.

In an embodiment of Formula If, m is 0, Y is SO₂, Z is NH, and R¹ is phenyl substituted with one or two R¹⁵.

In another embodiment of Formula If,
m is 0;
Y is SO₂;
Z is NH;
R¹ is phenyl substituted with one or two R¹⁵;
R² is aryl, carbocyclyl, or heterocyclyl, wherein R² is optionally substituted with R¹⁵; and wherein two R¹⁵, together with the atoms to which they are attached, may form a heterocyclic ring;
R¹⁵ is independently, at each occurrence, alkyl, alkoxy, halogen, carboxy, alkylsulfonyl, and cyano, wherein R¹⁵ is optionally substituted with R¹⁶; and
R¹⁶ is alkyl, alkoxy, aryl, carbocyclyl, alkenyl, hydroxy, or halogen.

In another embodiment of Formula If,
m is 0; Y is SO₂; Z is NH; R¹ is phenyl substituted with one or two R¹⁵; R² is aryl, carbocyclyl, or heterocyclyl, wherein R² is optionally substituted with R¹⁵; and wherein two R¹⁵, together with the atoms to which they are attached, may form a heterocyclic ring; R¹⁵ is independently, at each occurrence, alkyl, halogen, C(O)alkyl, C(O)carbocyclyl, C(O)aryl, CO₂alkyl, or alkylsulfonyl.

In yet another embodiment of Formula If,
m is 0; Y is absent; Z is NH; R¹ is arylsulfonyl optionally substituted with one or two R¹⁵; R² is aryl, carbocyclyl, or heterocyclyl, wherein R² is optionally substituted with one R¹⁵; and R¹⁵ is independently, at each occurrence, alkyl, halogen, C(O)alkyl, C(O)carbocyclyl, C(O)aryl, CO₂alkyl, or alkylsulfonyl.

In another embodiment, the compound of Formula I is a compound of Formula Ig:

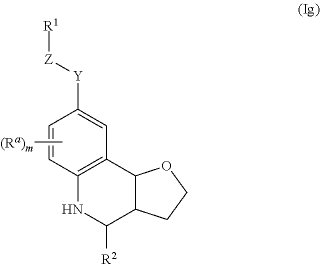

or pharmaceutically acceptable salts and prodrugs thereof, where Y, Z, R¹, R², R^{a}, and m are as defined above for Formula I.

In an embodiment of Formula Ig, m is 0, Y is SO₂, Z is NH, and R¹ is phenyl substituted with one or two R¹⁵.

In another embodiment of Formula Ig,
m is 0;
Y is SO₂;
Z is NH;
R¹ is phenyl substituted with one or two R¹⁵;
R² is aryl, carbocyclyl, or heterocyclyl, wherein R² is optionally substituted with R¹⁵;
R¹⁵ is independently, at each occurrence, alkyl, halogen, carboxy, alkylsulfonyl, and cyano, wherein R¹⁵ is optionally substituted with R¹⁶; and
R¹⁶ is alkyl, alkoxy, aryl, carbocyclyl, alkenyl, hydroxy, or halogen.

In yet another embodiment of Formula Ig,
m is 0; Y is absent; Z is NH; R¹ is arylsulfonyl optionally substituted with one or two R¹⁵; R² is aryl, carbocyclyl, or heterocyclyl, wherein R² is optionally substituted with one R¹⁵; and R¹⁵ is independently, at each occurrence, alkyl, halogen, C(O)alkyl, C(O)carbocyclyl, C(O)aryl, CO₂alkyl, or alkylsulfonyl.

In yet another embodiment, the compound of Formula I is a compound of Formula Ih:

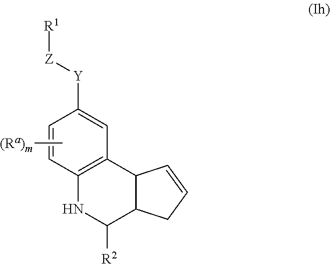

or pharmaceutically acceptable salts and prodrugs thereof, where Y, Z, R¹, R², R^{a}, and m are as defined above for Formula I.

In an embodiment of Formula Ih, m is 0, Y is SO₂, Z is NH, and R¹ is phenyl substituted with one or two R¹⁵.

In another embodiment of Formula Ih,
m is 0;
Y is SO₂;
Z is NH;
R¹ is phenyl substituted with one or two R¹⁵;
R² is aryl, carbocyclyl, or heterocyclyl, wherein R² is optionally substituted with R¹⁵;
R¹⁵ is independently, at each occurrence, alkyl, halogen, carboxy, alkylsulfonyl, and cyano, wherein R¹⁵ is optionally substituted with R¹⁶; and
R¹⁶ is alkyl, alkoxy, aryl, carbocyclyl, alkenyl, hydroxy, or halogen.

In yet another embodiment of Formula Ih,
m is 0; Y is absent; Z is NH; R¹ is arylsulfonyl optionally substituted with one or two R¹⁵; R² is aryl, carbocyclyl, or heterocyclyl, wherein R² is optionally substituted with one R¹⁵; and R¹⁵ is independently, at each occurrence, alkyl, halogen, C(O)alkyl, C(O)carbocyclyl, C(O)aryl, CO₂alkyl, or alkylsulfonyl.

In still another embodiment, the compound of Formula I is a compound of Formula II:

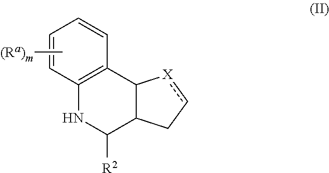

or pharmaceutically acceptable salts and prodrugs thereof; where X, R², R^{a}, and m are as defined above for Formula I.

In an embodiment of Formula II, X is O. In another embodiment of Formula II, X is NH. In yet another embodiment of Formula II, X is CH. In still another embodiment of Formula II, X is CH₂.

In an embodiment of Formula II,
m is 1, 2, or 3;
X is O or CH;
R² is aryl optionally substituted with R¹⁵;
R^{a} is hydroxy, cyano, halogen, alkoxy, and nitro; and
R¹⁵ is halogen, alkyl, cyano, and CO₂H.

In another aspect, provided herein are compounds of Formula III

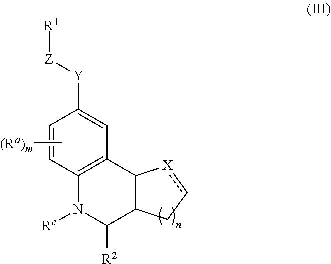

or pharmaceutically acceptable salts and prodrugs thereof wherein,
[symbol] is an optional double bond;
n is 1 or 2;
m is 0, 1, 2, or 3;
X is O, S, CH, CH₂, NR^{b}, or NH;
Y is absent, SO or SO₂;
Z is absent, O, S, NH, or N-alkyl;
R^{a} is independently, at each occurrence, halogen, hydroxy, nitro, cyano, or alkoxy;
R^{b} is alkyl or C(O)O-alkyl;
R^{c} is alkyl;
R¹ is absent, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵, and wherein two R¹⁵, together with the atoms to which they are attached, may form a heterocyclic ring;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), alkenyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In an embodiment, the compound of Formula I is selected from the group consisting of the following in Table 1.

TABLE 1
Compounds of Formula I

| | |
|---|---|
| 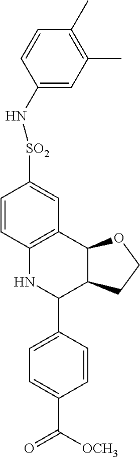 [SMILES: COC(=O)c1ccc(C2Nc3ccc(S(=O)(=O)Nc4ccc(C)c(C)c4)cc3[C@H]3OCC[C@@H]23)cc1] | 69L1 |
| 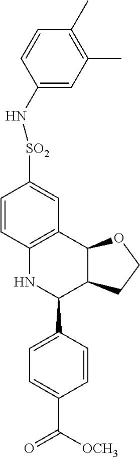 [SMILES: COC(=O)c1ccc([C@H]2Nc3ccc(S(=O)(=O)Nc4ccc(C)c(C)c4)cc3[C@H]3OCC[C@@H]23)cc1] | 69L28 |
| 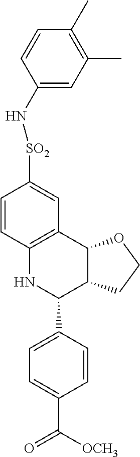 [SMILES: COC(=O)c1ccc([C@@H]2Nc3ccc(S(=O)(=O)Nc4ccc(C)c(C)c4)cc3[C@@H]3OCC[C@H]23)cc1] | 69L29 |
| 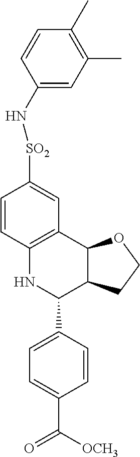 [SMILES: COC(=O)c1ccc([C@@H]2Nc3ccc(S(=O)(=O)Nc4ccc(C)c(C)c4)cc3[C@H]3OCC[C@H]32)cc1] | 69L2 |
| 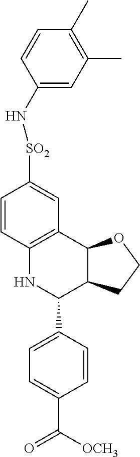 [SMILES: COC(=O)c1ccc([C@@H]2Nc3ccc(S(=O)(=O)Nc4ccc(C)c(C)c4)cc3[C@H]3OCC[C@H]32)cc1] | 69L13 |
| 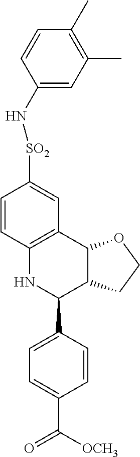 | 69L12 |
| 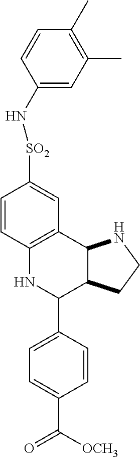 | 69L34 |
| 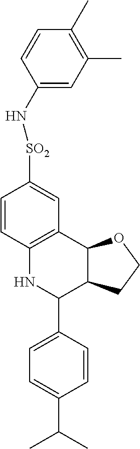 [SMILES: Cc1ccc(NS(=O)(=O)c2ccc3c(c2)[C@H]2OCC[C@H]2C(c2ccc(C(C)C)cc2)N3)cc1C] | 69L31 |
| 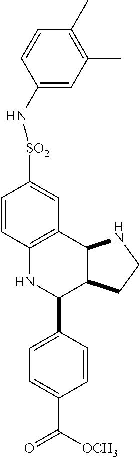 | 69L35 |
| 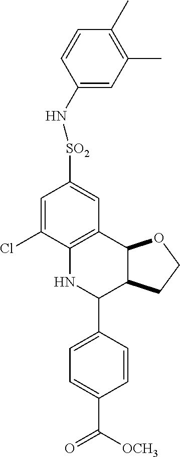 | 69L33 |
| 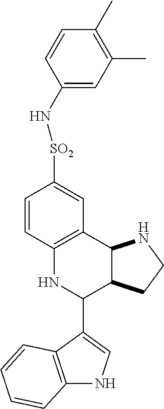 | 69L37 |
| 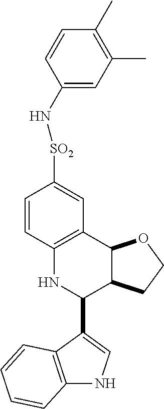 | 69L36 |
| 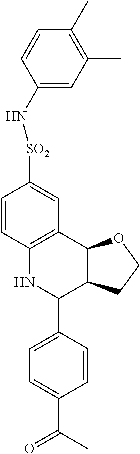 [SMILES: CC(=O)c1ccc(C2Nc3ccc(S(=O)(=O)Nc4ccc(C)c(C)c4)cc3[C@H]3OCC[C@@H]23)cc1] | 69L38 |
| 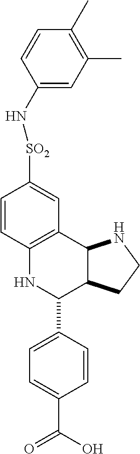 | 69L41 |
| 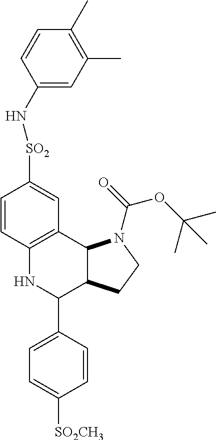 | 69L42 |
| 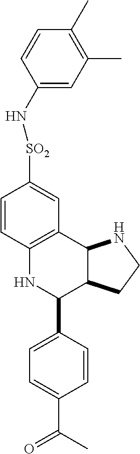 | 69L49 |
| 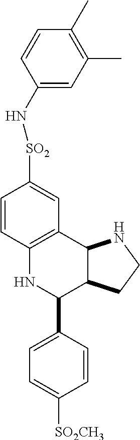 | 69L43 |
| 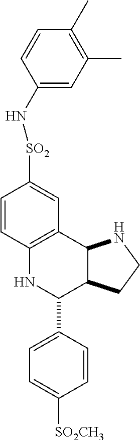 | 69L44 |
| 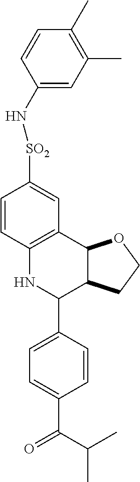 | 69L52 |
| 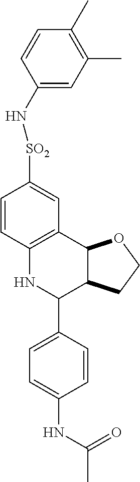 | 69L51 |
| 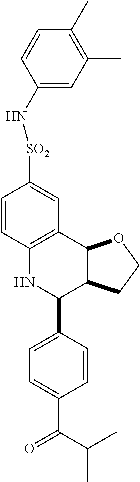 | 69L52b |
| 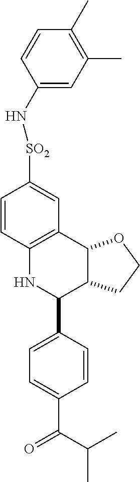 | 69L52c |
| 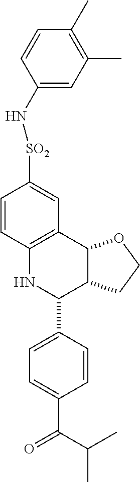 | 69L52d |
| 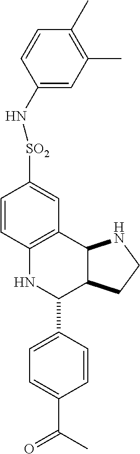 | 69L50 |
| 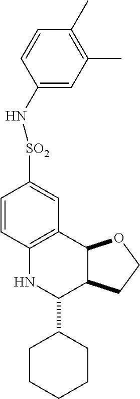 | 69L53 |
| 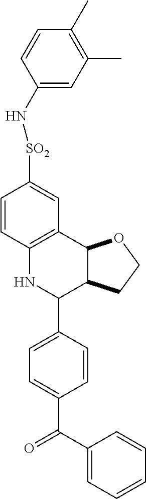 | 69L54 |
| 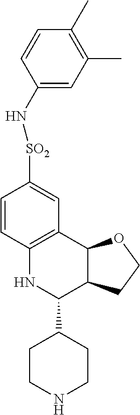 [SMILES: Cc1ccc(NS(=O)(=O)c2ccc3c(c2)[C@H]2OCC[C@H]2[C@H](C2CCNCC2)N3)cc1C] | 69L55 |
| 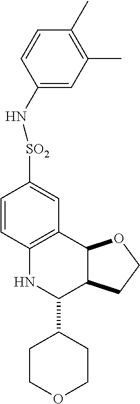 | 69L58 |
| 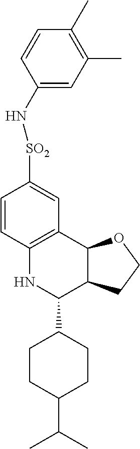 [SMILES: Cc1ccc(NS(=O)(=O)c2ccc3c(c2)[C@H]2OCC[C@H]2[C@H](C2CCC(C(C)C)CC2)N3)cc1C] | 69L56 |
| 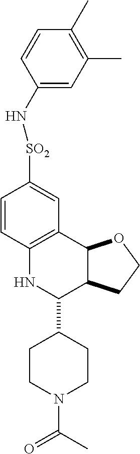 | 69L57 |
| 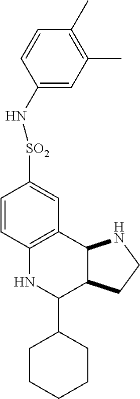 | 69L61 |
| 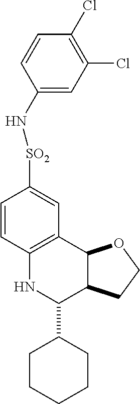 | 69L60 |
| 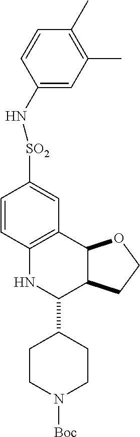 | 69L64 |
| 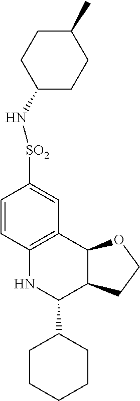 [SMILES: C[C@H]1CC[C@H](NS(=O)(=O)c2ccc3c(c2)[C@H]2OCC[C@H]2[C@H](C2CCCCC2)N3)CC1] | 69L65 |
| 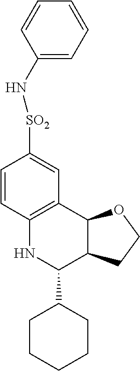 [SMILES: O=S(=O)(Nc1ccccc1)c1ccc2c(c1)[C@H]1OCC[C@H]1[C@H](C1CCCCC1)N2] | 69L66 |
| 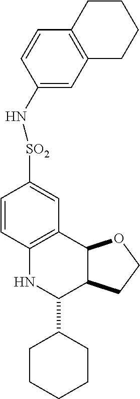 | 69L59 |
| 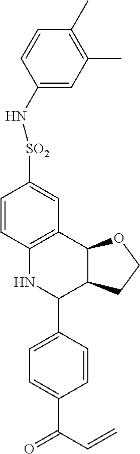 [SMILES: C=CC(=O)c1ccc(C2Nc3ccc(S(=O)(=O)Nc4ccc(C)c(C)c4)cc3[C@H]3OCC[C@@H]23)cc1] | 69L71 |
| 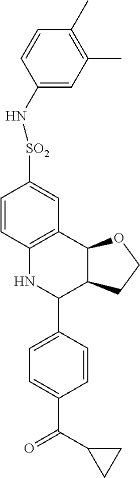 [SMILES: Cc1ccc(NS(=O)(=O)c2ccc3c(c2)[C@H]2OCC[C@H]2C(c2ccc(C(=O)C4CC4)cc2)N3)cc1C] | 69L67 |
| 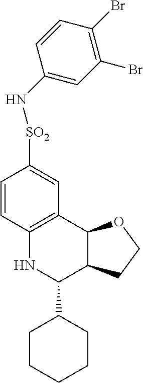 [SMILES: O=S(=O)(Nc1ccc(Br)c(Br)c1)c1ccc2c(c1)[C@H]1OCC[C@H]1[C@H](C1CCCCC1)N2] | 69L68 |
| 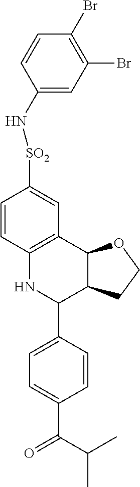 [SMILES: CC(C)C(=O)c1ccc(C2Nc3ccc(S(=O)(=O)Nc4ccc(Br)c(Br)c4)cc3[C@H]3OCC[C@@H]23)cc1] | 69L74 |
| 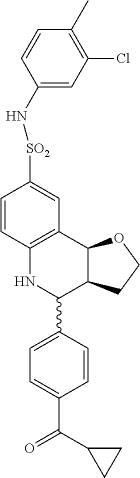 [SMILES: Cc1ccc(NS(=O)(=O)c2ccc3c(c2)[C@H]2OCC[C@H]2C(c2ccc(C(=O)C4CC4)cc2)N3)cc1Cl] | 69L69 |
| 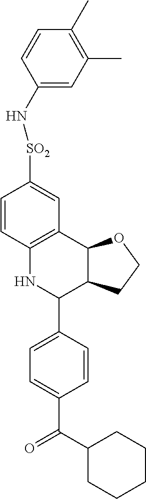 [SMILES: Cc1ccc(NS(=O)(=O)c2ccc3c(c2)[C@H]2OCC[C@H]2C(c2ccc(C(=O)C4CCCCC4)cc2)N3)cc1C] | 69L70 |
| 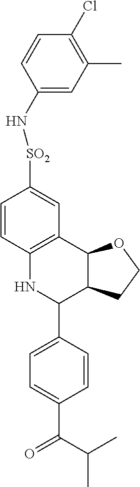 [SMILES: Cc1cc(NS(=O)(=O)c2ccc3c(c2)[C@H]2OCC[C@H]2C(c2ccc(C(=O)C(C)C)cc2)N3)ccc1Cl] | 69L73 |
| 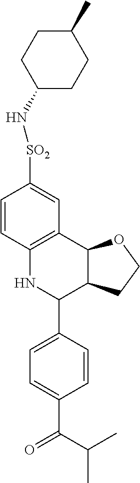 [SMILES: CC(C)C(=O)c1ccc(C2Nc3ccc(S(=O)(=O)N[C@H]4CC[C@H](C)CC4)cc3[C@H]3OCC[C@@H]23)cc1] | 69L78 |
| 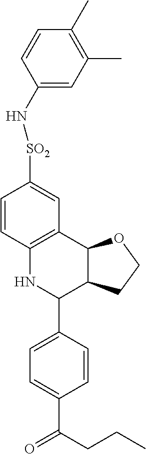 [SMILES: CCCC(=O)c1ccc(C2Nc3ccc(S(=O)(=O)Nc4ccc(C)c(C)c4)cc3[C@H]3OCC[C@@H]23)cc1] | 69L72 |
| 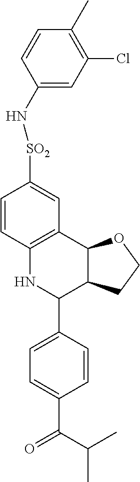 [SMILES: Cc1ccc(NS(=O)(=O)c2ccc3c(c2)[C@H]2OCC[C@H]2C(c2ccc(C(=O)C(C)C)cc2)N3)cc1Cl] | 69L73 |
| 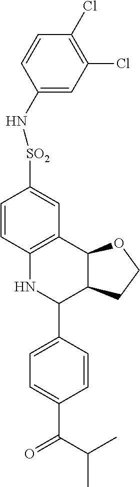 [SMILES: CC(C)C(=O)c1ccc(C2Nc3ccc(S(=O)(=O)Nc4ccc(Cl)c(Cl)c4)cc3[C@H]3OCC[C@@H]23)cc1] | 69L76 |
| 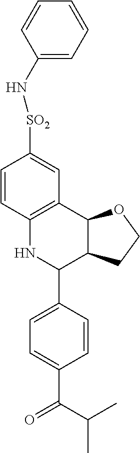 [SMILES: CC(C)C(=O)c1ccc(C2Nc3ccc(S(=O)(=O)Nc4ccccc4)cc3[C@H]3OCC[C@@H]23)cc1] | 69L75 |
| 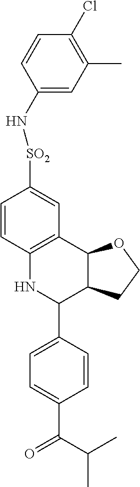 [SMILES: Cc1cc(NS(=O)(=O)c2ccc3c(c2)[C@H]2OCC[C@H]2C(c2ccc(C(=O)C(C)C)cc2)N3)ccc1Cl] | 69L77 |
| 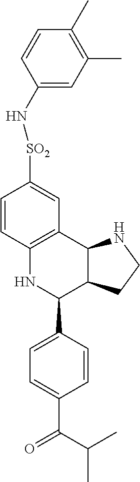 [SMILES: Cc1ccc(NS(=O)(=O)c2ccc3c(c2)[C@H]2NCC[C@H]2[C@@H](c2ccc(C(=O)C(C)C)cc2)N3)cc1C] | 69L81 |
| 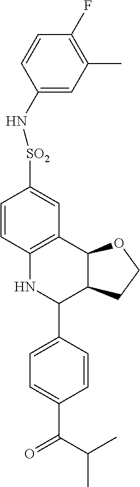 [SMILES: Cc1cc(NS(=O)(=O)c2ccc3c(c2)[C@H]2OCC[C@H]2C(c2ccc(C(=O)C(C)C)cc2)N3)ccc1F] | 69L79 |
| 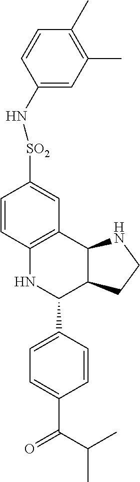 [SMILES: Cc1ccc(NS(=O)(=O)c2ccc3c(c2)[C@H]2NCC[C@H]2[C@H](c2ccc(C(=O)C(C)C)cc2)N3)cc1C] | 69L82 |
| 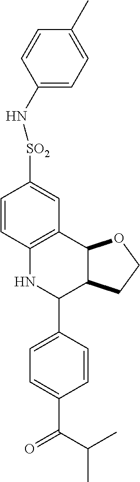 | 69L85 |
| 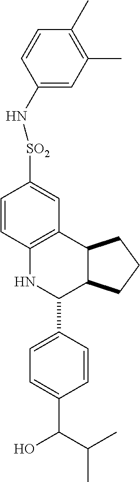 | 69L86 |
| 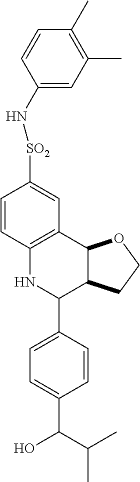 | 69L87 |
| 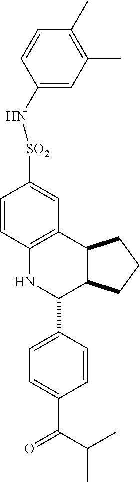 | 69L88 |
| 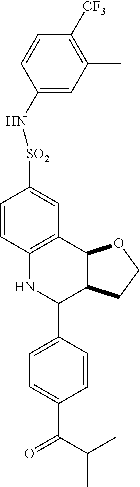 | 69L89 |
| 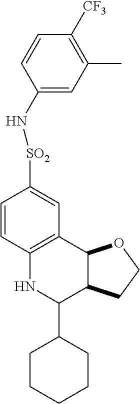 | 69L90 |
| 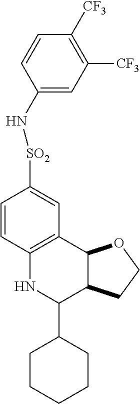 | 69L91 |
| 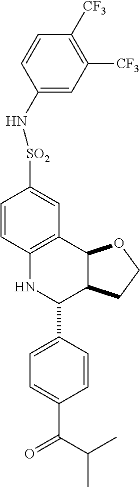 | 69L92 |
| 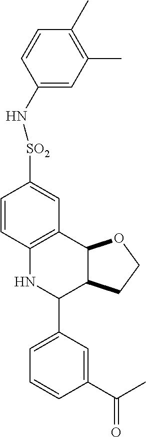 | 69L93 |
| 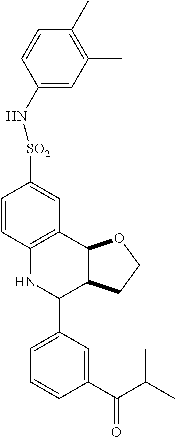 | 69L94 |
| 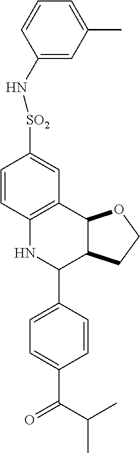 | 69L95 |
| 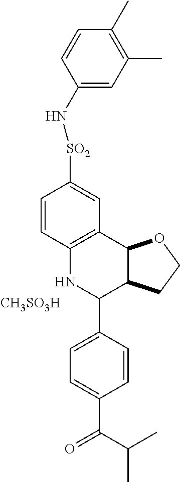 | 69L96 |
| 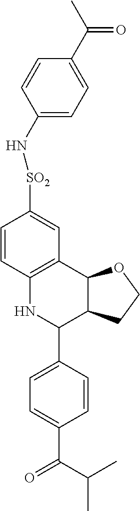 [SMILES: CC(=O)c1ccc(NS(=O)(=O)c2ccc3c(c2)[C@H]2OCC[C@H]2C(c2ccc(C(=O)C(C)C)cc2)N3)cc1] | 69L98 |
| 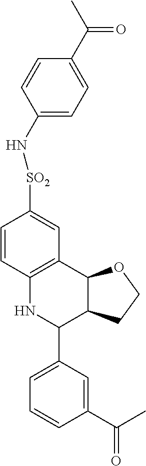 [SMILES: CC(=O)c1ccc(NS(=O)(=O)c2ccc3c(c2)[C@H]2OCC[C@H]2C(c2cccc(C(C)=O)c2)N3)cc1] | 69L101 |
| 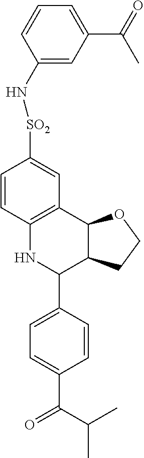 [SMILES: CC(=O)c1cccc(NS(=O)(=O)c2ccc3c(c2)[C@H]2OCC[C@H]2C(c2ccc(C(=O)C(C)C)cc2)N3)c1] | 69L102 |
| 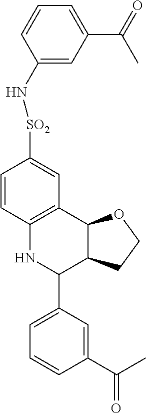 [SMILES: CC(=O)c1cccc(NS(=O)(=O)c2ccc3c(c2)[C@H]2OCC[C@H]2C(c2cccc(C(C)=O)c2)N3)c1] | 69L103 |
| 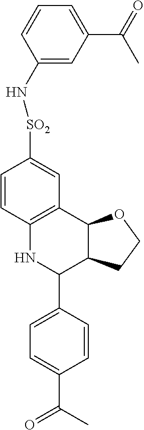 [SMILES: CC(=O)c1ccc(C2Nc3ccc(S(=O)(=O)Nc4cccc(C(C)=O)c4)cc3[C@H]3OCC[C@@H]23)cc1] | 69L110 |
| 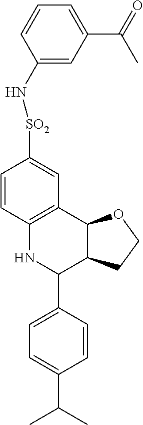 [SMILES: CC(=O)c1cccc(NS(=O)(=O)c2ccc3c(c2)[C@H]2OCC[C@H]2C(c2ccc(C(C)C)cc2)N3)c1] | 69L115 |
| 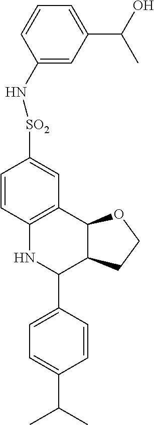 [SMILES: CC(C)c1ccc(C2Nc3ccc(S(=O)(=O)Nc4cccc(C(C)O)c4)cc3[C@H]3OCC[C@@H]23)cc1] | 69L116 |
| 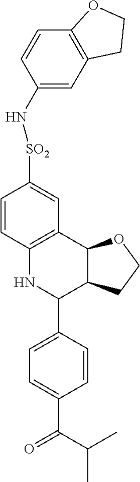 [SMILES: CC(C)C(=O)c1ccc(C2Nc3ccc(S(=O)(=O)Nc4ccc5c(c4)CCO5)cc3[C@H]3OCC[C@@H]23)cc1] | 69L117 |
| 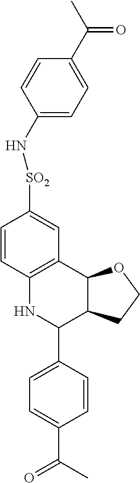 [SMILES: CC(=O)c1ccc(NS(=O)(=O)c2ccc3c(c2)[C@H]2OCC[C@H]2C(c2ccc(C(C)=O)cc2)N3)cc1] | 69L111 |
| 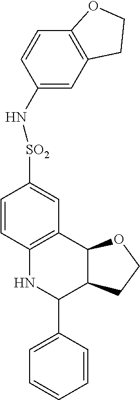 [SMILES: O=S(=O)(Nc1ccc2c(c1)CCO2)c1ccc2c(c1)[C@H]1OCC[C@H]1C(c1ccccc1)N2] | 69L118 |
| 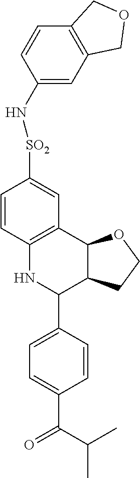 [SMILES: CC(C)C(=O)c1ccc(C2Nc3ccc(S(=O)(=O)Nc4ccc5c(c4)COC5)cc3[C@H]3OCC[C@@H]23)cc1] | 69L119 |
| 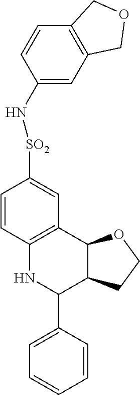 [SMILES: O=S(=O)(Nc1ccc2c(c1)COC2)c1ccc2c(c1)[C@H]1OCC[C@H]1C(c1ccccc1)N2] | 69L120 |
| 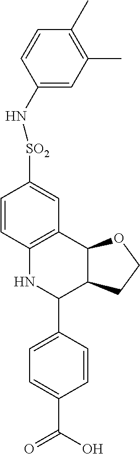 [SMILES: Cc1ccc(NS(=O)(=O)c2ccc3c(c2)[C@H]2OCC[C@H]2C(c2ccc(C(=O)O)cc2)N3)cc1C] | 69L4 |
| 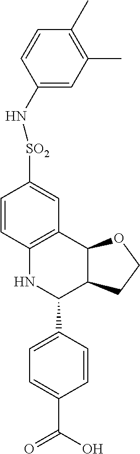 [SMILES: Cc1ccc(NS(=O)(=O)c2ccc3c(c2)[C@H]2OCC[C@H]2[C@H](c2ccc(C(=O)O)cc2)N3)cc1C] | 69L5 |
| 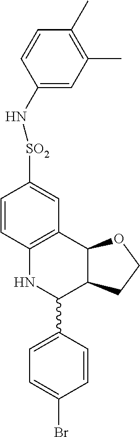 [SMILES: Cc1ccc(NS(=O)(=O)c2ccc3c(c2)[C@H]2OCC[C@H]2C(c2ccc(Br)cc2)N3)cc1C] | 69L8 |
| 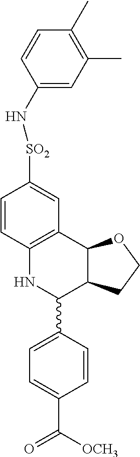 [SMILES: COC(=O)c1ccc(C2Nc3ccc(S(=O)(=O)Nc4ccc(C)c(C)c4)cc3[C@H]3OCC[C@@H]23)cc1] | 69L9 |
| 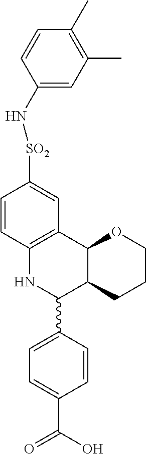 [SMILES: Cc1ccc(NS(=O)(=O)c2ccc3c(c2)[C@H]2OCCC[C@H]2C(c2ccc(C(=O)O)cc2)N3)cc1C] | 69L10 |
| 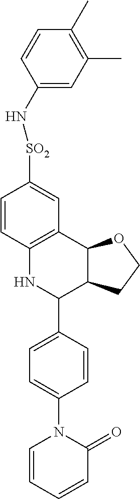 [SMILES: Cc1ccc(NS(=O)(=O)c2ccc3c(c2)[C@H]2OCC[C@H]2C(c2ccc(-n4ccccc4=O)cc2)N3)cc1C] | 69L15 |
| 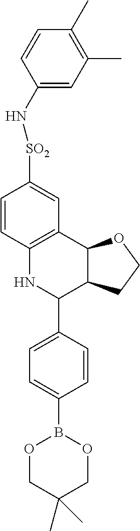 [SMILES: Cc1ccc(NS(=O)(=O)c2ccc3c(c2)[C@H]2OCC[C@H]2C(c2ccc(B4OCC(C)(C)CO4)cc2)N3)cc1C] | 69L17 |
| 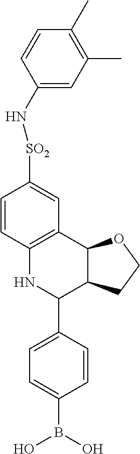 [SMILES: Cc1ccc(NS(=O)(=O)c2ccc3c(c2)[C@H]2OCC[C@H]2C(c2ccc(B(O)O)cc2)N3)cc1C] | 69L18 |
| 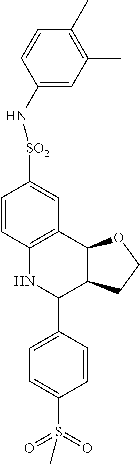 [SMILES: Cc1ccc(NS(=O)(=O)c2ccc3c(c2)[C@H]2OCC[C@H]2C(c2ccc(S(C)(=O)=O)cc2)N3)cc1C] | 69L19 |
| 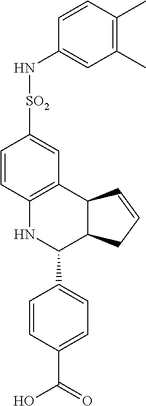 [SMILES: Cc1ccc(NS(=O)(=O)c2ccc3c(c2)[C@H]2C=CC[C@H]2[C@H](c2ccc(C(=O)O)cc2)N3)cc1C] | MX69 |
| 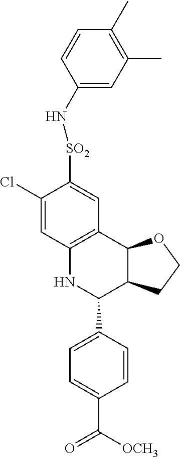 [SMILES: COC(=O)c1ccc([C@@H]2Nc3cc(Cl)c(S(=O)(=O)Nc4ccc(C)c(C)c4)cc3[C@H]3OCC[C@H]32)cc1] | 69L26 |
| 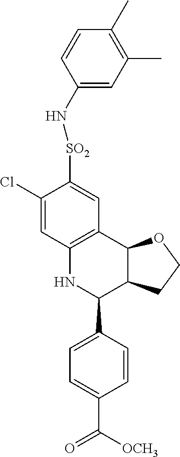 [SMILES: COC(=O)c1ccc([C@H]2Nc3cc(Cl)c(S(=O)(=O)Nc4ccc(C)c(C)c4)cc3[C@H]3OCC[C@@H]23)cc1] | 69L25 |
| 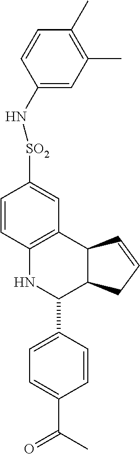 [SMILES: CC(=O)c1ccc([C@@H]2Nc3ccc(S(=O)(=O)Nc4ccc(C)c(C)c4)cc3[C@H]3C=CC[C@H]32)cc1] | 69L48 |
| 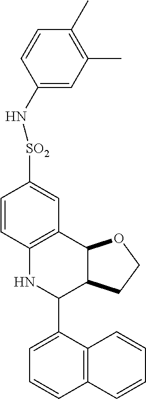 | 69L63 |
| 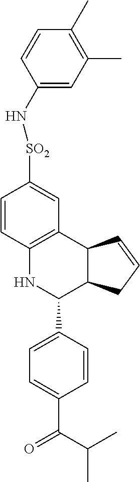 [SMILES: Cc1ccc(NS(=O)(=O)c2ccc3c(c2)[C@H]2C=CC[C@H]2[C@H](c2ccc(C(=O)C(C)C)cc2)N3)cc1C] | 69L80 |
| 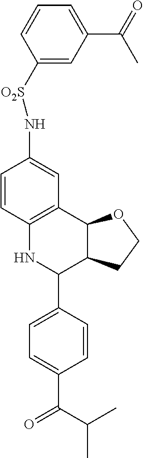 [SMILES: CC(=O)c1cccc(S(=O)(=O)Nc2ccc3c(c2)[C@H]2OCC[C@H]2C(c2ccc(C(=O)C(C)C)cc2)N3)c1] | 69L163 |
| 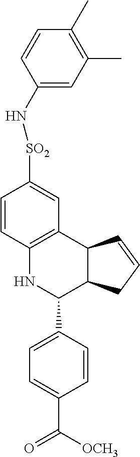 [SMILES: COC(=O)c1ccc([C@@H]2Nc3ccc(S(=O)(=O)Nc4ccc(C)c(C)c4)cc3[C@H]3C=CC[C@H]32)cc1] | 69L16 |
| 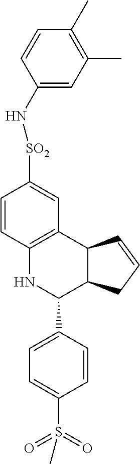 [SMILES: Cc1ccc(NS(=O)(=O)c2ccc3c(c2)[C@H]2C=CC[C@H]2[C@H](c2ccc(S(C)(=O)=O)cc2)N3)cc1C] | 69L21 |
| Intentionally left blank | |
| 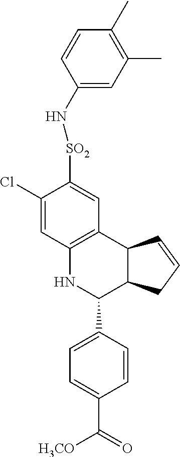 [SMILES: COC(=O)c1ccc([C@@H]2Nc3cc(Cl)c(S(=O)(=O)Nc4ccc(C)c(C)c4)cc3[C@H]3C=CC[C@H]32)cc1] | 69L22 |
| 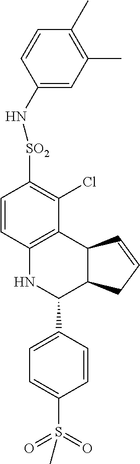 [SMILES: Cc1ccc(NS(=O)(=O)c2ccc3c(c2Cl)[C@H]2C=CC[C@H]2[C@H](c2ccc(S(C)(=O)=O)cc2)N3)cc1C] | 69L23 |
| 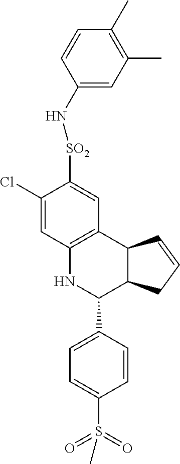 [SMILES: Cc1ccc(NS(=O)(=O)c2cc3c(cc2Cl)N[C@@H](c2ccc(S(C)(=O)=O)cc2)[C@@H]2CC=C[C@H]32)cc1C] | 69L24 |
| 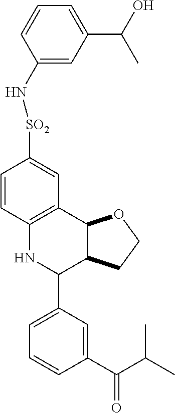 | 69L122 |
| 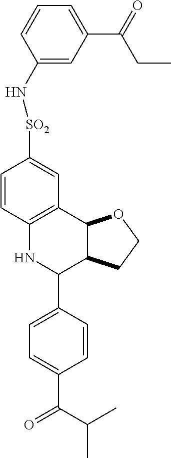 | 69L125 |
| 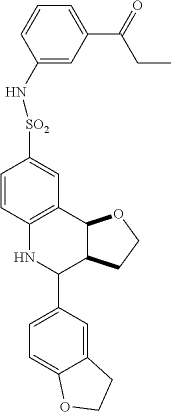 | 69L127 |
| 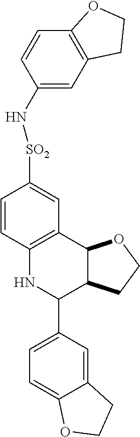 | 69L128 |
| 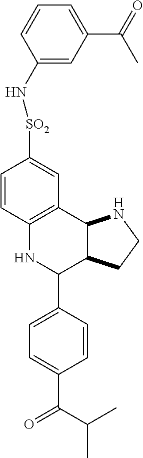 | 69L130 |
| 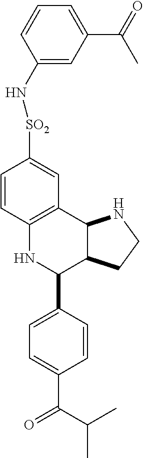 | 69L130 trans |
| 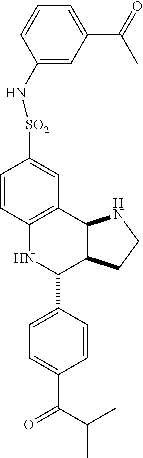 | 69L130 cis |
| 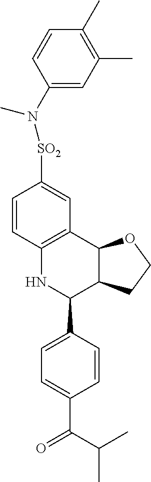 [SMILES: Cc1ccc(N(C)S(=O)(=O)c2ccc3c(c2)[C@H]2OCC[C@H]2[C@@H](c2ccc(C(=O)C(C)C)cc2)N3)cc1C] | 69L132 |
| 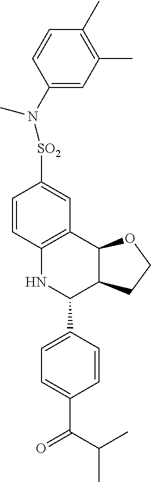 [SMILES: Cc1ccc(N(C)S(=O)(=O)c2ccc3c(c2)[C@H]2OCC[C@H]2[C@H](c2ccc(C(=O)C(C)C)cc2)N3)cc1C] | 69L133 |
| 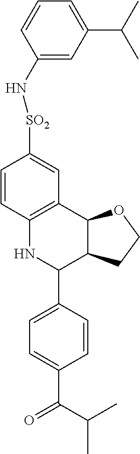 [SMILES: CC(C)C(=O)c1ccc(C2Nc3ccc(S(=O)(=O)Nc4cccc(C(C)C)c4)cc3[C@H]3OCC[C@@H]23)cc1] | 69L134 |
| 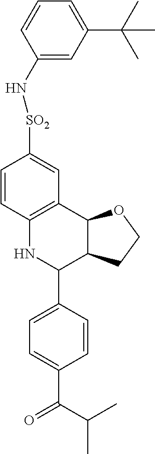 [SMILES: CC(C)C(=O)c1ccc(C2Nc3ccc(S(=O)(=O)Nc4cccc(C(C)(C)C)c4)cc3[C@H]3OCC[C@@H]23)cc1] | 69L136 |
| 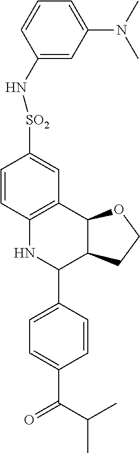 [SMILES: CC(C)C(=O)c1ccc(C2Nc3ccc(S(=O)(=O)Nc4cccc(N(C)C)c4)cc3[C@H]3OCC[C@@H]23)cc1] | 69L141 |
| 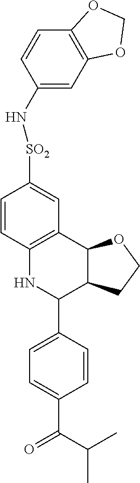 [SMILES: CC(C)C(=O)c1ccc(C2Nc3ccc(S(=O)(=O)Nc4ccc5c(c4)OCO5)cc3[C@H]3OCC[C@@H]23)cc1] | 69L142 |
| 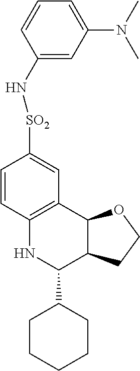 [SMILES: CN(C)c1cccc(NS(=O)(=O)c2ccc3c(c2)[C@H]2OCC[C@H]2[C@H](C2CCCCC2)N3)c1] | 69L143 |
| 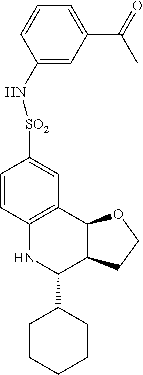 [SMILES: CC(=O)c1cccc(NS(=O)(=O)c2ccc3c(c2)[C@H]2OCC[C@H]2[C@H](C2CCCCC2)N3)c1] | 69L144 |
| 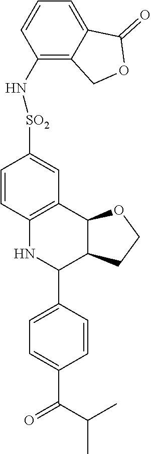 [SMILES: CC(C)C(=O)c1ccc(C2Nc3ccc(S(=O)(=O)Nc4cccc5c4COC5=O)cc3[C@H]3OCC[C@@H]23)cc1] | 69L146 |
| 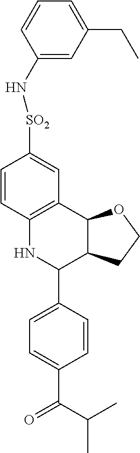 [SMILES: CCc1cccc(NS(=O)(=O)c2ccc3c(c2)[C@H]2OCC[C@H]2C(c2ccc(C(=O)C(C)C)cc2)N3)c1] | 69L148 |
| 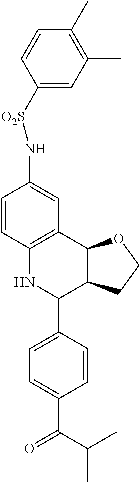 [SMILES: Cc1ccc(S(=O)(=O)Nc2ccc3c(c2)[C@H]2OCC[C@H]2C(c2ccc(C(=O)C(C)C)cc2)N3)cc1C] | 69L162 |

or pharmaceutically acceptable salts and prodrugs thereof.

In an aspect, provided herein is a compound of Formula IV′ or Formula IV:

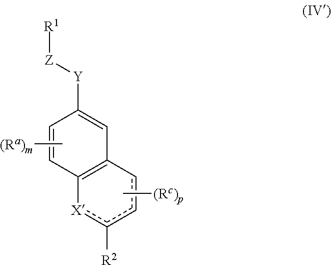

or pharmaceutically acceptable salts and prodrugs thereof;
   wherein
[symbol] is an optional double bond; m is 0, 1, 2, or 3; p is 0, 1, or 2; X′ is O or NH; Y is absent, SO or SO₂; Z is absent, O, S, or NH; R^{a} is independently, at each occurrence, halogen, hydroxy, nitro, cyano, or C₁-C₆ alkoxy; R^{c} is independently, at each occurrence, hydroxy, alkoxy, caroboxy (C(O)), oxo (═O), thione (═S), imino (═NH), N(R¹⁵)₂, OR¹⁵, halogen, or aryl, wherein aryl is optionally substituted with one of more halogen; or alternatively, two R^{c}, together with the atoms to which they are attached, form a heterocyclic ring optionally fused to an aryl ring;
R¹ is absent, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵; and wherein two R¹⁵, together with the atoms to which they are attached, may form a heterocyclic ring;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In some embodiments of Formula IV′, m is 0; p is 0, 1, or 2; X′ is O or NH; Y is SO₂; Z is NH; R^{c} is independently, at each occurrence, oxo (═O), thione (═S), imino (═NH), N(R¹⁵)₂, OR¹⁵; R¹ is aryl optionally substituted with one or two R¹⁵; R² is aryl or heterocyclyl wherein R² is optionally substituted with R¹⁵; and R¹⁵ is independently, at each occurrence, alkyl, halogen, C(O)alkyl, C(O)carbocyclyl, C(O)aryl, CO₂alkyl, or alkylsulfonyl.

In some embodiments of Formula IV′, m is 0; p is 0, 1, or 2; X′ is O or NH; Y is absent; Z is NH; R^{c} is independently, at each occurrence, oxo (═O), thione (═S), imino (═NH), N(R¹⁵)₂, OR¹⁵; R¹ is arylsulfonyl optionally substituted with one or two R¹⁵; R² is aryl, carbocyclyl, or heterocyclyl, wherein R² is optionally substituted with one R¹⁵; and R¹⁵ is independently, at each occurrence, alkyl, halogen, C(O)alkyl, C(O)carbocyclyl, C(O)aryl, CO₂alkyl, or alkylsulfonyl.

In another aspect, the compound of Formula IV′ is compound of Formula IV:

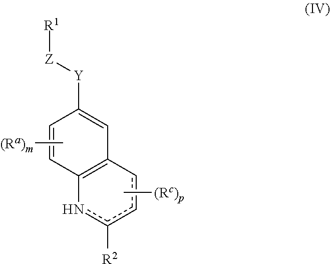

or pharmaceutically acceptable salts and prodrugs thereof;
   wherein
[symbol] is an optional double bond; m, p, Y, Z, R^{a}, R^{c}, R¹, R², R¹⁵, R¹⁶, R¹⁷ are as defined above for Formula IV′.

In an embodiment of Formula IV, p is 1, and R^{c} is hydroxy. In another embodiment, p is 1, and R^{c} is alkoxy. In yet another embodiment, Y is SO₂ and Z is NH. In still another embodiment, Y is SO₂, Z is NH, and R¹ is aryl. In another embodiment, Y is SO₂, Z is NH, and R¹ is aryl substituted with two R¹⁵.

In yet another embodiment of Formula IV, Y is SO₂, Z is NH, p is 1, and R¹ is phenyl substituted with one or two R¹⁵.

In still another embodiment of Formula IV, m is 0; p is 0, 1, or 2; Y is 502; Z is NH; R¹ is aryl optionally substituted with one or two R¹⁵; and R² is aryl or heterocyclyl wherein R² is optionally substituted with R¹⁵.

In some embodiments of Formula IV, m is 0; p is 0, 1, or 2; Y is SO₂; Z is NH; R^{c} is independently, at each occurrence, oxo (═O), thione (═S), imino (═NH), N(R¹⁵)₂, OR¹⁵; R¹ is aryl optionally substituted with one or two R¹⁵; R² is aryl or heterocyclyl wherein R² is optionally substituted with R¹⁵; and R¹⁵ is independently, at each occurrence, alkyl, halogen, C(O)alkyl, C(O)carbocyclyl, C(O)aryl, CO₂alkyl, or alkylsulfonyl.

In some embodiments of Formula IV, m is 0; p is 0, 1, or 2; Y is absent; Z is NH; R^{c} is independently, at each occurrence, oxo (═O), thione (═S), imino (═NH), N(R¹⁵)₂, OR¹⁵; R¹ is arylsulfonyl optionally substituted with one or two R¹⁵; R² is aryl, carbocyclyl, or heterocyclyl, wherein R² is optionally substituted with one R¹⁵; and R¹⁵ is independently, at each occurrence, alkyl, halogen, C(O)alkyl, C(O)carbocyclyl, C(O)aryl, CO₂alkyl, or alkylsulfonyl.

In an embodiment, the compound of Formula IV is a compound of Formula IVa:

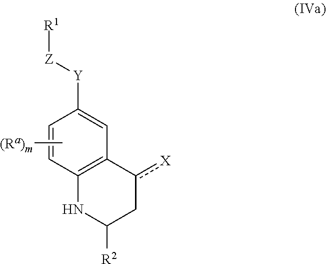

or a pharmaceutically acceptable salt thereof;
   wherein
[symbol] is an optional double bond; m is 0, 1, 2, or 3; X is O, S, NH, N(R¹⁵)₂, OR¹⁵, or halogen; Y is SO or SO₂; Z is absent, O, S, or NH; R^{a} is independently, at each occurrence, halogen, hydroxy, nitro, cyano, or C₁-C₆ alkoxy;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In an embodiment, the compound of Formula IV is a compound of Formula IVb:

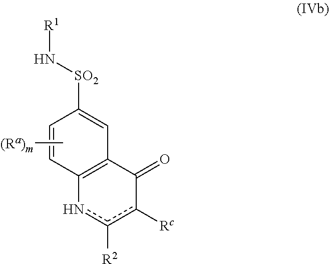

or pharmaceutically acceptable salts and prodrugs thereof;
   wherein
[symbol] is an optional double bond; m is 0, 1, 2, or 3; R^{a} is independently, at each occurrence, halogen, hydroxy, nitro, cyano, or C₁-C₆ alkoxy; R^{c} is absent, hydroxy, alkoxy, C(O), halogen, or aryl, wherein aryl is optionally substituted with one of more halogen;
R¹ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵ wherein two R¹⁵, together with the atoms to which they are attached, form a heterocyclic ring;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In an embodiment, R¹ is aryl substituted with two R¹⁵. In another embodiment, R² is aryl optionally substituted with one R¹⁵.

In yet another embodiment of Formula IIIa, R¹ is phenyl substituted with one or two R¹⁵, R^{c} is hydrogen, and m is 0.

In an embodiment, the compound of Formula IV′ is selected from the group consisting of compounds in Table 2.

TABLE 2
Compounds of Formula IV′

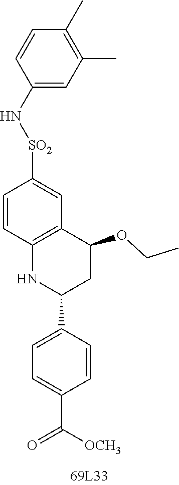 [SMILES: CCO[C@H]1C[C@H](c2ccc(C(=O)OC)cc2)Nc2ccc(S(=O)(=O)Nc3ccc(C)c(C)c3)cc21]

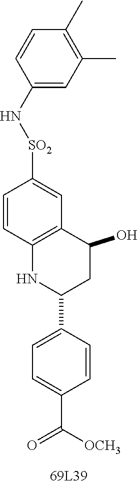 [SMILES: COC(=O)c1ccc([C@H]2C[C@H](O)c3cc(S(=O)(=O)Nc4ccc(C)c(C)c4)ccc3N2)cc1]

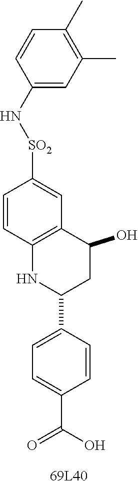 [SMILES: Cc1ccc(NS(=O)(=O)c2ccc3c(c2)[C@@H](O)C[C@H](c2ccc(C(=O)O)cc2)N3)cc1C]

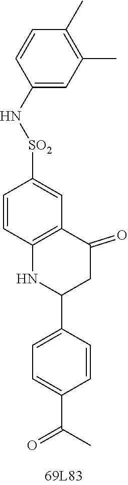 [SMILES: CC(=O)c1ccc(C2CC(=O)c3cc(S(=O)(=O)Nc4ccc(C)c(C)c4)ccc3N2)cc1]

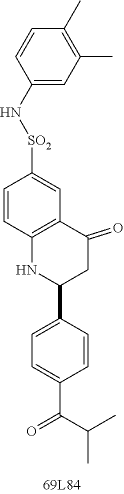 [SMILES: Cc1ccc(NS(=O)(=O)c2ccc3c(c2)C(=O)C[C@@H](c2ccc(C(=O)C(C)C)cc2)N3)cc1C]

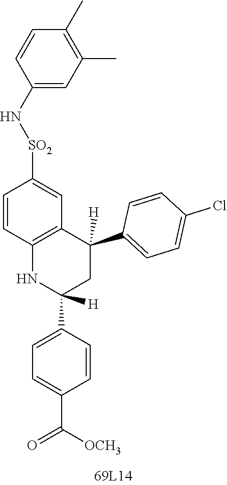 [SMILES: COC(=O)c1ccc([C@H]2C[C@H](c3ccc(Cl)cc3)c3cc(S(=O)(=O)Nc4ccc(C)c(C)c4)ccc3N2)cc1]

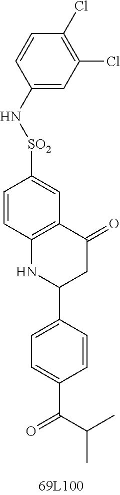 [SMILES: CC(C)C(=O)c1ccc(C2CC(=O)c3cc(S(=O)(=O)Nc4ccc(Cl)c(Cl)c4)ccc3N2)cc1]

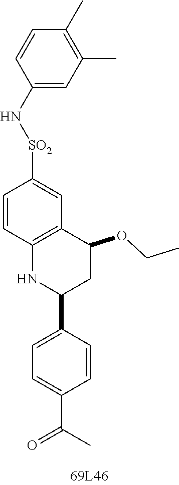 [SMILES: CCO[C@H]1C[C@@H](c2ccc(C(C)=O)cc2)Nc2ccc(S(=O)(=O)Nc3ccc(C)c(C)c3)cc21]

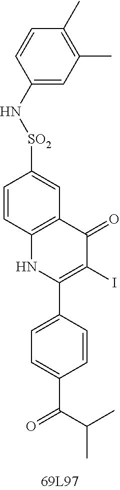 [SMILES: Cc1ccc(NS(=O)(=O)c2ccc3[nH]c(-c4ccc(C(=O)C(C)C)cc4)c(I)c(=O)c3c2)cc1C]

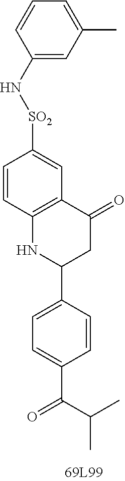 [SMILES: Cc1cccc(NS(=O)(=O)c2ccc3c(c2)C(=O)CC(c2ccc(C(=O)C(C)C)cc2)N3)c1]

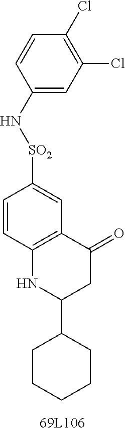 [SMILES: O=C1CC(C2CCCCC2)Nc2ccc(S(=O)(=O)Nc3ccc(Cl)c(Cl)c3)cc21]

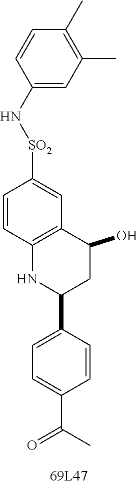 [SMILES: CC(=O)c1ccc([C@@H]2C[C@H](O)c3cc(S(=O)(=O)Nc4ccc(C)c(C)c4)ccc3N2)cc1]

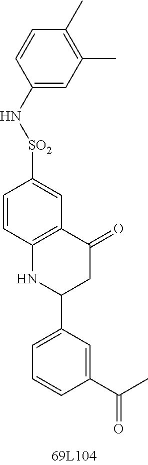 [SMILES: CC(=O)c1cccc(C2CC(=O)c3cc(S(=O)(=O)Nc4ccc(C)c(C)c4)ccc3N2)c1]

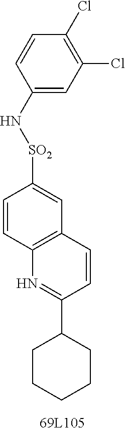 [SMILES: O=S(=O)(Nc1ccc(Cl)c(Cl)c1)c1ccc2nc(C3CCCCC3)ccc2c1]

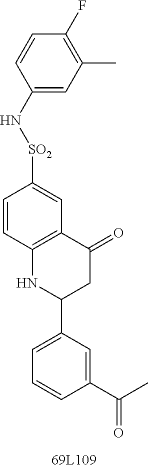 [SMILES: CC(=O)c1cccc(C2CC(=O)c3cc(S(=O)(=O)Nc4ccc(F)c(C)c4)ccc3N2)c1]

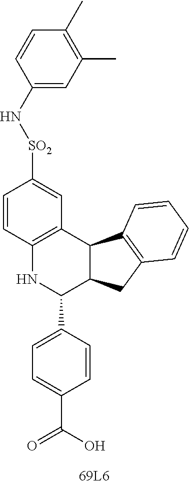 [SMILES: Cc1ccc(NS(=O)(=O)c2ccc3c(c2)[C@H]2c4ccccc4C[C@H]2[C@H](c2ccc(C(=O)O)cc2)N3)cc1C]

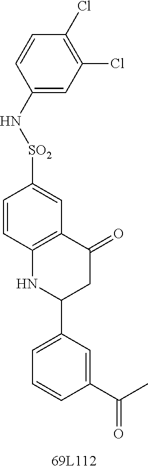 [SMILES: CC(=O)c1cccc(C2CC(=O)c3cc(S(=O)(=O)Nc4ccc(Cl)c(Cl)c4)ccc3N2)c1]

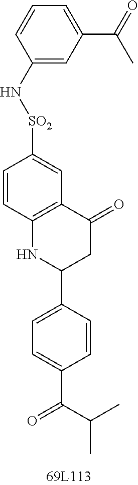 [SMILES: CC(=O)c1cccc(NS(=O)(=O)c2ccc3c(c2)C(=O)CC(c2ccc(C(=O)C(C)C)cc2)N3)c1]

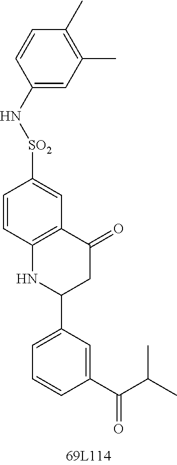 [SMILES: Cc1ccc(NS(=O)(=O)c2ccc3c(c2)C(=O)CC(c2cccc(C(=O)C(C)C)c2)N3)cc1C]

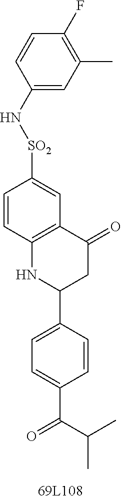 [SMILES: Cc1cc(NS(=O)(=O)c2ccc3c(c2)C(=O)CC(c2ccc(C(=O)C(C)C)cc2)N3)ccc1F]

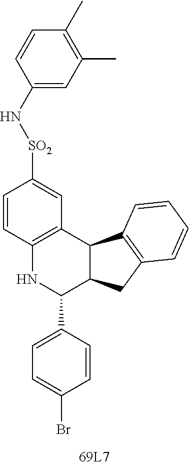 [SMILES: Cc1ccc(NS(=O)(=O)c2ccc3c(c2)[C@H]2c4ccccc4C[C@H]2[C@H](c2ccc(Br)cc2)N3)cc1C]

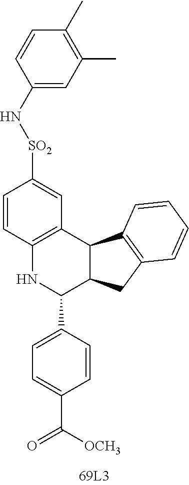 [SMILES: COC(=O)c1ccc([C@@H]2Nc3ccc(S(=O)(=O)Nc4ccc(C)c(C)c4)cc3[C@H]3c4ccccc4C[C@H]32)cc1]

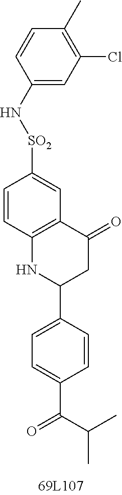 [SMILES: Cc1ccc(NS(=O)(=O)c2ccc3c(c2)C(=O)CC(c2ccc(C(=O)C(C)C)cc2)N3)cc1Cl]

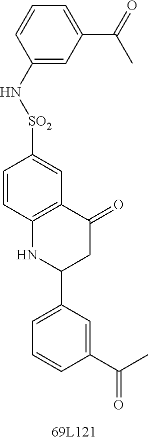 [SMILES: CC(=O)c1cccc(NS(=O)(=O)c2ccc3c(c2)C(=O)CC(c2cccc(C(C)=O)c2)N3)c1]

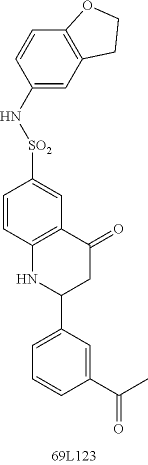 [SMILES: CC(=O)c1cccc(C2CC(=O)c3cc(S(=O)(=O)Nc4ccc5c(c4)CCO5)ccc3N2)c1]

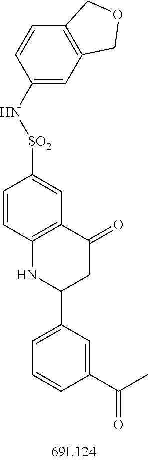 [SMILES: CC(=O)c1cccc(C2CC(=O)c3cc(S(=O)(=O)Nc4ccc5c(c4)COC5)ccc3N2)c1]

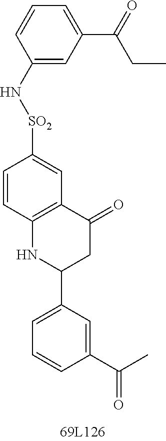 [SMILES: CCC(=O)c1cccc(NS(=O)(=O)c2ccc3c(c2)C(=O)CC(c2cccc(C(C)=O)c2)N3)c1]

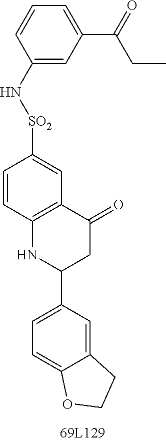 [SMILES: CCC(=O)c1cccc(NS(=O)(=O)c2ccc3c(c2)C(=O)CC(c2ccc4c(c2)CCO4)N3)c1]

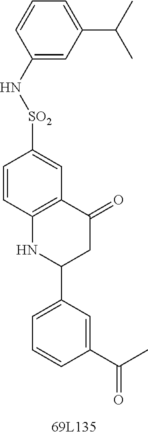 [SMILES: CC(=O)c1cccc(C2CC(=O)c3cc(S(=O)(=O)Nc4cccc(C(C)C)c4)ccc3N2)c1]

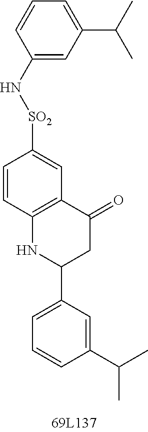 [SMILES: CC(C)c1cccc(NS(=O)(=O)c2ccc3c(c2)C(=O)CC(c2cccc(C(C)C)c2)N3)c1]

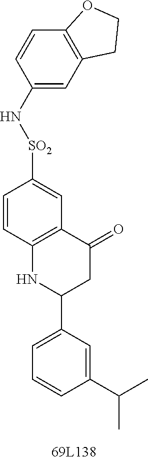 [SMILES: CC(C)c1cccc(C2CC(=O)c3cc(S(=O)(=O)Nc4ccc5c(c4)CCO5)ccc3N2)c1]

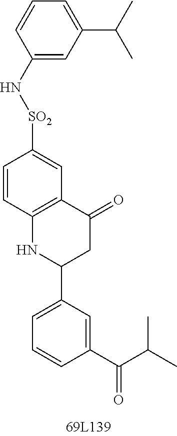 [SMILES: CC(C)C(=O)c1cccc(C2CC(=O)c3cc(S(=O)(=O)Nc4cccc(C(C)C)c4)ccc3N2)c1]

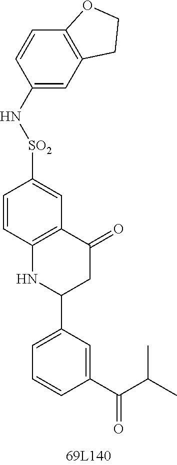 [SMILES: CC(C)C(=O)c1cccc(C2CC(=O)c3cc(S(=O)(=O)Nc4ccc5c(c4)CCO5)ccc3N2)c1]

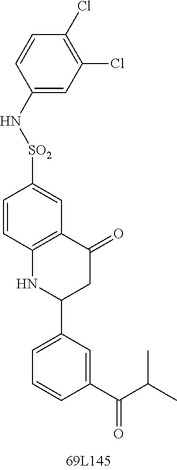 [SMILES: CC(C)C(=O)c1cccc(C2CC(=O)c3cc(S(=O)(=O)Nc4ccc(Cl)c(Cl)c4)ccc3N2)c1]

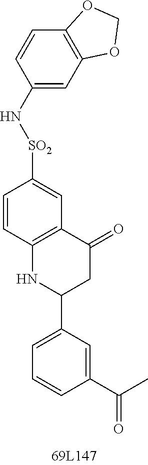 [SMILES: CC(=O)c1cccc(C2CC(=O)c3cc(S(=O)(=O)Nc4ccc5c(c4)OCO5)ccc3N2)c1]

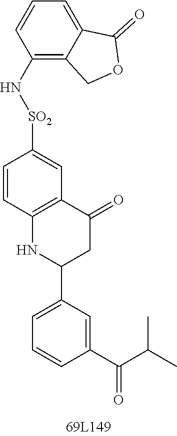 [SMILES: CC(C)C(=O)c1cccc(C2CC(=O)c3cc(S(=O)(=O)Nc4cccc5c4COC5=O)ccc3N2)c1]

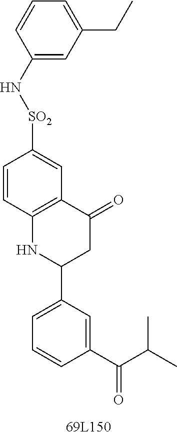 [SMILES: CCc1cccc(NS(=O)(=O)c2ccc3c(c2)C(=O)CC(c2cccc(C(=O)C(C)C)c2)N3)c1]

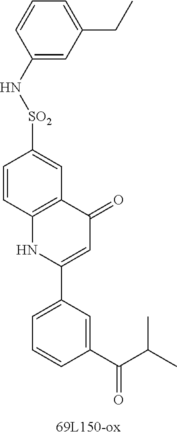 [SMILES: CCc1cccc(NS(=O)(=O)c2ccc3[nH]c(-c4cccc(C(=O)C(C)C)c4)cc(=O)c3c2)c1]

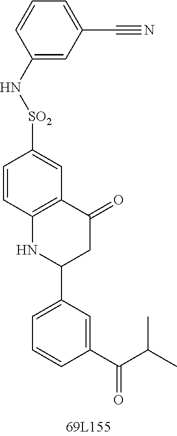 [SMILES: CC(C)C(=O)c1cccc(C2CC(=O)c3cc(S(=O)(=O)Nc4cccc(C#N)c4)ccc3N2)c1]

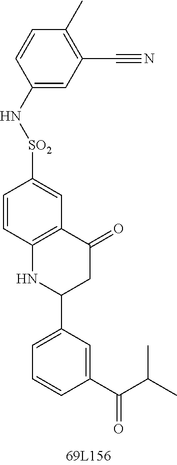 [SMILES: Cc1ccc(NS(=O)(=O)c2ccc3c(c2)C(=O)CC(c2cccc(C(=O)C(C)C)c2)N3)cc1C#N]

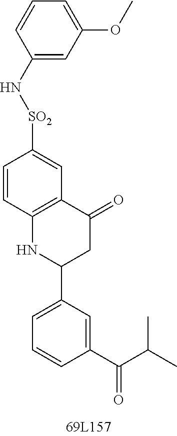 [SMILES: COc1cccc(NS(=O)(=O)c2ccc3c(c2)C(=O)CC(c2cccc(C(=O)C(C)C)c2)N3)c1]

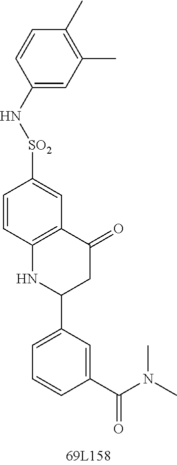 [SMILES: Cc1ccc(NS(=O)(=O)c2ccc3c(c2)C(=O)CC(c2cccc(C(=O)N(C)C)c2)N3)cc1C]

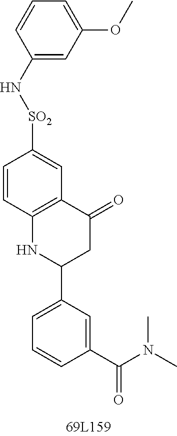 [SMILES: COc1cccc(NS(=O)(=O)c2ccc3c(c2)C(=O)CC(c2cccc(C(=O)N(C)C)c2)N3)c1]

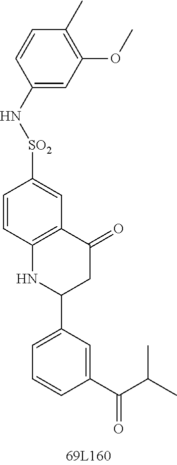 [SMILES: COc1cc(NS(=O)(=O)c2ccc3c(c2)C(=O)CC(c2cccc(C(=O)C(C)C)c2)N3)ccc1C]

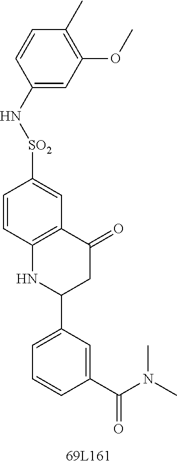 [SMILES: COc1cc(NS(=O)(=O)c2ccc3c(c2)C(=O)CC(c2cccc(C(=O)N(C)C)c2)N3)ccc1C]

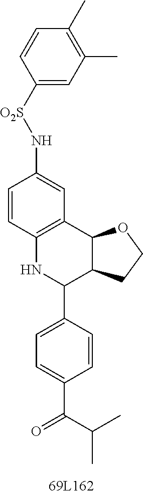 [SMILES: Cc1ccc(S(=O)(=O)Nc2ccc3c(c2)[C@H]2OCC[C@H]2C(c2ccc(C(=O)C(C)C)cc2)N3)cc1C]

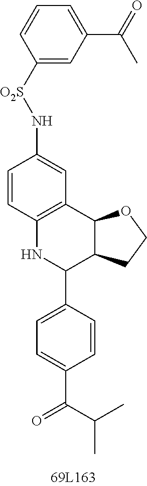 [SMILES: CC(=O)c1cccc(S(=O)(=O)Nc2ccc3c(c2)[C@H]2OCC[C@H]2C(c2ccc(C(=O)C(C)C)cc2)N3)c1]

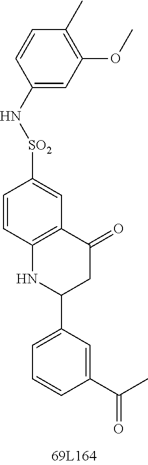 [SMILES: COc1cc(NS(=O)(=O)c2ccc3c(c2)C(=O)CC(c2cccc(C(C)=O)c2)N3)ccc1C]

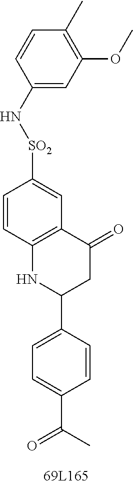 [SMILES: COc1cc(NS(=O)(=O)c2ccc3c(c2)C(=O)CC(c2ccc(C(C)=O)cc2)N3)ccc1C]

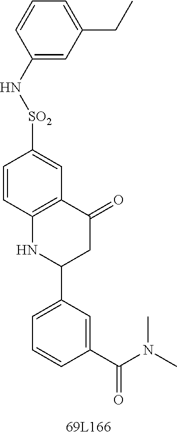 [SMILES: CCc1cccc(NS(=O)(=O)c2ccc3c(c2)C(=O)CC(c2cccc(C(=O)N(C)C)c2)N3)c1]

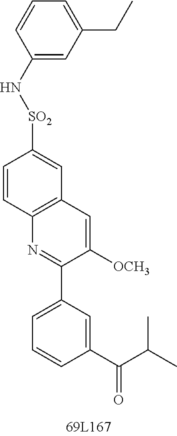 [SMILES: CCc1cccc(NS(=O)(=O)c2ccc3nc(-c4cccc(C(=O)C(C)C)c4)c(OC)cc3c2)c1]

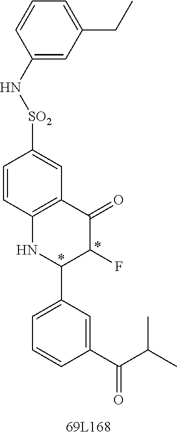 [SMILES: CCc1cccc(NS(=O)(=O)c2ccc3c(c2)C(=O)C(F)C(c2cccc(C(=O)C(C)C)c2)N3)c1]

or pharmaceutically acceptable salts and prodrugs thereof.

In another aspect, provided herein is a compound of formula:

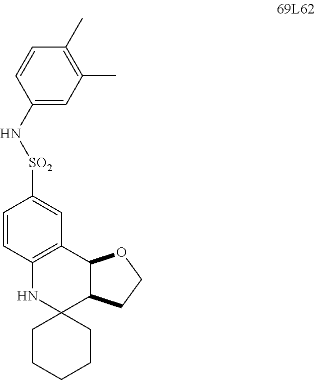

or pharmaceutically acceptable salts and prodrugs thereof.

In another aspect, provided herein is a compound selected from the group consisting of the compounds of Table 3.

TABLE 3

| No. | Structure |
|---|---|
| MX69 | 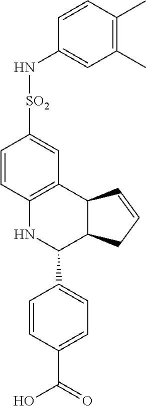 [SMILES: Cc1ccc(NS(=O)(=O)c2ccc3c(c2)[C@H]2C=CC[C@H]2[C@H](c2ccc(C(=O)O)cc2)N3)cc1C] |
| 69L1 | 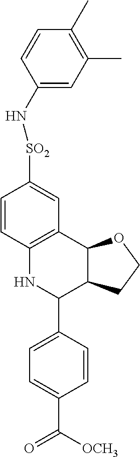 [SMILES: COC(=O)c1ccc(C2Nc3ccc(S(=O)(=O)Nc4ccc(C)c(C)c4)cc3[C@H]3OCC[C@@H]23)cc1] |
| 69L28 | 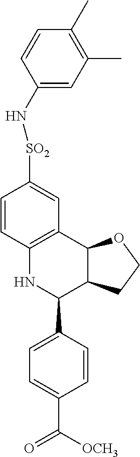 [SMILES: COC(=O)c1ccc([C@H]2Nc3ccc(S(=O)(=O)Nc4ccc(C)c(C)c4)cc3[C@H]3OCC[C@@H]23)cc1] |
| 69L29 | 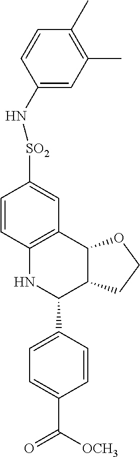 [SMILES: COC(=O)c1ccc([C@@H]2Nc3ccc(S(=O)(=O)Nc4ccc(C)c(C)c4)cc3[C@@H]3OCC[C@H]23)cc1] |
| 69L2 | 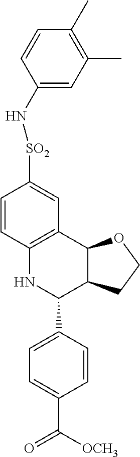 [SMILES: COC(=O)c1ccc([C@@H]2Nc3ccc(S(=O)(=O)Nc4ccc(C)c(C)c4)cc3[C@H]3OCC[C@H]32)cc1] |
| 69L12 | 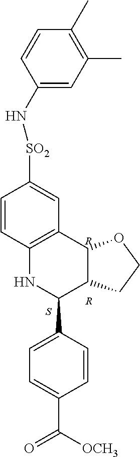 |
| 69L13 | 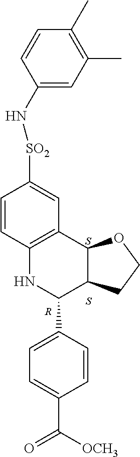 |
| 69L31 | 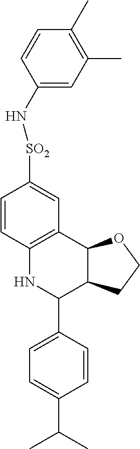 [SMILES: Cc1ccc(NS(=O)(=O)c2ccc3c(c2)[C@H]2OCC[C@H]2C(c2ccc(C(C)C)cc2)N3)cc1C] |
| 69L32 | 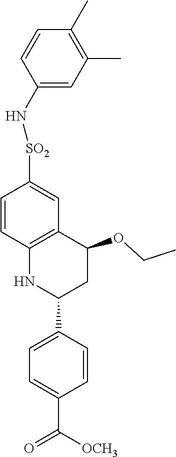 [SMILES: CCO[C@H]1C[C@H](c2ccc(C(=O)OC)cc2)Nc2ccc(S(=O)(=O)Nc3ccc(C)c(C)c3)cc21] |
| 69L33 | 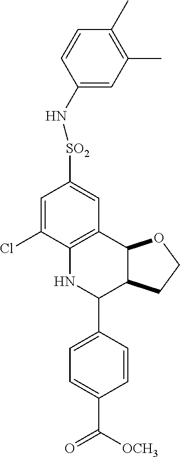 |
| 69L34 | 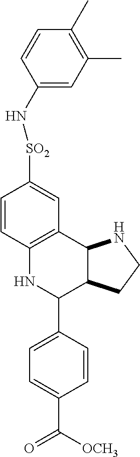 |
| 69L35 | 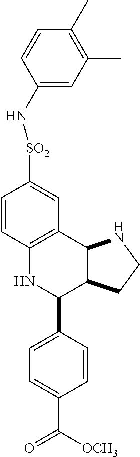 |
| 69L36 | 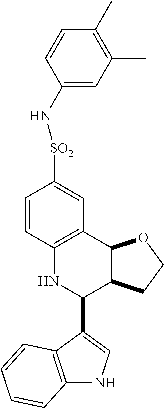 |
| 69L37 | 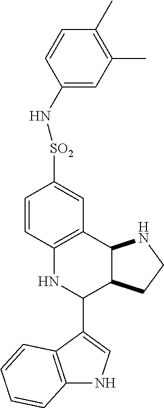 |
| 69L38 | 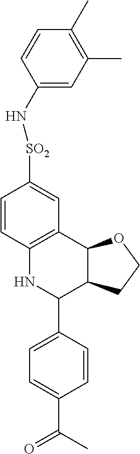 [SMILES: CC(=O)c1ccc(C2Nc3ccc(S(=O)(=O)Nc4ccc(C)c(C)c4)cc3[C@H]3OCC[C@@H]23)cc1] |
| 69L39 | 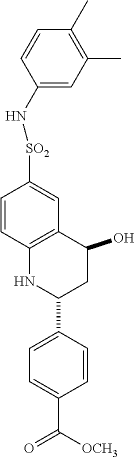 |
| 69L40 | 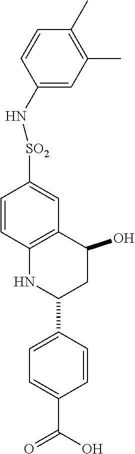 |
| 69L41 | 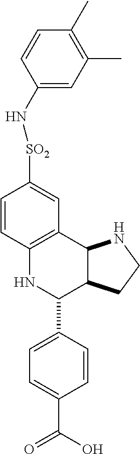 |
| 69L42 | 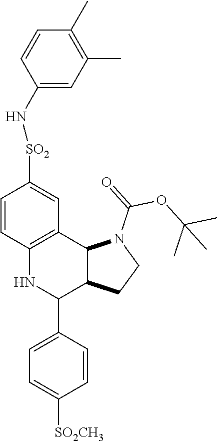 |
| 69L43 | 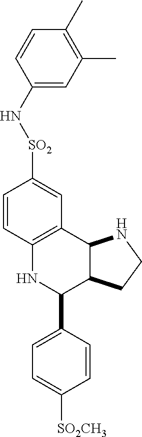 |
| 69L44 | 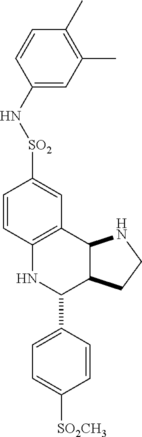 |
| 69L46 | 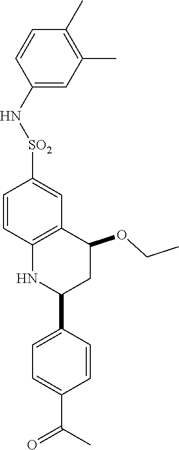 |
| 69L47 | 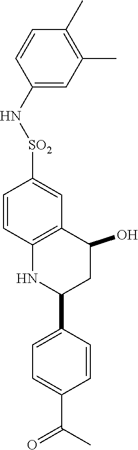 |
| 69L48 | 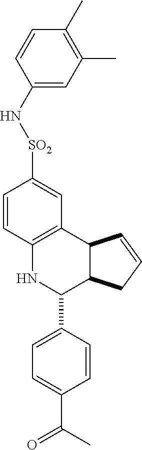 |
| 69L49 | 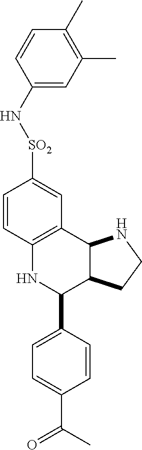 |
| 69L50 | 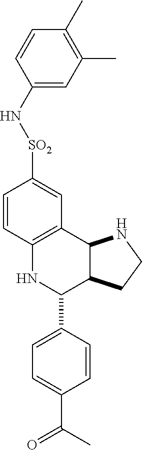 |
| 69L51 | 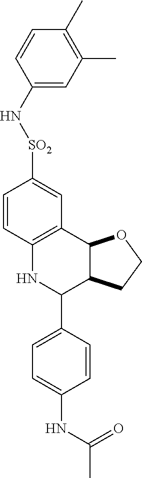 |
| 69L52 | 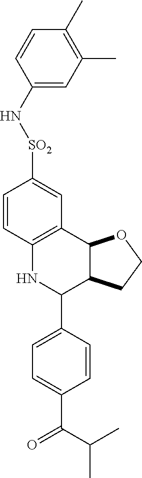 |
| 69L52b | 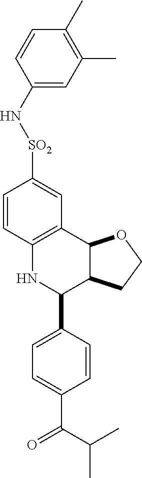 |
| 69L52c | 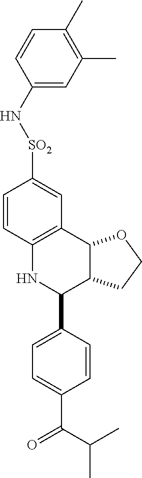 |
| 69L52d | 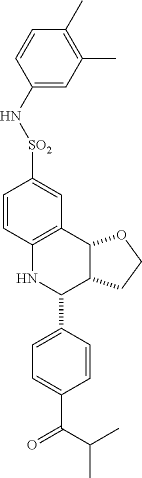 |
| 69L53 | 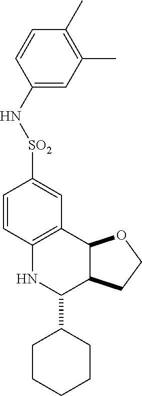 |
| 69L54 | 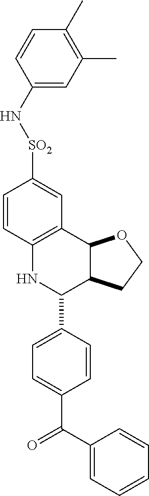 |
| 69L55 | 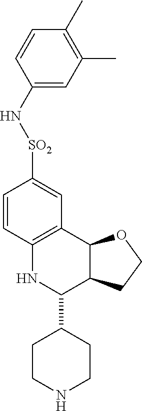 [SMILES: Cc1ccc(NS(=O)(=O)c2ccc3c(c2)[C@H]2OCC[C@H]2[C@H](C2CCNCC2)N3)cc1C] |
| 69L56 | 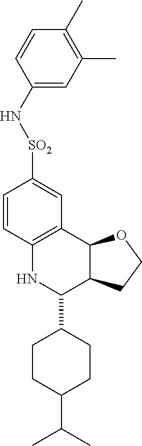 [SMILES: Cc1ccc(NS(=O)(=O)c2ccc3c(c2)[C@H]2OCC[C@H]2[C@H](C2CCC(C(C)C)CC2)N3)cc1C] |
| 69L57 | 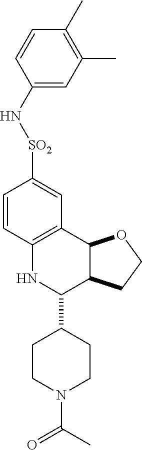 |
| 69L58 | 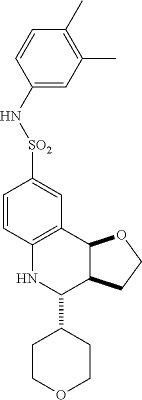 |
| 69L59 | 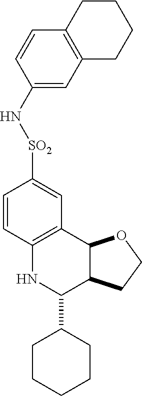 |
| 69L60 | 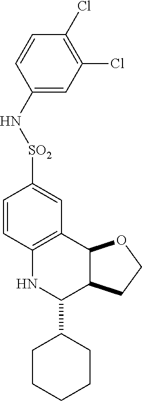 |
| 69L61 | 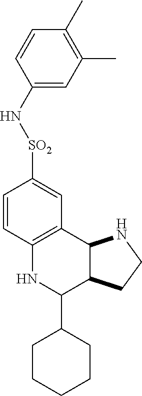 |
| 69L62 | 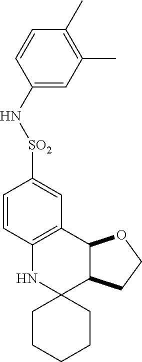 |
| 69L63 | 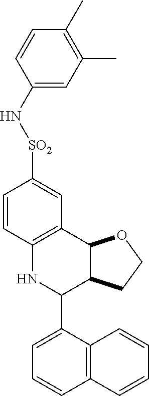 |
| 69L64 | 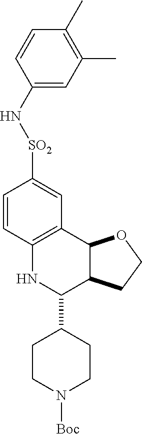 |
| 69L65 | 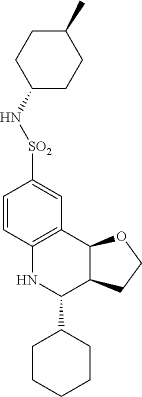 [SMILES: C[C@H]1CC[C@H](NS(=O)(=O)c2ccc3c(c2)[C@H]2OCC[C@H]2[C@H](C2CCCCC2)N3)CC1] |
| 69L66 | 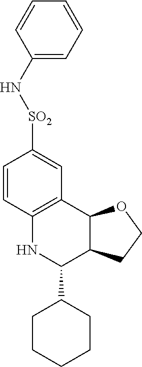 [SMILES: O=S(=O)(Nc1ccccc1)c1ccc2c(c1)[C@H]1OCC[C@H]1[C@H](C1CCCCC1)N2] |
| 69L67 | 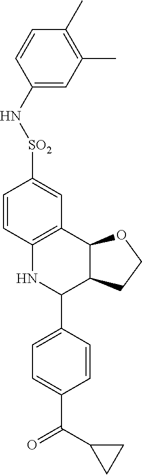 [SMILES: Cc1ccc(NS(=O)(=O)c2ccc3c(c2)[C@H]2OCC[C@H]2C(c2ccc(C(=O)C4CC4)cc2)N3)cc1C] |
| 69L68 | 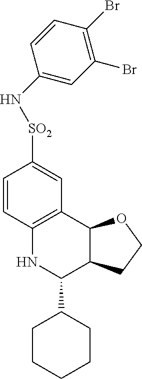 [SMILES: O=S(=O)(Nc1ccc(Br)c(Br)c1)c1ccc2c(c1)[C@H]1OCC[C@H]1[C@H](C1CCCCC1)N2] |
| 69L69 | 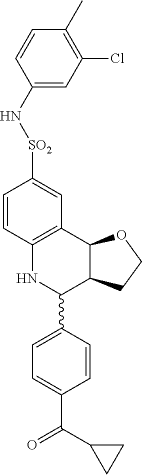 [SMILES: Cc1ccc(NS(=O)(=O)c2ccc3c(c2)[C@H]2OCC[C@H]2C(c2ccc(C(=O)C4CC4)cc2)N3)cc1Cl] |
| 69L70 | 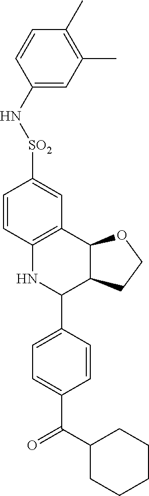 [SMILES: Cc1ccc(NS(=O)(=O)c2ccc3c(c2)[C@H]2OCC[C@H]2C(c2ccc(C(=O)C4CCCCC4)cc2)N3)cc1C] |
| 69L71 | 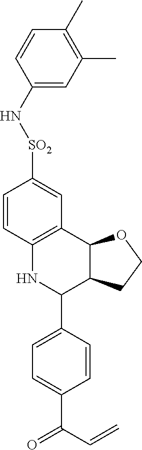 [SMILES: C=CC(=O)c1ccc(C2Nc3ccc(S(=O)(=O)Nc4ccc(C)c(C)c4)cc3[C@H]3OCC[C@@H]23)cc1] |
| 69L72 | 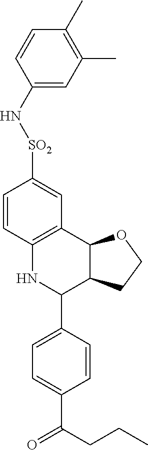 [SMILES: CCCC(=O)c1ccc(C2Nc3ccc(S(=O)(=O)Nc4ccc(C)c(C)c4)cc3[C@H]3OCC[C@@H]23)cc1] |
| 69L73 | 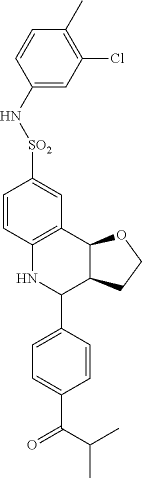 [SMILES: Cc1ccc(NS(=O)(=O)c2ccc3c(c2)[C@H]2OCC[C@H]2C(c2ccc(C(=O)C(C)C)cc2)N3)cc1Cl] |
| 69L74 | 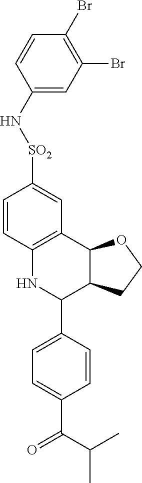 [SMILES: CC(C)C(=O)c1ccc(C2Nc3ccc(S(=O)(=O)Nc4ccc(Br)c(Br)c4)cc3[C@H]3OCC[C@@H]23)cc1] |
| | Intentionally left |
| | blank |
| 69L75 | 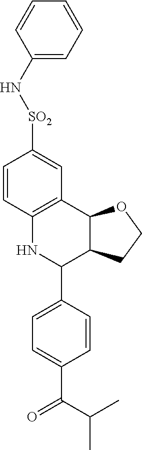 [SMILES: CC(C)C(=O)c1ccc(C2Nc3ccc(S(=O)(=O)Nc4ccccc4)cc3[C@H]3OCC[C@@H]23)cc1] |
| 69L76 | 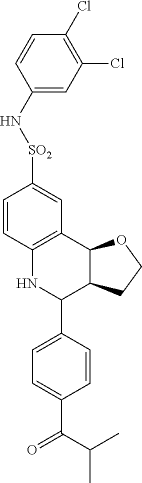 [SMILES: CC(C)C(=O)c1ccc(C2Nc3ccc(S(=O)(=O)Nc4ccc(Cl)c(Cl)c4)cc3[C@H]3OCC[C@@H]23)cc1] |
| 69L77 | 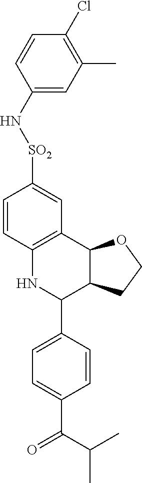 [SMILES: Cc1cc(NS(=O)(=O)c2ccc3c(c2)[C@H]2OCC[C@H]2C(c2ccc(C(=O)C(C)C)cc2)N3)ccc1Cl] |
| 69L78 | 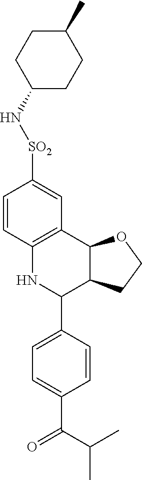 [SMILES: CC(C)C(=O)c1ccc(C2Nc3ccc(S(=O)(=O)N[C@H]4CC[C@H](C)CC4)cc3[C@H]3OCC[C@@H]23)cc1] |
| 69L79 | 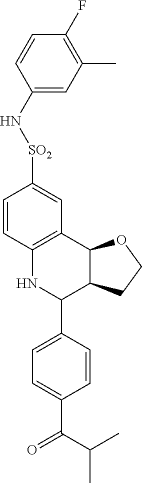 [SMILES: Cc1cc(NS(=O)(=O)c2ccc3c(c2)[C@H]2OCC[C@H]2C(c2ccc(C(=O)C(C)C)cc2)N3)ccc1F] |
| 69L80 | 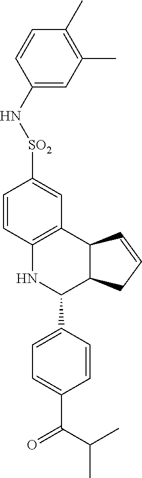 [SMILES: Cc1ccc(NS(=O)(=O)c2ccc3c(c2)[C@H]2C=CC[C@H]2[C@H](c2ccc(C(=O)C(C)C)cc2)N3)cc1C] |
| 69L81 | 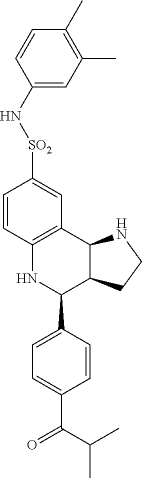 [SMILES: Cc1ccc(NS(=O)(=O)c2ccc3c(c2)[C@H]2NCC[C@H]2[C@@H](c2ccc(C(=O)C(C)C)cc2)N3)cc1C] |
| 69L82 | 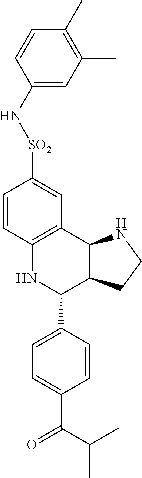 [SMILES: Cc1ccc(NS(=O)(=O)c2ccc3c(c2)[C@H]2NCC[C@H]2[C@H](c2ccc(C(=O)C(C)C)cc2)N3)cc1C] |
| 69L83 | 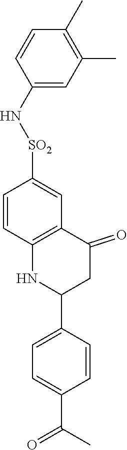 [SMILES: CC(=O)c1ccc(C2CC(=O)c3cc(S(=O)(=O)Nc4ccc(C)c(C)c4)ccc3N2)cc1] |
| 69L84 | 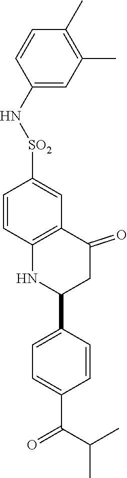 [SMILES: Cc1ccc(NS(=O)(=O)c2ccc3c(c2)C(=O)C[C@@H](c2ccc(C(=O)C(C)C)cc2)N3)cc1C] |
| 69L85 | 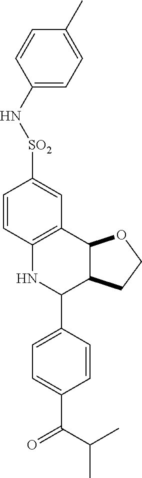 |
| 69L86 | 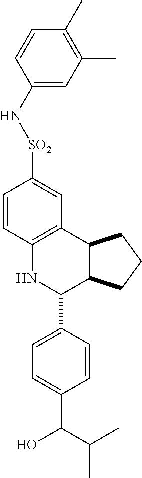 |
| 69L87 | 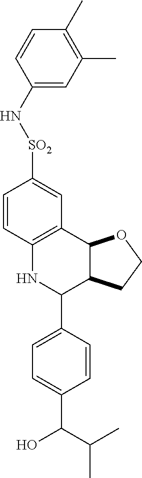 |
| 69L88 | 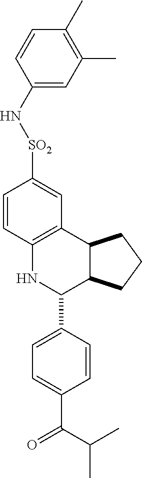 |
| 69L89 | 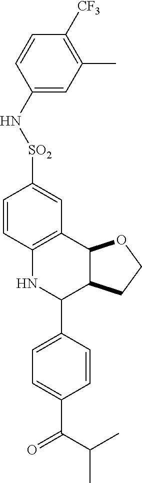 |
| 69L90 | 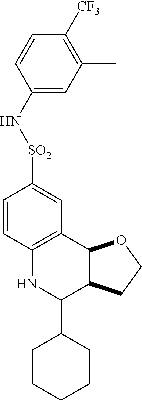 |
| 69L91 | 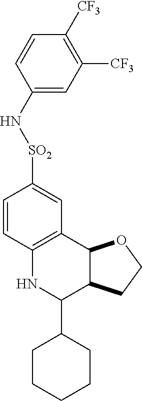 |
| 69L92 | 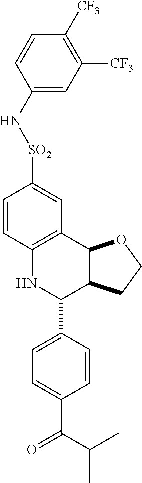 |
| 69L93 | 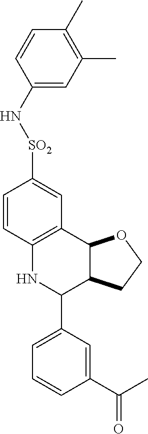 |
| 69L94 | 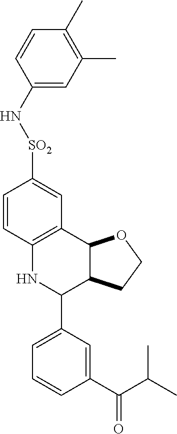 |
| 69L95 | 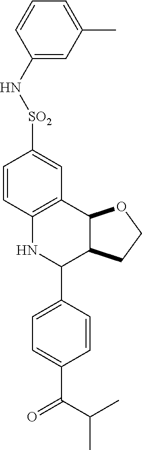 |
| 69L96 | 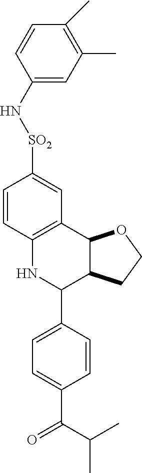 |
| 69L97 | 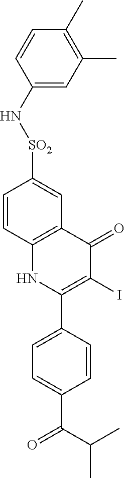 [SMILES: Cc1ccc(NS(=O)(=O)c2ccc3[nH]c(-c4ccc(C(=O)C(C)C)cc4)c(I)c(=O)c3c2)cc1C] |
| 69L98 | 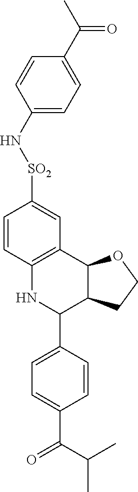 [SMILES: CC(=O)c1ccc(NS(=O)(=O)c2ccc3c(c2)[C@H]2OCC[C@H]2C(c2ccc(C(=O)C(C)C)cc2)N3)cc1] |
| 69L99 | 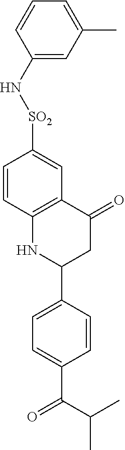 [SMILES: Cc1cccc(NS(=O)(=O)c2ccc3c(c2)C(=O)CC(c2ccc(C(=O)C(C)C)cc2)N3)c1] |
| 69L100 | 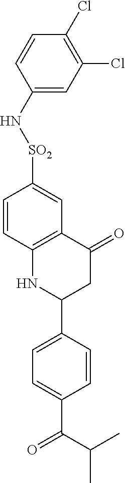 [SMILES: CC(C)C(=O)c1ccc(C2CC(=O)c3cc(S(=O)(=O)Nc4ccc(Cl)c(Cl)c4)ccc3N2)cc1] |
| 69L101 | 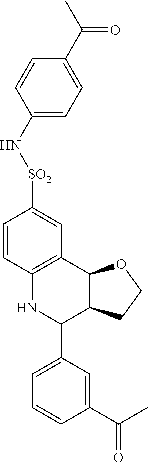 [SMILES: CC(=O)c1ccc(NS(=O)(=O)c2ccc3c(c2)[C@H]2OCC[C@H]2C(c2cccc(C(C)=O)c2)N3)cc1] |
| 69L102 | 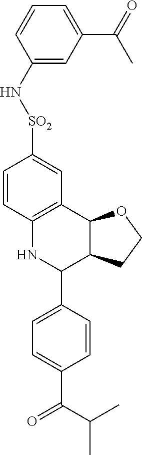 [SMILES: CC(=O)c1cccc(NS(=O)(=O)c2ccc3c(c2)[C@H]2OCC[C@H]2C(c2ccc(C(=O)C(C)C)cc2)N3)c1] |
| 69L103 | 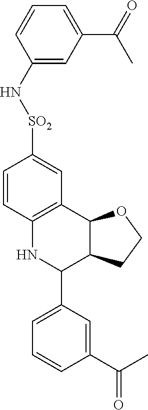 [SMILES: CC(=O)c1cccc(NS(=O)(=O)c2ccc3c(c2)[C@H]2OCC[C@H]2C(c2cccc(C(C)=O)c2)N3)c1] |
| 69L104 | 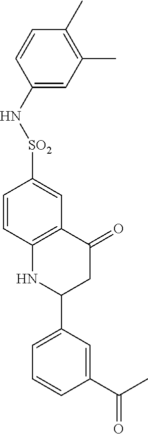 [SMILES: CC(=O)c1cccc(C2CC(=O)c3cc(S(=O)(=O)Nc4ccc(C)c(C)c4)ccc3N2)c1] |
| 69L105 | 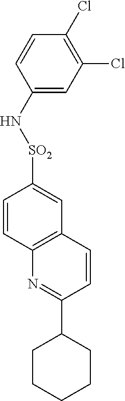 [SMILES: O=S(=O)(Nc1ccc(Cl)c(Cl)c1)c1ccc2nc(C3CCCCC3)ccc2c1] |
| 69L106 | 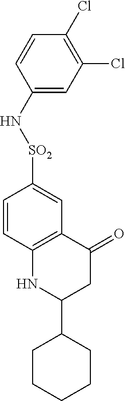 [SMILES: O=C1CC(C2CCCCC2)Nc2ccc(S(=O)(=O)Nc3ccc(Cl)c(Cl)c3)cc21] |
| 69L107 | 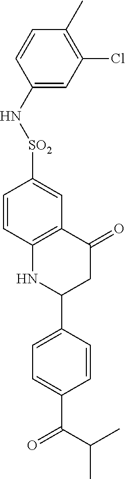 [SMILES: Cc1ccc(NS(=O)(=O)c2ccc3c(c2)C(=O)CC(c2ccc(C(=O)C(C)C)cc2)N3)cc1Cl] |
| 69L108 | 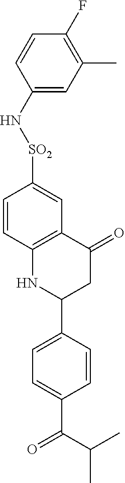 [SMILES: Cc1cc(NS(=O)(=O)c2ccc3c(c2)C(=O)CC(c2ccc(C(=O)C(C)C)cc2)N3)ccc1F] |
| 69L109 | 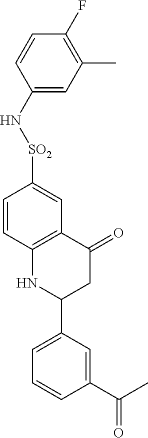 [SMILES: CC(=O)c1cccc(C2CC(=O)c3cc(S(=O)(=O)Nc4ccc(F)c(C)c4)ccc3N2)c1] |
| 69L110 | 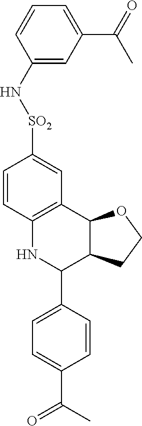 [SMILES: CC(=O)c1ccc(C2Nc3ccc(S(=O)(=O)Nc4cccc(C(C)=O)c4)cc3[C@H]3OCC[C@@H]23)cc1] |
| 69L111 | 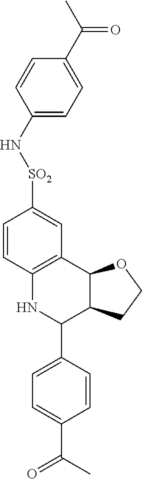 [SMILES: CC(=O)c1ccc(NS(=O)(=O)c2ccc3c(c2)[C@H]2OCC[C@H]2C(c2ccc(C(C)=O)cc2)N3)cc1] |
| 69L112 | 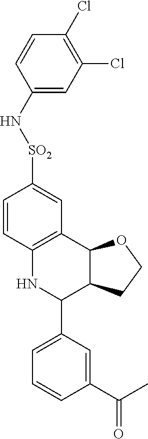 [SMILES: CC(=O)c1cccc(C2Nc3ccc(S(=O)(=O)Nc4ccc(Cl)c(Cl)c4)cc3[C@H]3OCC[C@@H]23)c1] |
| 69L113 | 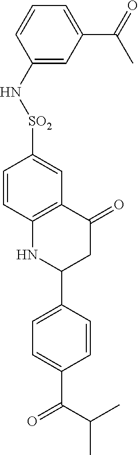 [SMILES: CC(=O)c1cccc(NS(=O)(=O)c2ccc3c(c2)C(=O)CC(c2ccc(C(=O)C(C)C)cc2)N3)c1] |
| 69L114 | 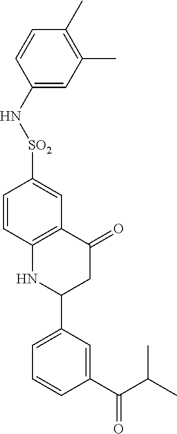 [SMILES: Cc1ccc(NS(=O)(=O)c2ccc3c(c2)C(=O)CC(c2cccc(C(=O)C(C)C)c2)N3)cc1C] |
| 69L115 | 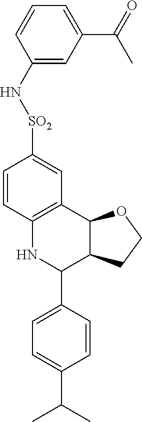 [SMILES: CC(=O)c1cccc(NS(=O)(=O)c2ccc3c(c2)[C@H]2OCC[C@H]2C(c2ccc(C(C)C)cc2)N3)c1] |
| 69L116 | 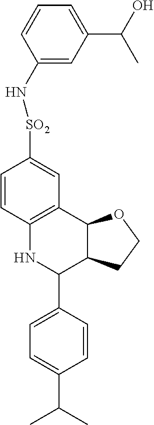 [SMILES: CC(C)c1ccc(C2Nc3ccc(S(=O)(=O)Nc4cccc(C(C)O)c4)cc3[C@H]3OCC[C@@H]23)cc1] |
| 69L117 | 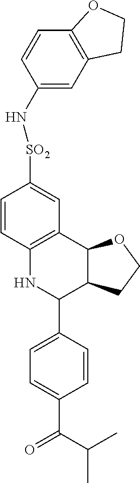 [SMILES: CC(C)C(=O)c1ccc(C2Nc3ccc(S(=O)(=O)Nc4ccc5c(c4)CCO5)cc3[C@H]3OCC[C@@H]23)cc1] |
| 69L118 | 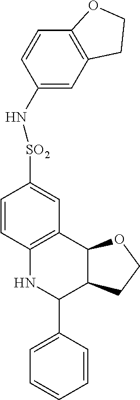 [SMILES: O=S(=O)(Nc1ccc2c(c1)CCO2)c1ccc2c(c1)[C@H]1OCC[C@H]1C(c1ccccc1)N2] |
| 69L119 | 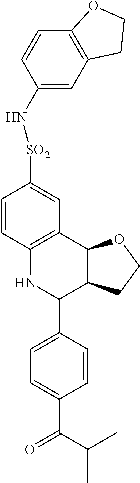 [SMILES: CC(C)C(=O)c1ccc(C2Nc3ccc(S(=O)(=O)Nc4ccc5c(c4)CCO5)cc3[C@H]3OCC[C@@H]23)cc1] |
| 69L120 | 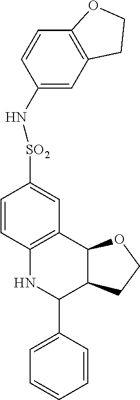 [SMILES: O=S(=O)(Nc1ccc2c(c1)CCO2)c1ccc2c(c1)[C@H]1OCC[C@H]1C(c1ccccc1)N2] |
| 69L3 | 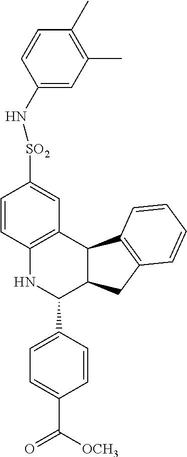 [SMILES: COC(=O)c1ccc([C@@H]2Nc3ccc(S(=O)(=O)Nc4ccc(C)c(C)c4)cc3[C@H]3c4ccccc4C[C@H]32)cc1] |
| 69L4 | 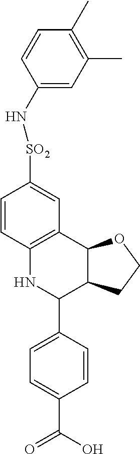 [SMILES: Cc1ccc(NS(=O)(=O)c2ccc3c(c2)[C@H]2OCC[C@H]2C(c2ccc(C(=O)O)cc2)N3)cc1C] |
| 69L5 | 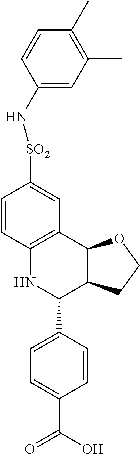 [SMILES: Cc1ccc(NS(=O)(=O)c2ccc3c(c2)[C@H]2OCC[C@H]2[C@H](c2ccc(C(=O)O)cc2)N3)cc1C] |
| 69L6 | 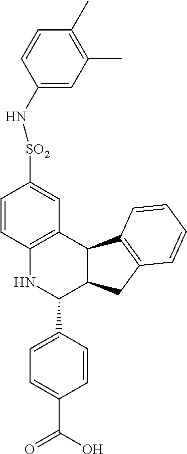 [SMILES: Cc1ccc(NS(=O)(=O)c2ccc3c(c2)[C@H]2c4ccccc4C[C@H]2[C@H](c2ccc(C(=O)O)cc2)N3)cc1C] |
| 69L7 | 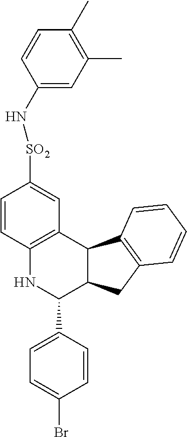 [SMILES: Cc1ccc(NS(=O)(=O)c2ccc3c(c2)[C@H]2c4ccccc4C[C@H]2[C@H](c2ccc(Br)cc2)N3)cc1C] |
| 69L8 | 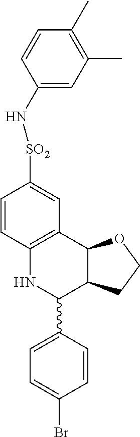 [SMILES: Cc1ccc(NS(=O)(=O)c2ccc3c(c2)[C@H]2OCC[C@H]2C(c2ccc(Br)cc2)N3)cc1C] |
| 69L9 | 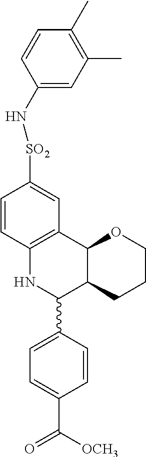 [SMILES: COC(=O)c1ccc(C2Nc3ccc(S(=O)(=O)Nc4ccc(C)c(C)c4)cc3[C@H]3OCCC[C@@H]23)cc1] |
| 69L10 | 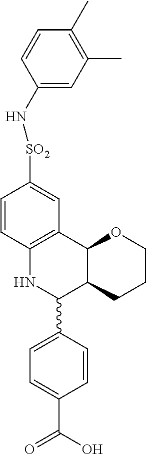 [SMILES: Cc1ccc(NS(=O)(=O)c2ccc3c(c2)[C@H]2OCCC[C@H]2C(c2ccc(C(=O)O)cc2)N3)cc1C] |
| 69L14 | 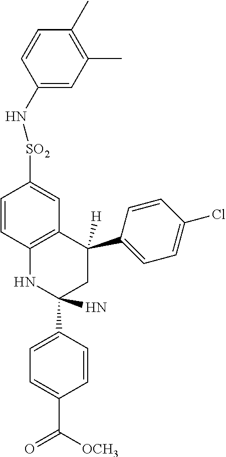 [SMILES: COC(=O)c1ccc([C@@]2(N)C[C@H](c3ccc(Cl)cc3)c3cc(S(=O)(=O)Nc4ccc(C)c(C)c4)ccc3N2)cc1] |
| 69L15 | 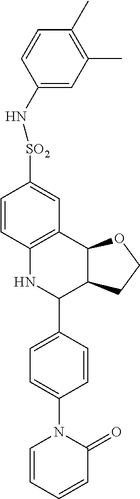 [SMILES: Cc1ccc(NS(=O)(=O)c2ccc3c(c2)[C@H]2OCC[C@H]2C(c2ccc(-n4ccccc4=O)cc2)N3)cc1C] |
| 69L16 | 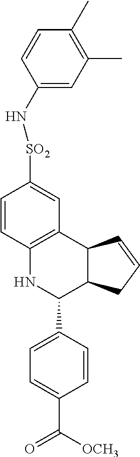 [SMILES: COC(=O)c1ccc([C@@H]2Nc3ccc(S(=O)(=O)Nc4ccc(C)c(C)c4)cc3[C@H]3C=CC[C@H]32)cc1] |
| 69L17 | 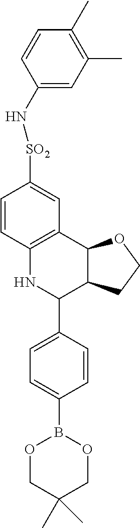 [SMILES: Cc1ccc(NS(=O)(=O)c2ccc3c(c2)[C@H]2OCC[C@H]2C(c2ccc(B4OCC(C)(C)CO4)cc2)N3)cc1C] |
| 69L18 | 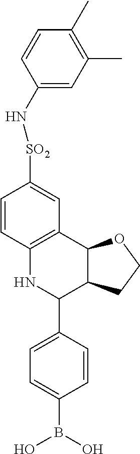 [SMILES: Cc1ccc(NS(=O)(=O)c2ccc3c(c2)[C@H]2OCC[C@H]2C(c2ccc(B(O)O)cc2)N3)cc1C] |
| 69L19 | 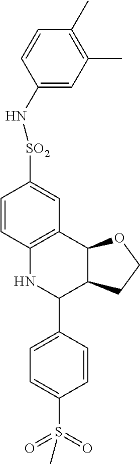 [SMILES: Cc1ccc(NS(=O)(=O)c2ccc3c(c2)[C@H]2OCC[C@H]2C(c2ccc(S(C)(=O)=O)cc2)N3)cc1C] |
| 69L21 | 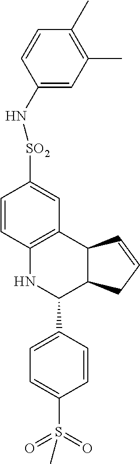 [SMILES: Cc1ccc(NS(=O)(=O)c2ccc3c(c2)[C@H]2C=CC[C@H]2[C@H](c2ccc(S(C)(=O)=O)cc2)N3)cc1C] |
| 69L22 | 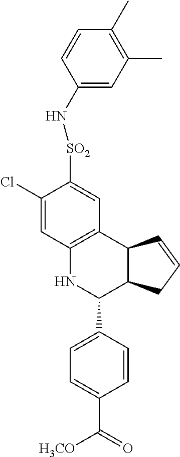 [SMILES: COC(=O)c1ccc([C@@H]2Nc3cc(Cl)c(S(=O)(=O)Nc4ccc(C)c(C)c4)cc3[C@H]3C=CC[C@H]32)cc1] |
| 69L23 | 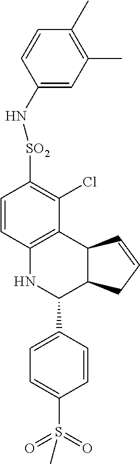 [SMILES: Cc1ccc(NS(=O)(=O)c2ccc3c(c2Cl)[C@H]2C=CC[C@H]2[C@H](c2ccc(S(C)(=O)=O)cc2)N3)cc1C] |
| 69L24 | 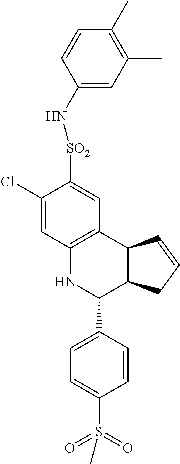 [SMILES: Cc1ccc(NS(=O)(=O)c2cc3c(cc2Cl)N[C@@H](c2ccc(S(C)(=O)=O)cc2)[C@@H]2CC=C[C@H]32)cc1C] |
| 69L25 | 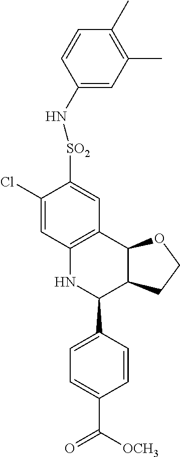 [SMILES: COC(=O)c1ccc([C@H]2Nc3cc(Cl)c(S(=O)(=O)Nc4ccc(C)c(C)c4)cc3[C@H]3OCC[C@@H]23)cc1] |
| 69L26 | 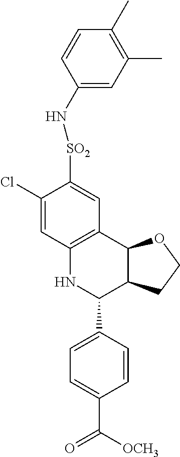 [SMILES: COC(=O)c1ccc([C@@H]2Nc3cc(Cl)c(S(=O)(=O)Nc4ccc(C)c(C)c4)cc3[C@H]3OCC[C@H]32)cc1] |
| | 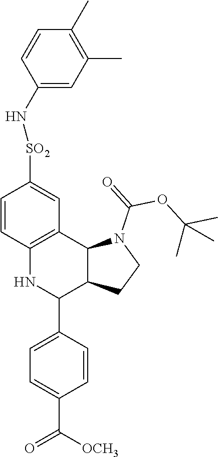 [SMILES: COC(=O)c1ccc(C2Nc3ccc(S(=O)(=O)Nc4ccc(C)c(C)c4)cc3[C@@H]3[C@H]2CCN3C(=O)OC(C)(C)C)cc1] |
| 69L121 | 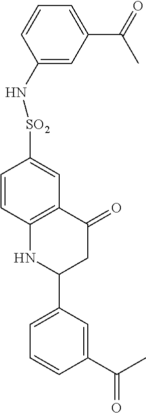 [SMILES: CC(=O)c1cccc(NS(=O)(=O)c2ccc3c(c2)C(=O)CC(c2cccc(C(C)=O)c2)N3)c1] |
| 69L122 | 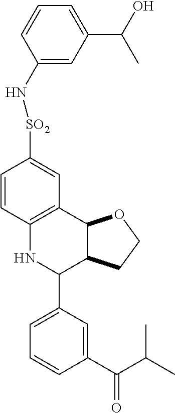 |
| 69L123 | 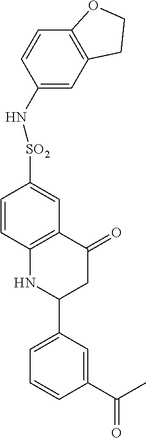 [SMILES: CC(=O)c1cccc(C2CC(=O)c3cc(S(=O)(=O)Nc4ccc5c(c4)CCO5)ccc3N2)c1] |
| 69L124 | 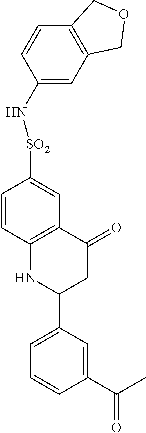 [SMILES: CC(=O)c1cccc(C2CC(=O)c3cc(S(=O)(=O)Nc4ccc5c(c4)COC5)ccc3N2)c1] |
| 69L125 | 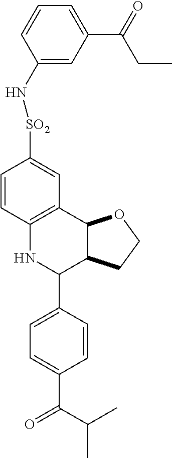 |
| 69L126 | 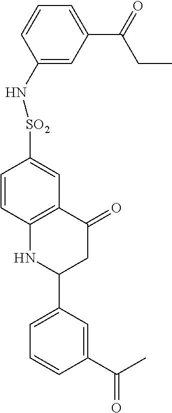 [SMILES: CCC(=O)c1cccc(NS(=O)(=O)c2ccc3c(c2)C(=O)CC(c2cccc(C(C)=O)c2)N3)c1] |
| 69L127 | 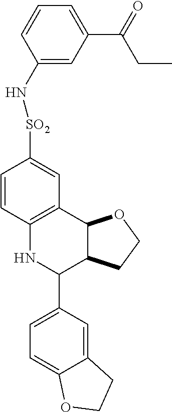 |
| 69L128 | 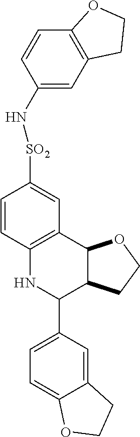 |
| 69L129 | 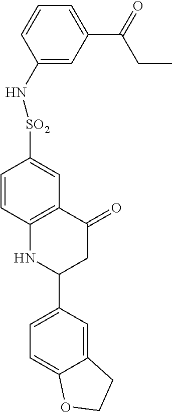 [SMILES: CCC(=O)c1cccc(NS(=O)(=O)c2ccc3c(c2)C(=O)CC(c2ccc4c(c2)CCO4)N3)c1] |
| 69L130 | 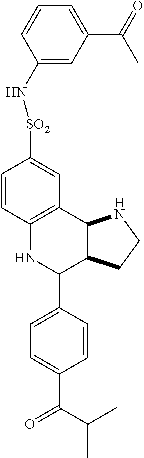 |
| 69L130 trans | 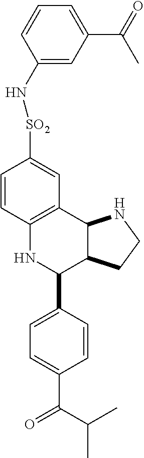 |
| 69L130 cis | 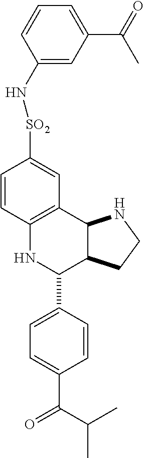 |
| 69L131 | 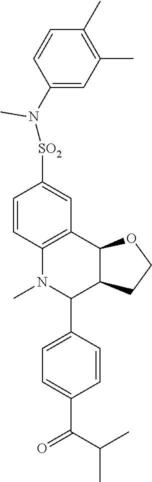 [SMILES: Cc1ccc(N(C)S(=O)(=O)c2ccc3c(c2)[C@H]2OCC[C@H]2C(c2ccc(C(=O)C(C)C)cc2)N3C)cc1C] |
| 69L132 | 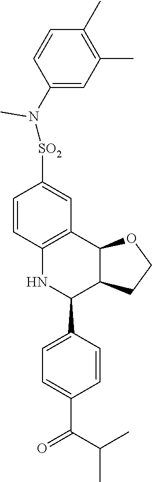 [SMILES: Cc1ccc(N(C)S(=O)(=O)c2ccc3c(c2)[C@H]2OCC[C@H]2[C@@H](c2ccc(C(=O)C(C)C)cc2)N3)cc1C] |
| 69L133 | 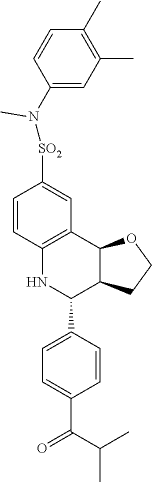 [SMILES: Cc1ccc(N(C)S(=O)(=O)c2ccc3c(c2)[C@H]2OCC[C@H]2[C@H](c2ccc(C(=O)C(C)C)cc2)N3)cc1C] |
| 69L134 | 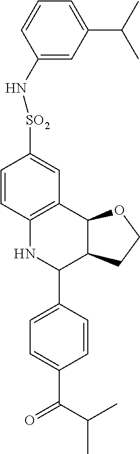 [SMILES: CC(C)C(=O)c1ccc(C2Nc3ccc(S(=O)(=O)Nc4cccc(C(C)C)c4)cc3[C@H]3OCC[C@@H]23)cc1] |
| 69L136 | 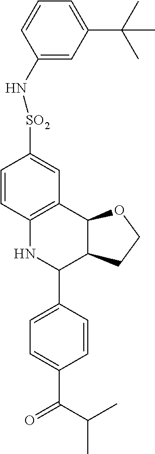 [SMILES: CC(C)C(=O)c1ccc(C2Nc3ccc(S(=O)(=O)Nc4cccc(C(C)(C)C)c4)cc3[C@H]3OCC[C@@H]23)cc1] |
| 69L135 | 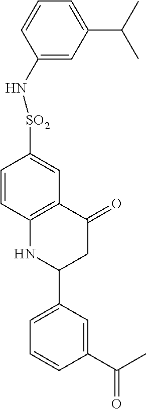 [SMILES: CC(=O)c1cccc(C2CC(=O)c3cc(S(=O)(=O)Nc4cccc(C(C)C)c4)ccc3N2)c1] |
| 69L141 | 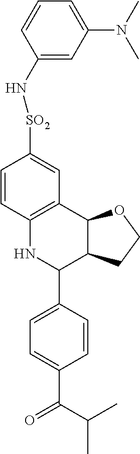 [SMILES: CC(C)C(=O)c1ccc(C2Nc3ccc(S(=O)(=O)Nc4cccc(N(C)C)c4)cc3[C@H]3OCC[C@@H]23)cc1] |
| 69L142 | 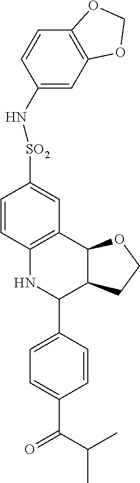 [SMILES: CC(C)C(=O)c1ccc(C2Nc3ccc(S(=O)(=O)Nc4ccc5c(c4)OCO5)cc3[C@H]3OCC[C@@H]23)cc1] |
| 69L143 | 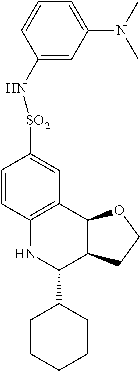 [SMILES: CN(C)c1cccc(NS(=O)(=O)c2ccc3c(c2)[C@H]2OCC[C@H]2[C@H](C2CCCCC2)N3)c1] |
| 69L144 | 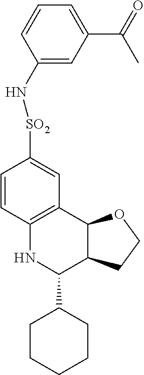 [SMILES: CC(=O)c1cccc(NS(=O)(=O)c2ccc3c(c2)[C@H]2OCC[C@H]2[C@H](C2CCCCC2)N3)c1] |
| 69L146 | 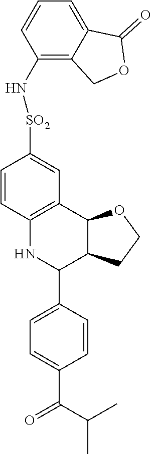 [SMILES: CC(C)C(=O)c1ccc(C2Nc3ccc(S(=O)(=O)Nc4cccc5c4COC5=O)cc3[C@H]3OCC[C@@H]23)cc1] |
| 69L148 | 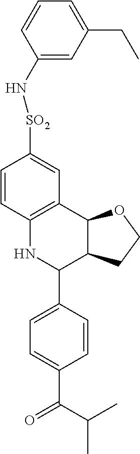 [SMILES: CCc1cccc(NS(=O)(=O)c2ccc3c(c2)[C@H]2OCC[C@H]2C(c2ccc(C(=O)C(C)C)cc2)N3)c1] |
| 69L162 | 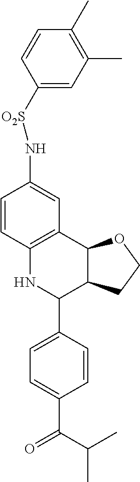 [SMILES: Cc1ccc(S(=O)(=O)Nc2ccc3c(c2)[C@H]2OCC[C@H]2C(c2ccc(C(=O)C(C)C)cc2)N3)cc1C] |
| 69L163 | 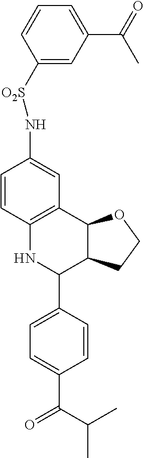 [SMILES: CC(=O)c1cccc(S(=O)(=O)Nc2ccc3c(c2)[C@H]2OCC[C@H]2C(c2ccc(C(=O)C(C)C)cc2)N3)c1] |
| 69L137 | 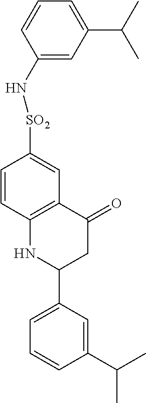 [SMILES: CC(C)c1cccc(NS(=O)(=O)c2ccc3c(c2)C(=O)CC(c2cccc(C(C)C)c2)N3)c1] |
| 69L138 | 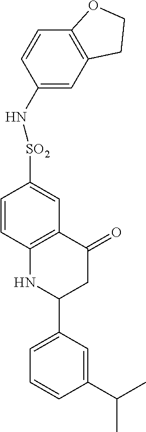 [SMILES: CC(C)c1cccc(C2CC(=O)c3cc(S(=O)(=O)Nc4ccc5c(c4)CCO5)ccc3N2)c1] |
| 69L139 | 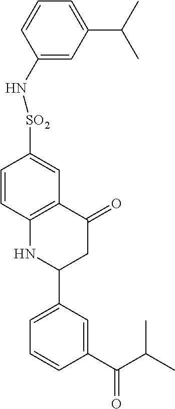 [SMILES: CC(C)C(=O)c1cccc(C2CC(=O)c3cc(S(=O)(=O)Nc4cccc(C(C)C)c4)ccc3N2)c1] |
| 69L140 | 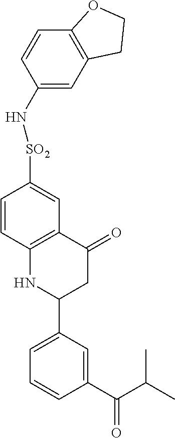 [SMILES: CC(C)C(=O)c1cccc(C2CC(=O)c3cc(S(=O)(=O)Nc4ccc5c(c4)CCO5)ccc3N2)c1] |
| 69L145 | 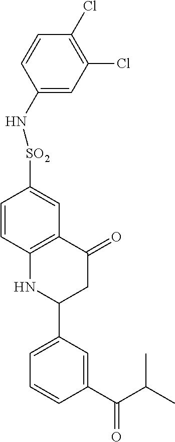 [SMILES: CC(C)C(=O)c1cccc(C2CC(=O)c3cc(S(=O)(=O)Nc4ccc(Cl)c(Cl)c4)ccc3N2)c1] |
| 69L147 | 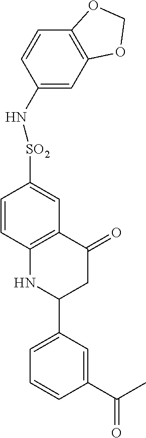 [SMILES: CC(=O)c1cccc(C2CC(=O)c3cc(S(=O)(=O)Nc4ccc5c(c4)OCO5)ccc3N2)c1] |
| 69L149 | 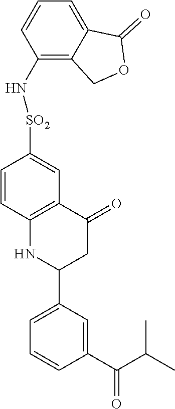 [SMILES: CC(C)C(=O)c1cccc(C2CC(=O)c3cc(S(=O)(=O)Nc4cccc5c4COC5=O)ccc3N2)c1] |
| 69L150 | 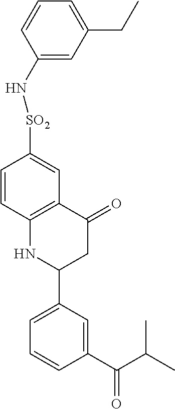 [SMILES: CCc1cccc(NS(=O)(=O)c2ccc3c(c2)C(=O)CC(c2cccc(C(=O)C(C)C)c2)N3)c1] |
| 69L150- ox | 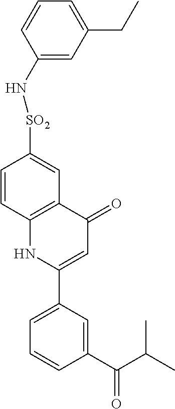 [SMILES: CCc1cccc(NS(=O)(=O)c2ccc3[nH]c(-c4cccc(C(=O)C(C)C)c4)cc(=O)c3c2)c1] |
| 69L155 | 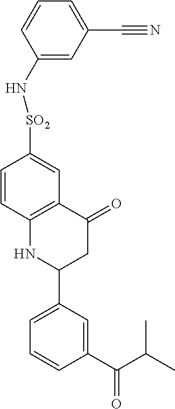 [SMILES: CC(C)C(=O)c1cccc(C2CC(=O)c3cc(S(=O)(=O)Nc4cccc(C#N)c4)ccc3N2)c1] |
| 69L156 | 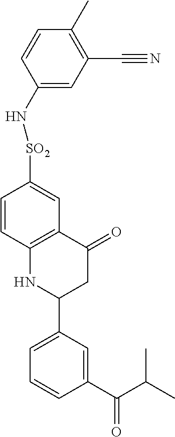 [SMILES: Cc1ccc(NS(=O)(=O)c2ccc3c(c2)C(=O)CC(c2cccc(C(=O)C(C)C)c2)N3)cc1C#N] |
| 69L157 | 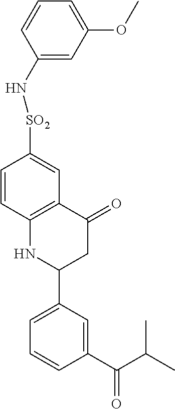 [SMILES: COc1cccc(NS(=O)(=O)c2ccc3c(c2)C(=O)CC(c2cccc(C(=O)C(C)C)c2)N3)c1] |
| 69L158 | 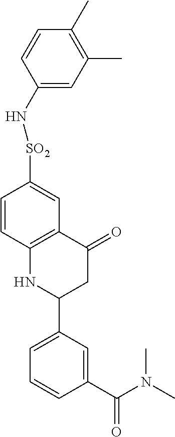 [SMILES: Cc1ccc(NS(=O)(=O)c2ccc3c(c2)C(=O)CC(c2cccc(C(=O)N(C)C)c2)N3)cc1C] |
| 69L159 | 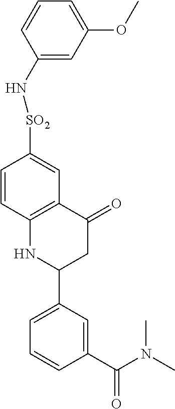 [SMILES: COc1cccc(NS(=O)(=O)c2ccc3c(c2)C(=O)CC(c2cccc(C(=O)N(C)C)c2)N3)c1] |
| 69L160 | 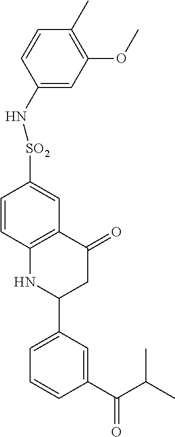 [SMILES: COc1cc(NS(=O)(=O)c2ccc3c(c2)C(=O)CC(c2cccc(C(=O)C(C)C)c2)N3)ccc1C] |
| 69L161 | 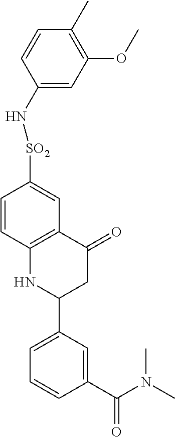 [SMILES: COc1cc(NS(=O)(=O)c2ccc3c(c2)C(=O)CC(c2cccc(C(=O)N(C)C)c2)N3)ccc1C] |
| 69L162 | 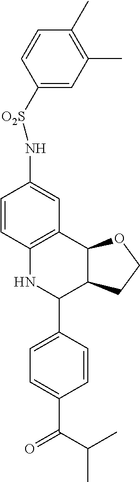 [SMILES: Cc1ccc(S(=O)(=O)Nc2ccc3c(c2)[C@H]2OCC[C@H]2C(c2ccc(C(=O)C(C)C)cc2)N3)cc1C] |
| 69L163 | 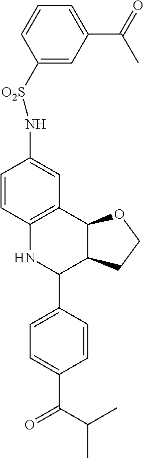 [SMILES: CC(=O)c1cccc(S(=O)(=O)Nc2ccc3c(c2)[C@H]2OCC[C@H]2C(c2ccc(C(=O)C(C)C)cc2)N3)c1] |
| 69L164 | 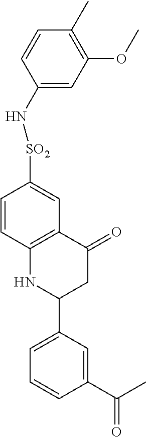 [SMILES: COc1cc(NS(=O)(=O)c2ccc3c(c2)C(=O)CC(c2cccc(C(C)=O)c2)N3)ccc1C] |
| 69L165 | 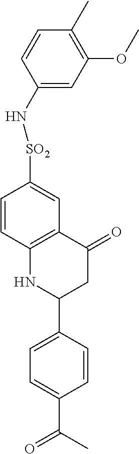 [SMILES: COc1cc(NS(=O)(=O)c2ccc3c(c2)C(=O)CC(c2ccc(C(C)=O)cc2)N3)ccc1C] |
| 69L166 | 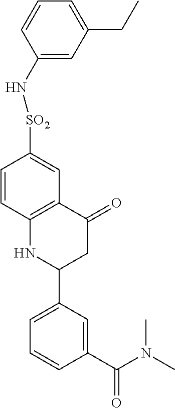 [SMILES: CCc1cccc(NS(=O)(=O)c2ccc3c(c2)C(=O)CC(c2cccc(C(=O)N(C)C)c2)N3)c1] |
| 69L167 | 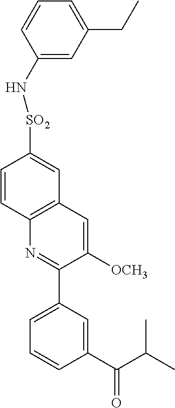 [SMILES: CCc1cccc(NS(=O)(=O)c2ccc3nc(-c4cccc(C(=O)C(C)C)c4)c(OC)cc3c2)c1] |
| 69L168 | 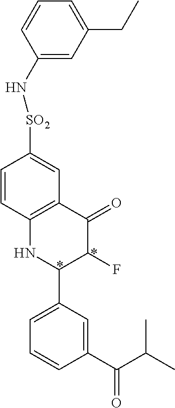 [SMILES: CCc1cccc(NS(=O)(=O)c2ccc3c(c2)C(=O)C(F)C(c2cccc(C(=O)C(C)C)c2)N3)c1] |

or a pharmaceutically acceptable salt thereof.

B. Pharmaceutical Compositions

In certain embodiment, this disclosure contemplates pharmaceutical compositions containing compounds disclosed herein in a pharmaceutically acceptable form. In certain embodiment, this disclosure contemplates pharmaceutical compositions containing compounds disclosed herein in a pharmaceutically acceptable form and pharmaceutically acceptable excipient. In certain embodiments, this disclosure contemplates the production of a medicament containing compounds disclosed herein and uses for methods disclosed herein.

As used herein, a “pharmaceutically acceptable form” of a disclosed compound includes, but is not limited to, pharmaceutically acceptable salts, hydrates, solvates, isomers, prodrugs, and isotopically labeled derivatives of disclosed compounds. In one embodiment, a “pharmaceutically acceptable form” includes, but is not limited to, pharmaceutically acceptable salts of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, naphthalene-m,n-bissulfonates, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, naphthalene-m,n-bissulfonic acids and the like.

Pharmaceutically acceptable salts derived from appropriate bases include alkali metal, alkaline earth metal, ammonium and quaternary ammonium, e.g., N⁺(R)₄, salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, about 3 or about 4, solvent or water molecules. It will be understood that the term “compound” as used herein encompasses the compound and solvates of the compound, as well as mixtures thereof.

Pharmaceutical compositions typically contain an effective amount of compounds and a suitable pharmaceutically acceptable carrier. The preparations can be prepared in a manner known per se, which usually involves mixing the compounds according to the disclosure with the one or more pharmaceutically acceptable carriers, and, if desired, in combination with other pharmaceutical active compounds, when necessary under aseptic conditions.

In certain embodiments, the disclosure relates to pharmaceutical compositions containing compounds disclosed herein and a pharmaceutically acceptable excipient. In certain embodiments, the composition is a pill, tablet, gel, granule, or in a capsule or the composition is an aqueous phosphate buffer, e.g., isotonic solution with a pH between 6 and 8. In certain embodiments, the pharmaceutically acceptable excipient is selected from a filler, glidant, binder, disintegrant, lubricant, and saccharide.

Compositions suitable for parenteral injection may contain physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil and viscoleo) and injectable organic esters such as ethyl oleate.

Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the compounds may be admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Liquid dosage forms for oral administration include pharmaceutically acceptable emulsions, solutions, suspensions, syrups, and elixirs. In addition to the compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, viscoleo, castor oil and sesame oil, glycerol, tetrahydrofurfuryl alcohol, polyethyleneglycols and fatty acid esters of sorbitan or mixtures of these substances, and the like.

In certain embodiments, production processes are contemplated which two components, compounds disclosed herein and a pharmaceutical carrier, are provided already in a combined dry form ready to be reconstituted together. In other embodiments, it is contemplated that compounds disclosed herein and a pharmaceutical carrier are admixed to provide a pharmaceutical composition.

Providing a pharmaceutic composition is possible in a one-step process, simply by adding a suitable pharmaceutically acceptable diluent to the composition in a container. In certain embodiments, the container is preferably a syringe for administering the reconstituted pharmaceutical composition after contact with the diluent. In certain embodiments, the coated compounds can be filled into a syringe, and the syringe can then be closed with the stopper. A diluent is used in an amount to achieve the desired end-concentration. The pharmaceutical composition may contain other useful component, such as ions, buffers, excipients, stabilizers, etc.

A “dry” pharmaceutical composition typically has only a residual content of moisture, which may approximately correspond to the moisture content of comparable commercial products, for example, has about 12% moisture as a dry product. Usually, the dry pharmaceutical composition according to the present invention has a residual moisture content preferably below 10% moisture, more preferred below 5% moisture, especially below 1% moisture. The pharmaceutical composition can also have lower moisture content, e.g. 0.1% or even below. In certain embodiments, the pharmaceutical composition is provided in dry in order to prevent degradation and enable storage stability.

A container can be any container suitable for housing (and storing) pharmaceutically compositions such as syringes, vials, tubes, etc. The pharmaceutical composition may then preferably be applied via specific needles of the syringe or via suitable catheters. A typical diluent comprises water for injection, and NaCl (preferably 50 to 150 mM, especially 110 mM), CaCl₂ (preferably 10 to 80 mM, especially 40 mM), sodium acetate (preferably 0 to 50 mM, especially 20 mM) and mannitol (preferably up to 10% w/w, especially 2% w/w). Preferably, the diluent can also include a buffer or buffer system so as to buffer the pH of the reconstituted dry composition, preferably at a pH of 6.2 to 7.5, especially at pH of 6.9 to 7.1.

In certain embodiments, the diluent is provided in a separate container. This can preferably be a syringe. The diluent in the syringe can then easily be applied to the container for reconstitution of the dry compositions. If the container is also a syringe, both syringes can be finished together in a pack. It is therefore preferred to provide the dry compositions in a syringe, which is finished with a diluent syringe with a pharmaceutically acceptable diluent for reconstituting, said dry and stable composition.

In certain embodiments, this disclosure contemplates a kit containing a pharmaceutical composition disclosed herein and a container with a suitable diluent. Further components of the kit may be instructions for use, administration means, such as syringes, catheters, brushes, etc. (if the compositions are not already provided in the administration means) or other components necessary for use in medical (surgical) practice, such as substitute needles or catheters, extra vials or further wound cover means. In certain embodiments, the kit contains a syringe housing the dry and stable hemostatic composition and a syringe containing the diluent (or provided to take up the diluent from another diluent container).

### Examples

### Molecular Modeling to Synthesize MX69 Analogs

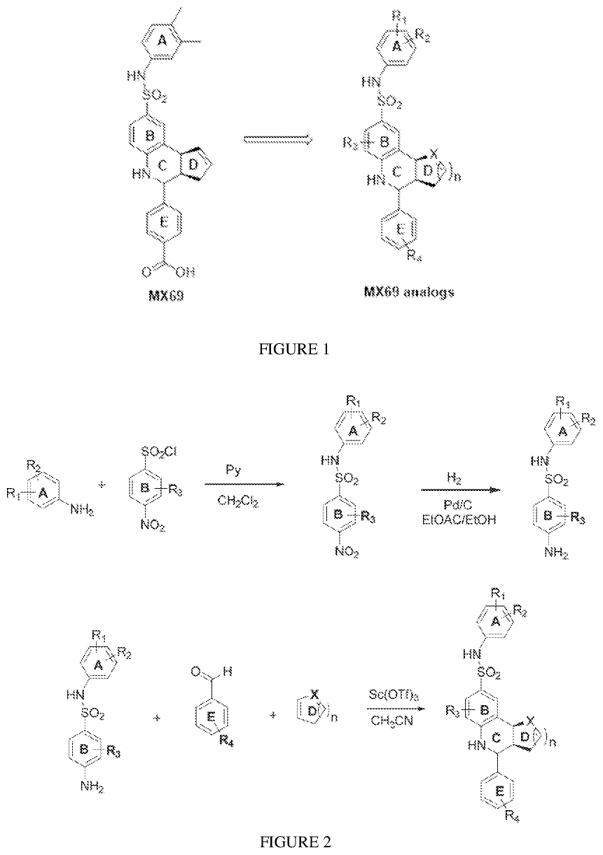

MX69 was selected for further drug development (Gu et al. Cancer Cell. 2016, 30(4):623-636). MX69 is a protein-binding compound that specifically binds to the RING domain of MDM2 and has minimal toxicity on normal cells/tissues (which typically do not express MDM2). It is desirable to identify improved anticancer potency with better PK/PD profiles. FIGS. 1 and 2 illustrate compounds and general methods of preparations.

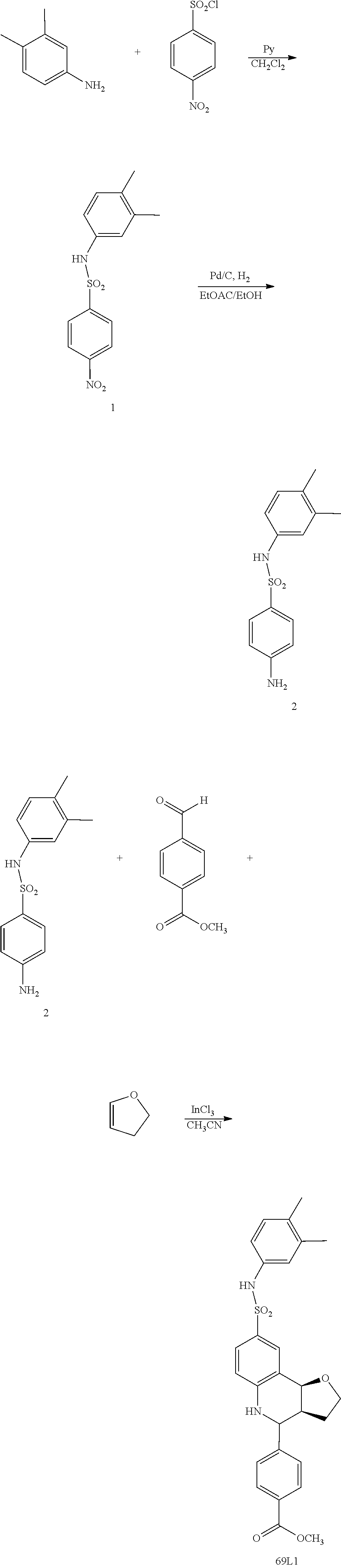

### Preparation of N-(3,4-dimethylphenyl)-4-nitrobenzenesulfonamide (1)

4-Nitrobenzenesulfonyl chloride (5.89 g, 26.58 mmol) and 3,4-Dimethylaniline (3.54 g, 29.23 mmol) were dissolved in anhydrous methylene chloride (100 mL) at room temperature under argon. Pyridine (2.31 g, 29.23 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂) to afford product as a pale yellow solid (8.07 g, 87% yield). ¹H NMR (400 MHz, DMSO-d₆) δ 10.37 (s, 1H); 8.36 (d, J=8.0 Hz, 2H), 7.96 (d, J=8.0 Hz, 2H), 6.99 (d, J=8.0 Hz, 1H), 6.88 (s, 1H), 6.80 (d, J=8.0 Hz, 1H), 2.11 (s, 3H), 2.09 (s, 3H).

### Preparation of 4-amino-N-(3,4-dimethylphenyl)benzenesulfonamide (2)

Compound N-(3,4-dimethylphenyl)-4-nitrobenzenesulfonamide (I) (5.00 g, 16.32 mmol) and Pd/C (10% Pd base, 1 g) were mixed together in MeOH (50 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (3.95 g, 87.6% yield). ¹H NMR (400 MHz, DMSO-d₆) a 9.60 (s, 1H) 7.37 (d, J=8.0 Hz, 2H), 6.93 (d, J=8.0 Hz, 1H), 6.84 (s, 1H), 6.80 (d, J=8.0 Hz, 1H), 6.52 (d, J=8.0 Hz, 2H), 5.91 (s, 2H), 109 (s, 3H), 108 (s, 3H).

### Preparation of methyl 8-(N-(3,4-dimethylphenyl)sulfomoyl)-2,3,3a,4,5,9b-hexahydrofuro[3,2-c]quinolin-4-yl)benzoate (69L1)

Compound 2 (0.50 g, 1.81 mmol), Methyl 4-formyllbenzaldehyde (0.30 g, 1.81 mmol) and InCl₃ (80 mg, 0.36 mmol) were mixed together and dissolved in anhydrous CH₃CN (10 mL) at room temperature under argon. The reaction mixture was stirred for one hour at room temperature. 2,3-Dihydrofuran (0.25 g, 3.62 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=19/1 v/v) to give a white solid product (0.61 g, 68.5% yield). Cis-69L1: ¹H NMR (400 MHz, CDCl₃) δ 8.09 (d, J=8.4 Hz, 2H), 7.81 (d, J=2.0 Hz, 1H), 7.51 (d, J=8.4 Hz, 2H), 7.45 (dd, J₁=8.4 Hz, J₂=2.0 Hz, 1H), 7.01 (d, J=8.0 Hz, 1H), 6.90 (d, J=2 Hz, 1H), 6.81 (dd, J₁=8.0 Hz, J₂=2.0 Hz, 1H), 6.56 (d, J=8.4 Hz, 1H), 6.27 (s, 1H), 5.20 (d, J=7.6 Hz, 1H), 4.88 (d, J=2.8 Hz, 1H), 4.30 (s, 1H), 3.95 (s, 3H), 3.77-3.72 (m, 1H), 3.69-3.67 (m, 1H), 2.80-2.74 (m, 1H), 2.21 (s, 3H), 2.20 (s, 3H), 2.09-1.97 (m, 1H), 1.51-1.47 (m, 1H). HRMS (ESI) calcd for C₁₈H₁₄F₃N₃O₃ 378.1066 [M+H]⁺, found 378.1078.

Trans-69L1: ¹H NMR (400 MHz, CDCl₃) δ 8.08 (d, J=8.4 Hz, 2H), 7.88 (d, J=2.0 Hz, 1H), 7.49 (d, J=8.4 Hz, 2H), 7.44 (dd, J₁=8.4 Hz, J₂=2.0 Hz, 1H), 6.98 (d, J=8.0 Hz, 1H), 6.89 (d, J=2 Hz, 1H), 6.79 (dd, J₁=8.0 Hz, J₂=2.0 Hz, 1H), 6.57 (d, J=8.4 Hz, 1H), 6.45 (s, 1H), 4.64 (s, 1H), 4.53 (d, J=4.8 Hz, 1H), 4.07-4.01 (m, 1H), 3.93 (s, 3H), 3.88-3.84 (m, 1H), 2.42-2.39 (m, 1H), 2.19 (s, 6H), 2.07-2.02 (m, 1H), 1.73-1.69 (m, 1H). HRMS (ESI) calcd for C₁₈H₁₄F₃N₃O₃ 378.1066 [M+H]⁺, found 378.1078.

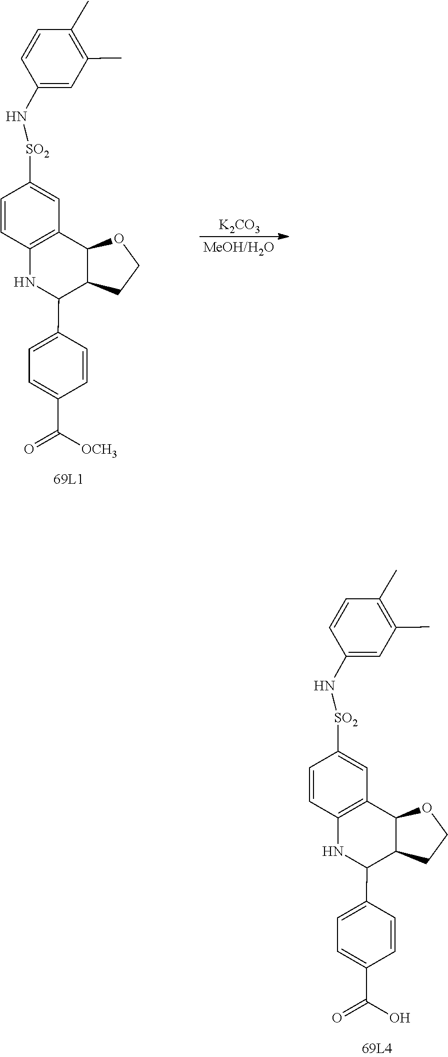 [SMILES: COC(=O)c1ccc(C2Nc3ccc(S(=O)(=O)Nc4ccc(C)c(C)c4)cc3[C@H]3OCC[C@@H]23)cc1 ; "K2CO3" ; Cc1ccc(NS(=O)(=O)c2ccc3c(c2)[C@H]2OCC[C@H]2C(c2ccc(C(=O)O)cc2)N3)cc1C]

Cis-69L4: ¹H NMR (400 MHz, DMSO-d⁶) δ 12.95 (s, 1H), 9.71 (s, 1H), 7.96 (d, J=8.4 Hz, 2H), 7.57-7.54 (m, 3H), 7.32 (dd, J₁=8.4 Hz, J₂=2.0 Hz, 1H), 6.96 (d, J=8.4 Hz, 1H), 6.87 (d, J=2 Hz, 1H), 6.82 (dd, J₁=8.0 Hz, J₂=2.0 Hz, 1H), 6.72 (d, J=8.4 Hz, 1H), 5.11 (d, J=7.6 Hz, 1H), 4.84 (d, J=3.2 Hz, 1H), 3.64-3.57 (m, 1H), 3.53-3.47 (m, 1H), 2.69-2.67 (m, 1H), 2.11 (s, 3H), 2.10 (s, 3H), 1.80-1.74 (m, 1H), 1.33-1.30 (m, 1H).

Trans-69L4: ¹H NMR (400 MHz, DMSO-d⁶) δ 12.97 (s, 1H), 9.74 (s, 1H), 7.96 (d, J=8.4 Hz, 2H), 7.60 (s, 1H), 7.57 (d, J=2.0 Hz, 1H), 7.38 (dd, J₁=8.4 Hz, J₂=2.0 Hz, 1H), 7.16 (s, 1H), 6.96 (d, J=8.4 Hz, 1H), 6.87 (d, J=2 Hz, 1H), 6.82 (dd, J₁=8.0 Hz, J₂=2.0 Hz, 1H), 6.73 (d, J=8.4 Hz, 1H), 4.43 (d, J=4.8 Hz, 1H), 3.90-3.87 (m, 1H), 3.79 (d, J=10.4 Hz, 1H), 3.74-3.69 (m, 1H), 2.31-2.26 (m, 1H), 2.11 (s, 3H), 2.10 (s, 3H), 1.97-1.93 (m, 1H), 1.56-1.52 (m, 1H).

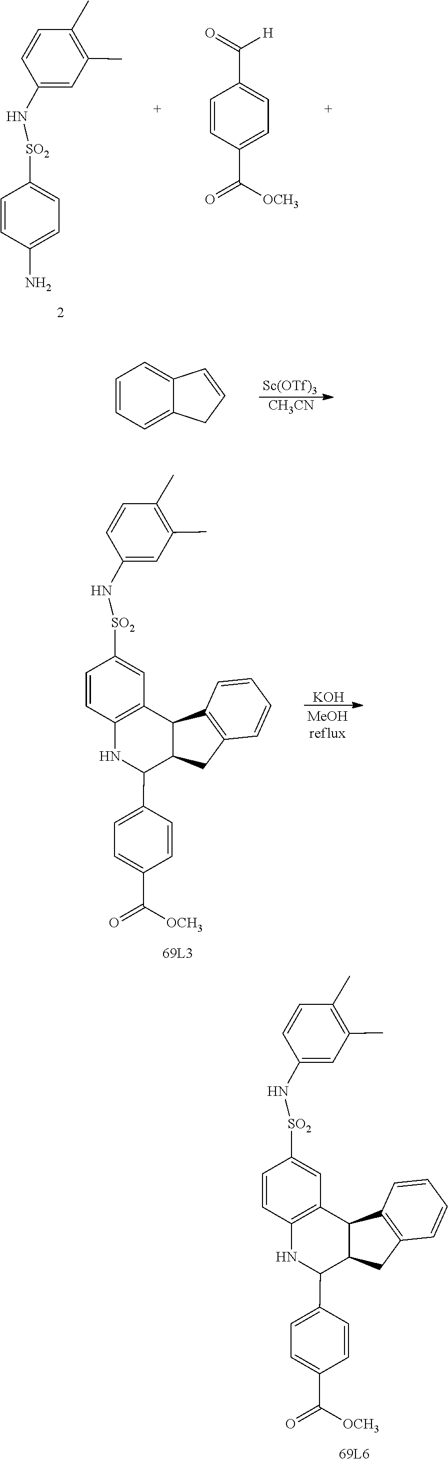

69L3: ¹H NMR (400 MHz, CDCl₃) δ 8.10 (d, J=7.6 Hz, 2H), 7.74 (s, 1H), 7.53 (d, J=7.6 Hz, 2H), 7.42 (s, 1H), 7.35 (d, J=8.4 Hz, 1H), 7.15-7.14 (m, 2H), 7.05 (s, 1H), 6.91 (d, J=7.2 Hz, 1H), 6.77 (s, 1H), 6.72 (d, J=8.0 Hz, 1H), 6.58 (d, J=8.4 Hz, 1H), 6.21 (s, 1H), 4.87 (s, 1H), 4.50 (d, J=4.8 Hz, 1H), 4.36 (s, 1H), 3.97 (s, 3H), 3.16-3.11 (m, 2H), 2.38-2.30 (m, 1H), 2.19 (s, 3H), 2.13 (s, 3H).

69L6: ¹H NMR (400 MHz, DMSO-d⁶) δ 12.94 (s, 1H), 9.77 (s, 1H), 7.97 (d, J=7.6 Hz, 2H), 7.65 (s, 1H), 7.58 (d, J=7.2 Hz, 2H), 7.50 (d, J=7.2 Hz, 1H), 7.26 (d, J=8.4 Hz, 1H), 7.19-7.15 (m, 1H), 7.13-7.09 (m, 1H), 7.03 (d, J=7.6 Hz, 1H), 6.89 (d, J=8.0 Hz, 1H), 6.81 (s, 3H), 4.81 (s, 1H), 4.50 (d, J=8.0 Hz, 1H), 3.12-3.10 (m, 1H), 2.95-2.88 (m, 1H), 2.20-2.14 (m, 1H), 2.07 (s, 3H), 2.05 (s, 3H).

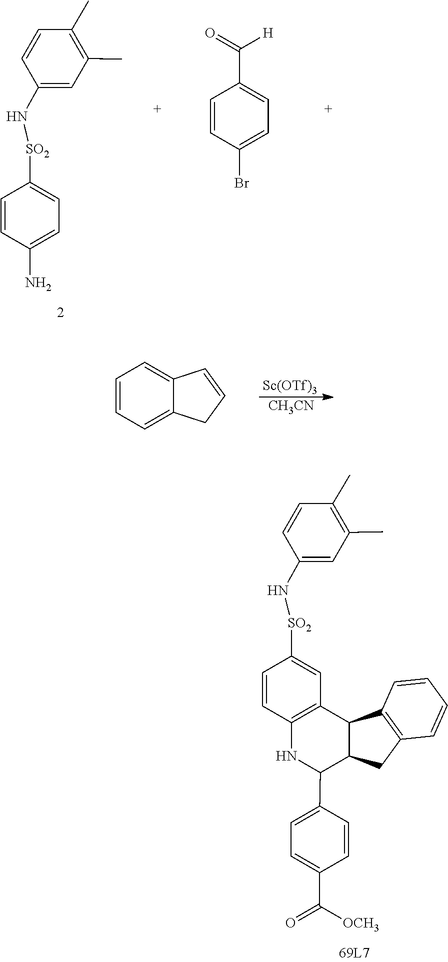

69L7: ¹H NMR (400 MHz, CDCl₃) δ 7.77 (s, 1H), 7.54 (d, J=7.2 Hz, 2H), 7.47-7.43 (m, 1H), 7.38-7.32 (m, 3H), 7.12 (d, J=3.6 Hz, 2H), 7.05 (s, 1H), 6.90 (d, J=8.0 Hz, 1H), 6.78-6.75 (m, 2H), 6.67 (s, 1H), 6.54 (d, J=8.4 Hz, 1H), 4.74 (s, 1H), 4.47 (d, J=6.0 Hz, 1H), 4.33 (s, 1H), 3.97 (s, 3H), 3.15-3.10 (m, 2H), 2.41-2.33 (m, 1H), 2.17 (s, 3H), 2.12 (s, 3H).

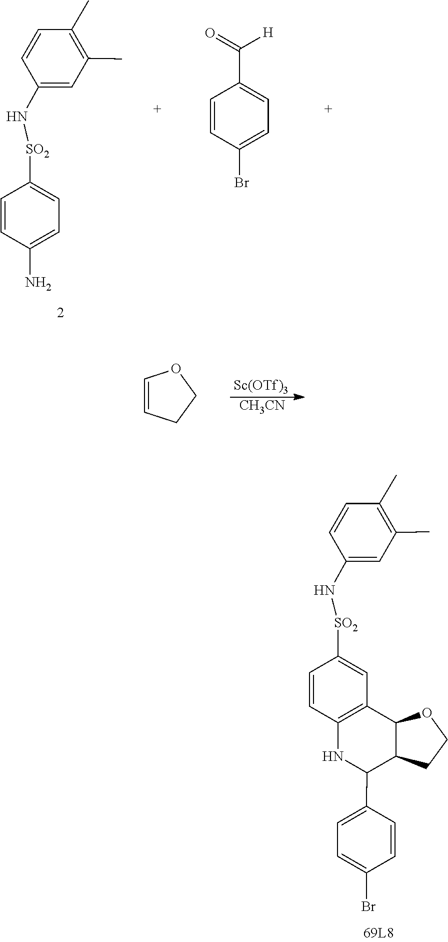

Cis-69L8: ¹H NMR (400 MHz, CDCl₃) δ 7.80 (d, J=2.0 Hz, 1H), 7.53 (d, J=8.4 Hz, 2H), 7.44 (dd, J₁=8.4 Hz, J₂=2.4 Hz, 1H), 7.31 (d, J=8.0 Hz, 2H), 6.98 (d, J=8.0 Hz, 1H), 6.89 (d, J=2.0 Hz, 1H), 6.80 (d, J=8.0 Hz, 1H), 6.56 (d, J=2.0 Hz, 1H), 5.18 (d, J=7.6 Hz, 1H), 4.77 (d, J=3.2 Hz, 1H), 4.31 (s, 1H), 3.95 (s, 3H), 3.75-3.71 (m, 1H), 3.69-3.65 (m, 1H), 2.72-2.70 (m, 1H), 2.20 (s, 3H), 2.19 (s, 3H), 2.03-1.97 (m, 1H), 1.55-1.51 (m, 1H).

Trans-69L8: ¹H NMR (400 MHz, CDCl₃) δ 7.88 (d, J=2.4 Hz, 1H), 7.54 (d, J=8.0 Hz, 2H),), 7.43 (dd, J₁=8.4 Hz, J₂=2.4 Hz, 1H), 7.32 (d, J=8.0 Hz, 2H), 6.97 (d, J=8.0 Hz, 1H), 6.88 (d, J=2 Hz, 1H), 6.80 (d, J=8.0 Hz, 1H), 6.57 (s, 1H), 6.53 (d, J=8.0 Hz, 1H), 4.61 (s, 1H), 4.52 (d, J=4.8 Hz, 1H), 4.04-4.01 (m, 1H), 3.84-3.81 (m, 2H), 2.33-2.32 (m, 1H), 2.20 (s, 3H), 2.19 (s, 3H), 2.03-1.97 (m, 1H), 1.61-1.58 (m, 1H).

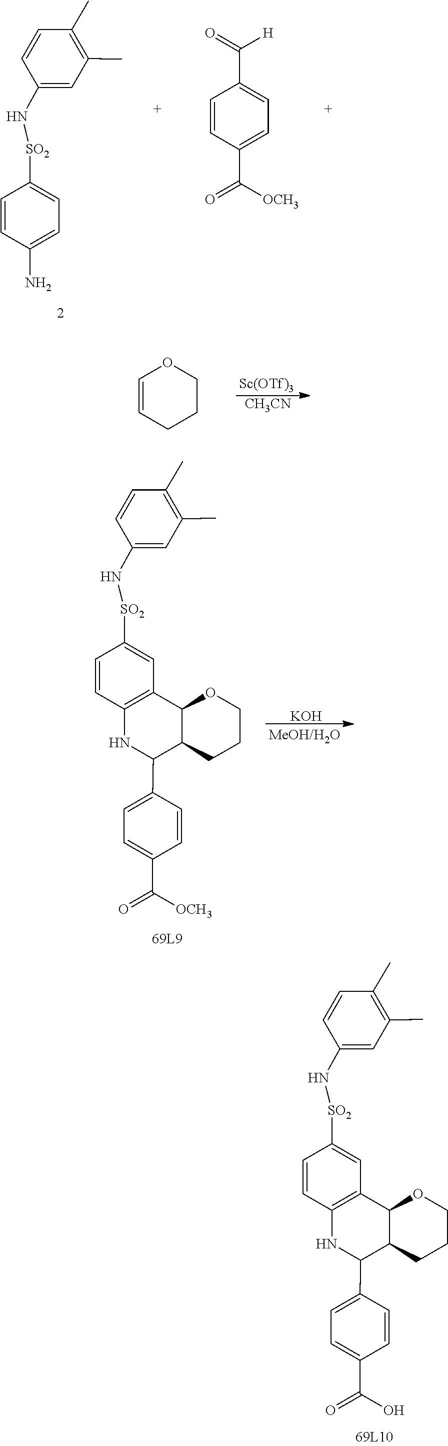

69L9 (mixtures of cis and trans isomers): ¹H NMR (400 MHz, CDCl₃) δ 8.07 (d, J=8.4 Hz, 2H), 7.78 (d, J=2.0 Hz, 0.3H), 7.72 (d, J=2.0 Hz, 0.7H), 7.53 (dd, J₁=8.4 Hz, J₂=2.0 Hz, 0.3H), 7.44 (d, J=8.0 Hz, 2H), 7.41 (d, J=8.0 Hz, 0.7H), 7.01-6.98 (m, 1H), 6.90 (s, 1H), 6.84-6.80 (m, 1H), 6.59 (d, J=8.8 Hz, 0.3H), 6.48 (d, J=8.8 Hz, 0.7H), 6.41 (s, 0.3H), 6.35 (s, 0.7H), 5.23 (d, J=5.6 Hz, 0.3H), 4.80 (s, 0.7H), 4.78 (s, 0.3H), 4.57 (s, 0.7H), 4.37-4.36 (m, 1H), 4.04-4.02 (m, 0.7H), 3.95 (s, 3H), 3.71-3.69 (m, 1H), 3.45-3.40 (m, 0.7H), 3.07-3.02 (m, 0.3H), 2.20 (s, 3H), 2.19 (s, 3H), 2.08-2.05 (m, 0.7H), 1.85-1.82 (m, 1H), 1.74-1.62 (m, 1H), 1.57-1.41 (m, 2H).

69L10 (mixtures of cis and trans isomers): ¹H NMR (400 MHz, DMSO-d⁶) δ 9.67-9.64 (m, 1H), 7.96-7.93 (m, 2H), 7.52-7.46 (m, 3H), 7.37-7.34 (m, 1H), 7.12 (s, 0.7H), 6.97-6.94 (m, 1H), 6.91 (s, 0.3H), 6.86-6.81 (m, 2H), 6.71 (d, J=8.4 Hz, 0.3H), 6.62 (d, J=8.4 Hz, 0.7H), 5.18 (d, J=5.6 Hz, 0.3H), 4.81 (d, J=2.4 Hz, 0.3H), 4.61 (d, J=9.6 Hz, 0.7H), 4.31 (d, J=2.8 Hz, 0.7H), 3.78-3.76 (m, 1H), 3.62-3.57 (m, 1H), 2.95-2.89 (m, 0.3H), 2.11 (s, 3H), 2.09 (s, 3H), 1.97-1.94 (m, 1H), 1.78-1.62 (m, 2H), 1.35-1.32 (m, 1H), 1.21-1.17 (m, 1H).

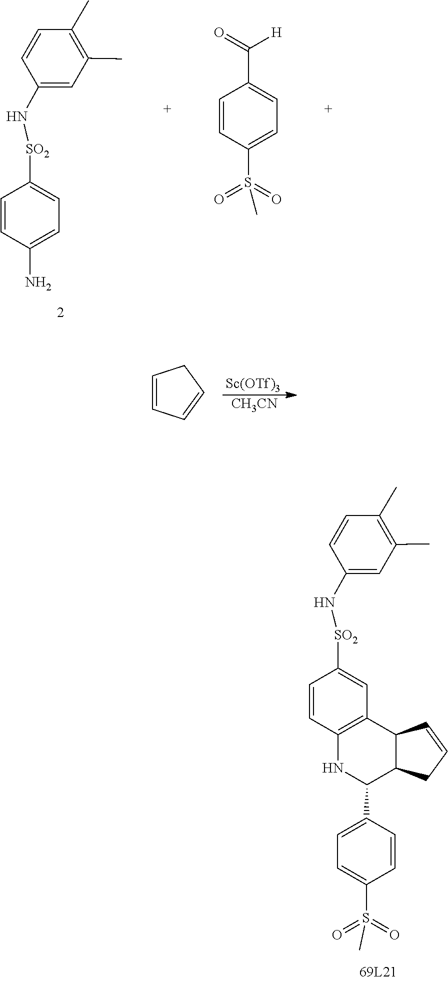

69L21: ¹H NMR (400 MHz, DMSO-d⁶) δ 9.67 (s, 1H), 7.94 (d, J=8.4 Hz, 2H), 7.69 (d, J=8.0 Hz, 2H), 7.35 (s, 1H), 7.26 (d, J=8.4 Hz, 1H), 6.96 (d, J=8.0 Hz, 1H), 6.86 (s, 1H), 6.82 (d, J=8.0 Hz, 1H), 6.75 (d, J=8.4 Hz, 1H), 6.58 (s, 1H), 5.77 (s, 1H), 5.58 (d, J=4.2 Hz, 1H), 4.74 (d, J=2.4 Hz, 1H), 4.04 (d, J=4.2 Hz, 1H), 3.22 (s, 3H), 2.99-2.94 (m, 1H), 2.33-2.27 (m, 1H), 2.11 (s, 6H), 1.62-1.57 (m, 1H).

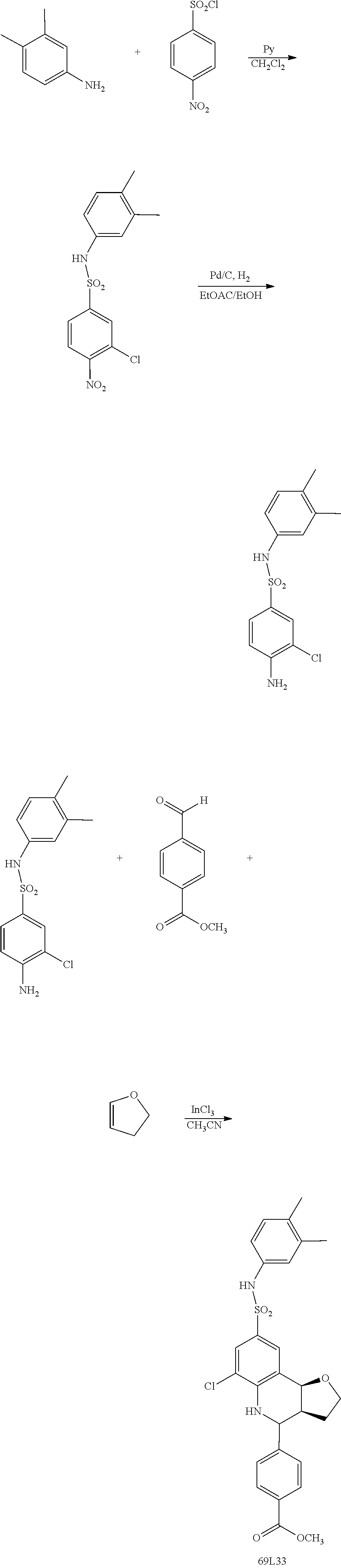

### Preparation of methyl 6-chloro-8-(N-(3,4-dimethylphenyl)sulfomoyl)-2,3,3a,4,5,9b-hexahydrofuro[3,2-c]quinolin-4-yl)benzoate (69L33)

Compound 4-amino-3-chloro-N-(3,4-dimethylphenyl)benzenesulfonamide (0.27 g, 0.87 mmol), Methyl 4-formyllbenzaldehyde (0.14 g, 0.87 mmol) and InCl₃ (38 mg, 0.17 mmol) were mixed together and dissolved in anhydrous CH₃CN (10 mL) at room temperature under argon. The reaction mixture was stirred for one hour at room temperature. 2,3-Dihydrofuran (0.12 g, 1.74 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=19/1 v/v) to give a white solid product (0.38 g, 83% yield). ¹H NMR (400 MHz, CDCl₃) δ 8.03-8.01 (m, 2H), 7.64 (s, 1H), 7.47 (m, 1H), 7.29 (d, J=8.4 Hz, 1H), 7.22 (d, J=8.0 Hz, 1H), 7.02-6.96 (m, 1H), 6.90-6.72 (m, 3H), 6.13, 5.99 (s, 1H), 5.15 (d, J=7.6 Hz, 0.37H), 4.49 (d, J=4.8 Hz, 1H), 4.00-3.97 (m, 1H), 3.95 (s, 3H), 3.86-3.84 (m, 0.73H), 3.71-3.65 (m, 1.35H), 2.64-2.72 (m, 0.39H), 2.31-2.27 (m, 0.68H), 2.23-2.09 (m, 6H), 2.07-1.97 (m, 0.77H), 1.74-1.70 (m, 1.67H), 1.45-1.36 (m, 0.4H).

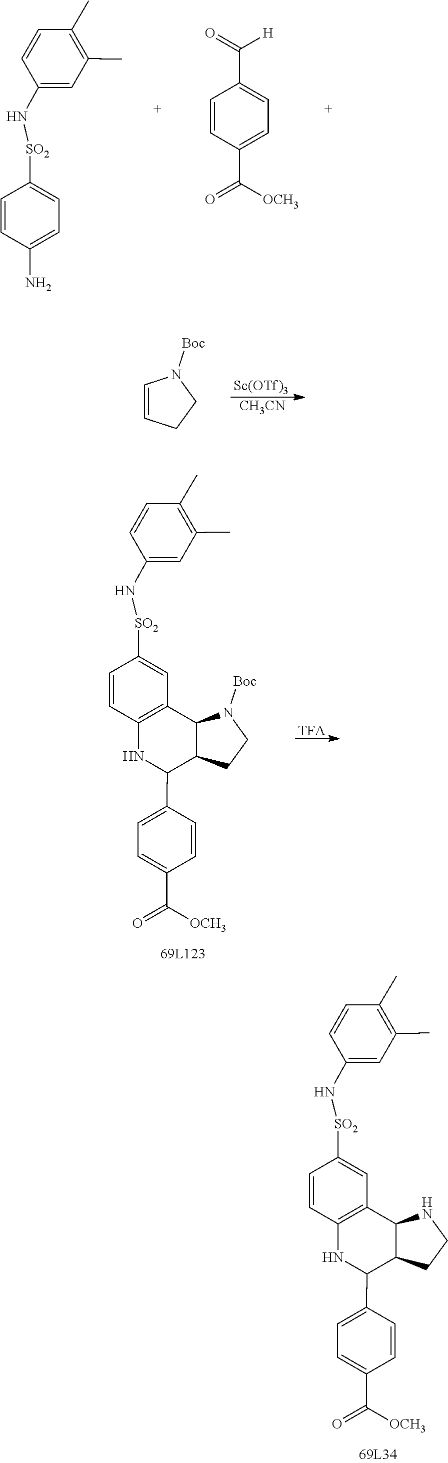

Cis-69L134: ¹H NMR (400 MHz, CDCl₃) δ 8.04 (d, J=8.0 Hz, 2H), 7.50 (d, J=8.0 Hz, 2H), 7.32 (d, J=8.0 Hz, 1H), 6.99-6.97 (m, 2H), 6.86 (d, J=8.0 Hz, 1H), 6.53 (d, J=8.4 Hz, 1H), 4.96 (s, 1H), 4.86 (s, 1H), 3.94 (s, 3H), 3.50 (s, 1H), 3.02 (s, 2H), 2.87 (s, 1H), 2.19 (s, 6H), 1.96-1.91 (m, 1H), 1.48-1.47 (m, 1H).

Trans-69L34: ¹H NMR (400 MHz, CDCl₃) δ 8.06 (d, J=8.0 Hz, 2H), 7.92 (s, 1H), 7.48 (d, J=8.0 Hz, 2H), 7.40 (d, J=8.4 Hz, 1H), 6.96 (d, J=8.4 Hz, 1H), 6.92 (s, 1H), 6.81 (d, J=8.0 Hz, 1H), 6.54 (d, J=8.4 Hz, 1H), 4.63 (s, 1H), 4.10 (d, J=2.0 Hz, 1H), 3.98 (s, 1H), 3.95 (s, 3H), 3.25-3.19 (m, 1H), 3.08-3.02 (m, 2H), 2.42-2.36 (m, 1H), 2.18 (s, 6H), 1.98-1.89 (m, 1H), 1.64-1.58 (m, 1H).

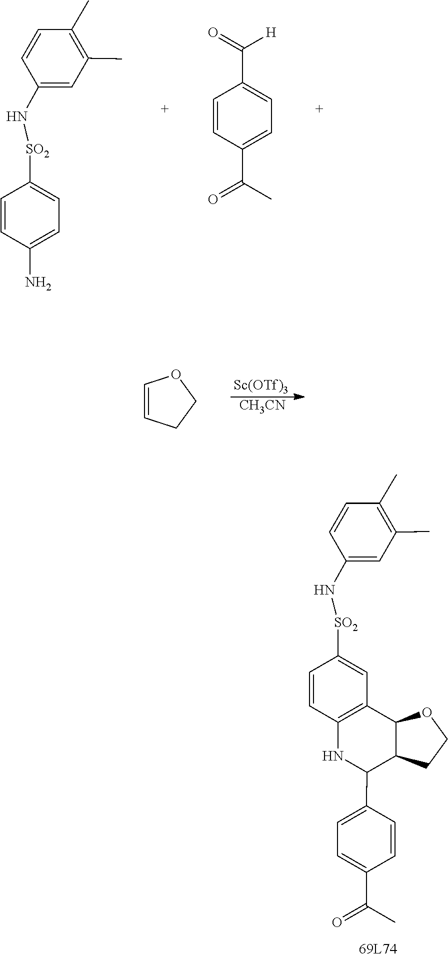

69L74: ¹H NMR (400 MHz, CDCl₃) δ 8.02-7.99 (m, 2H), 7.89 (d, J=2.0 Hz, 0.5H), 7.81 (d, J=2.0 Hz, 0.5H), 7.54-7.51 (m, 2H), 7.47-7.43 (m, 1H), 7.00 (s, 0.5H), 6.98 (s, 0.5H), 6.90 (s, 1H), 6.82-6.80 (m, 1H), 6.57 (dd, J₁=8.4 Hz, J₂=2.4 Hz, 1H), 6.39 (s, 0.5H), 6.35 (s, 0.5H), 5.20 (d, J=7.2 Hz, 0.5H), 4.88 (d, J=3.2 Hz, 0.5H), 4.61 (s, 0.5H), 4.54 (d, J=4.8 Hz, 0.5H), 4.34 (s, 0.5H), 4.17-4.01 (m, 1H), 3.93-3.85 (m, 1H), 3.77-3.63 (m, 1H), 2.78-2.76 (m, 0.5H), 2.64 (s, 3H), 2.45-2.39 (m, 0.5H), 2.19 (s, 6H), 2.08-2.00 (m, 0.5H), 1.75-1.70 (m, 0.5H), 1.54-1.47 (m, 0.5H).

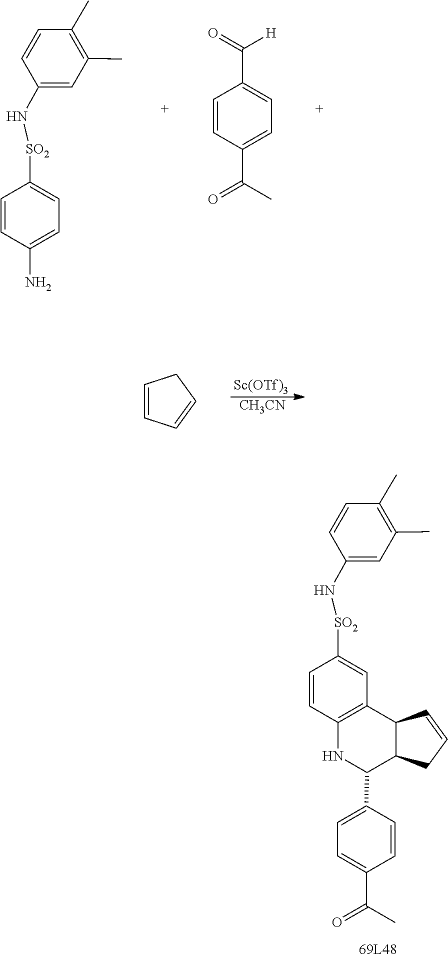

69L48: ¹H NMR (400 MHz, CDCl₃) δ 7.97 (d, J=8.4 Hz, 2H), 7.48 (d, J=8.4 Hz, 2H), 7.37 (dd, J₁=8.4 Hz, J₂=2.0 Hz, 1H), 7.35 (s, 1H), 6.99 (d, J=8.0 Hz, 1H), 6.84 (d, J=2 Hz, 1H), 6.79 (dd, J₁=8.0 Hz, J₂=2.0 Hz, 1H), 6.59 (d, J=8.4 Hz, 1H), 6.26 (s, 1H), 5.63-5.59 (m, 1H), 4.73 (d, J=3.2 Hz, 1H), 4.18 (s, 1H), 4.03 (d, J=8.8 Hz, 1H), 3.04-2.97 (m, 1H), 2.62 (s, 3H), 2.52-2.46 (m, 1H), 2.19 (s, 6H), 1.78-1.72 (m, 1H).

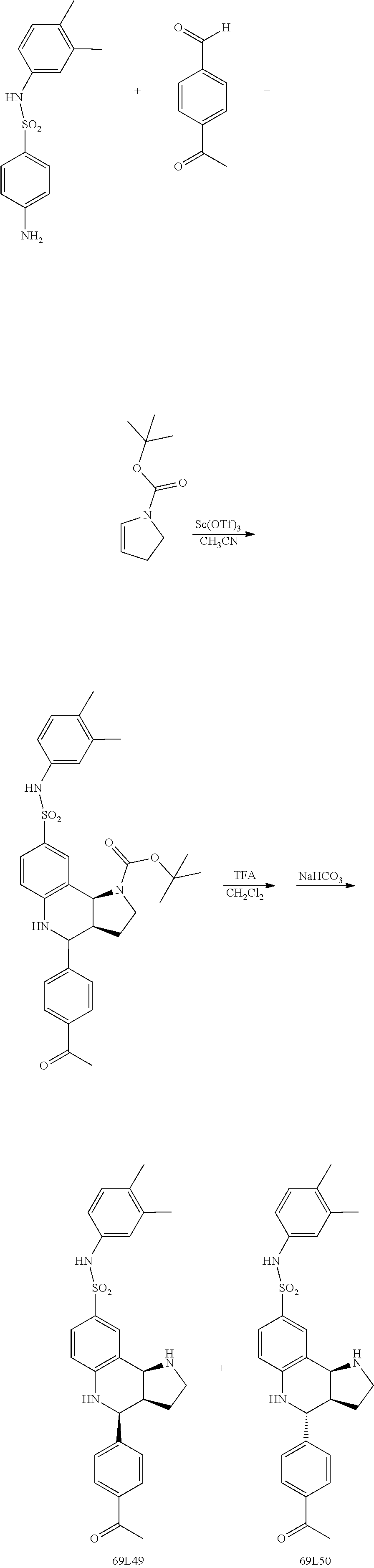

69L49: ¹H NMR (400 MHz, CDCl₃) δ 8.14 (s, 1H), 7.94 (d, J=8.0 Hz, 2H), 7.51 (d, J=8.0 Hz, 2H), 7.24 (s, 1H), 6.97-6.95 (m, 2H), 6.86 (d, J=8.0 Hz, 1H), 6.51 (d, J=8.4 Hz, 1H), 5.15 (s, 1H), 4.87 (s, 1H), 4.45 (s, 1H), 3.48 (s, 3H), 3.16 (s, 2H), 2.96 (s, 1H), 2.60 (s, 3H), 2.17 (s, 6H), 2.06-1.96 (m, 1H), 1.54-1.52 (m, 1H).

69L50: ¹H NMR (400 MHz, CDCl₃) δ 8.01 (d, J=1.2 Hz, 1H), 7.95 (d, J=8.4 Hz, 2H), 7.48 (d, J=8.0 Hz, 2H), 7.33 (dd, J₁=8.8 Hz, J₂=2.0 Hz, 1H), 6.92-6.90 (m, 2H), 6.80 (dd, J₁=8.0 Hz, J₂=2.0 Hz, 1H), 6.51 (d, J=8.8 Hz, 1H), 4.70 (s, 1H), 4.22 (d, J=6.0 Hz, 1H), 4.04 (d, J=10.8 Hz, 1H), 3.30-3.24 (m, 1H), 3.14-3.07 (m, 1H), 2.61 (s, 3H), 2.44-2.38 (m, 1H), 2.14 (s, 6H), 2.08-1.92 (m, 1H), 1.66-1.63 (m, 1H).

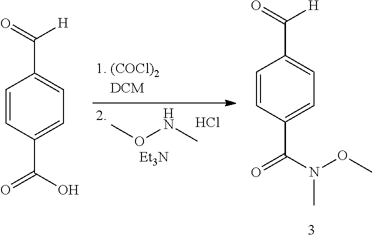

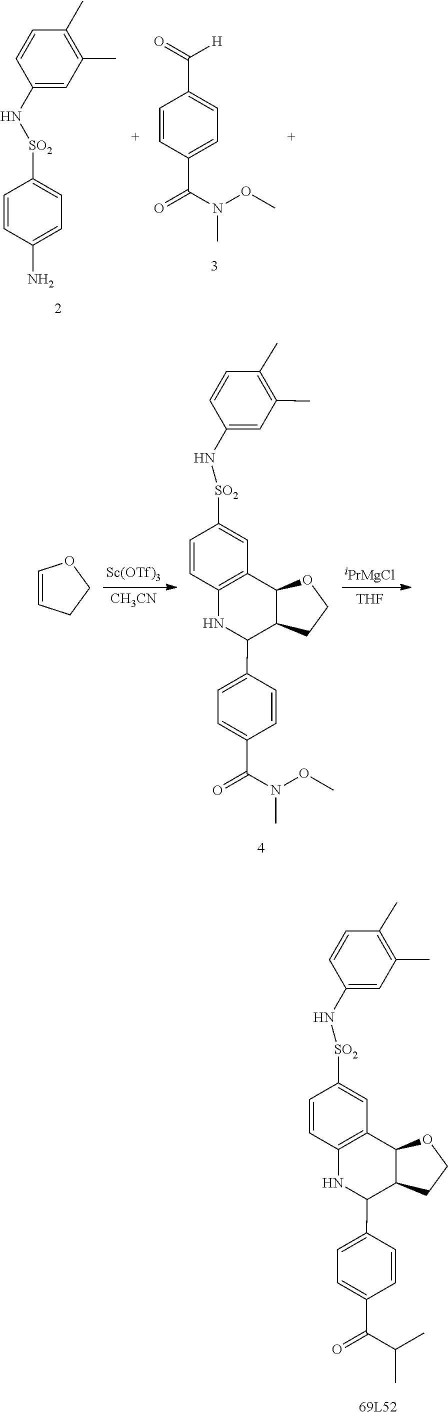

### Preparation of 4-formyl-N-methoxy-N-methylbenzamide (3)

4-Formylbenzoic acid (10.00 g, 66.61 mmol) was dissolved in anhydrous methylene chloride (100 mL) at room temperature under argon Oxalyl chloride (10.15 g, 79.93 mmol) was added dropwise via a syringe at room temperature. The reaction mixture was cooled to 0° C. and 0.5 mL of anhydrous DMF was added. The resulted mixture was stirred at room temperature for 4 hours. Then, N, O-dimethylhydroxyamine hydrochloride (9.75 g, 99.92 mmol) was added at room temperature. Triethylamine (20.22 g, 199.83 mmol) was added to the reaction mixture at 0° C. After stirred at room temperature overnight, the reaction was quenched by adding water. The organic layer was separated, dried over anhydrous MgSO₄, filtered and concentrated under reduced pressure. The yellow oil residue was subjected to flash column chromatography (silica gel, CH₂Cl₂/MeOH=19/1 v/v) to afford product as a pale yellow oil product (10.58 g, 82.2% yield). ¹H NMR (400 MHz, DMSO-d₆) δ 10.07 (s, 1H); 7.98 (d, J=8.0 Hz, 2H), 7.76 (d, J=8.0 Hz, 2H), 3.54 (s, 3H), 3.29 (s, 3H).

### Preparation of 8-N-(3,4-dimethylphenyl)-sulfonyl-2,3,3a,4,5,9b-hexahydrofuran[3,2-c]quinolin-4-yl)-N-methoxy-N-methylbenzamide (4)

Compound 4-amino-N-(3,4-dimethylphenyl)benzenesulfonamide (2) (3.76 g, 13.61 mmol), 4-formyl-N-methoxy-N-methylbenzamide (3) (2.63 g, 13.61 mmol), Sc(OTf)₃ (1.34 g, 2.72 mmol) and 4 Å molecular sieves (5 g) were mixed together and dissolved in anhydrous CH₃CN (40 mL) at room temperature under argon. The reaction mixture was stirred for one hour at room temperature. 2,3-Dihydrofuran (1.91 g, 27.22 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/MeOH=19/1 v/v) to give a white solid product (6.20 g, 87.3% yield). ¹H NMR (400 MHz, DMSO-d⁶) δ 9.75, 9.71 (s, 1H), 7.61 (d, J=8.0 Hz, 2H), 7.55-7.50 (m, 3H), 7.38 (dd, J₁=8.4 Hz, J₂=4.4 Hz, 0.38H), 7.32 (dd, J₁=8.4 Hz, J₂=2.4 Hz, 1H), 7.17 (s, 0.38H), 6.96 (d, J=8.0 Hz, 1H), 6.87 (d, J=2.0 Hz, 1H), 6.84-6.80 (m, 1H), 6.75-6.70 (m, 1H), 5.11 (d, J=7.6 Hz, 0.67H), 4.81 (d, J=2.8 Hz, 0.66H), 4.33 (d, J=28 Hz, 0.43H), 3.90-3.88 (m, 0.5H), 3.77-3.70 (m, 1H), 3.52 (s, 3H), 3.26 (s, 3H), 2.69-2.67 (s, 0.76H), 2.32-2.26 (m, 0.65H), 2.11-2.09 (m, 6H), 1.99-1.931 (m, 0.85H), 1.82-1.75 (m, 1H), 1.57-1.55 (m, 0.67H), 1.37-1.33 (m, 0.72H).

### Preparation of N-(3,4-dimethylphenyl)-4-(4-isobutyrylphenyl)-2,3,3a,4,5,9b-hexahydrofuran[3,2-c]quinolone-8-sulfonamide (69L52)

Compound 4-((3aS,9bS)-8-N-(3,4-dimethylphenyl)-sulfonyl-2,3,3a,4,5,9b-hexahydrofuran[3,2-c]quinolin-4-yl)-N-methoxy-N-methylbenzamide (4) (1.05 g, 2.01 mmol) was dissolved in 50 mL of anhydrous THF at room temperature under argon. Isopropyl magnesium chloride (3.32 mL of 2M THF solution, 6.64 mmol) was added via a syringe at room temperature under argon. The reaction solution was stirred at room temperature overnight. Then, the reaction was quenched by adding 100 mL of water. The resulted solution was extracted with ethyl acetate (3×50 mL). The organic layer was separated, dried over anhydrous MgSO₄, filtered and concentrated under reduced pressure. The crude solid was purified by flash column chromatography (silica gel, CH₂Cl₂/Acetone=19/1 v/v) to give a white solid product, 0.43 g (41.7% yield). Cis-trans mixture: ¹H NMR (400 MHz, DMSO-d⁶) δ 9.78, 9.75 (s, 1H), 7.99 (d, J=8.4 Hz, 2H), 7.62-7.55 (M, 3H), 7.39 (dd, J₁=8.4 Hz, J₂=20 Hz, 0.55H), 7.33 (dd, J₁=8.4 Hz, J₂=2 0 Hz, 0.45H), 7.18 (s, 0.55H), 6.96 (d, J=8.4 Hz, 1H), 6.89-6.86 (m, 1.451H), 6.84-6.80 (m, 1H), 6.74-6.70 (m, 1H), 5.11 (d, J=7.2 Hz, 0.45-1), 4.84 (d, J=2.8 Hz, 0.45H), 4.43 (d, J=4.8 Hz, 0.55H), 3.90-3.88 (m, 0.55H), 3.79 (d, J=10.8 Hz, 0.55H), 3.72-3.59 (m, 2H), 3.51-3.49 (m, 0.55H), 2.69-2.67 (m, 0.45H), 2.30-2.2.7 (m, 0.55H), 2.11 (s, 3H), 2.09 (s, 3H), 1.96-1.93 (m, 0.55H), 1.83-1.72 (m, 0.55H), 1.56-1.52 (In, 0.55H), 1.32-1.30 (m, 0.45H), 1.11 (d, J=6.8 Hz, 6H). HRMS (EST) calcd for C₂₉H₃₃N₂O₄S: 505.2161 [M+H]⁺, found 505.2160.

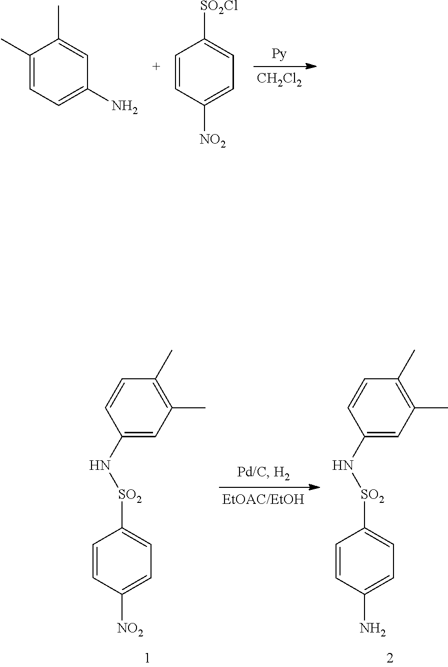

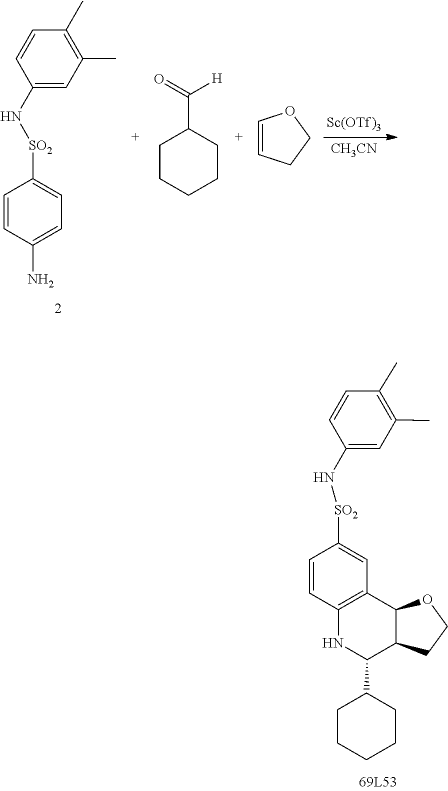

### Preparation of N-(3,4-dimethylphenyl)-4-nitrobenzenesulfonamide (1)

4-Nitrobenzenesulfonyl chloride (5.89 g, 26.58 mmol) and 3,4-Dimethylaniline (3.54 g, 29.23 mmol) were dissolved in anhydrous methylene chloride (100 mL) at room temperature under argon. Pyridine (2.31 g, 29.23 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂) to afford product as a pale yellow solid (8.07 g, 87% yield). ¹H NMR (400 MHz, DMSO-d₆) δ 10.37 (s, 1H); 8.36 (d, J=8.0 Hz, 2H), 7.96 (d, J=8.0 Hz, 2H), 6.99 (d, J=8.0 Hz, 1H), 6.88 (s, 1H), 6.80 (d, J=8.0 Hz, 1H), 2.11 (s, 3H), 2.09 (s, 3H).

### Preparation of 4-amino-N-(3,4-dimethylphenyl)benzenesulfonamide (2)

Compound N-(3,4-dimethylphenyl)₄-nitrobenzenesulfonamide (1) (5.00 g, 16.32 mmol) and Pd/C (10% Pd base, 1 g) were mixed together in MeOH (50 mL) at room temperature. Hydrogen gas was introduced via a H₂ balloon. The reaction mixture was stirred under H2 atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (3.95 g, 87.6% yield). ¹H NMR (400 MHz, DMSO-d⁶) δ 9.60 (s, 1H); 7.37 (d, J=8.0 Hz, 2H), 6.93 (d, J=8.0 Hz, 1H), 6.84 (s, 1H), 6.80 (d, J=8.0 Hz, 1H), 6.52 (d, J=8.0 Hz, 2H), 5.91 (s, 2H), 2.09 (s, 3H), 2.08 (s, 3H).

### Preparation of 4-cyclohexyl-N-(3,4-dimethylphenyl)-2,3,3a,4,5,9b-hexahydrofuran[3,2-c]quinolone-8-sulfonamine (69L53)

Compound 4-amino-N-(3,4-dimethylphenyl)benzenesulfonamide (2) (0.52 g, 1.89 mmol), cyclohexanecarboxaldehyde (0.21 g, 1.89 mmol), Sc(OTf)₃ (0.19 g, 0.38 mmol) and 4 Å molecular sieves (1 g) were mixed together and dissolved in anhydrous CH₃CN (10 mL) at room temperature under argon. The reaction mixture was stirred for one hour at room temperature. 2,3-Dihydrofuran (0.27 g, 3.78 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.63 g, 76% yield). (92% trans-isomer): ¹H NMR (400 MHz, DMSO-d⁶) δ 9.64 (s, 1H), 7.46 (d, J=1.6 Hz, 1H), 7.23 (dd, J₁=8.4 Hz, J₂=2.0 Hz, 1H), 6.93 (d, J=8.0 Hz, 1H), 6.83 (d, J=2.0 Hz, 1H), 6.78 (dd, J₁=8.0 Hz, J₂=2.0 Hz, 1H), 6.69 (d, J=8.4 Hz, 1H), 6.06 (s, 1H), 4.94 (d, J=7.6 Hz, 1H), 3.72-3.66 (m, 1H), 3.48-3.43 (m, 1H), 3.16 (dd, J₁=8.4 Hz, J₂=2.0 Hz, 1H), 2.09 (s, 6H), 1.81-1.57 (m, 7H), 1.33-1.13 (m, 5H), 1.00-0.91 (m, 2H). HRMS (ESI) calcd for C₂₅H₃₃NO₃S: 441.2212 [M+H]⁺, found 441.2219.

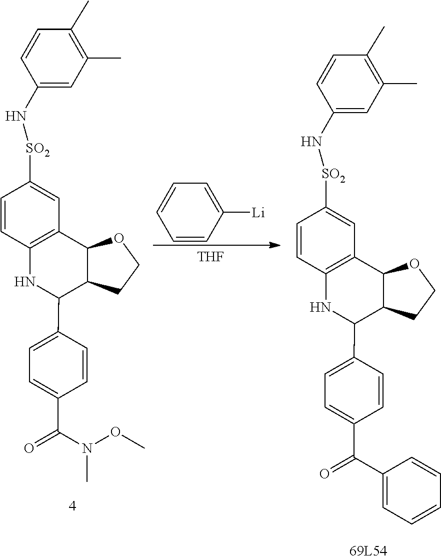

Cis-trans mixture of 69L54: ¹H NMR (400 MHz, CDCl₃) δ 7.88-7.79 (m, 5H), 7.64-7.60 (m, 1H), 7.55-7.48 (m, 4H), 7.46-7.42 (m, 1H), 6.98 (d, J=8.0 Hz, 2H), 6.88 (s, 1H), 6.81-6.78 (m, 1H), 6.58-6.55 (m, 1H), 6.25-6.22 (m, 1H), 5.20 (d, J=7.6 Hz, 0.55H), 4.88 (d, J=2.8 Hz, 0.55H), 4.59 (s, 0.45H), 4.54 (d, J=5.2 Hz, 0.45H), 4.31 (s, 0.55H), 4.05-4.03 (m, 0.55H), 3.94 (d, J=10.8 Hz, 0.45H), 3.90-3.86 (m, 0.55H), 3.76-3.72 (m, 0.55H), 3.70-3.66 (m, 0.55H), 2.80-2.77 (m, 0.55H), 2.45-2.43 (m, 0.45H), 2.18-2.17 (m, 6H), 2.09-2.01 (m, 1H), 1.76-1.73 (m, 0.55H).

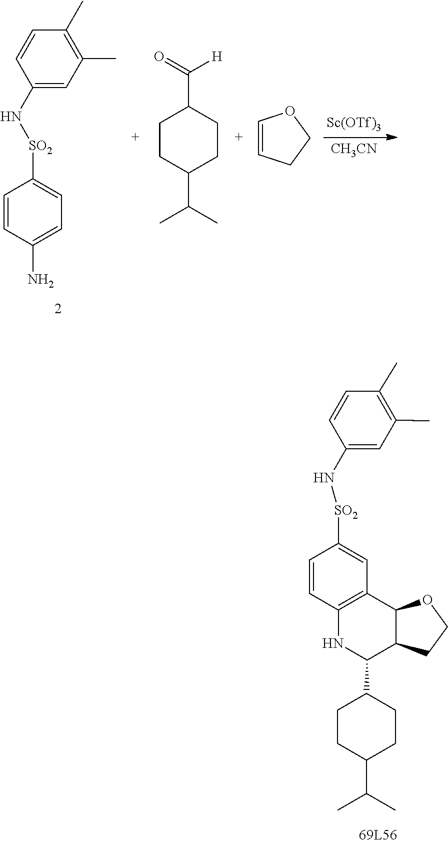

### Preparation of N-(3,4-dimethylphenyl)-4-(4-isopropylcyclohexyl)-2,3,3a,4,5,9b-hexahydrofuran[3,2-c]quinolone-8-sulfonamine (69L56)

Compound 4-amino-N-(3,4-dimethylphenyl)benzenesulfonamide (2) (0.51 g, 1.85 mmol), 4-isopropylcyclohexane-1-carbaldehyde (0.29 g, 1.85 mmol), Sc(OTf)₃ (0.18 g, 0.37 mmol) and 4 Å molecular sieves (1 g) were mixed together and dissolved in anhydrous CH₃CN (10 mL) at room temperature under argon. The reaction mixture was stirred for one hour at room temperature. 2,3-Dihydrofuran (0.26 g, 3.70 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.74 g, 83% yield). (76% trans-isomer): ¹H NMR (400 MHz, DMSO-d⁶) δ 9.63 (s, 1H), 7.45 (d, J=2.0 Hz, 1H), 7.23 (dd, J₁=8.4 Hz, J₂=2.0 Hz, 1H), 6.93 (d, J=8.0 Hz, 1H), 6.81 (d, J=2.0 Hz, 1H), 6.77 (dd, J₁=8.4 Hz, J₂=2.0 Hz, 1H), 6.69 (d, J=8.4 Hz, 1H), 6.06 (s, 1H), 4.93 (d, J=7.6 Hz, 1H), 3.69-3.65 (m, 1H), 3.48-3.43 (m, 1H), 3.13 (dd, J₁=8.4 Hz, J₂=2.0 Hz, 1H), 2.08 (s, 6H), 1.85-0.97 (m, 13H), 0.84 (d, J=6.4 Hz, 6H). HRMS (ESI) calcd for C₂₈H₃₉N₂O₃S: 483.2681 [M+H]⁺, found 483.2684.

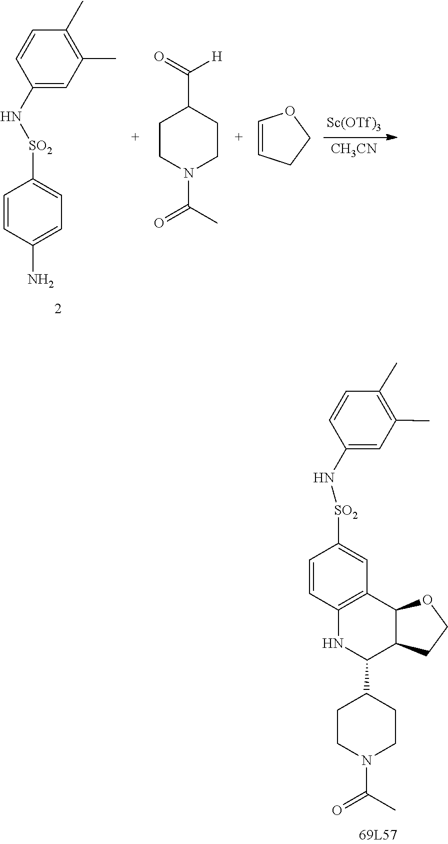

### Preparation of 4-(1-acetylpiperidin-4-yl)-N-(3,4-dimethylphenyl)-2,3,3a,4,5,9b-hexahydrofuran[3,2-c]quinolone-8-sulfonamine (69L57)

Compound 4-amino-N-(3,4-dimethylphenyl)benzenesulfonamide (2) (0.41 g, 1.48 mmol), 1-acetylpiperidine-4-carbaldehyde (0.23 g, 1.48 mmol), Sc(OTf)₃ (0.15 g, 0.30 mmol) and 4 Å molecular sieves (1 g) were mixed together and dissolved in anhydrous CH₃CN (10 mL) at room temperature under argon. The reaction mixture was stirred for one hour at room temperature. 2,3-Dihydrofuran (0.21 g, 2.97 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/MeOH=19/1 v/v) to give a white solid product (0.62 g, 86% yield). (90% trans-isomer): ¹H NMR (400 MHz, DMSO-d⁶) δ 9.64 (s, 1H), 7.47 (d, J=1.6 Hz, 1H), 7.24 (dd, J₁=8.4 Hz, J₂=2.0 Hz, 1H), 6.93 (d, J=8.0 Hz, 1H), 6.82 (s, 1H), 6.77 (d, J=8.0 Hz, 1H), 6.69 (d, J=8.4 Hz, 1H), 6.12 (d, J=6.4 Hz, 1H), 4.94 (d, J=7.6 Hz, 1H), 4.42 (d, J=8.4 Hz, 1H), 3.787-3.82 (m, 1H), 3.72-3.64 (m, 1H), 3.49-3.44 (m, 1H), 3.19 (dd, J=8.4 Hz, J=1.6 Hz, 1H), 3.03-2.97 (m, 1H), 2.09 (s, 6H), 1.99 (s, 3H), 1.86-1.73 (m, 3H), 1.63-1.52 (m, 3H), 1.14-1.07 (m, 2H). HRMS (ESI) calcd for C₂₆H₃₄N₃O₄S: 484.2270 [M+H]⁺, found 484.2268.

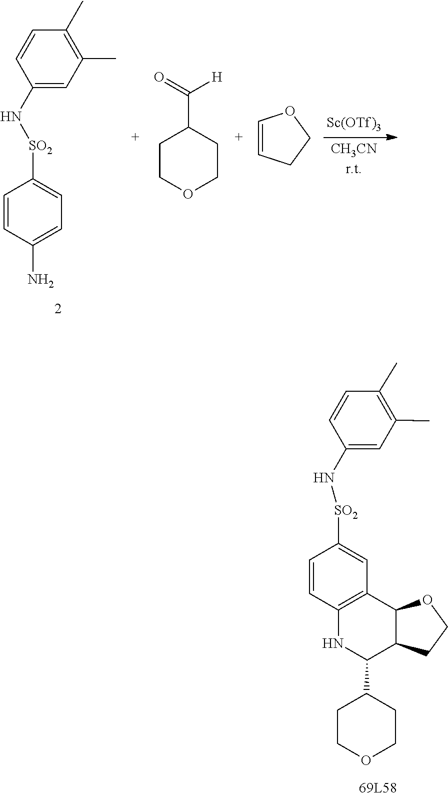

### Preparation of N-(3,4-dimethylphenyl)-4-(tetrahydro-2H-pyran-4-yl)-2,3,3a,4,5,9b-hexahydrofuran[3,2-c]quinolone-8-sulfonamine (69L58)

Compound 4-amino-N-(3,4-dimethylphenyl)benzenesulfonamide (2) (0.55 g, 1.99 mmol), tetrahydro-2H-pyran-4-carbaldehyde (0.23 g, 1.99 mmol), Sc(OTf)₃ (0.20 g, 0.40 mmol) and 4 Å molecular sieves (1 g) were mixed together and dissolved in anhydrous CH₃CN (10 mL) at room temperature under argon. The reaction mixture was stirred for one hour at room temperature. 2,3-Dihydrofuran (0.28 g, 4.00 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=17/3 v/v) to give a white solid product (0.76 g, 86.4% yield). (98% trans-isomer): ¹H NMR (400 MHz, DMSO-d⁶) δ 9.65 (s, 1H), 7.47 (d, J=1.6 Hz, 1H), 7.24 (dd, J=8.4 Hz, J₂=2.0 Hz, 1H), 6.93 (d, J=8.4 Hz, 1H), 6.82 (d, J=L6 Hz, 1H), 6.77 (dd, J₁=8.4 Hz, J₂=2.0 Hz, 1H), 6.69 (d, J=8.4 Hz, 1H), 6.08 (s, 1H), 4.94 (d, J=7.6 Hz, 1H), 3.91-3.88 (m, 2H), 3.70-3.67 (m, 1H), 3.48-3.45 (m, 1H), 3.30-317 (m, 2H), 2.09 (s, 6H), 2.00-1.84 (m, 1H), 1.83-1.80 (m, 1H), 1.65-1.54 (m, 3H), 1.29-1.18 (m, 2H). HRMS (ESI) calcd for C₂₄H₃₁N₂O₄S: 443.2005 [M+H]⁺, found 443.2204.

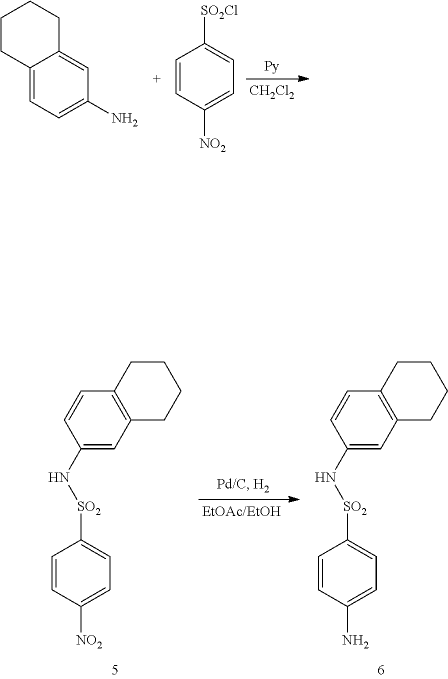

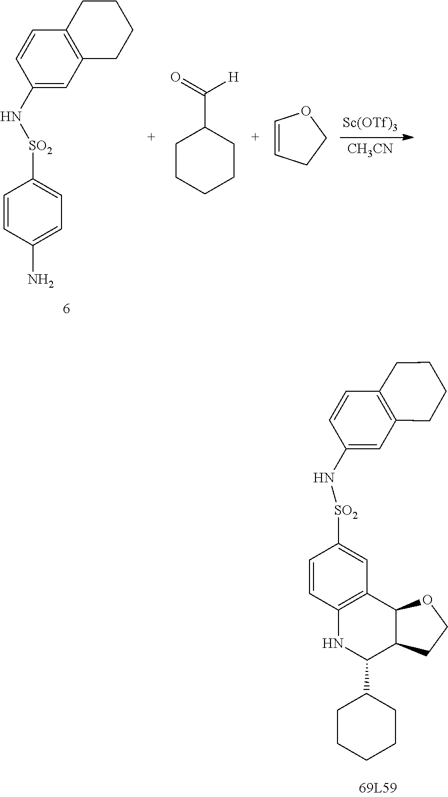

### Preparation of 4-nitro-N-(5,6,7,8-tetrahydronaphthalen-2yl)-benzenesulfonamide (5)

4-Nitrobenzenesulfonyl chloride (6.78 g, 30.57 mmol) and 5,6,7,8-tetrahydronaphthalen-2-amine (4.50 g, 30.57 mmol) were dissolved in anhydrous methylene chloride (50 mL) at room temperature under argon. Pyridine (7.25 g, 91.71 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=19/1 v/v)) to afford product as a pale yellow solid (10.0 g, 98.8% yield). ¹H NMR (400 MHz, DMSO-d⁶) δ 10.38 (s, 1H); 8.37 (d, J=8.8 Hz, 2H), 7.97 (d, J=8.8 Hz, 2H), 6.91 (d, J=8.0 Hz, 1H), 6.82-6.78 (m, 2H), 2.156 (m, 4H), 1.65 (m, 4H).

### Preparation of 4-amino-N-(5,6,7,8-tetrahydronaphthalen-2-yl)benzenesulfonamide (6)

Compound 4-nitro-N-(5,6,7,8-tetrahydronaphthalen-2-yl)-benzenesulfonamide (5) (10.40 g, 31.29 mmol) and Pd/C (10% Pd base, 2 g) were mixed together in EtOAc/MeOH (1:1, 150 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (9.10 g, 926% yield). ¹H NMR (400 MHz, DMSO-d⁶) δ 9.62 (s, 1H); 7.36 (d, J=8.4 Hz, 2H), 6.84 (d, J=8.4 Hz, 1H), 6.80-6.77 (m, 1H), 6.73 (s, 1H), 6.52 (d, J=8.4 Hz, 2H), 5.93 (s, 2H), 2.57 (s, 4H), 1.65 (s, 4H).

### Preparation of 4-cyclohexyl-N-(5,6,7,8-tetrahydronaphthalen-2-yl)-2,3,3a,4,5,9b-hexahydrofuran[3,2-c]quinolone-8-sulfonamine (69L59)

Compound 4-amino-N-(5,6,7,8-tetrahydronaphthalen-2-yl)benzenesulfonamide (6) δ 0.50 g, 1.65 mmol), cyclohexanecarboxaldehyde (0.19 g, 1.65 mmol), Sc(OTf)₃ (0.16 g, 0.33 mmol) and 4 Å molecular sieves (1 g) were mixed together and dissolved in anhydrous CH₃CN (10 mL) at room temperature under argon. The reaction mixture was stirred for one hour at room temperature. 2,3-Dihydrofuran (0.23 g, 3.30 mmol) was then added via a syringe. The resulted mixture was stirred at room temperature under argon overnight. The reaction was quenched by adding 50 mL of water at room temperature. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.38 g, 49.4% yield). (trans-isomer): 1H NMR (400 MHz, DMSO-d⁶) δ 9.64 (s, 1H), 7.46 (d, J=1.6 Hz, 1H), 7.24 (dd, J₁=8.8 Hz, J₂=2.0 Hz, 1H), 6.85 (d, J=8.4 Hz, 1H), 6.77 (dd, J₁=8.4 Hz, J₂=2.0 Hz, 1H), 6.73 (s, 1H), 6.70 (d, J=8.4 Hz, 1H), 6.06 (s, 1H), 4.94 (d, J=7.2 Hz, 1H), 3.68 (q, J=8.0 Hz, 1H), 3.47-3.44 (m, 1H), 3.16 (dd, J₁=8.4 Hz, J₂=3.0 Hz, 1H), 2.58 (s, 4H), 1.82-1.71 (m, 4H), 1.59 (s, 4H), 1.57-1.53 (m, 2H), 1.32-1.14 (m, 5H), 1.00-0.91 (m, 2H). HRMS (EST) calcd for C₂₇H₃₅N₂O₃S: 467-2368 [M+H]⁺, found 467.2362.

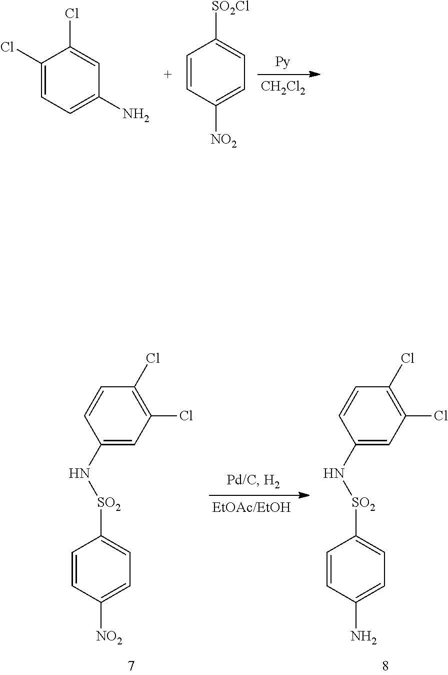

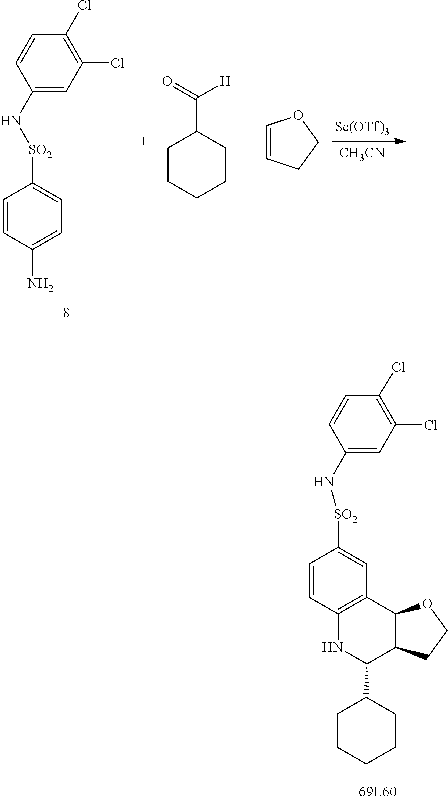

### Preparation of N-(3,4-dichlorophenyl)-4-nitrobenzenesulfonamide (7)

4-Nitrobenzenesulfonyl chloride (6.84 g, 30.86 mmol) and 3,4-dichloroaniline (5.00 g, 30.86 mmol) were dissolved in anhydrous methylene chloride (100 mL) at room temperature under argon. Pyridine (7.32 g, 92.58 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂) to afford product as a pale yellow solid (10.677 g, 99.6% yield). ¹H NMR (400 MHz, DMSO-d⁶) δ 11.03 (s, 1H); 8.40 (d, J=8.4 Hz, 2H), 8.03 (d, J=8.4 Hz, 2H), 7.54 (d, J=8.8 Hz, 1H), 7.32 (d, J=2.4 Hz, 1H), 7.11 (dd, J₁=8.8 Hz, J₂=2.4 Hz, 1H).

### Preparation of 4-amino-N-(3,4-dichlorophenyl)benzenesulfonamide (8)

Compound N-(3,4-dichlorophenyl)-4-nitrobenzenesulfonamide (7) (10.00 g, 28.81 mmol) and Pd/C (10% Pd base, 2 g) were mixed together in MeOH (50 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (9.07 g, 99.2% yield). ¹H NMR (400 MHz, DMSO-d⁶) δ 10.28 (s, 1H); 7.47 (d, J=8.8 Hz, 1H), 7.40 (d, J=8.4 Hz, 2H), 7.23 (d, J=2.0 Hz, 1H), 7.05 (d, J₁=8.8 Hz, J₂=2.4 Hz, 1H), 6.55 (d, J=8.4 Hz, 2H), 6.07 (s, 2H).

### Preparation of 4-cyclohexyl-N-(3,4-dichlorophenyl)-2,3,3a,4,5,9b-hexahydrofuran[3,2-c]quinolone-8-sulfonamine (69L60)

Compound 4-amino-N-(3,4-dichlorophenyl)benzenesulfonamide (8) (0.50 g, 1.58 mmol), cyclohexanecarboxaldehyde (0.18 g, 1.58 mmol), Sc(OTf)₃ (0.16 g, 0.32 mmol) and 4 Å molecular sieves (1 g) were mixed together and dissolved in anhydrous CH₃CN (10 mL) at room temperature under argon. The reaction mixture was stirred for one hour at room temperature. 2,3-Dihydrofuran (0.22 g, 3.16 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.52 g, 68.4% yield). ¹H NMR (400 MHz, DMSO-d⁶) δ 10.27 (s, 1H), 7.49-7.47 (m, 3H), 7.30 (dd, J₁=8.4 Hz, J₂=2.0 Hz, 1H), 7.22 (d, J=2.4 Hz, 1H), 7.05 (dd, J₁=8.4 Hz, J₂=2.4 Hz, 1H), 6.73 (d, J=8.4 Hz, 1H), 6.19 (s, 1H), 4.94 (d, J=7.6 Hz, 1H), 3.72-3.66 (m, 2H), 3.46-3.42 (m, 1H), 3.19 (d, J=6.8 Hz, 1H), 2.58-2.50 (m, 1H), 1.82-1.55 (m, 8H), 1.33-1.13 (m, 5H), 1.03-0.92 (m, 2H).

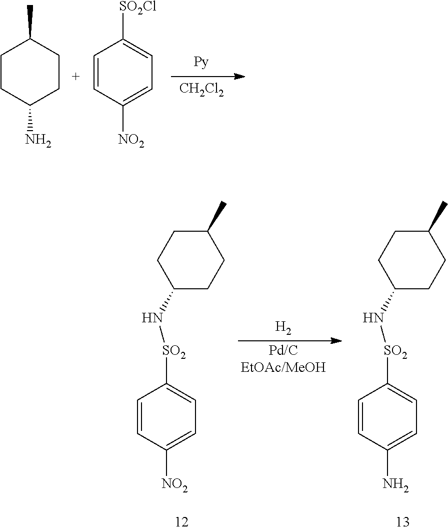

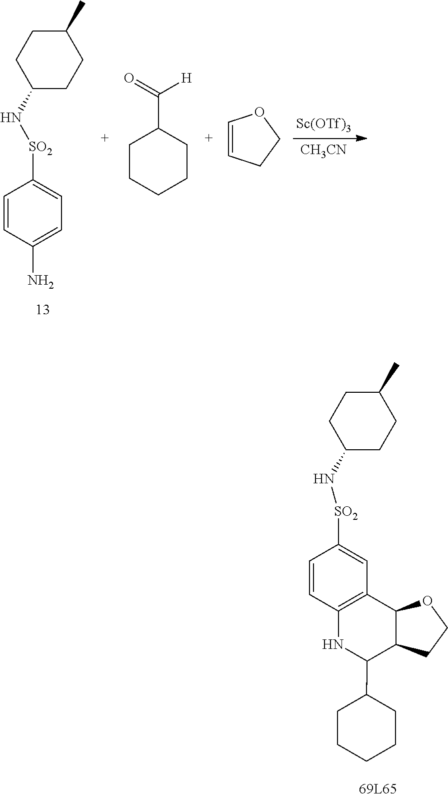

### Preparation of N-(4-methylcyclohexyl)-4-nitrobenzenesulfonamide (12)

4-Nitrobenzenesulfonyl chloride (15.66 g, 70.67 mmol) and trans-4-methylcyclohexamine (8.00 g, 70.67 mmol) were dissolved in anhydrous methylene chloride (100 mL) at room temperature under argon. Pyridine (8.38 g, 106 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂) to afford product as a pale yellow solid (18.5 g, 87.7% yield). ¹H NMR (400 MHz, DMSO-d⁶) δ 8.41 (d, J=8.0 Hz, 2H), 8.07-8.04 (m, 3H), 2.98-2.89 (m, 1H), 1.60-1.53 (m, 4H), 1.20-1.10 (m, 3H), 0.88-0.82 (m, 2H), 0.78 (d, J=6.8 Hz, 3H).

### Preparation of 4-amino-N-(4-methylcyclohexyl)benzenesulfonamide (13)

Compound N-(4-methylcyclohexyl)-4-nitrobenzenesulfonamide (12) (10.00 g, 30.60 mmol) and Pd/C (10% Pd base, 2 g) were mixed together in MeOH (50 mL) and ethyl acetate (50 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (7.8 g, 86.7% yield). ¹H NMR (400 MHz, DMSO-d⁶) δ 7.41 (d, J=8.8 Hz, 2H), 7.10 (d, J=7.2 Hz, 1H), 6.58 (d, J=8.4 Hz, 2H), 5.89 (s, 2H), 2.75-2.66 (m, 1H), 1.59-1.53 (m, 4H), 1.20-1.04 (m, 3H), 0.84-0.74 (m, 5H).

### Preparation of 4-cyclohexyl-N-(3-chloro-4-methylphenyl)-2,3,3a,4,5,9b-hexahydrofuran[3,2-c]quinolone-8-sulfonamine (69L65)

Compound 4-amino-N-(3-chloro-4-methylphenyl)benzenesulfonamide (10) (0.53 g, 1.77 mmol), cyclohexanecarboxaldehyde (0.20 g, 1.77 mmol), Sc(OTf)₃ (0.17 g, 0.35 mmol) and 4 Å molecular sieves (1 g) were mixed together and dissolved in anhydrous CH₃CN (10 mL) at room temperature under argon. The reaction mixture was stirred for one hour at room temperature. 2,3-Dihydrofuran (0.25 g, 3.54 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.65 g, 79.6% yield). ¹H NMR (400 MHz, DMSO-d⁶) δ 10.00 (s, 0.2H), 9.97 (s, 0.8H), 7.49 (s, 0.2H), 7.46 (s, 0.8H), 7.30 (dd, J₁=8.8 Hz, J₂=2.0 Hz, 0.2H), 7.25 (dd, J₁=8.8 Hz, J₂=2.0 Hz, 0.8H), 7.17 (d, J=80 Hz, 1H), 7.07 (s, 0.2H), 7.05 (d, J=1.6 Hz, 0.8H), 6.96-6.93 (m, 1H), 6.75-6.71 (m, 1H), 6.56 (s, 0.2H), 6.14 (s, 0.8H), 4.94 (d, J=7.6 Hz, 0.8H), 4.47 (d, J=6.0 Hz, 0.2H), 3.72-3.66 (m, 1H), 3.48-3.46 (m, 1H), 3.18 (d, J=8.4 Hz, 0.8H), 2.64-2.62 (m, 0.2H), 2.09 (s, 3H), 1.82-1.55 (m, 8H), 1.33-1.13 (m, 5H), 1.03-0.91 (m, 2H).

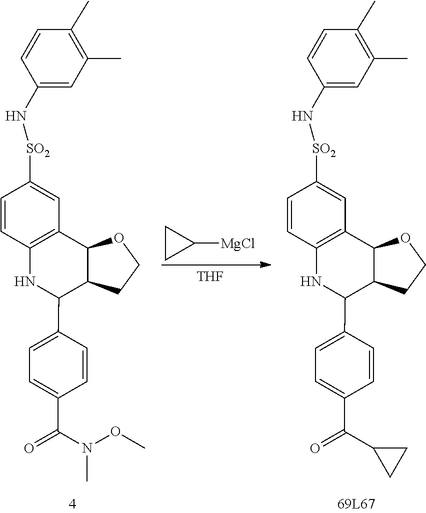

Cis-69L67: ¹H NMR (400 MHz, DMSO-d⁶): δ 9.74 (s, 1H), 8.07 (d, J=8.0 Hz, 2H), 7.60 (d, J=8.0 Hz, 2H), 7.54 (s, 1H), 7.32 (d, J=8.4 Hz, 1H), 6.96 (d, J=8.4 Hz, 1H), 6.91 (s, 1H), 6.86 (s, 1H), 6.80 (d, J=8.0 Hz, 1H), 5.11 (d, J=7.6 Hz, 0.45H), 4.86 (d, J=2.4 Hz, 1H), 3.67-3.73 (m, 1H), 3.62-3.56 (m, 1H), 3.52-3.49 (m, 1H), 2.95-2.90 (m, 1H), 2.71-2.68 (m, 1H), 2.11 (s, 3H), 2.09 (s, 3H), 1.80-1.73 (m, 1H), 1.33-1.31 (1H), 1.17-1.09 (m, 2H), 0.86-0.76 (2H).

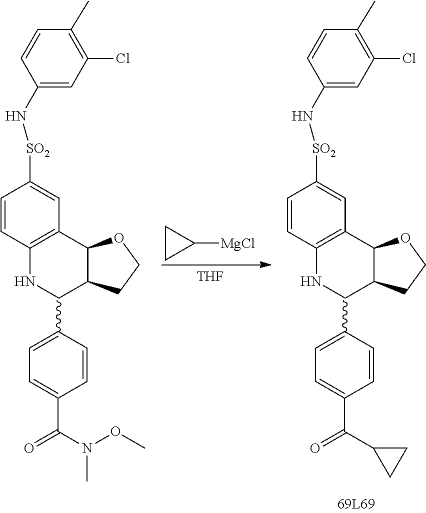

Cis-69L69: ¹H NMR (400 MHz, DMSO-d⁶): δ 10.08 (s, 1H), 8.07 (d, J=8.0 Hz, 2H), 7.61 (d, J=8.4 Hz, 2H), 7.55 (s, 1H), 7.35 (d, J=8.8 Hz, 1H), 7.21 (d, J=8.4 Hz, 1H), 7.09 (s, 1H), 6.99-6.96 (m, 2H), 6.75 (d, J=8.4 Hz, 1H), 5.12 (d, J=7.2 Hz, 1H), 4.88 (d, J=2.4 Hz, 1H), 3.64-3.58 (m, 1H), 3.52-3.48 (m, 1H), 2.95-2.89 (m, 1H), 2.71-2.68 (m, 1H), 2.20 (s, 3H), 1.81-1.74 (m, 1H), 1.33-1.30 (m, 1H), 1.14-0.99 (m, 4H).

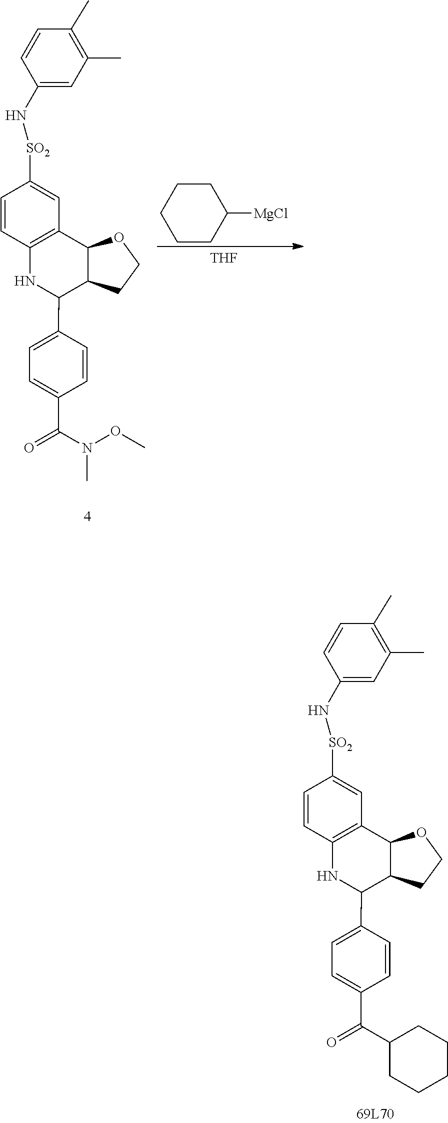

Cis-69L70: ¹H NMR (400 MHz, DMSO-d⁶): δ 9.74 (s, 1H), 8.07 (d, J=8.0 Hz, 2H), 7.98 (d, J=8.0 Hz, 2H), 7.58 (d, J=8.0 Hz, 2H), 7.54 (s, 1H), 7.32 (d, J=8.8 Hz, 1H), 6.96 (d, J=8.0 Hz, 1H), 6.89 (s, 1H), 6.86 (d, J=6.4 Hz, 1H), 6.80 (d, J=8.0 Hz, 1H), 6.71 (d, J=8.4 Hz, 1H), 5.11 (d, J=7.6 Hz, 1H), 4.86 (d, J=2.4 Hz, 1H), 3.63-3.57 (m, 1H), 3.52-3.48 (m, 1H), 2.71-2.68 (m, 1H), 2.11 (s, 3H), 2.09 (s, 3H), 1.76-1.62 (m, 7H), 1.47-1.31 (m, 3H), 1.24-1.09 (m, 3H).

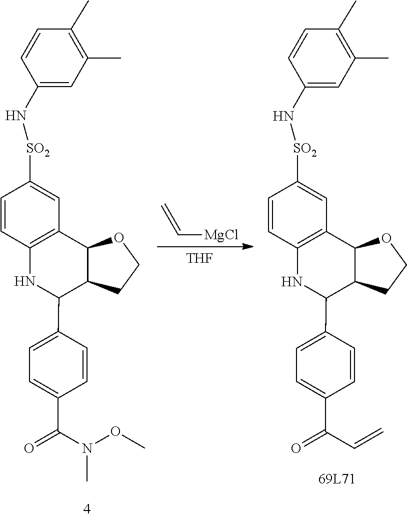

Cis-trans mixture of 69L71: ¹H NMR (400 MHz, DMSO-d⁶) δ 9.78 (s, 0.4H), 9.74 (s, 0.6H), 8.04 (d, J=8.0 Hz, 2H), 7.65-7.54 (m, 3H), 7.47-7.40 (m, 1.6H), 7.33 (dd, J₁=8.4 Hz, J₂=2.4 Hz, 0.1H), 7.20 (s, 0.4H), 6.96 (d, J=8.0 Hz, 1H), 6.91 (s, 0.6H), 6.86 (d, J=6.4 Hz, 1H), 6.82-6.80 (m, 1H), 6.74-6.38 (m, 1H), 6.36 (d, J=17.2 Hz, 1H), 6.01 (d, J=2.8 Hz, 1H), 5.12 (d, J=7.2 Hz, 0.6H), 4.87 (d, J=2.8 Hz, 0.6H), 4.43 (d, J=4.8 Hz, 0.4H), 3.92-3.88 (m, 0.6H), 3.82 (d, J=10.8 Hz, 0.4H), 3.72-3.69 (m, 0.6H), 3.65-3.59 (m, 0.6H), 3.52-3.48 (m, 0.6H), 3.02-2.98 (m, 2H), 2.72-2.68 (m, 0.6H), 2.33-2.25 (m, 0.4H), 2.11 (s, 3H), 2.09 (s, 3H), 1.96-1.91 (m, 0.4H), 1.80-1.72 (m, 0.6H), 1.59-1.52 (m, 0.4H), 1.34-1.28 (m, 1H).

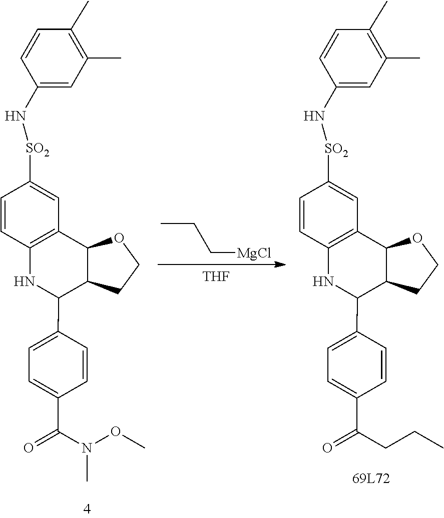

Cis-trans mixture of 69L72: ¹H NMR (400 MHz, DMSO-d⁶) δ 9.78, 9.74 (s, 1H), 7.99 (d, J=8.0 Hz, 2H), 7.61-7.54 (m, 3H), 7.39 (dd, J₁=8.4 Hz, J₂=2.0 Hz, 0.4H), 7.32 (dd, J₁=8.4 Hz, J₂=2.0 Hz, 0.6H), 7.17 (s, 0.40H), 6.96 (d, J=8.4 Hz, 1H), 6.89-6.86 (m, 1.6H), 6.84-6.80 (m, 1H), 6.77-6.70 (m, 1H), 5.11 (d, J=7.6 Hz, 0.6H), 4.84 (d, J=2.8 Hz, 0.6H), 4.43 (d, J=4.4 Hz, 0.4H),), 3.91-3.88 (m, 0.6H), 3.79 (d, J=10.4 Hz, 0.4H), 3.72-3.68 (m, 0.6H), 3.63-3.59 (m, 0.6H), 3.51-3.49 (m, 0.6H), 3.02-2.98 (m, 2H), 2.71-2.67 (m, 0.6H), 2.30-2.26 (m, 0.4H), 2.11 (s, 3H), 2.09 (s, 3H), 1.96-1.91 (m, 0.4H), 1.81-1.72 (m, 0.6H), 1.65 (q, J=7.6 Hz, 2H), 1.59-1.54 (m, 0.4H), 1.34-1.28 (m, 1H), 0.93 (t, J=7.6 Hz, 3H).

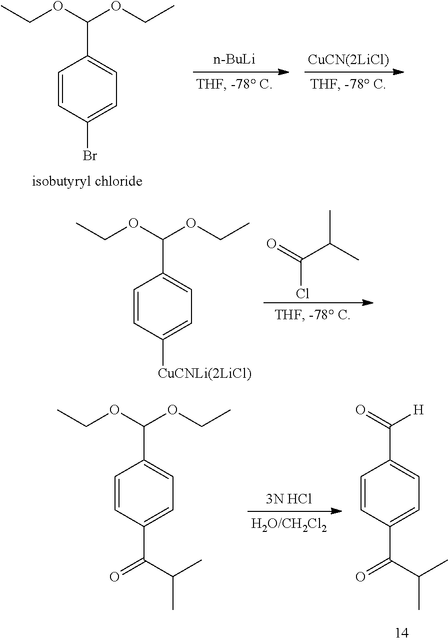

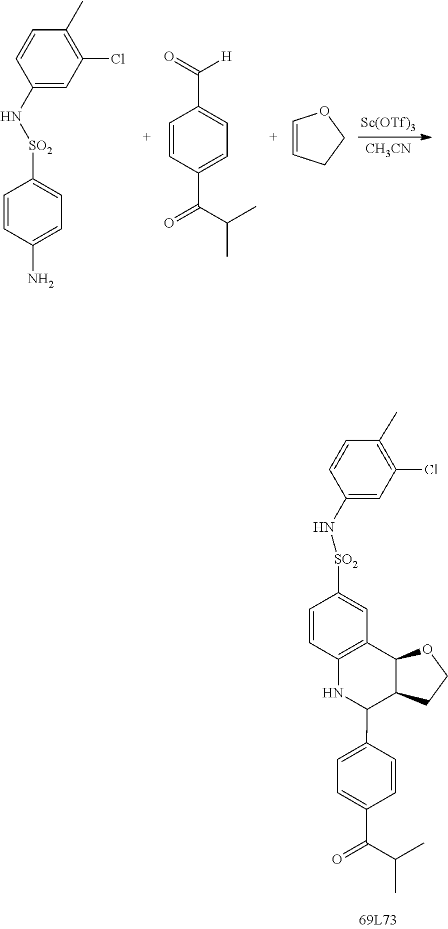

### Preparation of 4-isobytyrylbenzaldehyde (14)

1-Bromo-4-(diethoxymethyl)benzene (10.00 g, 38.59 mmol) was dissolved in anhydrous THF (150 mL) at room temperature under argon. The solution was cooled to −78° C. in an acetone-dry ice bath. n-BuLi (16.98 mL of 2.5 M in hexanes, 42.24 mmol) was added dropwise via a syringe at −78° C. under argon. The reaction solution was stirred at −78° C. for 2 hours. Then, a solution of CuCN (3.46 g, 38.59 mmol) and LiCl (3.27 g, 77.18 mmol) generated in situ in 50 mL of THF was added via a syringe at −78° C. After reaction mixture was stirred at −78° C. for one hour, isobutyryl chloride (4.50 g, 42.24 mmol) was added via a syringe at −78° C. The resulted mixture was stirred at −78° C. for 30 minutes and then at room temperature for one hour. The reaction was quenched by adding 100 mL of saturated NH₄Cl solution. THF solvent was removed under reduced pressure. The oil residue was extracted with CH₂Cl₂ (3×50 mL). The organic layer was separated, dried over anhydrous MgSO₄, filtered and concentrated under reduced pressure. The residue oil was purified by flash column chromatography (silica gel, CH₂Cl₂/acetone=99/1 v/v)) to give desired compound 14 as a pale yellow liquid (4.6 g, 67.6% yield). ¹H NMR (400 MHz, DMSO-d⁶) δ 10.11 (s, 1H), 8.14 (d, J=8.0 Hz, 2H), 8.05 (d, J=8.0 Hz, 2H), 3.70 (sep, J=6.8 Hz, 1H), 1.12 (d, J=6.8 Hz, 6H).

### Preparation of N-(3-chloro-4-methylphenyl)-4-(4-isobutyrylphenyl)-2,3,3a,4,5,9b-hexahydrofuran[3,2-c]quinolone-8-sulfonamide (69L73)

Compound 4-amino-N-(3-chloro-4-dimethylphenyl)benzenesulfinamide (0.98 g, 3.29 mmol), 4-isobutyrylbenzaldehyde (0.58 g, 3.29 mmol), Sc(OTf)₃ (0.32 g, 0.66 mmol) and 4 Å molecular sieves (1 g) were mixed together and dissolved in anhydrous CH₃CN (30 mL) at room temperature under argon. The reaction mixture was stirred for one hour at room temperature. 2,3-Dihydrofuran (0.46 g, 6.58 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.98 g, 56.6% yield).

Cis-trans mixture of 69L73: ¹H NMR (400 MHz, DMSO-d⁶) δ 10.13 (s, 0.6H), 10.08 (s, 0.4H), 8.00 (d, J=8.0 Hz, 2H), 7.62-7.58 (m, 3H), 7.41 (dd, J₁=8.8 Hz, J₂=2.4 Hz, 0.6H), 7.35 (dd, J₁=8.8 Hz, J₂=1.6 Hz, 0.4H), 7.26 (s, 0.6H), 7.21 (d, J=8.4 Hz, 1H), 7.10 (dd, J₁=8.0 Hz, J₂=1.6 Hz, 1H), 7.01-6.99 (m, 1.4H), 6.77-6.72 (m, 1H), 5.12 (d, J=7.2. Hz, 0.4H), 4.87 (d, J=3.2 Hz, 0.4H), 4.44 (d, J=4.8 Hz, 0.6H), 3.92-3.88 (m, 0.6H), 3.81 (d, J=10.8 Hz, 0.6H), 3.74-3.60 (m, 2H), 3.52-3.49 (m, 0.4H), 2.72-2.67 (m, 0.4H), 2.33-2.27 (m, 0.6H), 2.21 (s, 3H), 1.99-1.91 (m, 0.6H), 1.82-1.72 (m, 0.6H), 1.57-1.53 (m, 0.4H), 1.34-1.31 (m, 0.4H), 1.11 (d, J=6.8 Hz, 6H). HRMS (ESI) calcd for C₂₉H₃₃N₂O₄S: 505.2161 [M+H]⁺, found 505.2160.

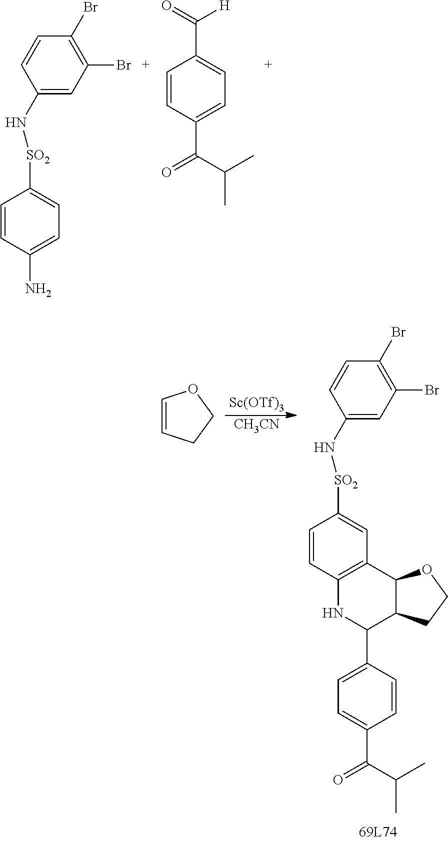

Cis-trans mixture of 69L74: ¹H NMR (400 MHz, DMSO-d⁶) δ 10.41 (s, 0.6H), 10.36 (s, 0.4H), 8.00 (d, J=7.6 Hz, 2H), 7.63-7.55 (m, 4H), 7.46-7.36 (m, 2H), 7.32 (s, 0.4H), 7.09-7.04 (m, 1.6H), 6.79-6.75 (m, 1H), 5.13 (d, J=7.6 Hz, 0.4H), 4.88 (d, J=2.8 Hz, 0.4H), 4.45 (d, J=4.8 Hz, 0.6H), 3.92-3.88 (m, 0.6H), 3.83 (d, J=10.8 Hz, 0.6H), 3.75-3.59 (m, 2H), 3.52-3.49 (m, 0.4H), 2.72-2.67 (m, 0.4H), 2.33-2.27 (m, 0.6H), 1.99-1.91 (m, 0.4H), 1.80-1.72 (m, 0.6H), 1.59-1.52 (m, 0.6H), 1.36-1.32 (m, 0.4H), 1.11 (d, J=6.8 Hz, 6H).

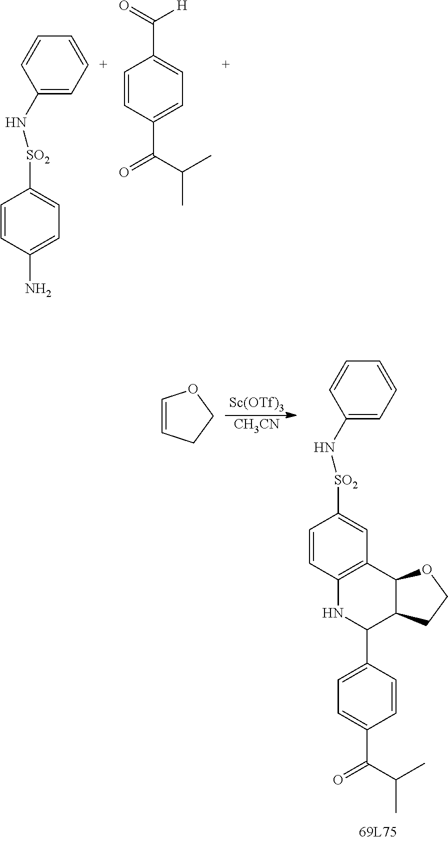

Cis-trans mixture of 69L75: ¹H NMR (400 MHz, DMSO-d⁶) δ 9.99, 9.97 (s, 1H), 8.00 (d, J=8.0 Hz, 2H), 7.62-7.56 (m, 3H), 7.41 (dd, J₁=8.4 Hz, J₂=2.0 Hz, 0.5H), 7.35 (dd, J₁=8.4 Hz, J₂=2.0 Hz, 0.5H), 7.24-7.21 (m, 2.5H), 7.11-7.08 (m, 2H), 7.01-6.98 (m, 1H), 6.92 (s, 0.5H), 6.73 (t, J=8.4 Hz, 1H), 5.11 (d, J=7.6 Hz, 0.5H), 4.85 (d, J=2.8 Hz, 0.5H), 4.43 (d, J=4.8 Hz, 0.5H), 3.91-3.88 (m, 0.5H), 3.80 (d, J=10.4 Hz, 0.5H), 3.72-3.59 (m, 2H), 3.52-3.49 (m, 0.5H), 2.72-2.67 (m, 0.5H), 2.33-2.27 (m, 0.5H), 1.99-1.92 (m, 0.5H), 1.82-1.72 (m, 0.5H), 1.57-1.52 (m, 0.5H), 1.34-1.29 (m, 0.5H), 1.11 (d, J=6.8 Hz, 6H).

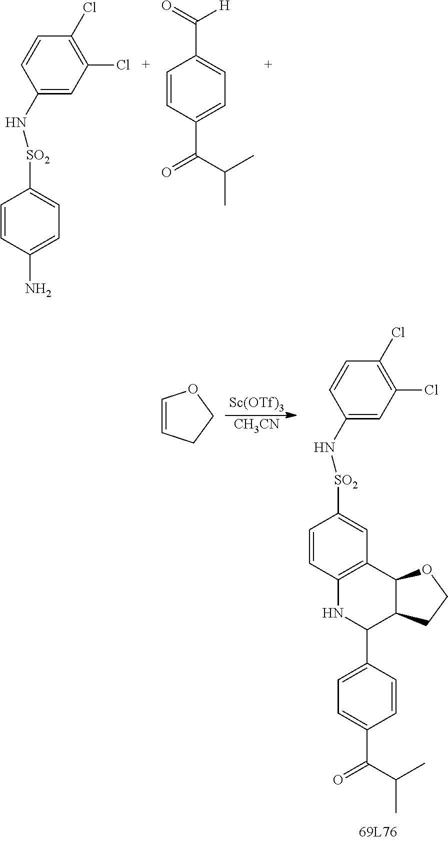

Cis-trans mixture of 69L76: ¹H NMR (400 MHz, DMSO-d⁶) δ 10.43, 10.38 (s, 1H), 8.00 (d, J=8.0 Hz, 2H), 7.61 (d, J=8.4 Hz, 2H), 7.58-7.51 (m, 2H), 7.44 (dd, J₁=8.8 Hz, J₂=2.0 Hz, 0.5H), 7.39-7.37 (m, 0.5H), 7.32-7.25 (m, 1H), 7.11-7.04 (m, 2H), 6.77 (t, J=8.4 Hz, 1H), 5.12 (d, J=7.2 Hz, 0.5H), 4.88 (d, J=2.8 Hz, 0.5H), 4.45 (d, J=4.8 Hz, 0.5H), 3.92-3.88 (m, 0.5H), 3.82 (d, J=10.8 Hz, 0.5H), 3.73-3.60 (m, 2H), 3.52-3.47 (m, 0.5H), 2.68-2.64 (m, 0.5H), 2.33-2.28 (m, 0.5H), 1.99-1.92 (m, 0.5H), 1.79-1.75 (m, 0.5H), 1.57-1.54 (m, 0.5H), 1.33-1.31 (m, 0.5H), 1.11 (d, J=6.8 Hz, 6H).

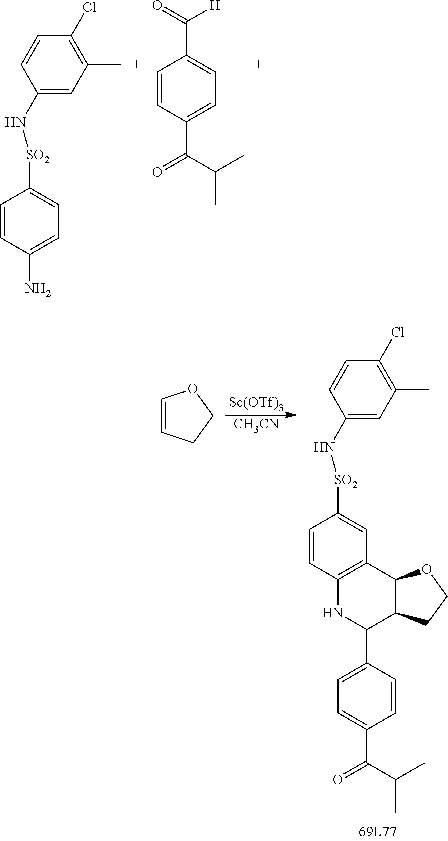

Cis-trans mixture of 69L77: ¹H NMR (400 MHz, DMSO-d⁶) δ 10.10 (s, 0.6H), 10.06 (s, 0.4H), 8.00 (d, J=7.6 Hz, 2H), 7.62-7.55 (m, 3H), 7.42 (dd, J₁=8.8 Hz, J₂=2.0 Hz, 0.6H), 7.36 (dd, J₁=8.8 Hz, J₂=2.0 Hz, 0.4H), 7.28-7.25 (m, 1.6H), 7.04 (s, 1H), 6.96-6.92 (m, 1.4H), 6.76-6.72 (m, 1H), 5.12 (d, J=7.2 Hz, 0.4H), 4.86 (d, J=2.8 Hz, 0.4H), 4.44 (d, J=4.8 Hz, 0.6H), 3.92-3.89 (m, 0.6H), 3.81 (d, J=10.8 Hz, 0.6H), 3.74-3.58 (m, 2H), 3.52-3.49 (m, 0.4H), 2.71-2.65 (m, 0.4H), 2.33-2.26 (m, 0.6H), 2.21 (s, 3H), 1.98-1.93 (m, 0.6H), 1.83-1.75 (m, 0.6H), 1.57-1.53 (m, 0.4H), 1.35-1.29 (m, 0.4H), 1.15 (d, J=6.8 Hz, 6H).

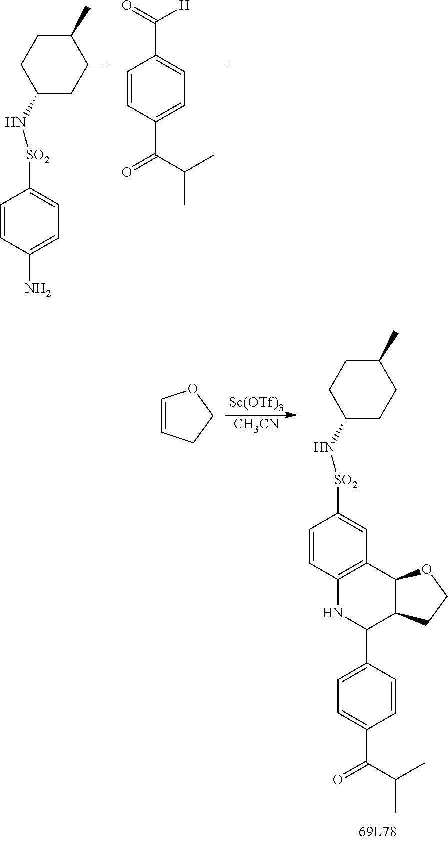

Cis-trans mixture of 69L78: ¹H NMR (400 MHz, DMSO-d⁶) δ 8.01 (d, J=8.0 Hz, 2H), 7.66-7.58 (m, 3H), 7.44 (dd, J₁=8.8 Hz, J₂=2.0 Hz, 0.5H), 7.39 (dd, J₁=8.8 Hz, J₂=1.2 Hz, 0.5H), 7.17 (t, J=6.8 Hz, 1H), 7.12 (s, 0.5H), 6.84 (s, 0.5H), 6.81-6.77 (m, 1H), 5.19 (d, J=6.8 Hz, 0.5H), 4.88 (d, J=2.8 Hz, 0.5H), 4.49 (d, J=4.8 Hz, 0.5H), 3.94-3.91 (m, 0.5H), 3.84 (d, J=10.8 Hz, 0.5H), 3.73-3.52 (m, 2.5H), 2.78-2.73 (m, 1.5H), 2.36-2.32 (m, 0.5H), 2.01-1.82 (m, 1H), 1.63-1.54 (m, 4.5H), 1.37-1.32 (m, 0.5H), 1.21-1.08 (m, 9H), 0.86-0.74 (m, 5H).

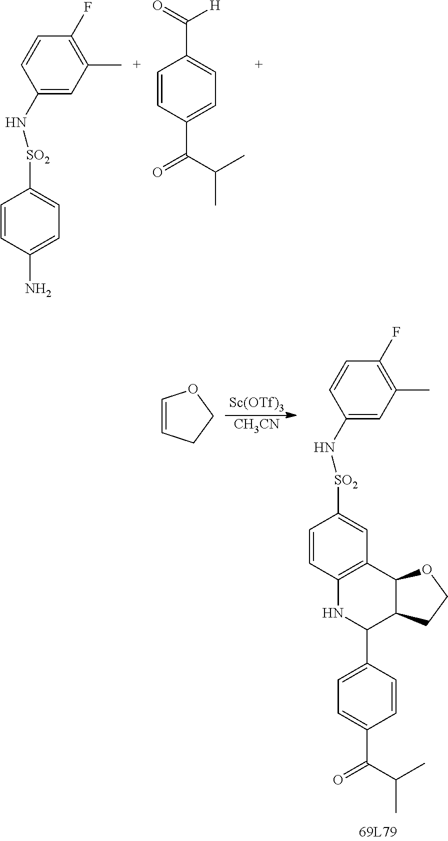

Cis-trans mixture of 69L79: ¹H NMR (400 MHz, DMSO-d⁶) δ 9.88 (s, 0.4H), 9.83 (s, 0.6H), 8.00 (d, J=8.0 Hz, 2H), 7.62-7.58 (m, 2.4H), 7.51 (s, 0.6H), 7.37 (d, J=8.8 Hz, 0.4H), 7.32 (d, J=8.4 Hz, 0.6H), 7.22 (s, 0.4H), 7.03-6.89 (m, 3.6H), 6.72 (t, J=8.4 Hz, 1H), 5.11 (d, J=7.6 Hz, 0.6H), 4.86 (d, J=2.8 Hz, 0.6H), 4.43 (d, J=4.8 Hz, 0.4H), 3.92-3.88 (m, 0.4H), 3.81 (d, J=10.4 Hz, 0.4H), 3.72-3.57 (m, 2H), 3.52-3.46 (m, 0.6H), 2.71-2.65 (m, 0.6H), 2.33-2.27 (m, 0.4H), 2.14 (s, 3H), 1.99-1.90 (m, 0.4H), 1.80-1.75 (m, 0.4H), 1.56-1.53 (m, 0.6H), 1.34-1.29 (m, 0.6H), 1.11 (d, J=6.4 Hz, 6H).

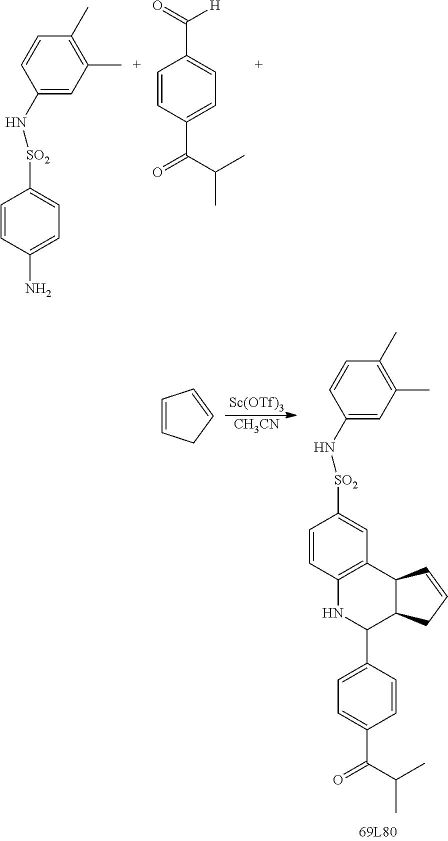

69L80: ¹H NMR (400 MHz, CDCl₃) δ 7.96 (d, J=8.0 Hz, 2H), 7.47 (d, J=8.0 Hz, 2H), 7.40-7.37 (m, 2H), 6.98 (d, J=8.0 Hz, 1H), 6.85 (s, 1H), 6.82 (d, J=8.0 Hz, 1H), 6.76-6.71 (m, 1H), 6.60 (d, J=8.4 Hz, 1H), 5.60 (d, J=10.4 Hz, 2H), 4.71 (s, 1H), 4.25 (s, 1H), 4.02 (d, J=8.4 Hz, 1H), 3.55 (sep, J=6.8 Hz, 1H), 3.02-2.96 (m, 1H), 2.51-2.45 (m, 1H), 2.17 (s, 3H), 1.78-1.72 (m, 2H), 1.22 (d, J=6.4 Hz, 6H).

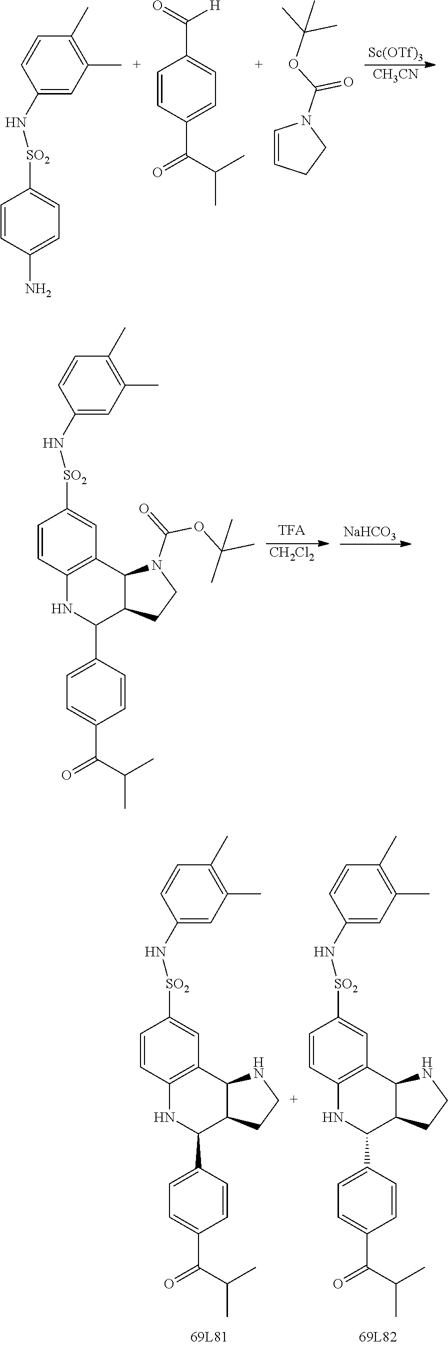

69L81: ¹H NMR (400 MHz, DMSO-d⁶) δ 9.78 (s, 1H), 8.00 (d, J=8.0 Hz, 2H), 7.74 (s, 1H), 7.56 (d, J=8.0 Hz, 2H), 7.34 (d, J=8.8 Hz, 1H), 6.95 (d, J=8.4 Hz, 1H), 6.90 (s, 1H), 6.87 (s, 1H), 6.82 (d, J=8.4 Hz, 1H), 6.74 (d, J=8.8 Hz, 1H), 4.82 (d, J=2.8 Hz, 1H), 4.59 (d, J=7.6 Hz, 1H), 3.67 (sep, J=6.8 Hz, 1H), 2.87-2.81 (m, 1H), 2.71-2.67 (m, 1H), 2.62-2.56 (m, 1H), 2.11 (s, 3H), 2.09 (s, 3H), 1.69-1.58 (m, 1H), 1.24-1.18 (m, 1H), 1.10 (d, J=6.8 Hz, 6H).

69L82: ¹H NMR (400 MHz, DMSO-d⁶) δ 9.82 (s, 1H), 8.00 (d, J=8.0 Hz, 2H), 7.72 (s, 1H), 7.56 (d, J=8.0 Hz, 2H), 7.39 (d, J=8.4 Hz, 1H), 7.21 (s, 1H), 6.97 (d, J=8.0 Hz, 1H), 6.89 (s, 1H), 6.84 (d, J=8.4 Hz, 1H), 6.74 (d, J=8.8 Hz, 1H), 4.00 (s, 1H), 3.91 (d, J=10.4 Hz, 1H), 3.10-3.08 (m, 1H), 2.92-2.88 (m, 1H), 2.31-2.25 (m, 1H), 2.12 (s, 3H), 2.10 (s, 3H), 1.88-1.79 (m, 1H), 1.48-1.40 (m, 1H), 1.11 (d, J=6.8 Hz, 6H).

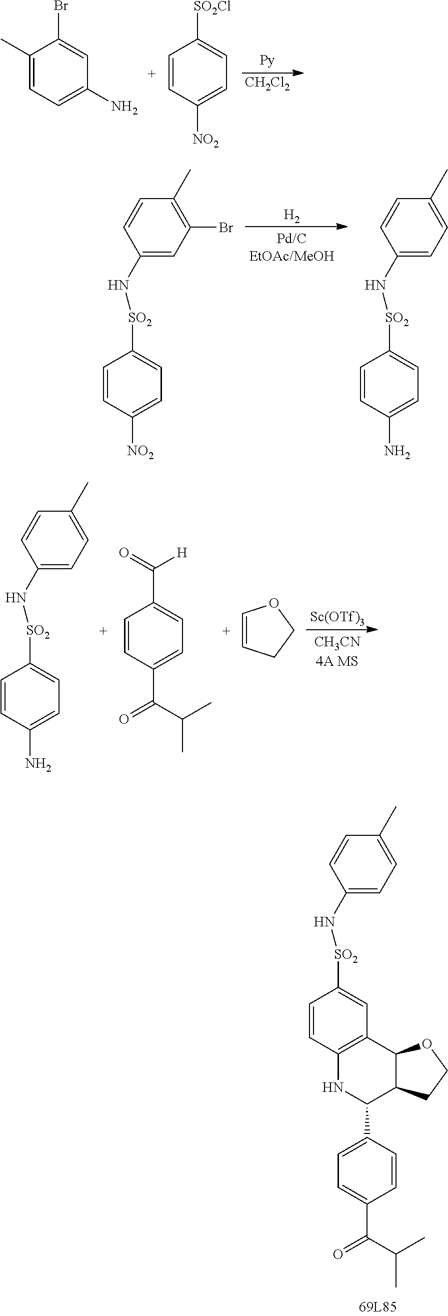

### Preparation of N-(3-bromo-4-methylphenyl)-4-nitrobenzenesulfonamide

4-Nitrobenzenesulfonyl chloride (10.00 g, 45.12 mmol) and 3-bromo-4-methylaniline (8.40 g, 45.12 m mol) were dissolved in anhydrous methylene chloride (100 mL) at room temperature under argon. Pyridine (5.35 g, 67.68 mmol) was added dropwise via a syringe at room temperature. The reaction mixture was stirred at room temperature overnight under argon atmosphere. Then, the volatile was removed under reduced pressure. The yellow solid crude was subjected to flash column chromatography (silica gel, CH₂Cl₂) to afford product as a pale yellow solid (1580 g, 94.3% yield). ¹H NMR (400 MHz, DMSO-d⁶) δ 10.75 (s, 1H), 8.39 (d, J=8.4 Hz, 2H), 8.00 (d, J=8.0 Hz, 2H), 7.30 (s, 1H), 7.24 (d, J=8.0 Hz, 1H), 7.04 (d, J=8.4 Hz, 1H), 2.23 (s, 3H).

### Preparation of 4-amino-N-(p-tolyl)benzenesulfonamide

N-(3-Bromo-4-methylphenyl)-4-nitrobenzenesulfonamide (10.00 g, 26.94 mmol) and Pd/C (10% Pd base, 2 g) were mixed together in MeOH (100 mL) and ethyl acetate (100 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (4.20 g, 59.4% yield). ¹H NMR (400 MHz, DMSO-d⁶) δ 9.69 (s, 1H), 7.34 (d, J=8.8 Hz, 2H), 6.99 (d, J=8.4 Hz, 2H), 6.94 (d, J=8.4 Hz, 2H), 6.50 (d, J=8.8 Hz, 2H), 5.95 (s, 2H), 2.17 (s, 3H).

### Preparation of 4-(4-isobutyrylphenyl)-N-(p-tolyl)-2,3,3a,4,5,9b-hexahydrofuro[3,2-c]quinoline-8-sulfonamide (69L85)

4-Amino-N-(p-tolyl)benzenesulfonamide (0.50 g, 1.47 mmol), 4-isobutyrylbenzaldehyde (0.26 g, 1.47 mmol), Sc(OTf)₃ (0.15 g, 0.29 mmol) and 4 Å molecular sieves (1 g) were mixed together in anhydrous CH₃CN (20 mL) at room temperature under argon. The reaction mixture was stirred for one hour at room temperature. 2,3-Dihydrofuran (0.21 g, 2.94 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.52 g, 72.2% yield). cis/trans diastereomers: ¹H NMR (400 MHz, CDCl₃) δ 7.98-7.95 (m, 2H), 7.86 (d, J=2.0 Hz, 0.6H), 7.76 (d, J=1.6 Hz, 0.4H), 7.52-7.48 (m, 2H), 7.42-7.38 (m, 1H), 7.03-6.93 (m, 4H), 6.84 (s, 0.4H), 6.78 (s, 0.6H), 6.56-6.53 (m, 1H), 5.18 (d, J=7.2 Hz, 0.4H), 4.84 (d, J=2.8 Hz, 0.4H), 4.74 (s, 0.6H), 4.50 (d, J=4.8 Hz, 0.6H), 4.44 (s, 0.4H), 4.05-4.01 (m, 0.6H), 3.90 (d, J=10.8 Hz, 0.6H), 3.87-3.83 (m, 0.6H), 3.72-3.64 (m, 0.8H), 2.56-2.51 (m, 1H), 2.76-2.72 (m, 0.4H), 2.42-2.38 (m, 0.6H), 2.26 (s, 3H), 2.05-1.99 (m, 1H), 1.71-1.68 (m, 0.6H), 1.52-1.43 (m, 0.4H), 1.22 (d, J=6.8 Hz, 6H).

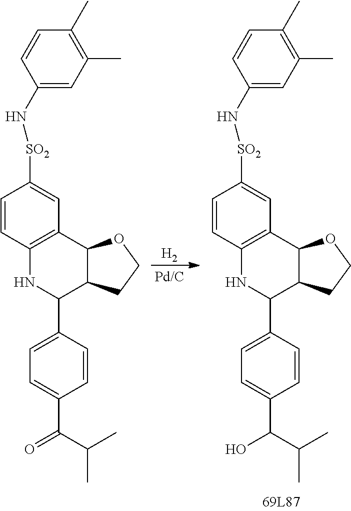

### Preparation of N-(3,4-dimethylphenyl)-4(4-(1-hydroxy-2-methylpropyl)phenyl)-2,3,3a,4,5,9b-hexahydrofuro[3,2-c]quinoline-8-sulfonamide (69L87)

N-(3,4-Dimethylphenyl)-4-(4-isobutyrylphenyl)-2,3,3a,4,5,9b-hexahydrofuro[3,2-c]quinoline-8-sulfonamide (0.20 g, 0.40 mmol) and Pd/C (10% Pd base, 0.10 g) were mixed together in MeOH (50 mL) and ethyl acetate (50 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a white solid product (0.18 g, 90% yield). cis/trans diastereomers: ¹H NMR (400 MHz, CDCl₃) δ 7.86 (d, J=2.0 Hz, 0.5H), 7.78 (d, J=2.0 Hz, 0.5H), 7.43-732 (m, 4H), 6.97 (d, J=8.0 Hz, 1H), 6.88 (s, 1H), 6.80-6.77 (m, 1H), 6.53-6.50 (m, 1H), 6.35 (s, 0.5H), 6.30 (s, 0.5H), 5.17 (d, J=7.6 Hz, 0.5H), 4.78 (d, J=2.8 Hz, 0.5H), 4.59 (s, 0.5H), 4.51 (d, J=4.8 Hz, 0.5H), 4.42-4.39 (m, 1H), 4.29 (s, 0.5H), 4.03-3.98 (m, 0.5H), 3.86-3.82 (m, 1H), 3.73-3.65 (m, 1H), 2.75-2.70 (m, 0.5H), 2.40-2.36 (m, 0.5H), 2.17 (s, 6H), 2.02-1.94 (m, 2H), 1.88 (s, 1H), 1.74-1.68 (m, 0.5H), 1.55-1.50 (m, 0.5H), 1.01-0.99 (m, 3H), 0.82 (t, J=6.8 Hz, 3H).

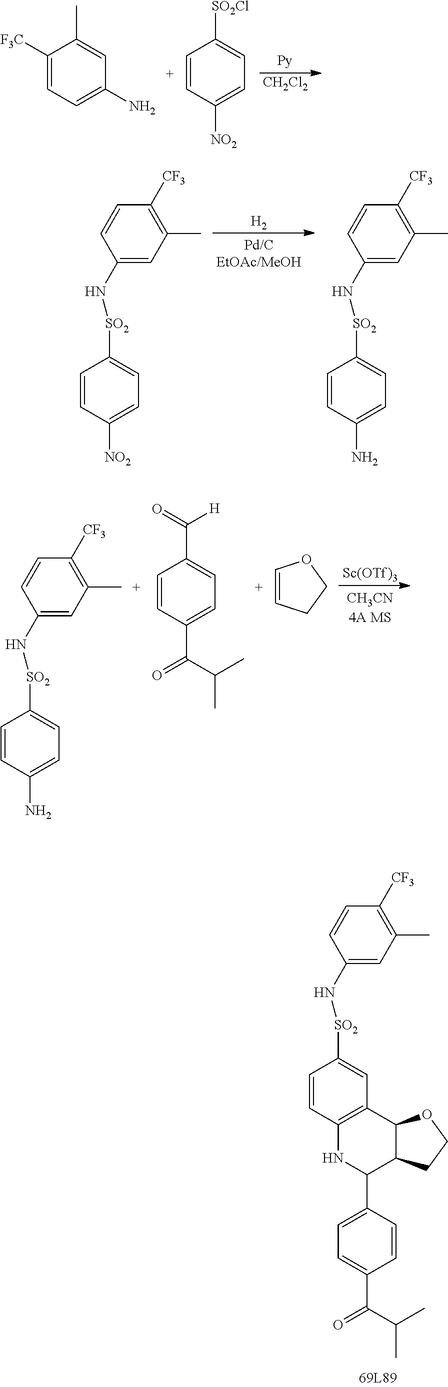

### Preparation of N-(3-methyl-4-(trifluoromethyl)phenyl)-4-nitrobenzenesulfonamide

4-Nitrobenzenesulfonyl chloride (1.39 g, 6.28 mmol) and 3-methyl-4-(trifluoromethyl)aniline (1.00 g, 571 mmol) were dissolved in anhydrous methylene chloride (20 mL) at room temperature under argon. Pyridine (1.35 g, 17.13 mmol) was added dropwise via a syringe at room temperature. The reaction mixture was stirred at room temperature overnight under argon atmosphere. Then, the volatile was removed under reduced pressure. The yellow solid crude was subjected to flash column chromatography (silica gel, CH₂Cl₂) to afford product as a pale yellow solid (1.85 g, 89.8% yield). ¹H NMR (400 MHz, DMSO-d⁶) δ 11.15 (s, 1H), 8.40 (d, J=88 Hz, 2H), 8.09 (d, J=8.8 Hz, 2H), 7.56 (d, J=8.8 Hz, 1H), 7.14 (s, 1H), 7.12 (d, J=8.0 Hz, 1H), 2.24 (s, 3H).

### Preparation of 4-amino-N-(3-methyl-4-(trifluoro methyl)phenyl)benzenesulfonamide

N-(3-Methyl-4-(trifluoromethyl)phenyl)-4-nitrobenzenesulfonamide (1.50 g, 2.78 mmol) and Pd/C (10% Pd base, 0.10 g) were mixed together in MeOH (50 mL) and ethyl acetate (50 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a white solid product (0.86 g, 93.8% yield). ¹H NMR (400 MHz, DMSO-d⁶) δ 10.42 (s, 1H), 7.51-7.46 (m, 3H), 7.07-7.04 (m, 2H), 6.55 (d, J=8.8 Hz, 2H), 6.07 (s, 2H), 2.32 (s, 3H).

### Preparation of 4-(trifluoromethyl)phenyl)-2,3,3a,4,5,9b-hexahydrofuro[3,2-c]quinoline-8-sulfonamide (69L89)

4-Amino-N-(3-methyl-4-(trifluoromethyl)phenyl)benzenesulfonamide (0.50 g, 1.51 mmol), 4-isobutyrylbenzaldehyde (0.27 g, 1.51 mmol), Sc(OTf)₃ (0.15 g, 0.30 mmol) and 4 Å molecular sieves (1 g) were mixed together in anhydrous CH₃CN (20 mL) at room temperature under argon. The reaction mixture was stirred for one hour at room temperature. 2,3-Dihydrofuran (0.21 g, 3.02 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.65 g, 77.1% yield). cis/trans diastereomers: ¹H NMR (400 MHz, CDCl₃) δ 7.97-7.94 (m, 2.5H), 7.88 (s, 0.5H), 7.70-7.67 (m, 1H), 7.57-7.47 (m, 3H), 7.43-7.38 (m, 1H), 7.01-6.99 (m, 2H), 6.64-6.60 (m, 1H), 5.20 (d, J=7.2 Hz, 0.5H), 4.89-4.85 (m, 1H), 4.58 (s, 0.5H), 4.52 (d, J=4.4 Hz, 0.5H), 4.02-3.98 (m, 0.5H), 3.91-3.84 (m, 1H), 2.74-2.68 (m, 0.5H), 3.63-3.51 (m, 1.5H), 2.76-2.74 (m, 0.5H), 2.37 (s, 3f), 2.07-1.98 (m, 1f), 1.72-1.69 (m, 0.5H), 1.50-1.48 (m, 0.5H), 1.21 (d, J=68 Hz, 6H).

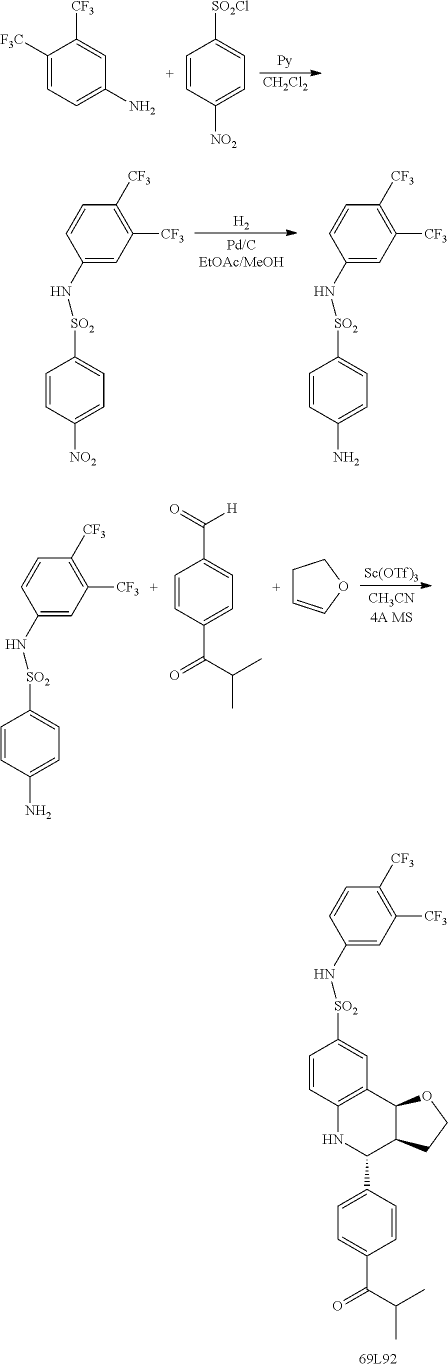

### Preparation of N-(3,4-bis(trifluoro methyl)phenyl)-4-nitrobenzenesulfonamide

4-Nitrobenzenesulfonyl chloride (1.01 g, 4.57 mmol) and 3,4-bis(trifluoromethyl)aniline (0.95 g, 4.15 mmol) were dissolved in anhydrous methylene chloride (20 mL) at room temperature under argon. Pyridine (0.99 g, 12.45 mmol) was added dropwise via a syringe at room temperature. The reaction mixture was stirred at room temperature overnight under argon atmosphere. Then, the volatile was removed under reduced pressure. The yellow solid crude was subjected to flash column chromatography (silica gel, CH₂Cl₂) to afford product as a pale yellow solid (1.58 g, 91.9% yield). ¹H NMR (400 MHz, CDCl₃) δ 8.36 (d, J=8.8 Hz, 2H), 8.06 (d, J=8.8 Hz, 2H), 7.78 (d, J=8.8 Hz, 1H), 7.52 (d, J=1.6 Hz, 1H), 7.47 (d, J=8.8 Hz, 1H), 7.31 (s, 1H).

### Preparation of 4-amino-N-(3,4-bis(trifluoromethyl)phenyl)benzenesulfonamide

N-(3,4-bis(Trifluoromethyl)phenyl)-4-nitrobenzenesulfonamide (1.50 g, 3.62 mmol) and Pd/C (10% Pd base, 0.10 g) were mixed together in MeOH (50 mL) and ethyl acetate (50 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a white solid product (1.30 g, 93.5% yield). ¹H NMR (400 MHz, DMSO-d⁶) δ 10.97 (s, 1H), 7.90 (d, J=8.8 Hz, 1H), 7.61 (d, J=1.6 Hz, 1H), 7.52-7.48 (m, 3H), 6.59-6.57 (m, 2H), 6.17 (s, 2H).

### Preparation of N-(3,4-bis(trifluoromethyl)phenyl)-4-(4-isobutyrylphenyl)-2,3,3a,4,5,9b-hexahydrofuro[3,2-c]quinoline-8-sulfonamide (69L92)

4-Amino-N-(3-methyl-4-(trifluoromethyl)phenyl)benzenesulfonamide (0.50 g, 1.51 mmol), 4-isobutyrylbenzaldehyde (0.27 g, 1.51 mmol), Sc(OTf)₃ (0.15 g, 0.30 mmol) and 4 Å molecular sieves (1 g) were mixed together in anhydrous CH₃CN (20 mL) at room temperature under argon. The reaction mixture was stirred for one hour at room temperature. 2,3-Dihydrofuran (0.21 g, 3.02 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.65 g, 77.1% yield). cis/trans diastereomers: ¹H NMR (400 MHz, CDCl₃) δ 7.97-7.94 (m, 2.5H), 7.88 (s, 0.5H), 7.70-7.67 (m, 1H), 7.57-7.47 (m, 3H), 7.43-7.38 (m, 1H), 7.01-6.99 (m, 2H), 6.64-6.60 (m, 1H), 5.20 (d, J=7.2 Hz, 0.5H), 4.89-4.85 (m, 1H), 4.58 (s, 0.5H), 4.52 (d, J=4.4 Hz, 0.5H), 4.02-3.98 (m, 0.5H), 3.91-3.84 (m, 1H), 2.74-2.68 (m, 0.5H), 3.63-3.51 (m, 1.5H), 2.76-2.74 (m, 0.5H), 2.37 (s, 3H), 2.07-1.98 (m, 1H), 1.72-1.69 (m, 0.5l), 1.50-1.48 (m, 0.5H), 121 (d, J=6.8 Hz, 6H).

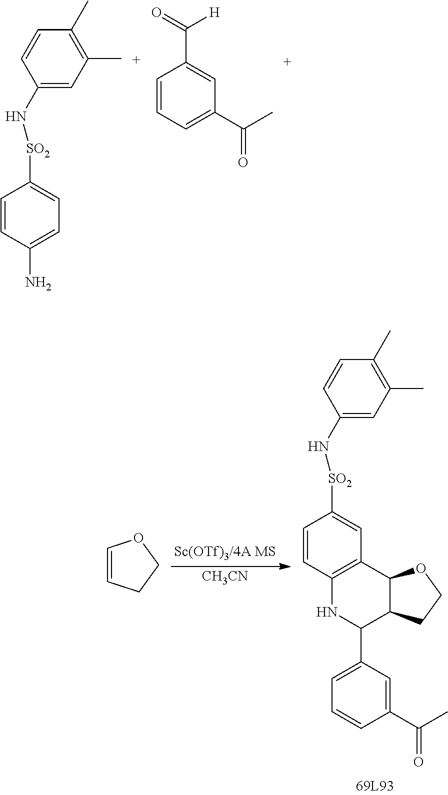

### Preparation of 4-(3-acetylphenyl)-N-(3,4-dimethylphenyl)-2,3,3a,4,5,9b-hexahydrofuro[3,2-c]quinoline-8-sulfonamide (69L93)

4-Amino-N-(3,4-dimethylphenyl)benzenesulfonamide (0.50 g, 1.81 mmol), 3-acetylbenzaldehyde (0.27 g, 1.81 mmol), Sc(OTf)₃ (0.18 g, 0.36 mmol) and 4 Å molecular sieves (1 g) were mixed together in anhydrous CH₃CN (20 mL) at room temperature under argon. The reaction mixture was stirred for one hour at room temperature. 2,3-Dihydrofuran (0.25 g, 3.62 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product as cis/trans diastereomers (0.58 g, 67.4% yield). ¹H NMR (400 MHz, DMSO-d⁶) δ 9.77 (s, 0.5H), 9.73 (s, 0.5H), 8.00 (d, J=3.6 Hz, 1H), 7.96-7.91 (m, 1H), 7.73-7.70 (m, 1H), 7.60-7.55 (m, 2H), 7.39 (d, J=8.8 Hz, 0.5H), 7.33 (d, J=8.4 Hz, 0.5H), 7.16 (s, 0.5H), 6.96 (d, J=8.0 Hz, 1H), 6.89-6.80 (m, 2.5H), 6.74-6.71 (m, 1H), 5.11 (d, J=7.2 Hz, 0.5H), 4.86 (s, 0.5H), 4.43 (d, J=4.8 Hz, 0.5H), 3.94-3.88 (m, 0.5H), 3.80 (d, J=10.8 Hz, 0.5H), 3.74-3.68 (m, 0.5H), 3.63-3.57 (m, 0.5H), 3.52-2.50 (m, 0.5H), 2.70-2.65 (m, 0.5H), 2.59 (s, 3H), 2.34-2.29 (m, 0.5H), 2.11 (s, 3H), 2.09 (s, 3H), 1.98-1.92 (m, 0.5H), 1.81-1.76 (m, 0.5H), 1.56-1.50 (m, 0.5H), 1.31-1.29 (m, 0.5H).

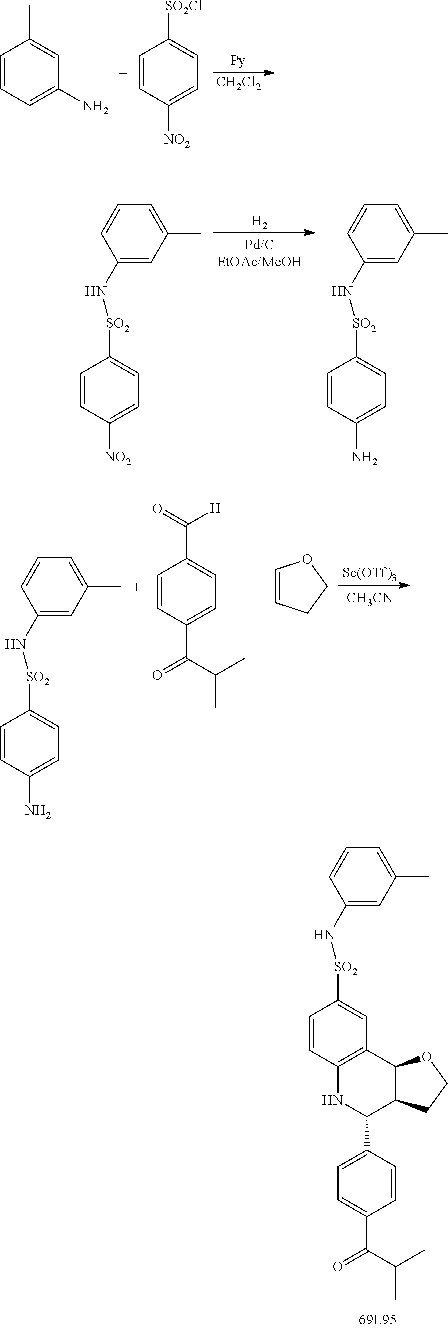

### Preparation of 4-nitro-N-(m-tolyl)benzenesulfonamide

4-Nitrobenzenesulfonyl chloride (20.68 g, 93.22 mmol) and m-toluidine1 (10.00 g, 93.22 mmol) were dissolved in anhydrous methylene chloride (100 mL) at room temperature under argon. Pyridine (11.07 g, 0.14 mol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=9/1 v/v) to afford the desired product as a pale yellow solid (27.0 g, 99% yield). ¹H NMR (400 MHz, DMSO-d⁶) δ 10.56 (s, 1H), 8.38 (d, J=8.0 Hz, 2H), 8.00 (d, J=8.0 Hz, 2H), 7.15-7.11 (m, 1H), 6.93-6.88 (m, 3H), 2.20 (s, 3H).

### Preparation of 4-amino-N-(n-tolyl)benzenesulfonamide

4-Nitro-N-(m-tolyl)benzenesulfonamide (20.00 g, 68.42 mmol) and Pd/C (10% Pd base, 2 g) were mixed together in MeOH (100 mL) and Ethyl acetate (100 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (15.6 g, 87.2% yield). ¹H NMR (400 MHz, DMSO-d⁶) δ 9.80 (s, 1H), 7.38 (d, J=4 Hz, 2H), 7.09-7.05 (m, 1H), 6.86 (s, 2H), 6.77 (d, J=7.2 Hz, 1H), 6.52 (d, J=8.4 Hz, 2H), 5.97 (s, 2H), 2.18 (s, 3H).

### Preparation of N-(4-acetylphenyl)-4-(4-isobutyrylphenyl)-2,3,3a,4,5,9b-hexahydrofuro[3,2-c]quinoline-8-sulfonamide (69L95)

4-Amino-N-(m-tolyl)benzenesulfonamide (0.57 g, 2.28 mmol), 4-isobutyrylbenzaldehyde (0.40 g, 2.28 mmol), Sc(OTf)₃ (0.23 g, 0.46 mmol) and 4 Å molecular sieves were mixed together in anhydrous CH₃CN (20 mL) at room temperature under argon. The reaction mixture was stirred for one hour at room temperature. 2,3-Dihydrofuran (0.32 g, 4.56 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.65 g, 58.7% yield). ¹H NMR (400 MHz, DMSO-d⁶, syn/anti-diastereomers) δ 9.93 (s, 0.5H), 9.90 (s, 0.5H), 7.99 (d, J=8.0 Hz, 2H), 7.60 (d, J=7.6 Hz, 2H), 7.57 (s, 1H), 7.42 (d, J=8.4 Hz, 0.5H), 7.35 (d, J=8.8 Hz, 0.5H), 7.21 (s, 0.5H), 7.11-7.07 (m, 1H), 6.91-6.88 (m, 2.5H), 6.80 (d, J U=7.2 Hz 1H), 6.75-6.71 (m, 1H), 5.11 (d, J=7.2. Hz, 0.5H), 4.85 (s, 0.5H), 4.43 (d, J=4.4 Hz, 0.5H), 3.91-3.87 (m, 0.5H), 3.80 (d, J=10.8 Hz, 0.5H), 3.74-3.45 (m, 3H), 2.71-2.65 (m, 0.5H), 2.31-2.24 (m, 0.5H), 2.20 (s, 3H), 1.97-1.92 (m, 0.5H), 1.80-1.75 (m, 0.5H), 1.58-1.53 (m, 0.5H), 1.32-1.30 (m, 0.5H), 1.10 (d, J=6.8 Hz, 6H).

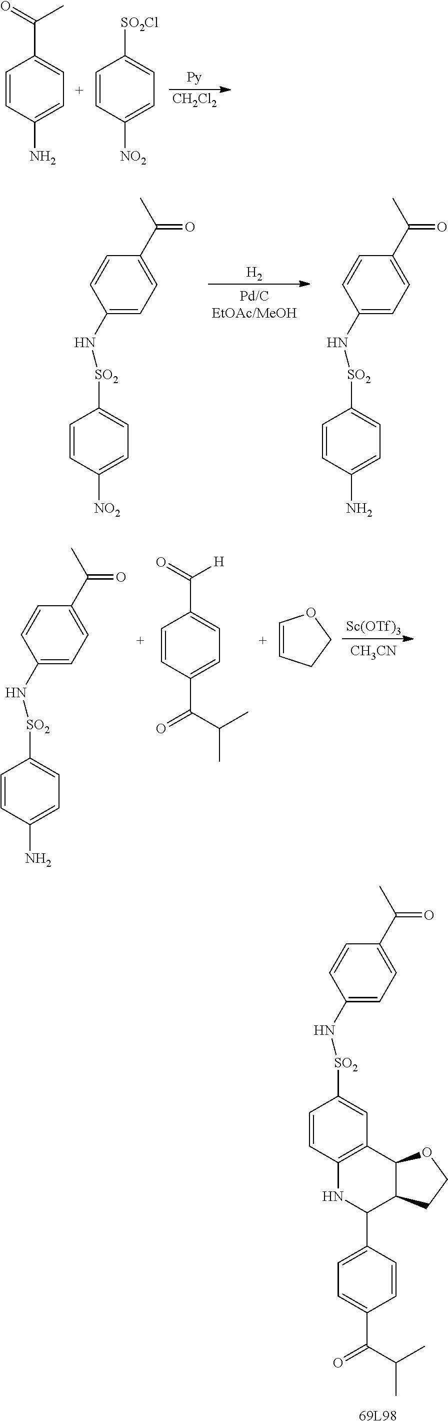

### Preparation of N-(4-acetylphenyl)-4-nitrobenzenesulfonamide

4-Nitrobenzenesulfonyl chloride. (10.00 g, 45.12 mmol) and 1-(4-aminophenyl)ethan-1-one (6.10 g, 45.12 mmol) were dissolved in anhydrous methylene chloride (100 mL) at room temperature under argon. Pyridine (5.35 g, 67.68 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel CH₂Cl₂/Acetone=9/1 v/v) to afford the desired product as a pale yellow solid (13.80 g, 95.5% yield). ¹H NMR (400 MHz, DMSO-d⁶) δ 11.20 (s, 1H), 8.39 (d, J=8.4 Hz, 2H), 8M08 (d, J=8.4 Hz, 2H), 7.86 (d, J=8.4 Hz, 1H), 7.24 (d, J=8.4 Hz, 1H), 2.48 (s, 3H).

### Preparation of N-(4-acetylphenyl)-4-aminobenzenesulfonamide

N-(3-Acetylphenyl)-4-nitrobenzenesulfonamide (10.00 g, 31.22 mmol) and Pd/C (10% Pd base, 1 g) were mixed together in MeOH (100 mL) and Ethyl acetate (100 mL) at room temperature. Hydrogen gas was introduced via a H₂ balloon. The reaction mixture was stirred under H₂ atmosphere 8 hours at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (7.18 g, 79.2% yield). ¹H NMR (400 MHz, DMSO-d⁶) δ 10.50 (s, 1H), 7.82 (d, J=8.4 Hz, 2H), 7.47 (d, J=8.4 Hz, 2H), 7.17 (d, J=8.0 Hz, 2H), 6.55 (d, J=8.0 Hz, l-H), 6.07 (s, 2H), 2.46 (s, 3H).

### Preparation of N-(4-acetylphenyl)-4-(4-isobutyrylphenyl)-2,3,3a,4,5,9b-hexahydrofuro[3,2-c]quinoline-8-sulfonamide (69L98)

N-(4-Acetylphenyl)-4-aminobenzenesulfonamide (0.50 g, 1.72 mmol), 4-isobutyryl-benzaldehyde (0.30 g, 1.72 mmol), Sc(OTf)₃ (0.17 g, 0.34 mmol) and 4 Å molecular sieves were mixed together in anhydrous CH₃CN (20 mL) at room temperature under argon. The reaction mixture was stirred for one hour at room temperature. 2,3-Dihydrofuran (0.24 g, 3.44 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.45 g, 50.6% yield). cis/trans diastereomers: ¹H NMR (400 MHz, DMSO-d⁶) δ 10.60 (s, 0.5H), 10.57 (s, 0.5H), 7.99 (d, J=8.0 Hz, 2H), 7.84 (d, J=8.4 Hz, 2H), 7.67-7.57 (m, 3H), 7.49 (d, J=9.2 Hz, 0.5H), 7.43 (d, J=9.2 Hz, 0.5H), 7.29 (s, 0.5H), 7.20 (dd, J₁=8.4 Hz, J₂=2.0 Hz, 2H), 7.00 (s, 0.5H), 6.77-6.73 (m, 1H), 5.12 (d, J=7.6 Hz, 0.5H), 4.85 (d, J=2.0 Hz, 0.5H), 4.45 (d, J=4.8 Hz, 0.5H), 3.91-187 (m, 0.5H), 3.79 (d, J=16 Hz, 0.5H), 3.74-3.59 (m, 2H), 3.52-3.50 (m, 0.5H), 2.71-2.65 (m, 0.5H), 2.47 (s, 3H), 2032-2.38 (m, 0.5H), 1.94-1.92 (m, 0.5H), 1.79-1.74 (m, 0.5H), 1.56-1.53 (m, 0.5H), 1.32-1.28 (m, 0.5H), 1.10 (d, J=6.4 Hz, 6H).

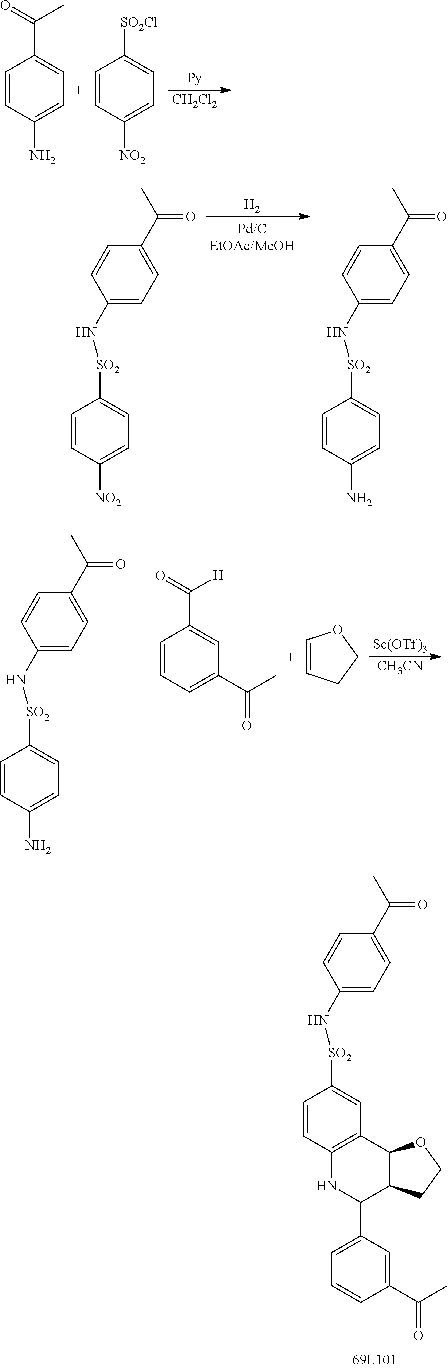

### Preparation of 4-(3-acetylphenyl)-N-(4-acetylphenyl)-2,3,3a,4,5,9b-hexahydrofuro[3,2-c]quinoline-8-sulfonamide (69L101)

N-(4-Acetylphenyl)-4-aminobenzenesulfonamide (0.50 g, 1.72 mmol), 3-acetylbenzaldehyde (0.26 g, 1.72 mmol), Sc(OTf)₃ (0.17 g, 0.34 mmol) and 4 Å molecular sieves were mixed together in anhydrous CH₃CN (20 mL) at room temperature under argon. The reaction mixture was stirred for one hour at room temperature. 2,3-Dihydrofuran (0.24 g, 3.44 mmol) was added via a syringe. The resulted mixture was stirred overnight at room temperature under argon. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The crude solid was purified by column chromatography (silica gel, CH₂Cl₂/acetone=9/1 i/v) to afford the desired product as a white solid (0.51 g, 60.7% yield). cis/trans diasteriomers: ¹H NMR (400 MHz, DMSO-d⁶) δ 10.60 (s, 0.5H), 10.57 (s, 0.5H), 8.00 (d, J=6.0 Hz, 1H), 7.95-7.91 (m, 1H), 7.84 (d, J=8.8 Hz, 2H), 7.73-7.67 (m, 1.5H), 7.62 (s, 0.5H), 7.57-7.53 (m, 1H), 7.49 (dd, J₁=8.8 Hz, J₂=2.0 Hz, 0.5), 7.43 (d, J=8.8 Hz, 0.5H), 7.27 (s, 0.5H), 7.20 (dd, J₁=8.8 Hz, J₂=2.8 Hz, 2H), 7.01 (s, 0.5H), 6.77-6.74 (m, 1H), 5.12 (d, J=7.6 Hz, 0.5H), 4.86 (d, J=2.4 Hz, 0.5H), 4.45 (d, J=4.8 Hz, 0.5H), 3.94-3.89 (r, 0.5H), 3.80 (d, J=10.8 Hz, 0.5H), 3.74-3.68 (m, 2H), 3.61-3.57 (m, 0.5H), 3.52-3.49 (ma, 0.5H), 2.70-2.65 (m, 0.5H), 2.59 (s, 3H), 2.47 (s, 3H), 2.33-2.29 (m, 0.5H), 1.96-1.91 (m, 0.5H), 1.79-1.74 (m, 0.5H), 1.54-1.52 (m, 0.5H), 1.32-1.28 (m, 0.5H).

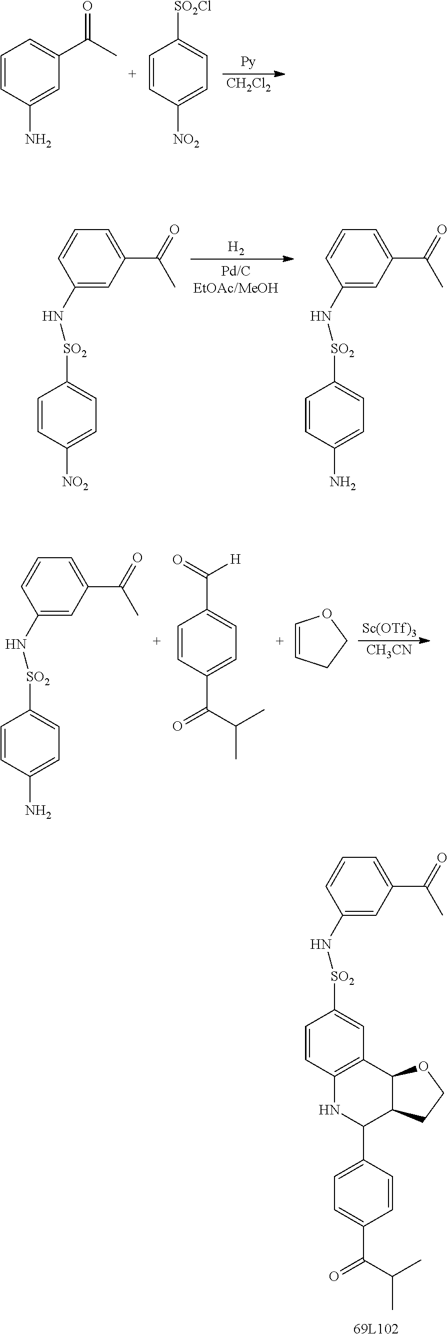

### Preparation of N-(3-acetylphenyl)-4-nitrobenzenesulfonamide

4-Nitrobenzenesulfonyl chloride (10.00 g, 45.12 mmol) and 1-(3-aminophenyl)ethan-1-one (6.10 g, 45.12 mmol) were dissolved in anhydrous methylene chloride (100 mL) at room temperature under argon. Pyridine (5.35 g, 67.68 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=9/1 v/v) to afford the desired product as a pale yellow solid (14.20 g, 98% yield). ¹H NMR (400 MHz, DMSO-d⁶) δ 10.99 (s, 1H), 8.38 (d, J=8.4 Hz, 2H), 8.02 (d, J=8.4 Hz, 2H), 7.71 (d, J=7.6 Hz, 1H), 7.67 (s, 1H), 7.46-7.42 (m, 11H), 7.38 (d, J=8.0 Hz, 1H), 2.52 (s, 3H).

### Preparation of N-(3-acetylphenyl)-4-aminobenzenesulfonamide

N-(3-Acetylphenyl)-4-nitrobenzenesulfonamide (10.00 g, 31.22 mmol) and Pd/C (10% Pd base, 1 g) were mixed together in MeOH (100 mL) and Ethyl acetate (100 mL) at room temperature. Hydrogen gas was introduced via a H₂ balloon. The reaction mixture was stirred under H₂ atmosphere 8 hours at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (5.60 g, 61.8% yield). ¹H NMR (400 MHz, DMSO-d⁶) δ 10.14 (s, 1H), 7.61-7.58 (m, 2H), 7.41-7.33 (m, 4H), 6.52 (d, J=8.4 Hz, 2H), 6.01 (s, 2H), 2.49 (s, 3H).

### Preparation of N-(3-acetylphenyl)-4-(4-isobutyrylphenyl)-2,3,3a,4,5,9b-hexahydrofuro[3,2-c]quinoline-8-sulfonamide (69L102)

N-(3-Acetylphenyl)-4-aminobenzenesulfonamide (0.51 g, 1.76 mmol), 4-isobutyryl-benzaldehyde (0.31 g, 1.76 mmol), Sc(OTf)₃ (0.17 g, 0.35 mmol) and 4 Å molecular sieves were mixed together in anhydrous CH₃CN (20 mL) at room temperature under argon. The reaction mixture was stirred for one hour at room temperature. 2,3-Dihydrofuran (0.25 g, 3.52 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.45 g, 49.5% yield). cis/trans diastereomers 69L102: ¹H NMR (400 MHz, CDCl₃) δ7.98 (d, J=8.0 Hz, 2H), 7.89 (s, 0.5H), 7.82. (s, 0.5H), 7.68 (d, J=7.2 Hz, 1H), 7.61 (s, 1H), 7.51-7.45 (m, 3H), 7.41-7.36 (m, 2H), 6.89 (s, 0.5H), 6.82 (s, 0.5H), 6.58-6.55 (m, 1H), 5.19 (d, J=7.2 Hz, 0.5H), 4.86 (s, 0.5H), 4.66 (s, 0.5H), 4.52 (d, J=4.4 Hz, 0.5H), 4.37 (s, 0.5H), 4.02-3.98 (m, 0.5H), 3.92-3.84 (m, 1H), 3.72-3.69 (m, 0.5H), 3.66-3.63 (m, 0.5H), 3.59-3.52 (m, 1H), 2.78-2.74 (m, 0.5H), 2.57 (s, 3H), 2.42-2.37 (in. 0.51), 2.06-1.97 (m, 1H), 1.72-1.69 (m, 0.5H), 1.52-1.49 (m, 0.5H), 1.23 (d, J=6.8 Hz, 6H).

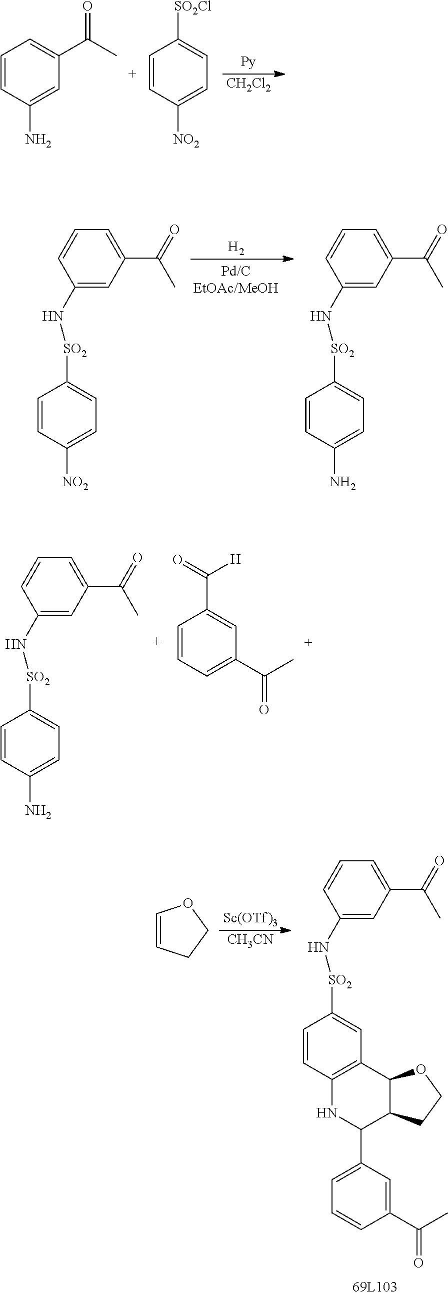

### Preparation of N,4-bis(3-acetylphenyl)-2,3,3a,4,5,9b-hexahydrofuro[3,2-c]quinoline-8-sulfonamide (69L103)

N-(3-Acetylphenyl)-4-aminobenzenesulfonamide (0.50 g, 1.72 mmol), 3-acetylbenzaldehyde (0.26 g, 1.72 mmol), Sc(OTf)₃ (0.17 g, 0.34 mmol) and 4 Å molecular sieves were mixed together in anhydrous CH₃CN (20 mL) at room temperature under argon. The reaction mixture was stirred for one hour at room temperature. 2,3-Dihydrofuran (0.24 g, 3.44 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.43 g, 51.2% yield). cis/trans diastereomers 69L103: ¹H NMR (400 MHz, CDCl₃) δ 8.00 (d, J=6.4 Hz, 1H), 7.96-7.88 (m, 1.5H), 7.81 (s, 0.5H), 7.67 (d, J=7.2 Hz, 1H), 7.53-7.45 (m, 2H), 7.43-7.34 (m, 2H), 7.01 (s, 0.5H), 6.92 (s, 0.5H), 6.58-6.55 (m, 1H), 5.18 (d, J=7.6 Hz, 0.5H), 4.87 Is, 0.5H), 4.68 (s, 0.5H), 4.51 (d, J=4.4 Hz, 0.5H), 4.41 (s, 0.5H), 4.05-3.99 (m, 0.5H), 3.91-3.82 (m, 1H), 3.72-3.68 (m, 0.5H), 3.65-3.61 (m, 0.5H), 2.76-2.74 (m, 0.5H), 2.63 (s, 3H), 2.57 (s, 3H), 2.44-2.38 (m, 0.5H), 2.08-1.98 (m, 1H), 1.71168 (m, 0.5H), 1.51-1.47 (m, 0.5H).

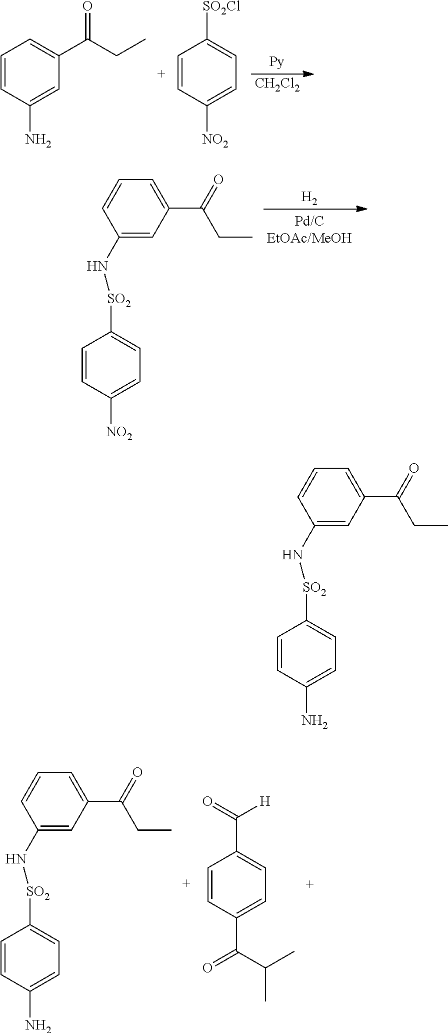

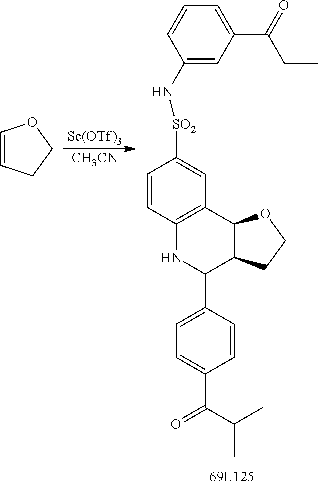

### Preparation of 4-nitro-N-(3-propionylphenyl)benzenesulfonamide

4-Nitrobenzenesulfonyl chloride (7.43 g, 33.51 mmol) and 1-(3-aminophenyl)propan-1-one (5.00 g, 33.51 mmol) were dissolved in anhydrous methylene chloride (100 mL) at room temperature under argon. Pyridine (3.97 g, 50.27 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=9/1 v/v) to afford the desired product as a pale yellow solid (11.00 g, 98.2% yield). ¹H NMR (400 MHz, DMSO-d⁶) δ 10.88 (s, 1H), 8.01 (d, J=8.8 Hz, 2H), 7.70 (dt, J₁=7.6 Hz, J₂=1.2 Hz, 1H), 7.67 (t, J=2.4 Hz, 1H), 7.45-7.41 (m, 1H), 7.37-7.34 (m, 1H), 2.96 (q, J=7.2 Hz, 2H), 1.04 (t, J=7.2 Hz, 3H).

### Preparation of 4-amino-N-(3-propionylphenyl)benzenesulfonamide

4-Nitro-N-(3-propionylphenyl)benzenesulfonamide (10.00 g, 29.91 mmol) and Pd/C (10% Pd base, 1 g) were mixed together in MeOH (100 mL) and Ethyl acetate (100 mL) at room temperature. Hydrogen gas was introduced via a H₂ balloon. The reaction mixture was stirred under H₂ atmosphere 8 hours at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (8.50 g, 93.4% yield). ¹H NMR (400 MHz, DMSO-d⁶) δ 10.12 (s, 1H), 7.62 (t, J=1.6 Hz, 1H), 7.59 (d, J=7.6 Hz, 1H), 7.40 (d, J=7.6 Hz, 2H), 7.38-7.29 (m, 2H), 6.52 (d, J=8.8 Hz, 2H), 6.01 (s, 2H), 2.94 (q, J=7.2 Hz, 2H), 1.04 (t, J=7.2 Hz, 3H).

### Preparation of 4-(4-isobutyrylphenyl)-N-(3-propionylphenyl)-2,3,3a,4,5,9b-hexahydrofuro[3,2-c]quinoline-8-sulfonimide (69L125)

4-Amino-N-(3-propionylphenyl)benzenesulfonamide (0.58 g, 1.91 mmol), 4-isobutyrylbenzaldehyde (0.34 g, 1.91 mmol), Sc(OTf)₃ (0.19 g, 0.38 mmol) and 4 Å molecular sieves were mixed together in anhydrous CH₃CN (20 mL) at room temperature under argon. The reaction mixture was stirred for one hour at room temperature. 2,3-Dihydrofuran (0.27 g, 3.81 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.56 g, 51.7% yield). cis/rans diastereomers: ¹H NMR (400 MHz, CDCl₃) δ 8.00 (d, J=8.0 Hz, J=2.8 Hz, 2H), 7.90 (d, 2.0 Hz, 0.5H), 7.83 (s, 0.5H), 7.72-7.70 (m, 1H), 7.64 (s, 1H), 7.53-7.47 (m, 3H), 7.41-7.37 (m, 2H), 6.60-6.56 (m, 1H), 5.20 (d, J=7.6 Hz, 0.5H), 4.88 (d, J=2.8 Hz, 0.5H), 4.65 (s, 0.5H), 4.54 (d, J=4.4 Hz, 0.5H), 4.37 (s, 0.5H), 4.04-4.02 (m, 0.5H), 3.94-3.87 (m, 1H), 3.74-3.65 (m, 1H), 3.59-3.55 (m, 1H), 3.01-2.95 (m, 2H), 2.80-2.76 (m, 0.5H), 2.43-2.38 (m, 0.5H), 2.11-1.97 (m, 1H), 1.74-1.69 (m, 0.5H), 1.55-1.49 (m, 0.5H), 1.26-1.20 (m, 9H).

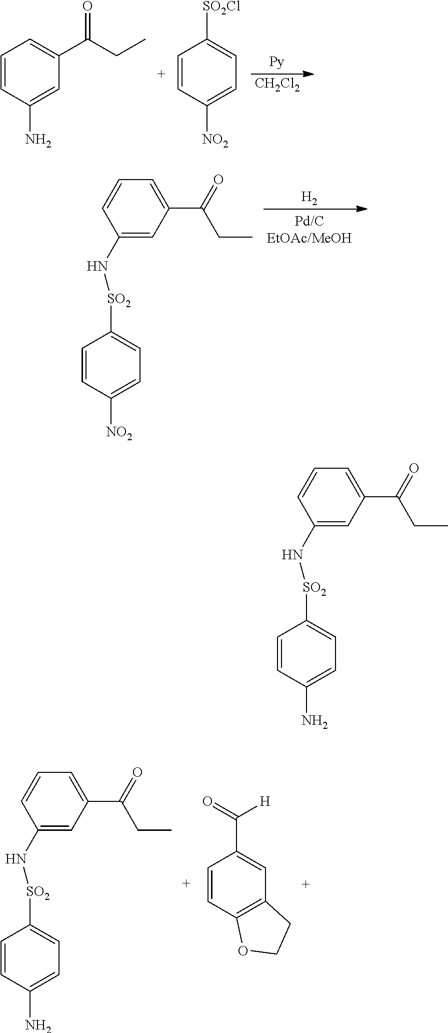

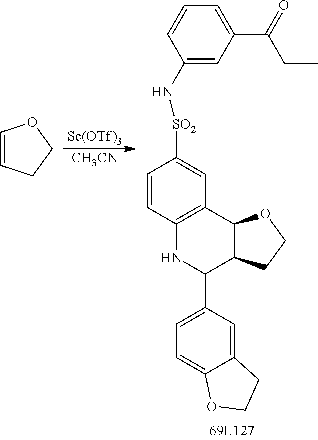

### Preparation of 4-(2,3-dihydrobenzofuran-5-yl)-N-(3-propionylphenyl)-2,3,3a,4,5,9b-hexahydrofuro[3,2-c]quinoline-8-sulfonamide (69L127)

4-Amino-N-(3-propionylphenyl)benzenesulfonamide (0.50 g, 1.64 mmol), 2,3-dihydrobenzofuran-5-carbaldehyde (0.24 g, 1.64 mmol), Sc(OTf)₃ (0.16 g, 0.33 mmol) and 4 Å molecular sieves were mixed together in anhydrous CH₃CN (20 mL) at room temperature under argon. The reaction mixture was stirred for one hour at room temperature. 2,3-Dihydrofuran (0.23 g, 3.28 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.40 g, 48.3% yield). syn/anti-diastereomers: 1H NMR (400 MHz, CDCl₃) δ 7.89 (d, J=2.4 Hz, 0.5H), 7.82 (d, J=2.0 Hz, 0.5H), 7.70-7.67 (m, 1H), 7.66 (s, 1H), 7.48-7.44 (m, 1H), 7.43-7.35 (m, 2H), 7.24 (s, 1H), 7.21 (s, 0.5H), 7.15-7.11 (m, 1H), 7.07 (s, 0.5H), 6.80-6.78 (m, 1H), 6.55-6.50 (s, 1H), 5.17 (d, J=7.6 Hz, 0.5H), 4.72 (d, J=3.2 Hz, 0.5H), 4.65-4.59 (m, 2.5H), 4.51 (d, J=4.8 Hz, 0.5H), 4.33 (s, 0.5H), 4.02-3.98 (m, 0.5H), 3.86-3.82 (m, 0.5H), 3.78-3.66 (m, 0.5H), 3.23=8.8 Hz, 2H), 3.00-2.94 (m, 2H), 2.71-2.67 (m, 0.5H), 2.38-2.32 (m, 0.5H), 2.02-1.97 (m, 1H), 1.76-1.70 (m, 0.5H), 1.64-1.56 (m, 0.5H), 1.23-1.19 (m, 3H).

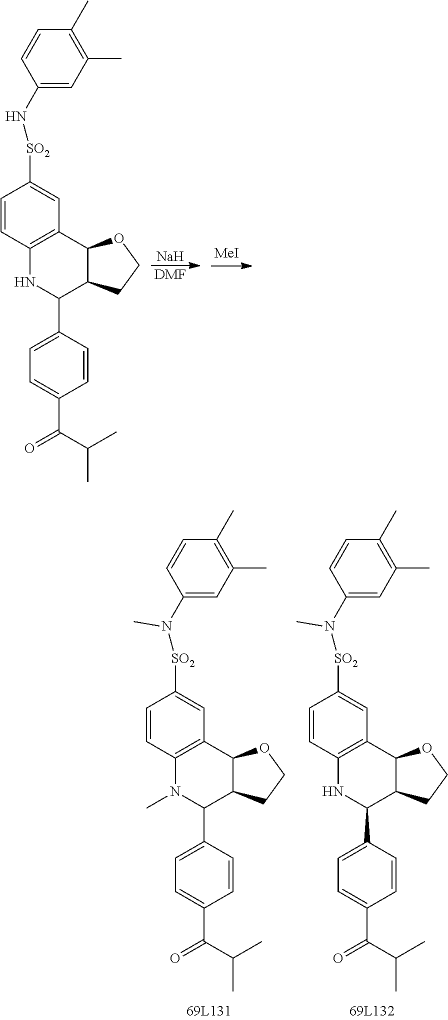

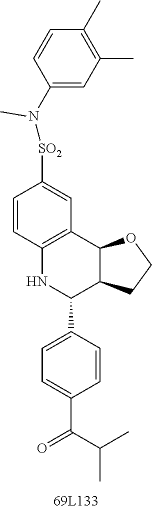 [SMILES: Cc1ccc(N(C)S(=O)(=O)c2ccc3c(c2)[C@H]2OCC[C@H]2[C@H](c2ccc(C(=O)C(C)C)cc2)N3)cc1C]

### Preparation of N-(3,4-dimethylphenyl)-4-(4-isobutyrylphenyl)-N,5-dimethyl-2,3,3a,4,5,9b-hexahydrofuro[3,2-c]quinoline-8-sulfonamide (69L131) and N-(3,4-dimethylphenyl)-4-(4-isobutyrylphenyl)-N-methyl-2,3,3a,4,5,9b-hexahydrofuro[3,2-c]quinoline-8-sulfonamide and (69L132, 69L133)

N-(3,4-Dimethylphenyl)-4-(4-isobutyrylphenyl)-2,3,3a,4,5,9b-hexahydrofuro[3,2-c]quinoline-8-sulfonamide (0.30 g, 0.59 mmol) was dissolved in 20 mL of anhydrous DMF at room temperature. NaH (26 mg, 60% weight in mineral oil, 0.65 mmol) was added at room temperature. After stirred at room temperature for one hour, Mel (0.13 g, 0.89 mmol) was added to the reaction mixture. After stirred at room temperature overnight, the reaction was quenched by adding 100 mL of saturated NH₄Cl solution. The white precipitate formed was isolated by filtration and purified by column chromatography (silica gel, CH₂Cl₂/Acetone=19/1 v/v) to afford three products as white solids. 69L131 (0.11 g, 35.5% yield). 69L132 (0.06 g, 19.6% yield) and 69L133 (0.10 g, 32.7% yield). ¹H NMR (400 MHz, DMSO-d⁶) for 69L131 (syn/anti-diastereomers): δ 7.95 (d, J=8.4 Hz, 1H), 7.90 (d, J=8.4 Hz, 1H), 7.37-7.30 (m, 3H), 7.25 (d, J=2.4 Hz, 0.5H), 7.18 (d, J=10 Hz, 0.5H), 7.08-7.04 (m, 1H), 6.87-6.75 (m, 3H), 4.77 (t, J=6.0 Hz, 1H), 4.51-4.47 (m, 1H), 3.73-3.60 (m, 2H), 3.52-3.48 (m, 0.5H), 3.03 (s, 1.5H), 3.01 (s, 1.5H), 2.94-2.88 (m, 0.5H), 2.84 (s, 1.5H), 2.80-2.77 (m, 0.5H), 2.76 (s, 1.5H), 2.67-2.60 (m, 0.5H), 2.18-2.13 (m, 6H), 1.79-1.72 (m, 1.5H), 1.09-1.07 (m, 6H), 41 NMR (400 MHz, DMSO-d⁶) for 69L132 (syn-diastereomers): δ 8.00 (d, J=8.4 Hz, 2H), 7.61 (d, J=8.4 Hz, 2H), 7.19 (d, J=2.0 Hz, 1H), 7.14 (dd, J₁=8.4 Hz, J₂=2.0 Hz, 1H), 7.07 (d, J=8.4 Hz, 1H), 7.01 (s, 1H), 6.88 (d, J=2.0 Hz, 1H), 6.77-6.74 (m, 2H), 5.09 (d, J=7.6 Hz, 1H), 4.89 (d, J=2.8 Hz, 1H), 3.70-3.50 (m, 3H), 3.00 (s, 3H), 2.70-2.66 (m, 1H), 2.18 (s, 3H), 2.16 (s, 3H), 1.87-1.76 (m, 1H), 1.36-1.30 (m, 1H), 1.10 (t, J=6.8 Hz, 3H). ¹H NMR (400 MHz, CDCl₃) for 69L133 (anti-diastereomers): δ 8.00 (d, J=8.4 Hz, 2H), 7.64 (d, J=8.0 Hz, 2H), 7.31 (d, J=2.0 Hz, 1H), 7.28 (s, 1H), 7.13 (dd, J₁=8.4 Hz, J₂=2.0 Hz, 1H), 7.07 (d, J=8.0 Hz, 1H), 6.89 (d, J=1.6 Hz, 1H), 6.78-6.76 (m, 2H), 4.42 (d, J=4.8 Hz, 1H), 3.94-3.88 (m, 1H), 3.85 (d, J=10.4 Hz, 1H), 3.73-3.64 (m, 2H), 3.00 (s, 3H), 2.34-2.29 (m, 1H), 2.19 (s, 3H), 2.16 (s, 3H), 2.00-1.91 (m, 1H), 1.60-1.53 (m, 1H), 1.11 (t, J=6.8 Hz, 3H).

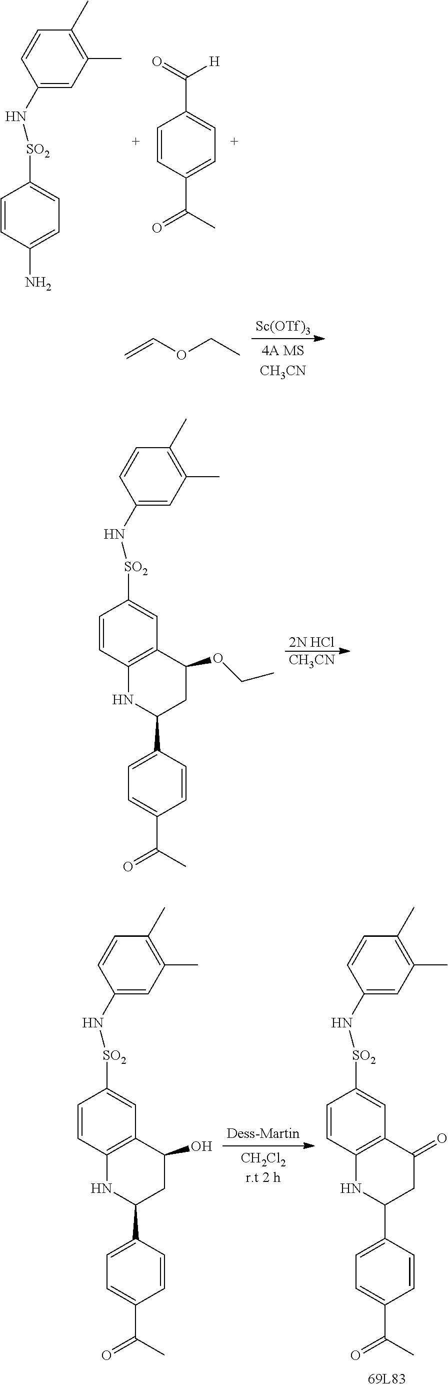

### Preparation of 2-(4-acetylphenyl)-N-(3,4-dimethylphenyl)-4-ethoxy-1,2,3,4-tetrahydroquinoline-6-sulfonamide

4-Amino-N-(3,4-dimethylphenyl)benzenesulfonamide (0.50 g, 1.81 mmol), 4-acetyl-benzaldehyde (0.27 g, 1.81 mmol), Sc(OTf)₃ (0.18 g, 0.36 mmol) and 4 Å molecular sieves (1 g) were mixed together in anhydrous CH₃CN (10 mL) at room temperature under argon. The reaction mixture was stirred for one hour at room temperature. Ethoxyethene (0.26 g, 3.62 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.36 g, 41.6% yield), trans-diastereomers: ¹H NMR (400 MHz, CDCl₃) δ 7.96 (d, J=8.4 Hz, 2H), 7.52-7.49 (m, 4H), 6.97 (d, J=8.0 Hz, 1H), 6.90 (d, J=2.0 Hz, 1H), 6.81 (dd, J₁=8.0 Hz, J₂=2.0 Hz, 1H), 6.54 (d, J=8.0 Hz, 1H), 6.33 (s, 1H), 4.72 (dd, J₁=12.0 Hz, J₂=3.2 Hz, 1H), 4.62 (s, 1H), 4.26 (t, J=2.8 Hz, 1H), 3.46-3.39 (m, 1H), 3.36-3.29 (m, 1H), 2.61 (s, 3H), 2.25-2.21 (m, 1H), 2.19-2.18 (ma. 6H), 1.85-1.77 (m, 1H), 1.67 (t, J=6.8 Hz, 3H).

### Preparation of 2-(4-acetylphenyl)-N-(3,4-dimethylphenyl)-4-hydroxy-1,2,3,4-tetrahydroquinoline-6-sulfonamide

2-(4-Acetylphenyl)-N-(3,4-dimethylphenyl)-4-ethoxy-1,2,3,4-tetrahydroquinoline-6-sulfonamide (0.15 g, 0.31 mmol) was dissolved in anhydrous CH₃CN (10 mL) at room temperature. To this solution was added 3 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=19/1 v/v) to give a pale-yellow solid product (60 mg, 42.6% yield). trans-diastereomers: ¹H NMR (400 MHz, DMSO-d⁶) δ 9.72 (s, 1H), 7.95 (d, J=8.4 Hz, 2H), 7.56 (d, J=2.4 Hz, 1H), 7.50 (d, J=8.4 Hz, 2H), 7.36 (dd, J₁=8.8 Hz, J₂=2.4 Hz, 1H), 7.18 (s, 1H), 6.96 (d, J=8.4 Hz, 1H), 6.87 (d, J=2.0 Hz, 1H), 6.83 (dd, J₁=8.0 Hz, J₂=2.0 Hz, 1H), 6.66 (d, J=8.4 Hz, 1H), 5.42 (d, J=5.2 Hz, 1H), 4.60 (dd, J₁=10.4 Hz, J₂=3.6 Hz, 1H), 4.43-4.39 (m, 1H), 2.57 (s, 3H), 2.11 (s, 3H), 2.09 (s, 3H), 1.97-1.91 (m, 1H), 1.84-1.77 (m, 1H).

### Preparation of 2-(4-acetylphenyl)-N-(3,4-dimethylphenyl)-4-oxo-1,2,3,4-tetrahydroquinoline-6-sulfonamide. (69L83)

2-(4-Acetylphenyl)-N-(3,4-dimethylphenyl)-4-hydroxy-1,2,3,4-tetrahydroquinoline-6-sulfonamide (30 mg, 0.067 mmol) was dissolved in anhydrous methylene chloride (3 mL) at room temperature. To this solution was added 42 mg (0.10 mmol) of Dess-Martin reagent at room temperature. The reaction solution was stirred at room temperature for 30 minutes, then diluted with 20 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=19/1 v/v) to give a brown solid product (15 mg, 50.3% yield). ¹H NMR (400 MHz, CDCl₃) δ 8.33 (s, 1H), 8.00 (d, J=8.01 Hz, 2H), 7.63 (d, J=8.8 Hz, 1H), 7.52 (d, J=8.0 Hz, 2H), 6.99 (d, J=8.4 Hz, 1H), 6.89 (s, 1H), 6.79 (d, J=7.2 Hz, 1H), 6.70 (d, J=8.8 Hz, 1H), 6.33 (s, 1H), 4.92 (s, 1H), 4.89 (dd, J₁=11.2 Hz, J₂=5.2 Hz, 1H), 2.90-2.86 (m, 2H), 2.19 (s, 3H), 2.18 (s, 3H).

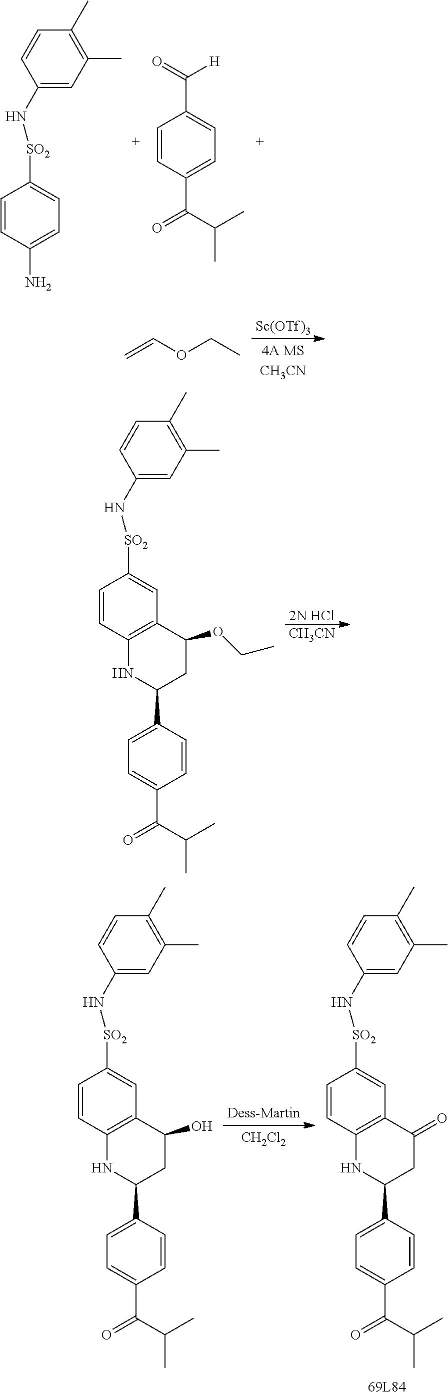

### Preparation of 4-isobutyrylbenzaldehyde

To a solution of 1-bromo-4-(diethoxymethyl)benzene (45.30 g, 174.81 mmol in 200 mL of anhdrous tetrahydrofuran under an argon atmosphere was added dropwise n-butyllithium (76.88 mL of 2.5 N hexanes solution, 1922 mmol) at −78° C. in a dry ice/acetone bath. After stirred at −78° C. for 2 hours, a solution of CuCN (15.66 g, 184.81 mmol) and LiCl (14.82 g, 349.62 mmol) in 200 mL anhydrous tetrahydrofuran was added dropwise with stirring at −78° C. under argon. The resulted solution was stirred at −78° C. for 30 minutes, then slowly warmed to the room temperature and stirred for another hour. The reaction was quenched by addition of 100 mL of water at the room temperature with vigorous stirring. THF solvent was removed under reduced pressure. The aqueous residue was extracted with methylene chloride (3×100 mL). The organic extracts were dried over anhydrous MgSO₄, filtered and concentrated under reduced pressure. The crude liquid was purified by column chromatography (silica gel, CH₂Cl₂) to give an oil product (28.00 g, 91.5% yield). ¹H NMR (400 MHz, DMSO-d⁶) δ 10.11 (s, 1H); 8.14 (d, J=8.0 Hz, 2H), 8.05 (d, J=8.0 Hz, 2H), 3.70 (m, 1H), 1.12 (d, J=6.8 Hz, 6H).

### Preparation of N-(3,4-dimethylphenyl)-4-ethoxy-2-(4-isobutyrylphenyl)-1,2,3,4-tetrahydroquinoline-6-sulfonamide

4-Amino-N-(3,4-dimethylphenyl)benzenesulfonamide (2.07 g, 7.49 mmol), 4-isobutyryl-benzaldehyde (1.32 g, 7.49 mmol), Sc(OTf)₃ (0.74 g, 1.50 mmol) and 4 Å molecular sieves (2 g) were mixed together in anhydrous CH₃CN (80 mL) at room temperature under argon. The reaction mixture was stirred for one hour at room temperature. Ethoxyethene (0.81 g, 11.24 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (1.52 g, 40.1% yield). trans-diastereomers: ¹H NMR (400 MHz, DMSO-d⁶) δ 9.71 (s, 1H), 7.97 (d, J=8.0 Hz, 2H), 7.56 (d, J=8.0 Hz, 2H), 7.42-7.38 (m, 2H), 6.93 (d, J=8.0 Hz, 1H), 6.83 (d, J=2.0 Hz, 1H), 6.78 (dd, J₁=8.0 Hz, J₂=2.0 Hz, 1H), 6.69 (d, J=8.4 Hz, 1H), 6.06 (s, 1H), 4.94 (d, J=7.6 Hz, 1H), 3.72-3.66 (m, 1H), 3.48-3.43 (m, 1H), 3.16 (dd, J₁=8.4 Hz, J₂=2.0 Hz, 1H), 2.09 (s, 6H), 1.81-1.57 (m, 7H), 1.33-1.13 (m, 5H), 1.00-091 (m, 2H).

### Preparation of N-(3,4-dimethylphenyl)-4-hydroxy-2-(4-isobutyrylphenyl)-1,2,3,4-tetrahydroquinoline-6-sulfonamide

N-(3,4-Dimethylphenyl)-4-ethoxy-2-(4-isobutyrylphenyl)-1,2,3,4-tetrahydroquinoline-6-sulfonamide (0.45 g, 0.89 mmol) was dissolved in anhydrous CH₃CN (10 mL) at room temperature. To this solution was added 6 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel. CH₂Cl₂/Methanol=19/1 v/v) to give a pale-yellow solid product (0.26 g, 61.0% yield). trans-diastereomers: ¹H NMR (400 MHz, CDCl₃) δ 7.95 (d, J=8.4 Hz, 2H), 7.62 (d, J=2.4 Hz, 1H), 7.49 (d, J=8.4 Hz, 2H), 7.45 (dd, J₁=8.4 Hz, J₂=2.0 Hz, 2H), 6.97 (d, J=8.0 Hz, 1H), 6.89 (d, J=2.0 Hz, 1H), 6.80 (dd, J₁=8.0 Hz, J₂ 2.0 Hz, 1), 6.64 (s, 1H), 6.54 (d, J=8.8 Hz, 1H), 4.73-4.69 (m, 3H), 3.56-3.53 (m, 1H), 2.33 (s, 1H), 2.17 (s, 6H), 1.87-1.82 (m, 1H), 1.22 (d, J=6.8 Hz, 6H).

### Preparation of N-(3,4-dimethylphenyl)-2-(4-isobutyrylphenyl)-4-oxo-1,2,3,4-tetrahydroquinoline-6-sulfonamide. (69L84)

N-(3,4-Dimethylphenyl)-4-hydroxy-2-(4-isobutyrylphenyl)-1,2,3,4-tetrahydroquinoline-6-sulfonamide (0.10 g, 0.21 mmol) was dissolved in anhydrous methylene chloride (10 mL) at room temperature. To this solution was added 0.13 g (0.31 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=19/1 v/v) to give a brown solid product (56 mg, 56.2% yield). ¹H NMR (400 MHz, CDCl₃) δ 8.33 (d, J=2.4 Hz, 1H), 7.99 (d, J=8.4 Hz, 2H), 7.63 (dd, J₁=8.4 Hz, J₂=2.0 Hz, 1H), 7.52 (d, J=8.4 Hz, 2H), 6.99 (d, J=8.4 Hz, 1H), 6.89 (d, J=2.4 Hz, 1H), 6.79 (dd, J₁=8.0 Hz, J₂=2.4 Hz, 1H), 6.70 (d, J=8.8 Hz, 1H), 6.38 (s, 1H), 4.94 (s, 1H), 4.88 (dd, J₁=11.6 Hz, J₂=5.6 Hz, 1H), 3.58-3.51 (m, 1H), 2.94-2.84 (m, 2H), 2.19 (s, 3H), 2.18 (s, 3H), 1.22 (d, J=6.8 Hz, 6H).

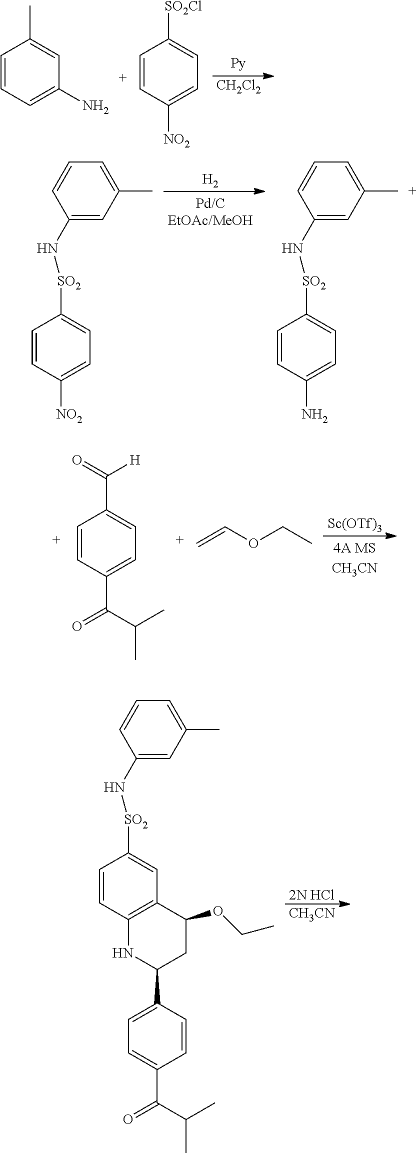

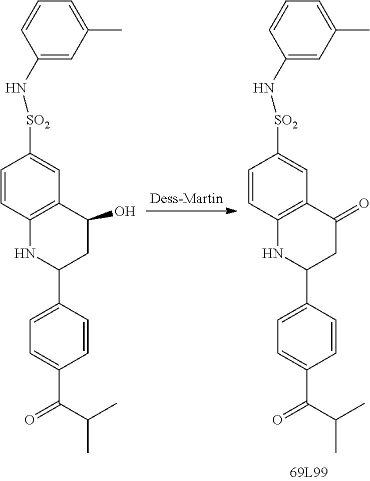 [SMILES: Cc1cccc(NS(=O)(=O)c2ccc3c(c2)[C@@H](O)CC(c2ccc(C(=O)C(C)C)cc2)N3)c1 ; Cc1cccc(NS(=O)(=O)c2ccc3c(c2)C(=O)CC(c2ccc(C(=O)C(C)C)cc2)N3)c1]

### Preparation of 4-nitro-N-(m-tolyl)benzenesulfonamide

4-Nitrobenzenesulfonyl chloride (20.68 g, 93.32 mmol) and m-toluidine (10.00 g, 93.32 mmol) were dissolved in anhydrous methylene chloride (100 mL) at room temperature under argon. Pyridine (11.07 g, 140 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂) to afford product as a pale yellow solid (27.0 g, 99% yield), ¹H NMR (400 MHz, DMSO-d⁶) δ 10.56 (s, 1H), 8.37 (d, J=8.0 Hz, 2H), 7.99 (d, J=8.0 Hz, 2H), 7.15-7.11 (m, 1H), 6.93-6.88 (m, 3H), 2.20 (s, 3H).

### Preparation of 4-amino-N-(m-tolyl)benzenesulfonamide

4-Nitro-N-(m-tolyl)benzenesulfonamide (20.00 g, 68.42 mmol) and Pd/C (10% Pd base, 2 g) were mixed together in MeOH (100 mL) and Ethyl acetate (100 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (15.6 g, 87.2% yield). ¹H NMR (400 MHz, DMSO-d⁶) δ 9.80 (s, 1H); 7.38 (d, J=8.4 Hz, 2H), 7.09-7.04 (m, 1H), 6.86 (s, 2H), 6.78 (d, J=7.2 Hz, 1H), 6.52 (d, J=8.4 Hz, 2H), 5.97 (s, 2H), 2.18 (s, 3H).

### Preparation of 4-ethoxy-2-(4-isobutyrylphenyl)-N-(m-tolyl)-1,2,3,4-tetrahydroquinoline-6-sulfonamide

4-Amino-N-(n-tolyl)benzenesulfonamide (1.57 g, 5.99 mmol), 4-isobutyrylbenzaldehyde (1.06 g, 5.99 mmol), Sc(OTf)₃ (0.59 g, 1.20 mmol) and 4 Å molecular sieves (2 g) were mixed together in anhydrous CH₃CN (50 mL) at room temperature under argon. The reaction mixture was stirred for one hour at room temperature. Ethoxyethene (0.43 g, 5.99 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a yellow solid product (1.12 g, 37.9% yield). cis/trans-diastereomers: ¹H NMR (400 MHz, CDCl₃) δ 7.96 (d, J=8.0 Hz, 2H), 7.54-7.51 (m, 2H), 7.45 (s, 1H), 7.11-7.07 (m, 1H), 6.94-6.87 (m, 4H), 6.66 (s, 0.5H), 6.59 (s, 0.5H), 6.53 (d, J=8.8 Hz, 0.5H), 6.43 (d, J=8.8 Hz, 0.5H), 4.74-4.65 (m, 1H), 4.37 (s, 0.5H), 4.26 (s, 0.5H), 4.19 (s, 0.5H), 3.68-3.62 (m, 0.5H), 3.58-3.52 (m, 1H), 3.28-1.23 (m, 2H), 2.28-2.26 (s, 3H), 1.81 (t, J=12 Hz, 0.5H), 1.39 (t, J=12 Hz, 0.5H), 1.27-1.10 (m, 91H).

### Preparation of 4-hydroxy-2-(4-isobutyrylphenyl)-N-(m-tolyl)-)-1,2,3,4-tetrahydroquinoline-6-sulfonamide

N-(3,4-Dimethylphenyl)-4-ethoxy-2-(4-isobutyrylphenyl)-1,2,3,4-tetrahydroquinoline-6-sulfonamide (0.45 g, 0.89 mmol) was dissolved in anhydrous CH₃CN (10 mL) at room temperature. To this solution was added 6 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=19/1 v/v) to give a pale-yellow solid product (0.26 g, 61.0% yield). (trans-isomer): ¹H NMR (400 MHz, CDCl₃) δ 7.95 (d, J=8.4 Hz, 2H), 7.62 (d, 1=2.4 Hz, 1H), 7.49 (d, J=8.4 Hz, 2H), 7.45 (dd, J₁=8.4 Hz, J₂=2.0 Hz, 2H), 6.97 (d, J=8.0 Hz, 1H), 6.89 (d, J=2.0 Hz, 1H), 6.80 (dd, J₁=8.0 Hz, J₂=2.0 Hz, 1H), 6.64 (s, 1H), 6.54 (d, J=8.8 Hz, 1H), 4.73-4.69 (m, 3H), 3.56-3.53 (m, 1H), 2.33 (s, 1H), 2.17 (s, 6H), 1.87-1.82 (m, 1H), 1.22 (d, J=6.8 Hz, 6H).

### Preparation of 2-(4-isobutyrylphenyl)-4-oxo-N-(m-tolyl)-1,2,3,4-tetrahydroquinoline-6-sulfonamide. (69L99)

2-(4-Isobutyrylphenyl)-4-hydroxy-N-(m-tolyl)-1,2,3,4-tetrahydroquinoline-6-sulfonamide (0.15 g, 0.32 mmol) was dissolved in anhydrous methylene chloride (10 mL) at room temperature. To this solution was added 0.21 g (0.48 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=19/1 v/v) to give a brown solid product (52 mg, 34.9% yield). ¹H NMR (400 MHz, CDCl₃) δ 8.34 (d, J=2.4 Hz, 1H), 7.96 (d, J=8.0 Hz, 2H), 7.61 (d, J=8.8 Hz, 1H), 7.51 (d, J=8.0 Hz, 2H), 7.13-7.09 (m, 2H), 6.94-6.89 (m, 3H), 6.71 (d, J=8.8 Hz, 1H), 5.18 (s, 1H), 4.87 (dd, J₁=11.6 Hz, J₂=5.6 Hz, 1H), 3.56-3.49 (m, 1H), 2.93-2.82 (m, 2H), 2.27 (s, 3H), 1.21 (d, J=6.8 Hz, 6H).

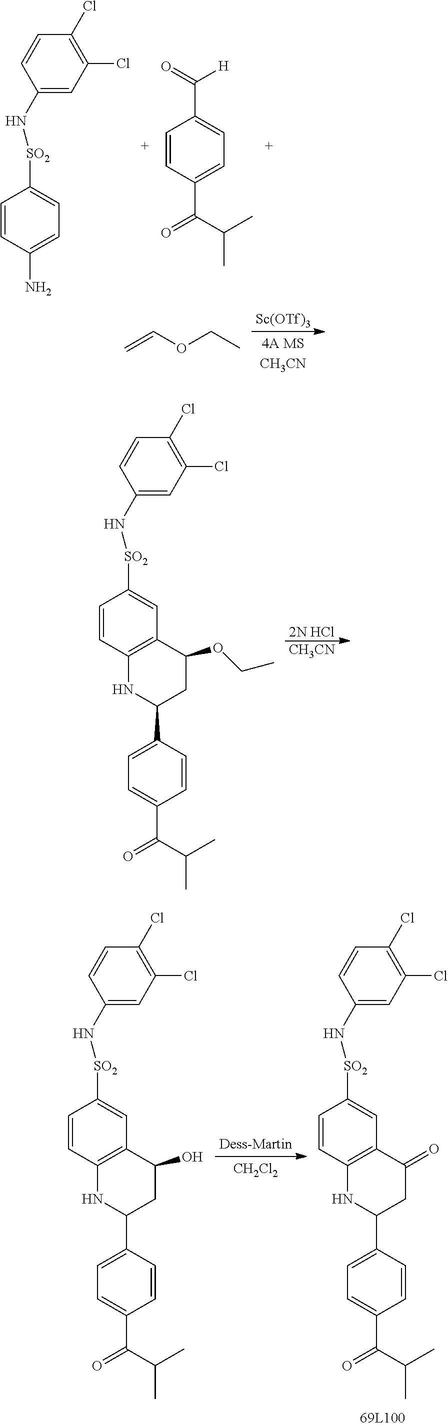

### Preparation of N-(3,4-dichlorophenyl)-2-(4-isobutyrylphenyl)-4-oxo-1,2,3,4-tetrahydroquinoline-6-sulfonamide. (69L100)

Compound N-(3,4-dichlorophenyl)-4-hydroxy-2-(4-isobutyrylphenyl)-1,2,3,4-tetrahydroquinoline-6-sulfonamide (0.12 g, 0.23 mmol) was dissolved in anhydrous methylene chloride (20 mL) at room temperature. To this solution was added 98 mg (0.23 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel. CH₂Cl₂/Acetone=19/1 v/v) to give a brown solid product (58 mg, 48.7% yield). ¹H NMR (400 MHz, CDCl₃) δ 8.33 (s, 1H), 7.99 (d, J=8.0 Hz, 2H), 7.64 (d, J=8.8 Hz, 1H), 7.52 (d, J=8.0 Hz, 2H), 7.39 (s, 1H), 7.31 (d, J=8.8 Hz, 1H), 7.25 (d, J=8.8 Hz, 1H), 7.04 (d, J=8.8 Hz, 1H), 6.75 (d, J=8.8 Hz, 1H), 5.13 (s, 1H), 4.92 (dd, J₁=11.6 Hz, J₂=5.2 Hz, 1H), 3.57-3.50 (m, 1H), 2.98-2.87 (m, 2H), 1.22 (d, J=6.8 Hz, 6H).

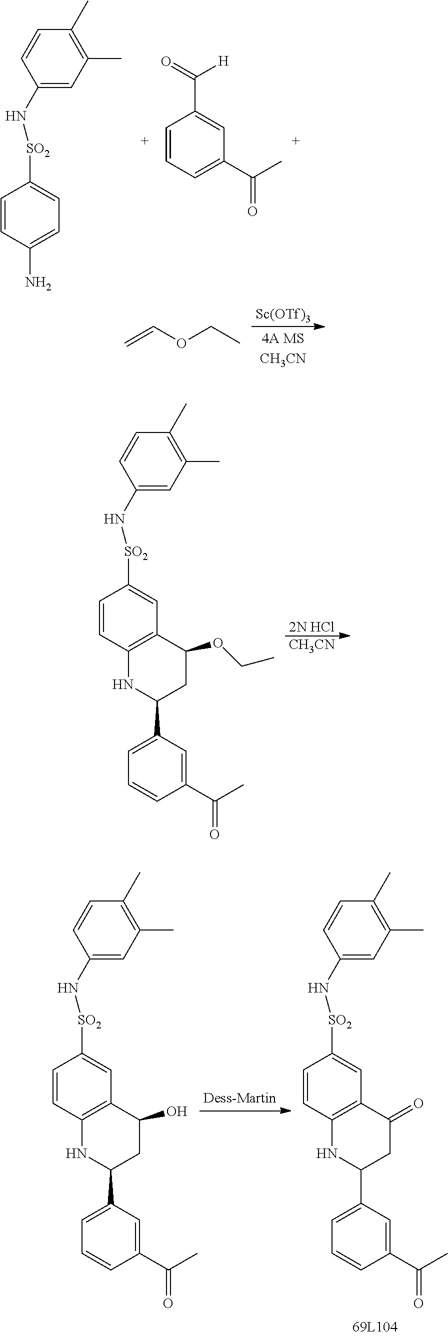

### Preparation of 2-(3-acetylphenyl)-N-(3,4-dimethylphenyl)-4-ethoxy-1,2,3,4-tetrahydroquinoline-6-sulfonamide

4-Amino-N-(3,4-dimethylphenyl)benzenesulfonamide (1.00 g, 3.62 mmol), 3-acetylbenzaldehyde (0.34 g, 3.62 mmol), Sc(OTf)₃ (0.36 g, 0.72 mmol) and 4 Å molecular sieves (1 g) were mixed together in anhydrous CH₃CN (20 mL) at room temperature under argon. The reaction mixture was stirred for one hour at room temperature. Ethoxyethene (0.39 g, 5.43 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.45 g, 26.0% yield). trans-diastereomers: ¹H NMR (400 MHz, CDCl₃) δ 8.03 (s, 1H), 7.95 (d, J=8.4 Hz, 1H), 7.62 (d, J=8.4 Hz, 1H), 7.54-7.46 (m, 3H), 6.97 (d, J=8.0 Hz, 1H), 6.92 (s, 1H), 6.85-6.81 (m, 2H), 6.53 (d, J=8.4 Hz, 1H), 4.74-4.71 (m, 2H), 4.25 (s, 1H), 3.44-3.40 (m, 1H), 3.34-3.30 (m, 1H), 2.61 (s, 3H), 2.25-2.21 (m, 1H), 1.81 (t, J=12.8 Hz, 1H), 1.16 (t, J=6.8 Hz, 3H).

### Preparation of 2-(3-acetylphenyl)-N-(3,4-dimethylphenyl)-4-hydroxy-1,2,3,4-tetrahydroquinoline-6-sulfonamide

2-(3-Acetylphenyl)-N-(3,4-dimethylphenyl)-4-ethoxy-1,2,3,4-tetrahydroquinoline-6-sulfonamide (0.15 g, 0.31 mmol) was dissolved in anhydrous CH₃CN (10 mL) at room temperature. To this solution was added 4 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=19/1 v/v) to give a pale-yellow solid product (58 mg, 41.1% yield). trans-diastereomers: ¹H NMR (400 MHz, CDCl₃) δ 7.95 (s, 1H), 7.82 (d, J=8.4 Hz, 1H), 7.68 (s, 1H), 7.52 (d, J=8.0 Hz, 1H), 7.49-7.39 (m, 2H), 7.35 (d, J=8.8 Hz, 1H), 6.87 (s, 2H), 6.79 (d, J=7.6 Hz, 1H), 6-6 (d, J=8.4 Hz, 1H), 5.00 (s, 1H), 4.65-4.62 (m, 2H), 2.54 (s, 3H), 2.11 (s, 3H), 2.09 (s, 3H), 1.75 (t, J=12 Hz, 1H).

### Preparation of 2-(3-acetylphenyl)-N-(3,4-dichlorophenyl)-4-oxo-1,2,3,4-tetrahydroquinoline-6-sulfonamide. (69L104)

2-(3-Acetylphenyl)-N-(3,4-dichlorophenyl)-4-hydroxy-1,2,3,4-tetrahydroquinoline-6-sulfonamide (0.16 g, 0.36 mmol) was dissolved in anhydrous methylene chloride (20 mL) at room temperature. To this solution was added 0.17 mg (0.39 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=19/1 v/v) to give a brown solid product (65 mg, 40.9% yield). ¹H NMR (400 MHz, CDCl₃) δ 8.34 (s, 1H), 8.06 (s, 1H), 7.95 (d, J=7.6 Hz, 1H), 7.61 (t, J=7.6 Hz, 2H), 7.52 (t, J=7.6 Hz, 1H), 6.98 (d, J=8.0 Hz, 1H), 6.90 (s, 1H), 6.80 (d, J=8.0 Hz, 1H), 6.69-6.67 (m, 2H), 5.03 (s, 1H), 4.88 (dd, J₁=12.8 Hz, J₂=4.4 Hz, 1H), 2.95-2.81 (m, 2H), 263 (s, 3H), 2.19 (s, 3H), 2.18 (s, 3H)

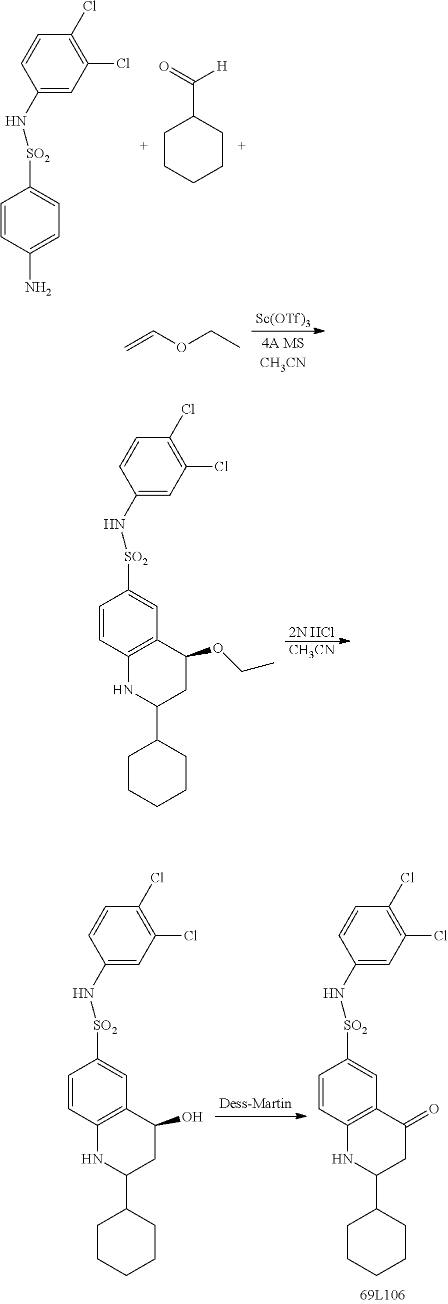

### Preparation of 2-cyclohexyl-N-(3,4-dimethylphenyl)-4-oxo-1,2,3,4-tetrahydroquinoline-6-sulfonamide (69L106)

2-Cyclohexyl-N-(3,4-dimethylphenyl)-4-hydroxy-1,2,3,4-tetrahydroquinoline-6-sulfonamide (0.10 g, 0.22 mmol) was dissolved in anhydrous methylene chloride (10 mL) at room temperature. To this solution was added 93 mg (0.22 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=19/1 v/v) to give a brown solid product (55 mg, 55.3% yield). 11 NMR (400 MHz, CDCl₃) δ 8.30 (s, 1H), 7.58 (dd, J₁=8.8 Hz, J₂=2.4 Hz, 1H), 7.31 (s, 1H), 7.20 (d, J=2.0 Hz, 1H), 7.00 (dd, J₁=8.4 Hz, J₂=2.10 Hz, 1H), 6.96 (s, 1H), 6.64 (d, J=8.8 Hz, 1H), 4.84 (s, 1H), 3.53-3.49 (m, 1H), 2.68-2.61 (m, 2H), 1.99-1.03 (m, 14H)

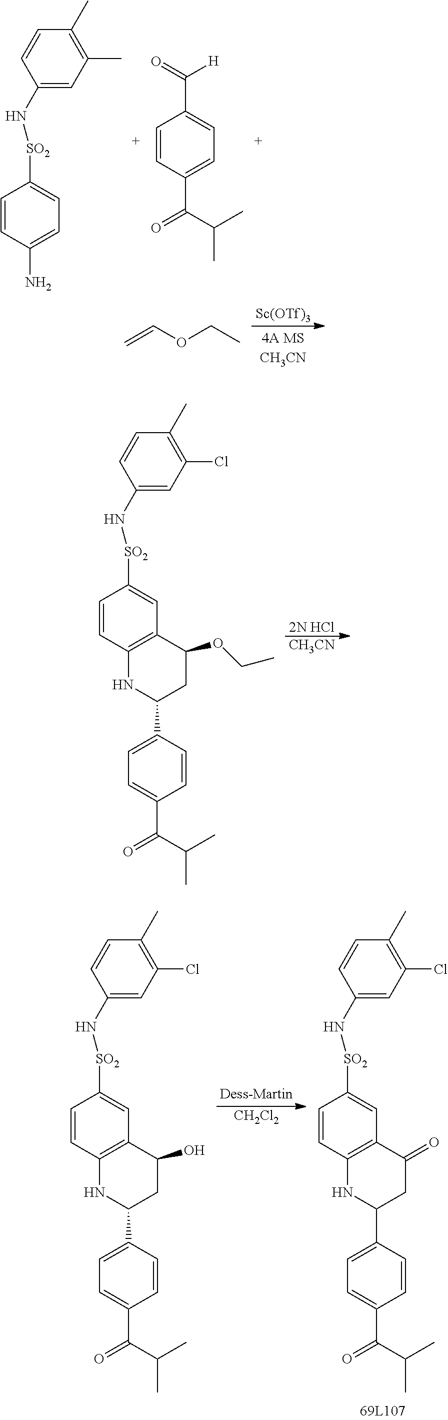

### Preparation of N-(3-choro-4-methylphenyl)-4-ethoxy-2-(4-isobutyrylphenyl)-1,2,3,4-tetrahydroquinoline-6-sulfonamide

4-Amino-N-(3-choro-4-methylphenyl)benzenesulfonamide (1.02 g, 3.44 mmol), 4-isobutyrylbenzaldehyde (0.61 g, 3.44 mmol), Sc(OTf)₃ (0.34 g, 0.69 mmol) and 4 Å molecular sieves (2 g) were mixed together in anhydrous CH₃CN (40 mL) at room temperature under argon. The reaction mixture was stirred for one hour at room temperature. Ethoxyethene (0.50 g, 6.88 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (1.06 g, 58.6% yield). trans-diastereomers: ¹H NMR (400 MHz, CDCl₃) δ 796 (d, J=0.0 Hz, 2H), 7.55-7.50 (m, 4H), 7.1.5 (s, 1H), 7.08 (d, J=8.0 Hz, 1H), 6.92 (d, J=8.0 Hz, 1H), 6.68 (s, 1H), 6.56 (d, J=8.8 Hz, 1H), 4.74-4.69 (m, 2H), 4.27 (s, 1H), 3.56-3.53 (m, 1H), 3.47-3.43 (m, 1H), 3.36-3.32 (m, 1H), 2.28 (s, 3H), 1.82 (t, J=12 Hz, 1H), 1.23-1.16 (m, 9H).

### Preparation of N-(3-chloro-4-methylphenyl)-4-hydroxy-2-(4-isobutyrylphenyl)-1,2,3,4-tetrahydroquinoline-6-sulfonamide

N-(3-Chloro-4-methylphenyl)-4-ethoxy-2-(4-isobutyrylphenyl)-1,2,3,4-tetrahydro-quinoline-6-sulfonamide (0.43 g, 0.82 mmol) was dissolved in anhydrous CH₃CN (20 mL) at room temperature. To this solution was added 6 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=19/1 v/v) to give a pale-yellow solid product (0.26 g, 63.9% yield). trans-diastereomers: ¹H NMR (400 MHz, CDCl₃) δ 7.96 (d, J=8.0 Hz, 2H), 7.68 (s, 1H), 7.50-7.46 (m, 3H), 7.10-7.06 (m, 2H), 6.96-6.92 (m, 1H), 6.87 (s, 1H), 6.55 (d, J=8.8 Hz, 1H), 4.78 (s, 1H), 4.72-4.70 (m, 2H), 3.58-3.49 (m, 1H), 2.28 (s, 3H), 1.88 (t, J=11.6 Hz, 1H), 1.22 (d, J=6.8 Hz, 6H).

### Preparation of N-(3-chloro-4-methylphenyl)-2-(4-isobutyrylphenyl)-4-oxo-1,2,3,4-tetrahydroquinoline-6-sulfonamide. (69L107)

N-(3-Chloro-4-methylphenyl)-4-hydroxy-2-(4-isobutyrylphenyl)-1,2,3,4-tetrahydro-quinoline-6-sulfonamide (0.16 g, 0.32 mmol) was dissolved in anhydrous methylene chloride (20 mL) at room temperature. To this solution was added 0.14 g (0.32 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=19/1 v/v) to give a brown solid product (0.10 mg, 62.9% yield). ¹H NMR (400 MHz, CDCl₃) δ 8.34 (s, 1H), 7.98 (d, J=8.0 Hz, 2H), 7.62 (d, J=88 Hz, 1H), 7.52 (d, J=8.0 Hz, 2H), 7.11-7.09 (m, 2H), 6.97 (d, J=5.6 Hz, 1H), 6.72 (d, J=8.8 Hz, 1H) 5.08 (s, 1H), 4.90 (dd, J₁=11.6 Hz, J₂=5.6 Hz, 1H), 3.57-350 (m, 1H), 2.96-2.85 (m, 2H), 2.29 (s, 3H), 1.22 (d, J=6.4 Hz, 6H).

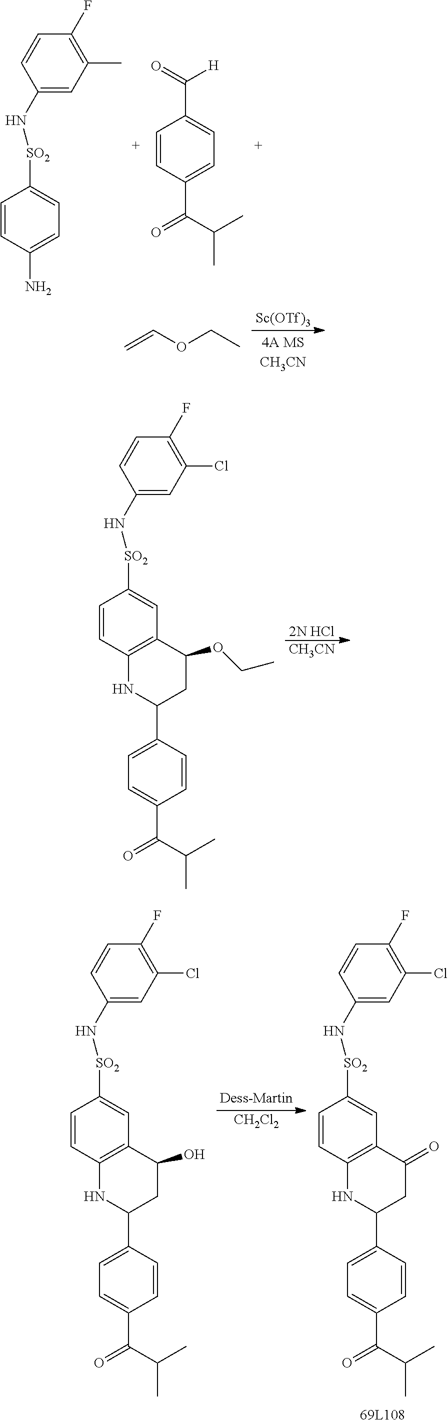

### Preparation of 4-ethoxy-N-(4-fluoro-3-methylphenyl)-2-(4-isobutyrylphenyl)-1,2,3,4-tetrahydroquinoline-6-sulfonamide

4-Amino-N-(4-fluoro-3-methylphenyl)benzenesulfonamide (1.01 g, 3.60 mmol), 4-isobutyrylbenzaldehyde (0.63 g, 3.60 mmol), Sc(OTf)₃ (0.35 g, 0.72 mmol) and 4 Å molecular sieves (2 g) were mixed together in anhydrous CH₃CN (40 mL) at room temperature under argon. The reaction mixture was stirred for one hour at room temperature. Ethoxyethene (0.52 g, 7.20 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.65 g, 35.4% yield). trans-diastereomers: ¹H NMR (400 MHz, CDCl₃) δ 97 (d, J=80 Hz, 2H), 7.51 (d, J=8.0 Hz, 2H), 7.48-7.49 (m, 2H), 6.97 (d, J=6.4 Hz, 1H), 6.87-6.85 (m, 2H), 6.55 (d, J=8.0 Hz, 1H), 6.34 (s, 1H), 4.72 (d, J=9.6 Hz, 1H), 4.66 (s, 1H), 4.25 (s, 1H), 3.57-3.51 (m, 1H), 3.45-3.41 (m, 1H), 3.31-3.28 (m, 1H), 2.21 (s, 3H), 1.81 (t, J=12 Hz, 1H), 1.26-1.67 (m, 9H).

### Preparation of N-(4-fluoro-3-methylphenyl)-4-hydroxy-2-(4-isobutyrylphenyl)-1,2,3,4-tetrahydroquinoline-6-sulfonamide

4-Ethoxy-N-(4-fluoro-3-methylphenyl)-2-(4-isobutyrylphenyl)-1,2,3,4-tetrahydroquinoline-6-sulfonamide (0.55 g, 1.08 mmol) was dissolved in anhydrous CH₃CN (20 mL) at room temperature. To this solution was added 8 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=19/1 v/v) to give a pale-yellow solid product (0.36 g, 69.2% yield). trans-diastereomers: =H NMR (400 MHz, CDCl₃) δ 7.97 (d, J=8.0 Hz, 2H), 7.63 (s, 1H), 7.51 (d, J=8.4 Hz, 2H), 7.42 (d, J=8.4 Hz, 1H), 6.96 (d, J=6.4 Hz, 1H), 6.88-6.85 (m, 2H), 6.55 (d, J=8.8 Hz, 1H), 6.51 (s, 1H), 4.74 (s, 3H), 3.58-3.52 (m, 1H), 2.21 (s, 3H), 1.89 (t, J=12 Hz, 1H), 1.22 (d, J=6.8 Hz, 6H).

### Preparation of N-(4-fluoro-3-methylphenyl)-2-(4-isobutyrylphenyl)-4-oxo-1,2,3,4-tetrahydroquinoline-6-sulfonamide. (69L108)

N-(4-Fluoro-3-methylphenyl)-4-hydroxy-2-(4-isobutyrylphenyl)-1,2,3,4-tetrahydro-quinoline-6-sulfonamide (0.18 g, 0.37 mmol) was dissolved in anhydrous methylene chloride (10 mL) at room temperature. To this solution was added 0.16 g (0.37 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=19/1 v/v) to give a brown solid product (65 mg, 36.3% yield). 1H NMR (400 MHz, CDCl₃) δ 8.32 (s, 1H), 7.99 (d, J=&0 Hz, 2H), 7.58 (d, J=8.8 Hz, 1H), 7.52 (d, J=8.0 Hz, 2H), 6.97 (d, J=5.6 Hz, 1H), 6.90-6.85 (m, 2H), 6.71 (d, J=8.8 Hz, 1H), 6.49 (s, 1H), 4.98 (s, 1H), 4.89 (dd, J₁=11.6 Hz, J₂=5.6 Hz, 1H), 3.54 (m, 1H), 2.96-2.85 (m, 2H), 2.22 (s, 3H), 1.22 (d, J=6.8 Hz, 6H).

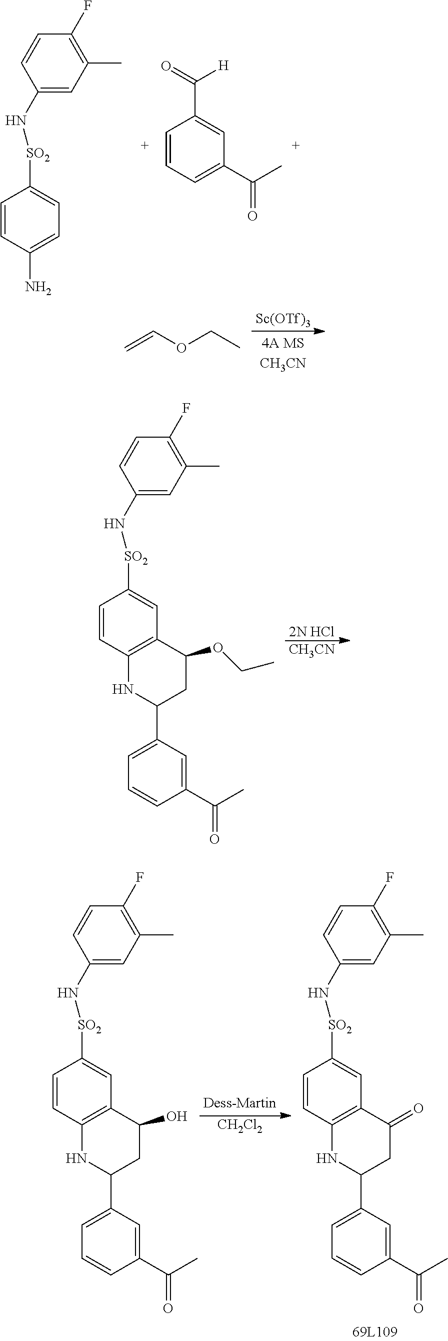

### Preparation of 2-(3-acetylphenyl)-4-ethoxy-N-(4-fluoro-3-methylphenyl)-1,2,3,4-tetrahydroquinoline-6-sulfonamide

4-Amino-(4-fluoro-3-methylphenyl)benzensulfonamide (1.00 g, 3.57 mmol), 3-acetylbenzaldehyde (0.33 g, 3.57 mmol), Sc(OTf)₃ (0.35 g, 0.71 mmol) and 4 Å molecular sieves (2 g) were mixed together in anhydrous CH₃CN (30 mL) at room temperature under argon. The reaction mixture was stirred for one hour at room temperature. Ethoxyethene (0.52 g, 7.14 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=19/1 v/v) to give a white solid product (0.68 g, 39.5% yield). trans-diastereomers: ¹H NMR (400 MHz, CDCl₃) δ 8.02 (s, 1H), 793 (d, J=2.8 Hz, 1H), 7.63 (d, J=7.6 Hz, 1H), 7.53-7.41 (m, 2H), 6.99 (d, J=6.4 Hz, 1H), 6.96 (s, 1H), 6.91-6.80 (m, 2H), 6.55 (d, J=8.8 Hz, 1H), 6.34 (d, J=8.4 Hz, 1H), 4.77 (s, 1H), 4.64 (d, J=9.6 Hz, 1H), 4.26 (s, 1H), 3.70-3.64 (m, 1H), 3.53-3.34 (m, 2H), 3.32-3.28 (m, 1H), 2.63 (s, 3H), 2.21 (s, 3H), 86-1.79 (m, 1H), 1.21 (t, J=6.0 Hz, 1H).

### Preparation of 2-(3-acetylphenyl)-N-(4-fluoro-3-methylphenyl)-4-hydroxy-1,2,3,4-tetrahydroquinoline-6-sulfonamide

2-(3-Acetylphenyl)-4-ethoxy-N-(4-fluoro-3-methylphenyl)-1,2,3,4-tetrahydroquinoline-6-sulfonamide (0.45 g, 0.93 mmol) was dissolved in anhydrous CH₃CN (20 mL) at room temperature. To this solution was added 6 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=9/1 v/v) to give a pale-yellow solid product (0.35 g, 83.3% yield). trans-diastereomers: ¹H NMR (400 MHz, CDCl₃) δ 7.95 (s, 1H), 7.86 (d, J=7.6 Hz, 1H), 7.69 (s, 1H), 7.55 (d, J=7.2 Hz, 1H), 7.47-7.42 (m, 2H), 7.33 (d, J=8.4 Hz, 1H), 6.95 (d, J=6.0 Hz, 1H), 6.83-6.76 (m, 2H), 6.49 (d, J=8.8 Hz, 1H), 4.97 (s, 1H), 4.66-4.64 (m, 2H), 2.57 (s, 3H), 2.14 (s, 3H), 1.81 (t, J=12 Hz, 1H).

### Preparation of 2-(3-acetylphenyl)-N-(4-fluoro-3-methylphenyl)-4-oxo-1,2,3,4-tetrahydroquinoline-6-sulfonamide. (69L109)

2-(3-Acetylphenyl)-N-(4-fluoro-3-methylphenyl)-4-hydroxy-1,2,3,4-tetrahydroquinoline-6-sulfonamide (0.30 g, 0.66 mmol) was dissolved in anhydrous methylene chloride (10 mL) at room temperature. To this solution was added 0.28 g (0.66 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a brown solid product (45 mg, 15% yield). ¹H NMR (400 MHz, CDCl₃) δ 8.31 (s, 1H), 8.06 (s, 1H), 7.95 (dt, J₁=8.0 Hz, J₂=1.2 Hz, 1H), 7.62 (d, J=8.0 Hz, 1H), 7.58 (d, J=2.4 Hz, 1H), 7.57-7.55 (m, 1H), 7.53 (s, 1H), 6.97 (dd, J₁=6.8 Hz, J₂=2.4 Hz, 1H), 6.90-6.84 (m, 2H), 6.70 (d, J=8.4 Hz, 1H), 6.53 (s, 1H), 4.99 (s, 1H), 4.89 (dd, J₁=11.6 Hz, J₂=5.6 Hz, 1H), 2.97-2.83 (m, 2H), 2.22 (s, 3H).

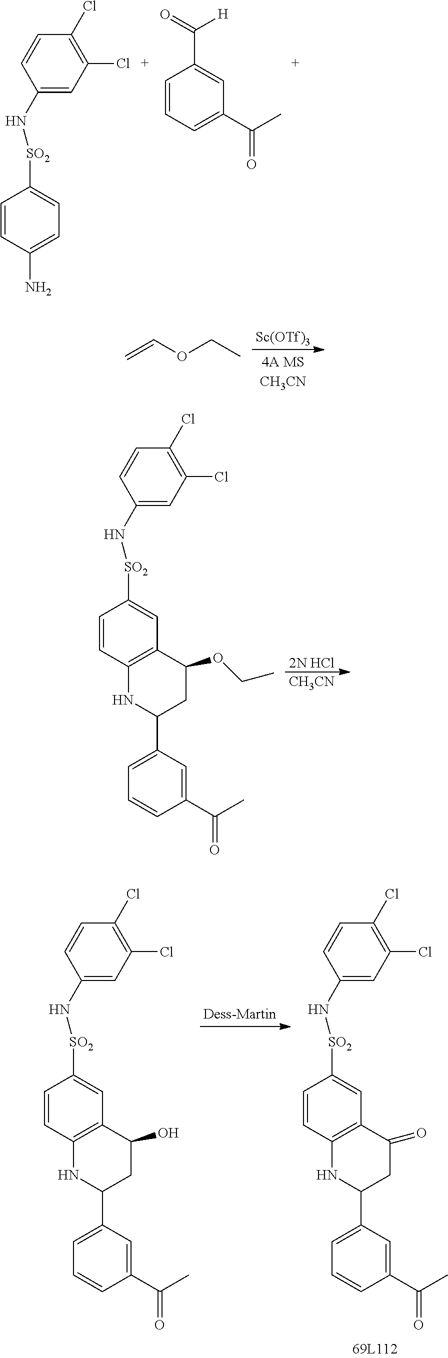

### Preparation of 2-(3-acetylphenyl)-N-(3,4-dichlorophenyl)-4-ethoxy-)-1,2,3,4-tetrahydroquinoline-6-sulfonamide

4-Amino-N-(3,4-dichlorophenyl)benzenesulfonamide (1.00 g, 3.15 mmol), 3-acetylbenzaldehyde (0.47 g, 3.15 mmol), Sc(OTf)₃ (0.31 g, 0.63 mmol) and 4 Å molecular sieves (2 g) were mixed together in anhydrous CH₃CN (30 mL) at room temperature under argon. The reaction mixture was stirred for one hour at room temperature. Ethoxyethene (0.45 g, 6.30 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.45 g, 27.5% yield). trans-diastereomers: ¹H NMR (400 MHz, DMSO-d⁶) δ 10.34 (s, 1H), 7.97 (s, 1H), 7.92 (d, J=8.0 Hz, 1H), 7.69 (d, J=7.6 Hz, 1H), 7.56-7.44 (m, 4H), 7.37 (s, 1H), 7.30 (d, J=2.4 Hz, 1H), 7.10 (dd, J₁=8.8 Hz, J₂=2.4 Hz, 1H), 6.72 (d, J=8.8 Hz, 1H), 4.55 (d, J=12 Hz, 1H), 4.30 (s, 1H), 3.45-3.35 (m, 2H), 3.20-3.16 (m, 1H), 2.60 (s, 3H), 1.82 (t, J=12 Hz, 1H), 1.09 (t, J=6.8 Hz, 1H).

### Preparation of 2-(3-acetylphenyl)-N-(3,4-dichlorophenyl)-4-hydroxy-1,2,3,4-tetrahydroquinoline-6-sulfonamide

2-(3-Acetylphenyl)-N-(3,4-dichlorophenyl)-4-ethoxy-)-1,2,3,4-tetrahydroquinoline-6-sulfonamide (0.25 g, 0.48 mmol) was dissolved in anhydrous CH₃CN (10 mL) at room temperature. To this solution was added 4 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel. CH₂Cl₂/Methanol=9/1 v/v) to give a pale-yellow solid product (0.15 g, 63.6% yield). trans-diastereomers: ¹H NMR (400 MHz, CDCl₃) δ 7.99 (s, 1H), 7.87 (s, 1H), 7.77 (d, J=7.6 Hz, 1H), 7.66 (s, 1H), 7.47 (d, J=7.6 Hz, 1H), 7.37-7.29 (m, 2H), 7.14 (d, J=2.4 Hz, 1H), 7.10 (d, J=8.4 Hz, 1H), 6.88 (d, J₁=8.8 Hz, J₂=2.0 Hz, 1H), 5.01 (s, 1H), 4.60-4.55 (m, 2H), 2.49 (s, 3H), 2.06-2.20 (m, 1H), 1.74 (t, J=12.3 Hz, 1H).

### Preparation of 2-(3-acetylphenyl)-N-(3,4-dichlorophenyl)-4-oxo-1,2,3,4-tetrahydro-quinoline-6-sulfonamide. (69L112)

2-(3-Acetylphenyl)-N-(3,4-dichlorophenyl)-4-hydroxy-1,2,3,4-tetrahydroquinoline-6-sulfonamide (0.10 g, 0.20 mmol) was dissolved in anhydrous methylene chloride (10 mL) at room temperature. To this solution was added 86 mg (0.20 mmol) of Dess-Martin reagent at room temperature. The reaction solution was stirred at room temperature for 30 minutes, then diluted with 50 mL of water and neutralized with NaHCO₃. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (25 mg, 25.3% yield). ¹H NMR (400 MHz, CDCl₃) δ 8.35 (d, J=2.4 Hz, 1H), 8.06 (s, 1H), 7.96 (d, J=76 Hz, 1H), 7.66-7.61 (m, 2H), 7.56-7.52 (m, 1H), 7.32 (d, J=8.8 Hz 1H), 7.22 (d, J=24 Hz, 1H), 7.02 (dd, J₁=8.8 Hz, J₂=2.4 Hz, 1H), 6.73 (d, J=8.8 Hz, 1H), 5.05 (s, 1H), 4.92 (dd, J₁=12.8 Hz, J₂=4.4 Hz, 1H), 2.99-284 (m, 2H), 2.63 (s, 3H).

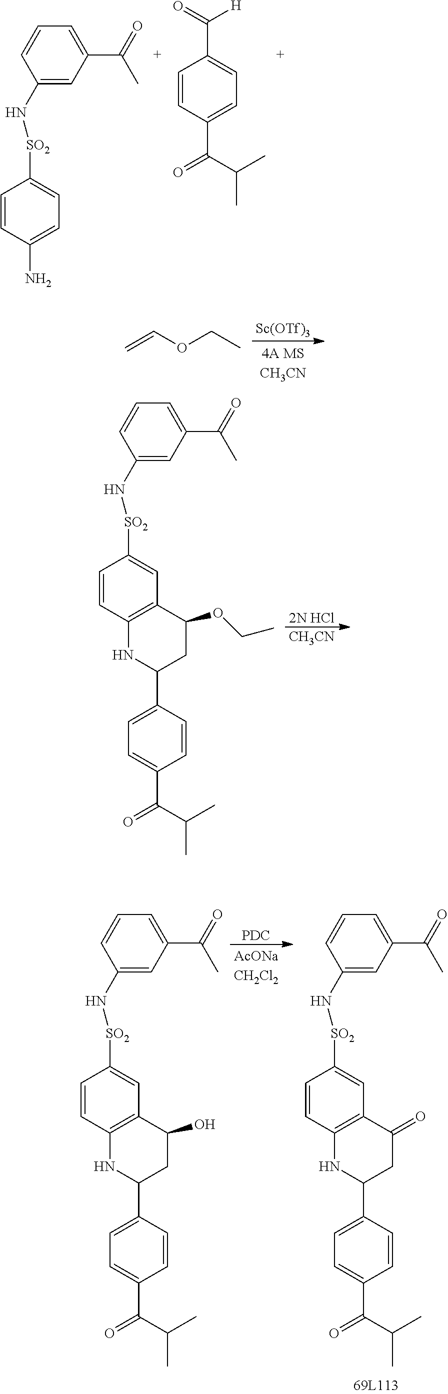

### Preparation of N-(3-acetylphenyl)-4-ethoxy-2-(4-isobutyrylphenyl)-1,2,3,4-tetrahydroquinoline-6-sulfonamide

N-(3-Acetylphenyl)-4-aminobenzenesulfonamide (0.52 g, 1.79 mmol), 3-acetyl-benzaldehyde (0.32 g, 1.79 mmol), Sc(OTf)₃ (0.18 g, 0.36 mmol) and 4 Å molecular sieves (1 g) were mixed together in anhydrous CH₃CN (20 mL) at room temperature under argon. The reaction mixture was stirred for one hour at room temperature. Ethoxyethene (0.26 g, 3.58 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.46 g, 49.5% yield). trans-diastereomers: ¹H NMR (400 MHz, CDCl₃) δ 7.94 (d, J=3.6 Hz, 1H), 7.71 (d, J=7.2 Hz, 1H), 7.65-7.63 (m, 2H), 7.57-7.50 (m, 4H), 7.41 (s, 2H), 6.55 (s, 1H), 4.74 (dd, J₁=12.0 Hz, J₂=2.4 Hz, 1H), 4.68 (s, 1H), 4.29 (t, J=2.8 Hz, 1H), 3.71-3.67 (m, 1H), 3.53-3.44 (m, 2H), 3.39-3.31 (m, 1H), 2.64 (s, 3H), 2.59 (s, 3H), 2.29-2.25 (m, 1H), 1.88-1.81 (m, 1H), 1.23 (t, J=7.6 Hz, 3H).

### Preparation of N-(3-acetylphenyl)-4-hydroxy-2-(4-isobutyrylphenyl)-1,2,3,4-tetrahydro-quinoline-6-sulfonamide

N-(3-Acetylphenyl)-4-ethoxy-2-(4-isobutyrylphenyl)-1,2,3,4-tetrahydroquinoline-6-sulfonamide (0.30 g, 0.58 mmol) was dissolved in anhydrous CH₃CN (10 mL) at room temperature. To this solution was added 6 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=9/1 v/v) to give a pale-yellow solid product (0.23 mg, 82.1% yield). trans-diastereomers: ¹H NMR (400 MHz, CDCl₃) δ 9.79 (s, 1H), 7.87 (d, J=7.2 Hz, 1H), 7.79 (s, 1H), 7.67 (s, 1H), 7.62-7.57 (m, 3H), 7.47-7.43 (m, 3H), 7.39 (d, J=7.2 Hz, 1H), 7.33 (d, J=8.0 Hz, 1H), 6.52 (d, J=8.8 Hz, 1H), 5.02 (s, 1H), 4.71-4.67 (m, 2H), 3.64 (s, 1H), 2.58 (s, 3H), 2.53 (s, 3H), 2.18-2.14 (m, 1H), 1.86-1.79 (m, 1H).

### Preparation of N-(3-acetylphenyl)-2-(4-isobutyrylphenyl)-4-oxo-1,2,3,4-tetrahydro-quinoline-6-sulfonamide. (69L113)

N-(3-Acetylphenyl)-4-hydroxy-2-(4-isobutyrylphenyl)-1,2,3,4-tetrahydroquinoline-6-sulfonamide (0.10 mg, 0.20 mmol) was dissolved in anhydrous methylene chloride (10 mL) at room temperature. To this solution were added 76 mg (0.20 mmol) of pyridinium dichromate and sodium acetate (17 mg, 0.20 mmol) at room temperature. The reaction solution was stirred at room temperature for one hour, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (35 mg, 35.4% yield). ¹H NMR (400 MHz, CDCl₃) δ 8.27 (d, J=2.0 Hz, 1H), 7.92 (d, J=8.4 Hz, 2H), 7.63 (d, J=7.6 Hz, 1H), 7.59 (dd, J₁=8.8 Hz, J₂=2.0 Hz, 1H), 7.54 (s, 1H), 7.44 (d, J=8.4 Hz, 2H), 7.39-7.31 (m, 2H), 6.85 (s, 1H), 6.65 (d, J=8.8 Hz, 1H), 4.94 (s, 1H), 4.81 (dd, J₁=12.0 Hz, J₂=1.6 Hz, 1H), 3.50-3.43 (m, 1H), 2.87-2.77 (m, 2H), 1.15 (d, J=6.8 Hz, 3H).

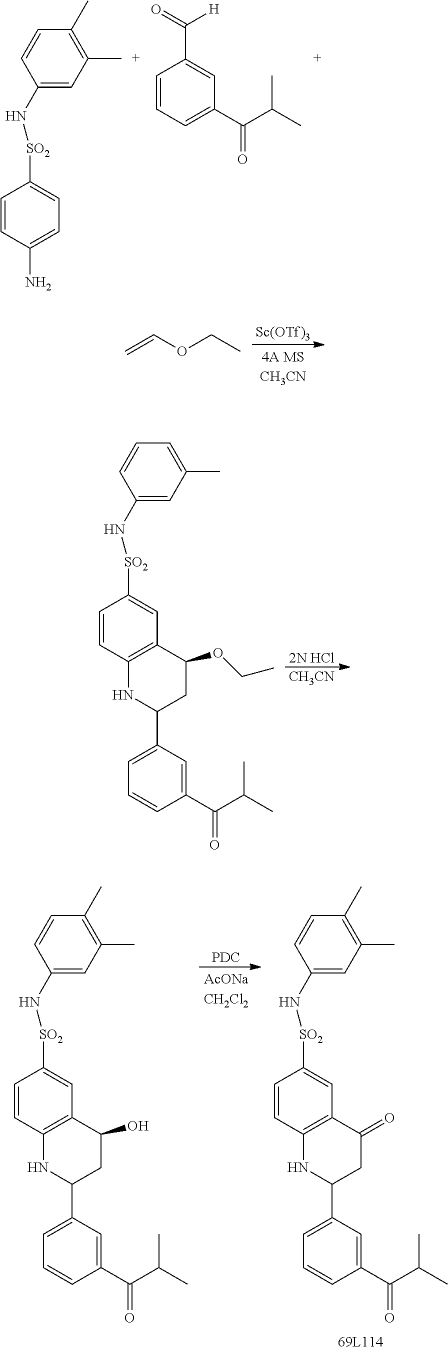

### Preparation of 3-isobutyrylbenzaldehyde

To a solution of 1-bromo-3-(diethoxymethyl)benzene (50.00 g, 192.95 mmol) in 200 mL of anhydrous tetrahydrofuran under an argon atmosphere was added dropwise n-butyllithium (84.90 mL of 2.5 N hexanes solution, 212.00 mmol) at −78° C. in a dry ice/acetone bath. After stirred at −78° C. for 2 hours, a solution of CuCN (17.28 g, 192.95 mmol) and LiCl (16.36 g, 385.90 mmol) in 200 mL anhydrous tetrahydrofuran was added dropwise with stirring at −78° C. under argon. The resulted solution was stirred at −78° C. for 30 minutes, then slowly warmed to the room temperature and stirred for another hour. The reaction was quenched by addition of 100 mL of water at the room temperature with vigorous stirring. THF solvent was removed under reduced pressure. The aqueous residue was extracted with methylene chloride (3×100 mL). The organic extracts were dried over anhydrous MgSO₄, filtered and concentrated under reduced pressure. The crude liquid was purified by column chromatography (silica gel, CH₂Cl₂) to give an oil product (32.00 g, 94.1% yield). ¹H NMR (400 MHz, DMSO-d⁶) δ 10.12 (s, 1H); 8.48 (s, 1H), 8.29 (d, J=7.6 Hz, 1H), 8.15 (d, J=7.6 Hz, 1H), 7.78 (t, J=7.6 Hz, 1H), 3.77-3.70 (m, 1H), 1.14 (d, J=6.8 Hz, 6H).

### Preparation of N-(3,4-dimethylphenyl)-4-ethoxy-2-(3-isobutyrylphenyl)-1,2,3,4-tetrahydroquinoline-6-sulfonamide

4-Amino-N-(3′,4-dimethylphenyl)benzenesulfonamide (0.53 g, 1.92 mmol), 3-isobutyrylbenzaldehyde (0.34 g, 1.92 mmol), Sc(OTf)₃ (0.19 g, 0.38 mmol) and 4 Å molecular sieves (1 g) were mixed together in anhydrous CH₃CN (20 mL) at room temperature under argon. The reaction mixture was stirred for one hour at room temperature. Ethoxyethene (0.28 g, 3.84 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.36 g, 37.1% yield). trans-diastereomers: ¹H NMR (400 MHz, CDCl₃) δ 8.00 (s, 1H), 7.90 (d, J=8.0 Hz, 1H), 7.61-7.54 (m, 1H), 7.51-7.26 (m, 2H), 6.99-6.77 (m, 4H), 6.52 (d, J=8.4 Hz, 1H), 6.26 (s, 1H), 4.72 (dd, J₁=12.0 Hz, J₂=2.4 Hz, 1H), 4.62 (s, 1H), 4.26 (s, 1H), 3.58-3.27 (m, 4H), 2.18 (s, 3H), 2.17 (s, 3H), 1.86-1.81 (m, 1H), 1.23-112 (m, 9H).

### Preparation of N-(3,4-dimethylphenyl)-4-hydroxy-2-(3-isobutyrylphenyl)-1,2,3,4-tetrahydroquinoline-6-sulfonamide

N-(3,4-Dimethylphenyl)-4-ethoxy-2-(3-isobutyrylphenyl)-1,2,3,4-tetrahydroquinoline-6-sulfonamide (0.28 g, 0.55 mmol) was dissolved in anhydrous CH₃CN (10 mL) at room temperature. To this solution was added 4 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=9/1 v/v) to give a pale-yellow solid product (0.18 g, 67.9% yield). trans-diastereomers: ¹H NMR (400 MHz, CDCl₃) δ 7.95 (d, J=8.4 Hz, 2H), 7.62 (d, J=2.4 Hz, 1H), 7.49 (d, J=8.4 Hz, 2H), 7.45 (dd, J₁=8.4 Hz, J₂=2.0 Hz, 2H), 6.97 (d, J=8.0 Hz, 1H), 6.89 (d, J=2.0 Hz, 1H), 6.80 (dd, J₁=8.0 Hz, J₂=2.0 Hz, 1H), 6.64 (s, 1H), 6.54 (d, J=8.8 Hz, 1H), 4.73-4.69 (m, 3H), 3.56-3.53 (m, 1H), 2.33 (s, 1H), 2.17 (s, 6H), 1.87-1.82 (m, 1H), 1.22 (d, J=6.8 Hz, 6H).

### Preparation of N-(3,4-dimethylphenyl)-2-(3-isobutyrylphenyl)-4-oxo-1,2,3,4-tetrahydroquinoline-6-sulfonamide. (69L114)

N-(3,4-Dimethylphenyl)-4-hydroxy-2-(3-isobutyrylphenyl)-1,2,3,4-tetrahydroquinoline-6-sulfonamide (43 mg, 0.09 mmol) was dissolved in anhydrous methylene chloride (10 mL) at room temperature. To this solution were added 34 mg (0.09 mmol) of pyridinium dichromate and sodium acetate (7 mg, 0.09 mmol) at room temperature. The reaction solution was stirred at room temperature for one hour, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (15 mg, 42.9% yield). ¹H NMR (400 MHz, CDCl₃) δ 8.33 (d, J=2.4 Hz, 1H), 8.04 (s, 1H), 7.94 (d, J=8.0 Hz, 1H), 7.62 (d, J=2.4 Hz, 1H), 7.61-7.59 (m, 1H), 7.54-7.50 (m, 1H), 6.99 (d, J=8.0 Hz, 1H), 6.89 (d, J=2.0 Hz, 1H), 6.79 (dd, J₁=8.0 Hz, J₂=2.4 Hz, 1H), 6.68 (d, J=8.8 Hz, 1H), 6.40 (s, 1H), 4.94 (s, 1H), 4.88 (dd, J₁=12.8 Hz, J₂=4.4 Hz, 1H), 3.58-3.51 (m, 1H), 2.96-2.81 (m, 2H), 2.16 (s, 3H), 2.15 (s, 3H), 1.21 (d, J=6.8 Hz, 6H).

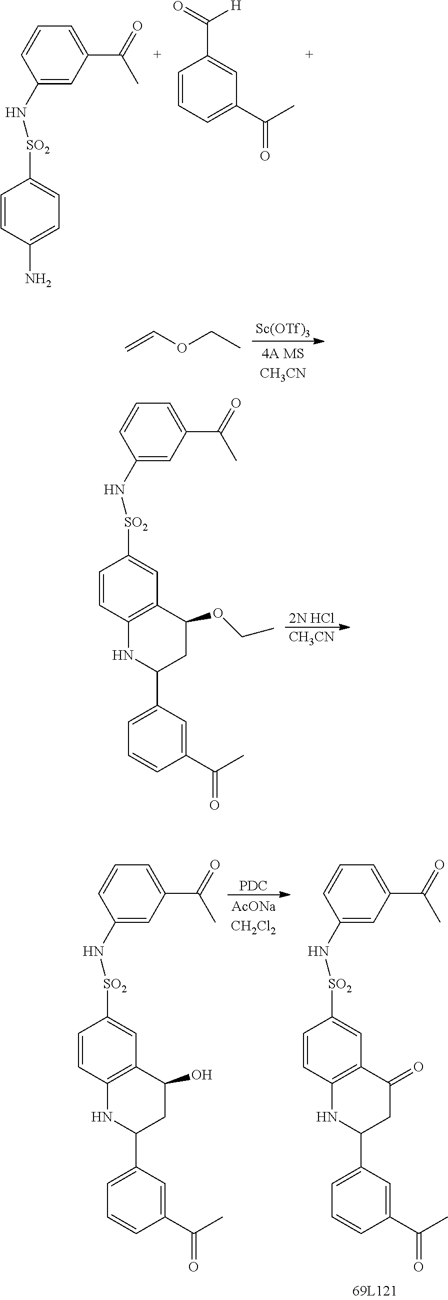

### Preparation of N,2-bis(3-acetylphenyl)-4-ethoxy-1,2,3,4-tetrahydroquinoline-6-sulfonamide

N-(3-Acetylphenyl)-4-aminobenzenesulfonamide (0.66 g, 2.27 mmol), 3-acetyl-benzaldehyde (0.34 g, 2.27 mmol), Sc(OTf)₃ (0.22 g, 0.45 mmol) and 4 Å molecular sieves (1 g) were mixed together in anhydrous CH₃CN (20 mL) at room temperature under argon. The reaction mixture was stirred for one hour at room temperature. Ethoxyethene (0.33 g, 4.54 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.46 g, 41.1% yield). trans-diastereomers: ¹H NMR (400 MHz, CDCl₃) δ 8.02 (s, 1H), 7.92 (s, 1H), 7.71 (d, J=7.2 Hz, 1H), 7.65-7.63 (m, 2H), 7.57-7.50 (m, 4H), 7.41 (s, 2H), 6.55 (s, 1H), 4.74 (dd, J₁=12.0 Hz, J₂=2.4 Hz, 1H), 4.68 (s, 1H), 4.29 (t, J=2.8 Hz, 1H), 3.71-3.67 (m, 1H), 3.53-3.44 (m, 2H), 3.39-3.31 (m, 1H), 2.64 (s, 3H), 2.59 (s, 3H), 2.29-2.25 (m, 1H), 1.88-1.81 (m, 1H), 1.23 (t, J=7.6 Hz, 3H).

### Preparation of N,2-bis(3-acetylphenyl)-4-hydroxy-1,2,3,4-tetrahydroquinoline-6-sulfonamide

N,2-bis(3-Acetylphenyl)-4-ethoxy-1,2,3,4-tetrahydroquinoline-6-sulfonamide (0.20 g, 0.41 mmol) was dissolved in anhydrous CH₃CN (10 mL) at room temperature. To this solution was added 3 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=9/1 v/v) to give a pale-yellow solid product (68 mg, 36.1% yield). trans-diastereomers: ¹H NMR (400 MHz, CDCl₃) δ 9.79 (s, 1H), 7.87 (d, J=7.2 Hz, 1H), 7.79 (s, 1H), 7.67 (s, 1H), 7.62-7.57 (m, 3H), 7.47-7.43 (m, 3H). 7.39 (d, J=7.2 Hz, 1H), 7.33 (d, J=8.0 Hz, 1H), 6.52 (d, J=8.8 Hz, 1H), 5.02 (s, 1H), 4.71-4.67 (m, 2H), 3.64 (s, 1H), 2.58 (s, 3H), 2.53 (s, 3H), 2.18-2.14 (m, 1H), 1.86-1.79 (m, 1H).

### Preparation of N,2-bis(3-acetylphenyl)-4-oxo-1,2,3,4-tetrahydroquinoline-6-sulfonamide. (69L121)

N,2-bis(3-Acetylphenyl)-4-hydroxy-1,2,3,4-tetrahydroquinoline-6-sulfonamide (52 mg, 0.11 mmol) was dissolved in anhydrous methylene chloride (10 mL) at room temperature. To this solution were added 42 mg (0.11 mmol) of pyridinium dichromate and sodium acetate (9 mg, 0.11 mmol) at room temperature. The reaction solution was stirred at room temperature for one hour, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (19 mg, 36.7% yield). 1H NMR (400 MHz, CDCl₃) δ 8.26 (d, J=2.4 Hz, 1H), 7.98 (s, 1H), 7.88 (d, J=8.0 Hz, 1H), 7.62 (d, J=7.6 Hz, 1H). 7.59-7.54 (m, 3H), 7.48-7.43 (M, 1H), 7.39-7.19 (m, 2H), 7.07 (s, 1H), 6.64 (d, J=8.8 Hz, 1H), 4.99 (s, 1H), 4.82 (dd, J₁=12.8 Hz, J₂=4.4 Hz, 1H), 2.89-2.74 (m, 2H), 2.56 (s, 3H), 2.51 (s, 3H).

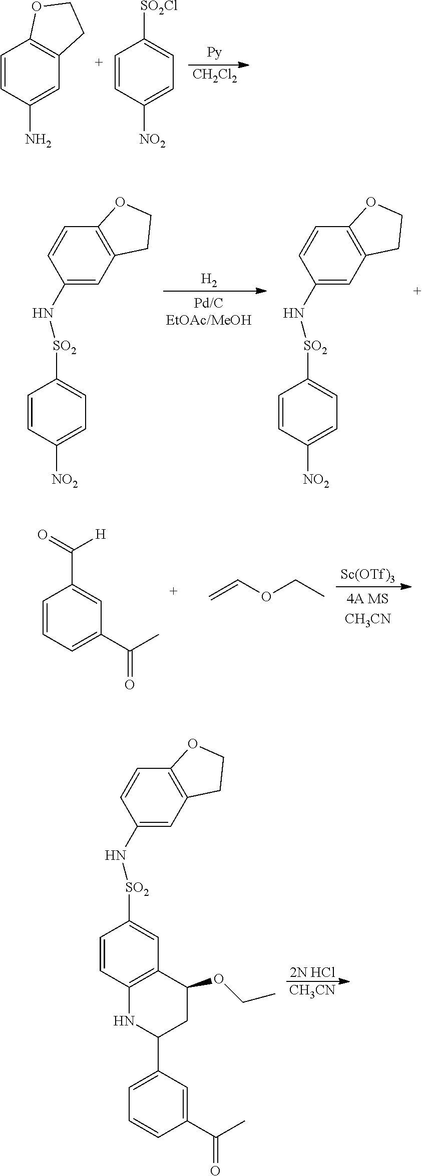

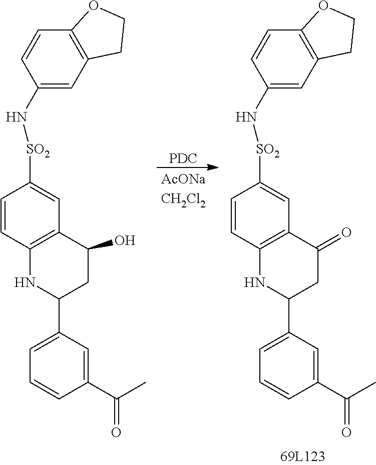 [SMILES: CC(=O)c1cccc(C2C[C@H](O)c3cc(S(=O)(=O)Nc4ccc5c(c4)CCO5)ccc3N2)c1 ; "PDC" ; CC(=O)c1cccc(C2CC(=O)c3cc(S(=O)(=O)Nc4ccc5c(c4)CCO5)ccc3N2)c1]

### Preparation of N-(2,3-dihydrobenzofuran-5-yl)-4-nitrobenzenesulfonamide

4-Nitrobenzenesulfonyl chloride (7.87 g, 35.51 mmol) and 2,3-dihydrobenzofuran-5-amine (4.80 g, 35.51 mmol) were dissolved in anhydrous methylene chloride (100 mL) at room temperature tinder argon. Pyridine (4.21 g, 53.27 mmol) was added dropwise via a syringe at room temperature. The reaction mixture was stirred at worn temperature overnight under argon atmosphere. Then, the volatile was removed under reduced pressure. The yellow solid residue was subjected to flash column chromatography (silica gel, CH₂Cl₂/Acetone=9/1 v/v) to afford the desired product as a pale yellow solid (10.88 g, 95.7% yield). ¹H NMR (400 MHz, DMSO-d⁶) δ 10.18 (s, 1H); 8.37 (d, J=8.8 Hz, 2H), 7.91 (d, J=8.8 Hz, 2H), 6.96 (d, J=2.0 Hz, 1H), 6.73-6.70 (m, 1H), 6.61 (d, J=8.4 Hz, 1H), 4.47 (t, J=8.8 Hz, 2H), 3.09 (t, J=8.8 Hz, 2H).

### Preparation of 4-amino-N-(2,3-dihydrobenzofuran-5-yl)benzenesulfonamide

N-(2,3-Dihydrobenzofuran-5-yl)-4-nitrobenzenesulfonamide (8.00 g, 24.98 mmol) and Pd/C (10% Pd base, 1 g) were mixed together in MeOH (100 mL) and Ethyl acetate (100 mL) at room temperature. Hydrogen gas was introduced via a H₂ balloon. The reaction mixture was stirred under H₂ atmosphere at room temperature for 8 hours. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (6.85 g, 94.5% yield). ¹H NMR (400 MHz, DMSO-d⁶) δ 9.39 (s, 1H), 7.30 (d, J=88 Hz, 2H), 6.91 (s, 1H), 6.71 (dd, J₁=8.4 Hz, J₂=1.6 Hz, 1H), 6.57 (d, J=8.4 Hz, 1H), 6.51 (d, J=8.4 Hz, 2H), 5.93 (s, 2H), 4.44 (t, J=8.8 Hz, 2H), 3.07 (t, J=8.8 Hz, 2H).

### Preparation of 2-(3-acetylphenyl)-N-(2,3-dihydrobenzofuran-5-yl)-4-ethoxy-1,2,3,4-tetrahydroquinoline-6-sulfonamide

4-Amino-N-(2,3-dihydrobenzofuran-5-yl)benzenesulfonamide (1.00 g, 3.44 mmol), 3-acetylbenzaldehyde (0.51 g, 3.44 mmol), Sc(OTf)₃ (0.34 g, 0.69 mmol) and 4 Å molecular sieves (2 g) were mixed together in anhydrous CH₃CN (50 mL) at room temperature under argon. The reaction mixture was stirred for one hour at room temperature. Ethoxyethene (0.50 g, 6.88 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.72 g, 42.6% yield). trans-diastereomers: ¹H NMR (400 MHz, CDCl₃) δ 8.04 (s, 1H), 7.95-7.92 (m, 1H), 7.66 (d, J=7.6 Hz, 1H), 7.53-7.49 (m, 1H), 7.47-7.44 (m, 2H), 7.09 (d, J=2.0 Hz, 1H), 6.72 (dd, J₁=8.4 Hz, J₂=2.0 Hz, 1H), 6.64-6.62 (m, 1H), 6.56 (d, J=8.8 Hz, 1H), 6.27 (s, 1H), 4.76 (dd, J₁=12.0 Hz, J₂=2.8 Hz, 1H), 4.67 (s, 1H), 4.58 (t, J=8.8 Hz, 2H), 4.28 (t, J=2.8 Hz, 1H), 3.48-3.44 (m, 1H), 3.38-3.34 (m, 1H), 3.19 (t, J=8.8 Hz, 2H), 2.65 (s, 3H), 2.26 (dd, J₁=13.6 Hz, J₂=1.2 Hz, 1H), 1.85 (td, J₁=13.6 Hz, J₂=2.8 Hz, 1H), 1.22 (t, J=6.8 Hz, 3H).

### Preparation of 2-(3-acetylphenyl)-N-(2,3-dihydrobenzofuran-5-yl)-4-hydroxy-1,2,3,4-tetrahydroquinoline-6-sulfonamide

2-(3-Acetylphenyl)-N-(2,3-dihydrobenzofuran-5-yl)-4-ethoxy-1,2,3,4-tetrahydroquinoline-6-sulfonamide (0.45 g, 0.91 mmol) was dissolved in anhydrous CH₃CN (20 mL) at room temperature. To this solution was added 6 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=9/1 v/s) to give a pale-yellow solid product (0.35 mg, 82.5% yield). trans-diastereomers: ¹H NMR (400 MHz, CDCl₃) δ 8.05 (s, 1H), 7.93 (d, J=7.2 Hz, 1H), 7.64 (d, J=7.6 Hz, 1H), 7.59 (s, 1H), 7.53-7.49 (m, 1H), 7.39 (d, J=8.4 Hz, 1H), 7.07 (s, 1H), 6.70 (dd, J₁=8.4 Hz, J₂=2.0 Hz, 1H), 6.30 (d, J=8.4 Hz, 1H), 6.56 (d, J=8.4 Hz, 1H), 4.77 (s, 1H), 4.73 (s, 1H), 4.58 (t, J=8.8 Hz, 2H), 3.19 (t, J=8.8 Hz, 2H), 2.64 (s, 3H), 2.26 (dd, J₁=13.6 Hz, J₂=1.6 Hz, 1H), 1.91 (t, J=13.6 Hz, 1H).

### Preparation of 2-(3-acetylphenyl)-N-(2,3-dihydrobenzofuran-5-yl)-4-oxo-1,2,3,4-tetrahydroquinoline-6-sulfonamide (69L123)

2-(3-Acetylphenyl)-N-(2,3-dihydrobenzofuran-5-yl)-4-hydroxy-1,2,3,4-tetrahydro-quinoline-6-sulfonamide (63 mg, 0.14 mmol) was dissolved in anhydrous methylene chloride (10 mL) at room temperature. To this solution were added 51 mg (0.14 mmol) of pyridinium dichromate at room temperature. The reaction solution was stirred at room temperature for one hour, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (22 mg, 35.1% yield). ¹H NMR (400 MHz, CDCl₃) δ 8.22 (d, J=2.4 Hz, 1H), 8.00 (s, 1H), 7.89 (dt, J₁=7.6 Hz, J₂=1.6 z, 1H), 7.56 (d, J=8.0 Hz, 1H), 7.49-7.45 (m, 1H), 7.02 (s, 1H), 6.62 (d, J=8.8 Hz, 1H), 6.58 (dd, J₁=8.8 Hz, J₂=2.0 Hz, 1H), 6.53 (d, J=8.4 Hz, 1H), 6.22 (s, 1H), 4.88 (s, 1H), 4.82 (dd, J₁=12.8 Hz, J₂=4.4 Hz, 1H), 4.52 (t, J=8.8 Hz, 2H), 3.12 (t, J=8.8 Hz, 2H), 2.86-2.80 (ma, 2H), 2.57 (s, 3H).

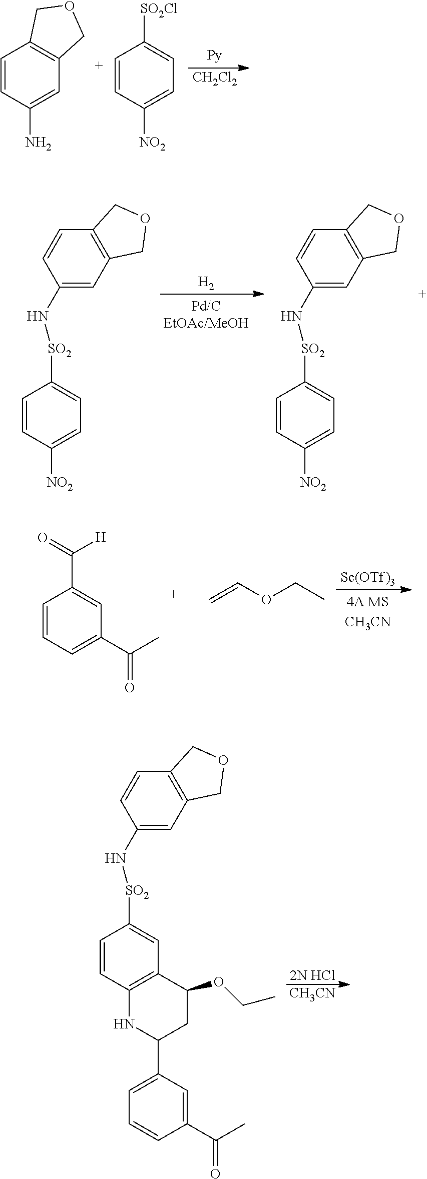

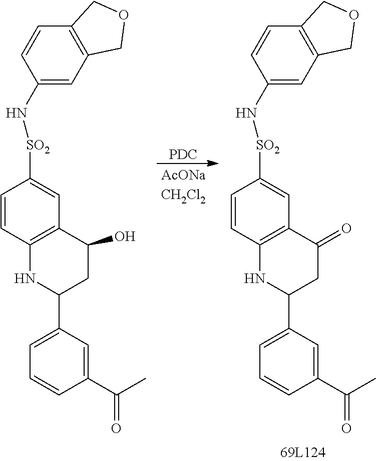 [SMILES: CC(=O)c1cccc(C2C[C@H](O)c3cc(S(=O)(=O)Nc4ccc5c(c4)COC5)ccc3N2)c1 ; "PDC" ; CC(=O)c1cccc(C2CC(=O)c3cc(S(=O)(=O)Nc4ccc5c(c4)COC5)ccc3N2)c1]

### Preparation of N-(1,3-dihydroisobenzofuran-5-yl)-4-nitrobenzenesulfonamide

4-Nitrobenzenesulfonyl chloride (10.16 g, 45.87 mmol) and 1,3-dihydroisobenzofuran-5-amine (6.20 g, 45.87 mmol) were dissolved in anhydrous methylene chloride (100 mL) at room temperature under argon.

Pyridine (5.44 g, 68.81 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=9/1 v/v) to afford the desired product as a pale yellow solid (13.55 j, 92.2% yield). ¹H NMR (400 MHz, DMSO-d⁶) δ 10.64 (s, 1H); 8.38 (d, J=8.8 Hz, 2H), 7.99 (d, J=8.8 Hz, 2H), 7.18 (d, J=8.0 Hz, 1H), 7.07 (d, J=1.2 Hz, 1H), 6.99 (d, J=8.4 Hz, 1H), 4.89 (s, 4H).

### Preparation of 4-amino-N-(1,3-dihydroisobenzofuran-5-yl)benzenesulfonamide

N-(1,3-Dihydroisobenzofuran-5-yl)-4-nitrobenzenesulfonamide (8.00 g, 24.98 mmol) and Pd/C (10% Pd base, 1 g) were mixed together in MeOH (100 mL) and Ethyl acetate (100 mL) at room temperature. Hydrogen gas was introduced via a H₂ balloon. The reaction mixture was stirred under H₂ atmosphere at room temperature for 8 hours. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (6.90 g, 95.2% yield). ¹H NMR (400 MHz, DMSO-d⁶) δ 9.89 (s, 1H), 7.38 (d, J=8.8 Hz, 2H), 7.11 (d, J=8.4 Hz, 1H), 7.00 (s, 1H), 6.96 (dd, J₁=8.0 Hz, J₂=1.6 Hz, 1H), 6.52 (d, J=8.8 Hz, 2H), 5.98 (s, 2H), 4.88 (s, 4H).

### Preparation of 2-(3-acetylphenyl)-N-(1,3-dihydroisobenzofuran-5-yl)-4-ethoxy-1,2,3,4-tetrahydroquinoline-6-sulfonamide

4-Amino-N-(1,3-dihydroisobenzofuran-5-yl)benzenesulfonamide (1.00 g, 3.44 mmol), 3-acetylbenzaldehyde (0.51 g, 3.44 mmol), Sc(OTf)₃ (0.34 g, 0.69 mmol) and 4 Å molecular sieves (2 g) were mixed together in anhydrous CH₃CN (50 mL) at room temperature under argon. The reaction mixture was stirred for one hour at room temperature. Ethoxyethene (0.50 g, 6.88 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.70 g, 41.4% yield). trans-diastereomers: ¹H NMR (400 MHz, CDCl₃) δ 8.03 (s, 1H), 7.94-7.92 (m, 1H), 7.64 (d, J=8.0 Hz, 1H), 7.56 (d, J=2.0 Hz, 1H), 7.52 (d, J=2.0 Hz, 1H), 7.50-7.43 (m, 2H), 7.13-7.08 (m, 2H), 6.98 (d, J=1.6 Hz, 1H), 6.55 (d, J=8.8 Hz, 1H), 5.06 (s, 4H), 5.04-5.01 (m, 2H), 4.74 (dd, J₁=12.0 Hz, J₂=2.81 Hz, 1H), 4.70 (s, 1H), 4.29 (t, J=2.8 Hz, 1H), 3.71-3.66 (m, 1H), 351-346 (m, 2H), 3.39-3.35 (m, 1H), 2.64 (s, 3H), 2.28-2.25 (m, 1H), 1.85 (td, J₁=13.6 Hz, J₂=2.8 Hz, 1H), 1.20 (t, J=7.2 Hz, 3H).

### Preparation of 2-(3-acetylphenyl)-N-(1,3-dihydroisobenzofuran-5-yl)-4-hydroxy-1,2,3,4-tetrahydroquinoline-6-sulfonamide

2-(3-Acetylphenyl)-N-(1,3-dihydroisobenzofuran-5-yl)-4-ethoxy-1,2,3,4-tetrahydro-quinoline-6-sulfonamide (0.45 g, 0.91 mmol) was dissolved in anhydrous CH₃CN (20 mL) at room temperature. To this solution was added 6 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=9/1 v/v) to give a pale-yellow solid product (0.28 mg, 66.7% yield). trans-diastereomers: ¹H NMR (400 MHz, CDCl₃) δ 7.99 (s, 1H), 7.89 (d, J=7.6 Hz, 1H), 7.71 (d, J=2.0 Hz, 1H), 7.59 (d, J=7.6 Hz, 1H), 7.50-7.42 (m, 2H), 7.37 (s, 1H), 7.08-6.98 (m, 3H), 6.53 (d, J=8.8 Hz, 1H), 5.02 (s, 2H), 4.99 (s, 2H), 4.92 (s, 1H), 4.70-4.68 (m, 2H), 3.11 (s, 1H), 2.61 (s, 3H), 2.18-2.16 (m, 1H), 1.85 (td, J₁=13.6 Hz, J₂=2.8 Hz, 1H).

### Preparation of 2-(3-acetylphenyl)-N-(1,3-dihydroisobenzofuran-5-yl)-4-oxo-1,2,3,4-tetrahydroquinoline-6-sulfonamide (69L124)

2-(3-Acetylphenyl)-N-(1,3-dihydroisobenzofuran-5-yl)-4-hydroxy-1,2,3,4-tetrahydro-quinoline-6-sulfonamide (0.10 g, 0.22 mmol) was dissolved in anhydrous methylene chloride (10 mL) at room temperature. To this solution were added 83 mg (0.22 mmol) of pyridinium dichromate and sodium acetate (18 mg, 0.22 mmol) at room temperature. The reaction solution was stirred at room temperature for one hour, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (35 mg, 354% yield). ¹H NMR (400 MHz, CDCl₃) δ 8.34 (d, J=2.0 Hz, 1H), 8.05 (s, 1H), 7.95 (dt, J₁=0.6 Hz, J₂=1.2 Hz, 1H), 7.63-7.61 (m, 2H), 7.56-7.51 (m, 2H), 7.10 (d, J=8.0 Hz, 1H), 7.04 (d, J=1.6 Hz, 1H), 6.95 (dd, J₁=8.0 Hz, J₂=10 Hz, 1H), 6.70 (d, J=8.8 Hz, 1H), 6.64 (s, 1H), 5.03 (s, 4H), 4.98 (s, 1H), 4.88 (dd, J₁=12.8 Hz, J₂=4.4 Hz, 1H), 2.92-2.86 (m, 2H), 2.63 (s, 3H).

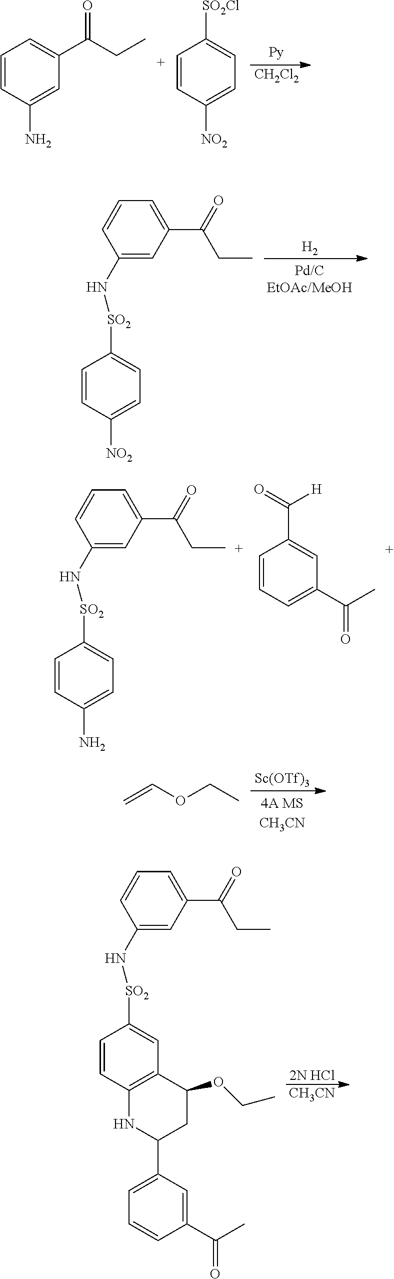

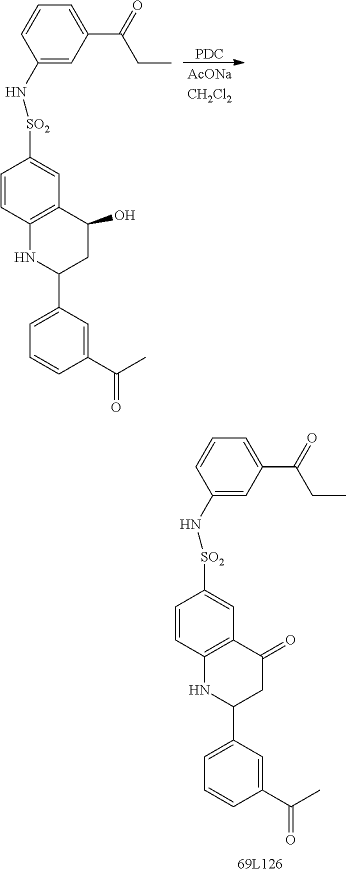

### Preparation of 4-nitro-N-(3-propionylphenyl)benzenesulfonamide

4-Nitrobenzenesulfonyl chloride (7.43 g, 3351 mmol) and 1-(3-aminophenyl)propan-1-one (5.00 g, 33.51 mmol) were dissolved in anhydrous methylene chloride (100 mL) at room temperature under argon. Pyridine (3.97 g, 50.27 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=9/1 v/v) to afford the desired product as a pale yellow solid (11.00 g, 98.2% yield). ¹H NMR (400 MHz, DMSO-d⁶) δ 10.88 (s, 1H), 8.01 (d, J=8.8 Hz, 2H), 7.70 (dt, J₁=7.6 Hz, J₂=1.2 Hz, 1H), 7.67 (t, J=2.4 Hz, 1H), 7.45-7.41 (m, 1H), 7.37-7.34 (m, 1H), 2.96 (q, J=7.2 Hz, 2H), 1.04 (t, J=7.2 Hz, 3H).

### Preparation of 4-amino-N-(3-propionylphenyl)benzenesulfonamide

Compound 4-nitro-N-(3-propionylphenyl)benzenesulfonamide (10.00 g, 29.91 mmol) and Pd/C (10% Pd base, 1 g) were mixed together in MeOH (100 mL) and Ethyl acetate (100 mL) at room temperature. Hydrogen gas was introduced via a H₂ balloon. The reaction mixture was stirred under H₂ atmosphere 8 hours at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (8.50 g, 93.4% yield). ¹H NMR (400 MHz, DMSO-d⁶) δ 10.12 (s, 1H), 7.62 (t, J=1.6 Hz, 1H), 7.59 (d, J=7.6 Hz, 1H), 7.40 (d, J=7.6 Hz, 2H), 7.38-7.29 (m, 2H), 6.52 (d, J=8.8 Hz, 2H), 6.01 (s, 2H), 2.94 (q. J=7.2 Hz, 2H), 1.04 (t, J=7.2 Hz, 3H).

### Preparation of 2-(3-acetylphenyl)-4-ethoxy-N-(3-propionylphenyl)-1,2,3,4-tetrahydroquinoline-6-sulfonamide

Compound 4-amino-N-(3-propionylphenyl)benzenesulfonamide (0.50 g, 1.64 mmol), 3-acetylbenzaldehyde (0.24 g, 1.64 mmol), Sc(OTf)₃ (0.16 g, 0.33 mmol) and 4 Å molecular sieves (1 g) were mixed together in anhydrous CH₃CN (20 mL) at room temperature under argon. The reaction mixture was stirred for one hour at room temperature. Ethoxyethene (0.24 g, 3.28 mmol) was then added via a syringe. The resulted mixture was stirred at room temperature under argon overnight. The reaction was quenched by adding 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Acetone=9/1 v/v) to give a white solid product (0.25 g, 30.1% yield). trans-diastereomers: ¹H NMR (400 MHz, CDCl₃) δ 8.02 (s, 1H), 7.92 (s, 1H), 7.71 (d, J=6.8 Hz, 1H), 7.67-7.63 (m, 2H), 7.57-7.36 (m, 4H), 7.40 (s, 2H), 6.70 (s, 1H), 6.55 (d, J=8.4 Hz, 1H), 4.74 (dd, J₁=12.0 Hz, J₂=2.4 Hz, 1H), 4.70 (s, 1H), 4.28 (t, J=2.8 Hz, H), 3.70-3.61 (m, 1H), 3.51-3.45 (m, 2H), 3.36-3.32 (m, 1H), 3.00-2.93 (m, 3H), 2.64 (s, 3H), 2.28-2.22 (m, 1H), 1.88-1.81 (m, 1H), 1.21-1.17 (m, 6H).

### Preparation of 2-(3-acetylphenyl)-4-hydroxy-N-(3-propionylphenyl)-1,2,3,4-tetrahydro-quinoline-6-sulfonamide

Compound 2-(3-acetylphenyl)-4-ethoxy-N-(3-propionylphenyl)-1,2,3,4-tetrahydro-quinoline-6-sulfonamide (0.25 g, 0.49 mmol) was dissolved in anhydrous CH₃CN (10 mL) at room temperature. To this solution was added 5 mL of 2N HCl solution at room temperature. The reaction solution was stirred at room temperature for one hour, then diluted with 50 mL of water and neutralized with NaHCO₃. The resulted solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/Methanol=9/1 v/v) to give a pale-yellow solid product (0.16 mg, 67.8% yield). trans-diastereomers: ¹H NMR (400 MHz, CDCl₃) δ 8.01 (s, 1H), 7.91 (d, J=7.6 Hz, MH), 7.79 (d, J=2.0 Hz, 1H), 7.68 (s, 1H), 7.66-7.61 (m, 2H), 7.51-7.49 (m, 2H), 7.37-7.36 (m, 2H), 7.27 (s, 1H), 6.55 (d, J=8.8 Hz, 1H), 4.87 (s, 1H), 4.78-4.72 (m, 2H), 2.99-2.92 (m, 4H), 3.64 (s, 1H), 2.62 (s, 3H), 2.23-2.20 (m, 1H), 1.92-1.85 (m, 1H), 1.21-1.18 (m, 6H).

### Preparation of 2-(3-acetylphenyl)-4-oxo-N-(3-propionylphenyl)-1,2,3,4-tetrahydro-quinoline-6-sulfonamide. (69L126)

Compound 2-(3-acetylphenyl)-4-hydroxy-N-(3-propionylphenyl)-1,2,3,4-tetrahydroquinoline-6-sulfonamide (0.15 mg, 0.31 mmol) was dissolved in anhydrous methylene chloride (10 mL) at room temperature. To this solution were added 0.12 g (0.31 mmol) of pyridinium dichromate and sodium acetate (25 mg, 0.31 mmol) at room temperature. The reaction solution was stirred at room temperature for one hour, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (35 mg, 23.7% yield). ¹H NMR (400 MHz, CDCl₃) δ 8.34 (d, J=2.4 Hz, 1H), 8.06 (s, 1H), 7.97-7.95 (m, 1H), 7.71 (dt, J₁=7.2 Hz, J₂=1.6 Hz, 1H), 7.66-7.61 (m, 3H), 7.55-7.51 (m, 1H), 7.45-7.37 (m, 2H), 7.06 (s, 1H), 6.71 (d, J=8.8 Hz, 1H), 5.03 (s, 1H), 4.89 (dd, J₁=12.8 Hz, J₂=4.4 Hz, 1H), 2.00-2.87 (m, 4H), 2.63 (s, 3H), 1.21 (t, J=7.2 Hz, 3H).

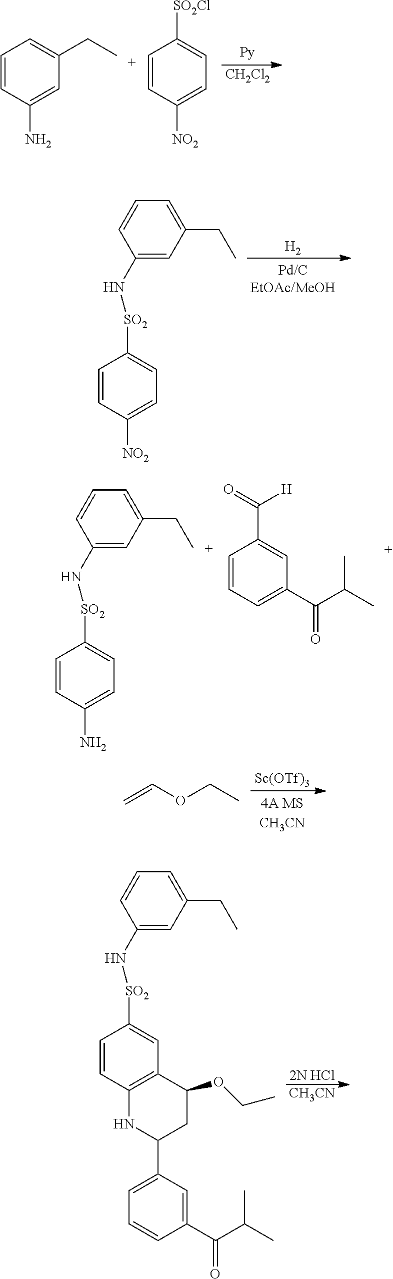

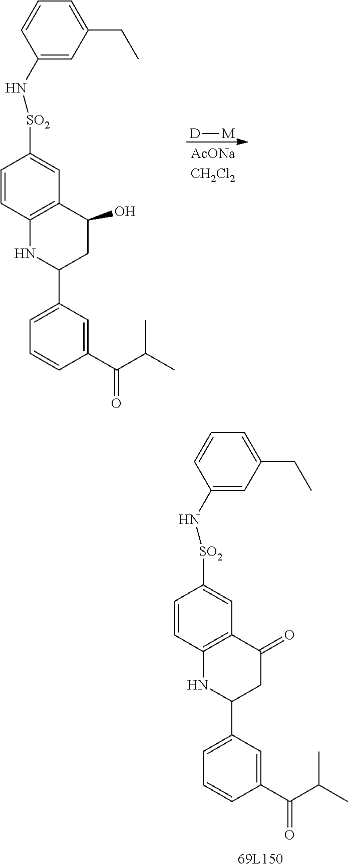

### Preparation of N-(3-ethylphenyl)-2-(3-isobutyrylphenyl)-4-oxo-1,2,3,4-tetrahydroquinoline-6-sulfonamide. (69L150)

Compound N-(3-ethylphenyl)-4-hydroxy-2-(3-isobutyrylphenyl)-1,2,3,4-tetrahydroquinoline-6-sulfonamide (0.34 mg, 0.70 mmol) was dissolved in anhydrous methylene chloride (2.0 mL) at room temperature. To this solution were added 0.30 g (0.70 mmol) of Dess-Martin reagent at room temperature. The reaction solution was stirred at room temperature for 10 minutes, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (110 ng, 23.1% yield). ¹H NMR (400 MHz, CDCl₃) δ 8.35 (d, J=2.2 Hz, 1H), 8.05 (s, 1H), 7.95 (d, J=7.7 Hz, 1H), 7.64 (dd, J₁=8.7 Hz, J₂=2.3 Hz, 1H), 7.61 (d, J=7.8 Hz, 1H), 7.53 (t, J=7.7 Hz, 1H), 7.16 (t, J=8.0 Hz, 1H), 6.98-6.93 (m, 2H), 6.89 (d, J=7.7 Hz, 1H), 6.69 (d, J=8.7 Hz, 1H), 6.61 (s, 1H), 4.98 (s, 1H), 4.88 (dd, J₁=12.7 Hz, J₂=4.5 Hz, 1H), 3.55 (dt, J₁=13.7 Hz, J₂=6.9 Hz, 1H), 2.99-2.81 (m, 2H) 2.59 (q, J=7.6 Hz, 2H), 1.23 (dd, J₁=6.8 Hz, J₂=1.3 Hz, 6H), 1.18 (t, J=7.6 Hz, 3H).

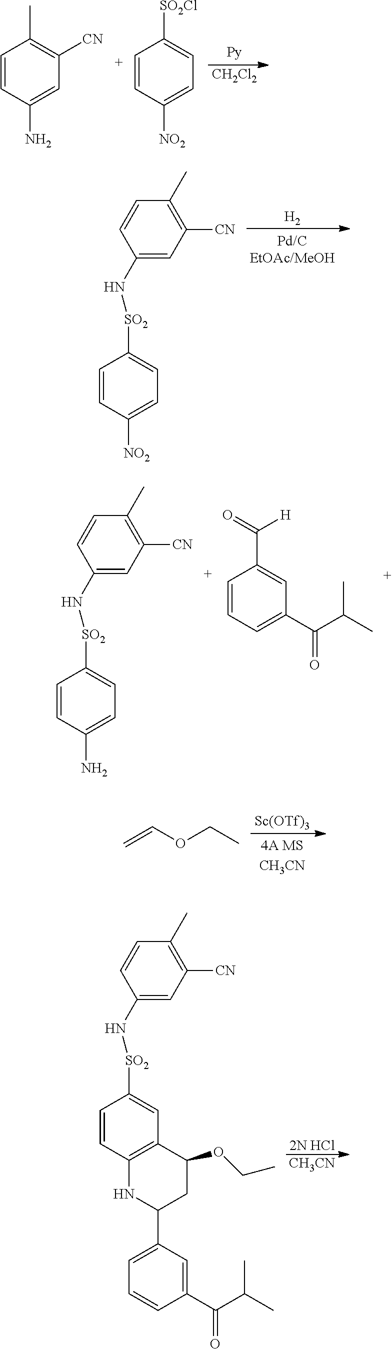

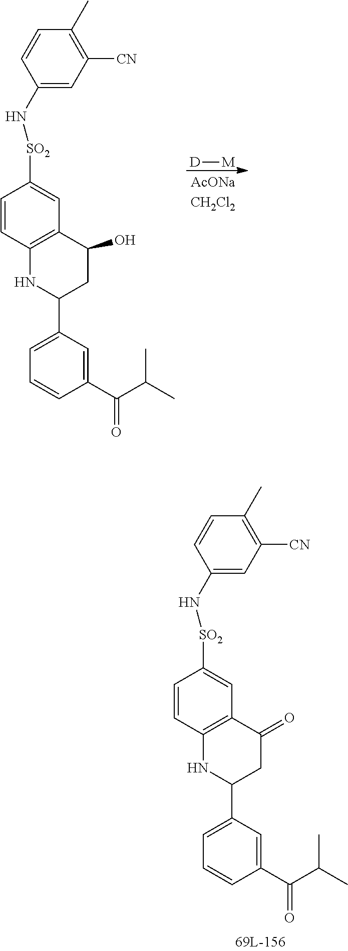

### Preparation of N-(3-cyano-4-methylphenyl)-4-nitrobenzenesulfonamide

4-Nitrobenzenesulfonyl chloride (10.0 g, 75.66 mmol) and 5-amino-2-methylbenzonitrile (16.77 g, 5.66 mmol) were dissolved in anhydrous methylene chloride (200 mL) at room temperature under argon. Pyridine (8.98 g, 113.49 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=9/1 v/O to afford the desired product as a pale yellow solid (23.5 g, 97.5% yield). ¹H NMR (400 MHz, DMSO) δ 10.92 (s, 1H), 8.36 (dd, J₁=9.1 Hz, J₂=2.1 Hz, 2H), 8.03-7.92 (m, 2H), 7.39 (d, J=2.2 Hz, 1H), 7.34 (d, J=8.5 Hz, 1H), 7.28 (dd, J₁=8.4, J₂=2.3 Hz, 1H), 2.35 (s, 3H).

### Preparation of 4-amino-N-(3-cyano-4-methylphenyl)benzenesulfonamide

Compound N-(3-cyano-4-methylphenyl)-4-nitrobenzenesulfonamide (13.00 g, 40.97 mmol) and Pd/C (10% Pd base, 2 g) were mixed together in MeOH (100 mL) and Ethyl acetate (100 mL) at room temperature. Hydrogen gas was introduced via a H₂ balloon. The reaction mixture was stirred under 1-2 atmosphere 8 hours at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (10.80 g, 91.8% yield). ¹H NMR (400 MHz, DMSO) δ 10.17 (s, 1H), 7.39 (d, J=8.6 Hz, 2H), 7.34-7.21 (m, 3H), 6.53 (d, J=8.7 Hz, 2H), 6.04 (s, 2H).

### Preparation of N-(3-cyano-4-methylphenyl-2-(3-isobutyrylphenyl)-4-oxo-1,2,3,4-tetrahydroquinoline-6-sulfonamide (69L156)

Compound N-(3-cyano-4-methylphenyl)-4-hydroxy-2-(3-isobutyrylphenyl)-1,2,3,4-tetrahydroquinoline-6-sulfonamide (0.22 mg, 0.45 mmol) was dissolved in anhydrous methylene chloride (10 mL) at room temperature. To this solution were added 0.19 g (0.45 mmol) of Dess-Martin reagent at room temperature. The reaction solution was stirred at room temperature for 10 minutes, then diluted with 20 mL of water. The resulted mixture was extracted with methylene chloride (3×50 m1L). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (70 mg, 32% yield). ¹H NMR (400 MHz, CDCl₃) δ 8.32 (d, J=2.2 Hz, 1H), 8.07 (s, 1H), 7.95 (d, J=7.8 Hz, 1H), 7.65-7.59 (m, 2H), 7.53 (t, J=7.7 Hz, 1H), 7.36 (dd, J₁=8.4 Hz, J₂=2.3 Hz, 1H), 7.32-7.29 (m, 2H), 7.24 (d, J=8.4 Hz, 1H), 6.73 (d, J=8.8 Hz, 1H), 5.14 (s, 1H), 4.93 (dd, J₁=13.2 Hz, J₂=4.3 Hz, 1H), 3.56 (dt, J₁=13.6 Hz, J₂=6.8 Hz, 1H), 3.05-2.84 (m, 2H), 2.48 (s, 3H), 1.23 (dd, J₁=6.8 Hz, J₂=1.7 Hz, 6H).

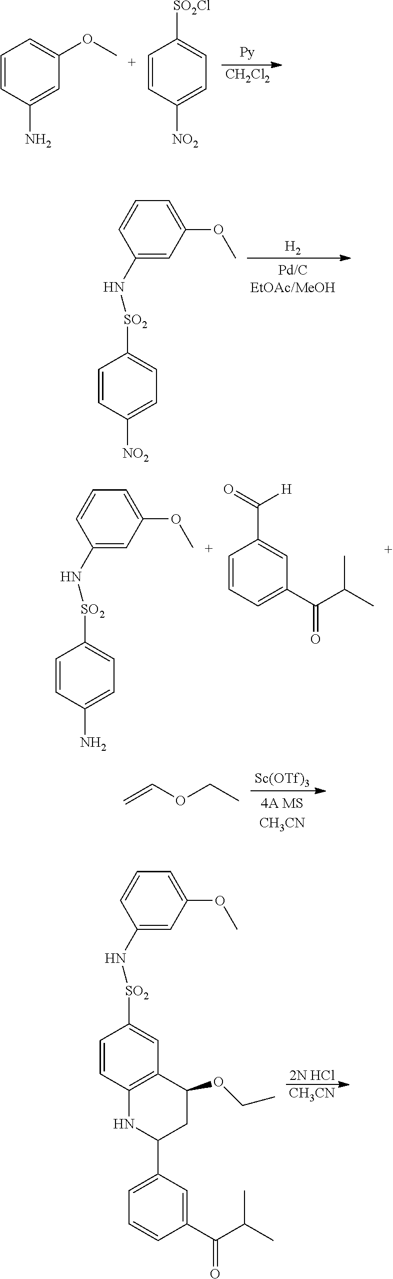

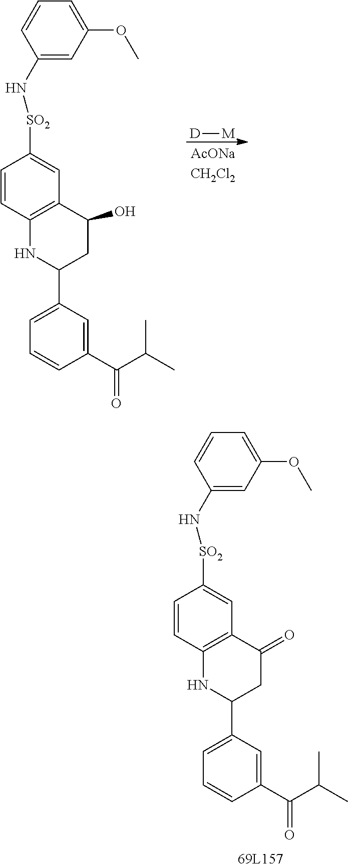

### Preparation of N-(3-methoxyphenyl)-4-nitrobenzenesulfonamide

4-Nitrobenzenesulfonyl chloride (23.77 g, 107.3 mmol) and 3-methoxyaniline (13.21 g, 107.3 mmol) were dissolved in anhydrous methylene chloride (200 mL) at room temperature under argon. Pyridine (12.73 g, 160.95 mmol) was added dropwise via a syringe at room temperature. The reaction mixture was stirred at room temperature overnight under argon atmosphere. Then, die volatile was removed under reduced pressure. The yellow solid residue was subjected to flash column chromatography (silica gel, CH₂Cl₂) to afford the desired product as a pale yellow solid (32.00 g, 96.7% yield). ¹H NMR (400 MHz, DMSO) δ 10.63 (s, 1H), 8.37 (d, J=8.8 Hz, 2H), 8.00 (d, Jr 8.8 Hz, 2H), 7.14 t, J=8.4 Hz, 1H), 6.72-6.59 (m, 3H), 3.65 (s, 3H).

### Preparation of 4-amino-N-(3-methoxyphenyl)benzenesulfonamide

Compound N-(3-methoxyphenyl)-4-nitrobenzenesulfonamide (17.00 g, 55.14 mmol) and Pd/C (10% Pd base, 2 g) were mixed together in MeOH (100 mL) and Ethyl acetate (100 mL) at room temperature. Hydrogen gas was introduced via a H₂ balloon. The reaction mixture was stirred under H₂ atmosphere 8 hours at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (14.5 g, 94.8% yield).

### Preparation of 2-(3-isobutyrylphenyl)-N-(3-methoxyphenyl)-4-oxo-1,2,3,4-tetrahydroquinoline-6-sulfonamide. (69L157)

Compound 4-hydroxy-2-(3-isobutyrylphenyl)-N-(3-methoxyphenyl)-1,2,3,4-tetrahydroquinoline-6-sulfonamide (0.10 mg, 0.21 mmol) was dissolved in anhydrous methylene chloride (10 ml) at room temperature. To this solution were added 0.09 g (0.21 mmol) of Dess-Martin reagent at room temperature. The reaction solution was stirred at room temperature for 10 minutes, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (31 mg, 31.1% yield). ¹H NMR (400 MHz, CDCl₃) δ 8.37 (d, J=2.1 Hz, 1H), 8.05 (s, 1H), 7.95 (d, J=7.7 Hz, 1H), 7.66 (dd, J₁=8.7 Hz, J₂=2.3 Hz, 1H), 7.61 (d, J=7.7 Hz, 1H), 7.53 (t, 1=7.7 Hz, 1H), 7.14 (t, J=8.1 Hz, 1H), 6.80 (s, 1H), 6.73 (t, J=2.1 Hz, 1H), 6.70 (d, J=8.7 Hz, 1H), 6.65 (dd, J₁=8.2 Hz, J₂=2.2 Hz, 2H), 5.02 (s, 1H), 4.89 (dd, J₁=12.9 Hz, J₂=4.5 Hz, 1H), 3.56 (dt, J₁=13.6 Hz, J₂=6.8 Hz, 1H), 2.95-2.83 (m, 2H), 1.23 (dd, J=6.8 Hz, J₂=1.2 Hz, 6H).

### Synthesis of Reverse Sulfonamide Analogs (69L162 and 69L163)

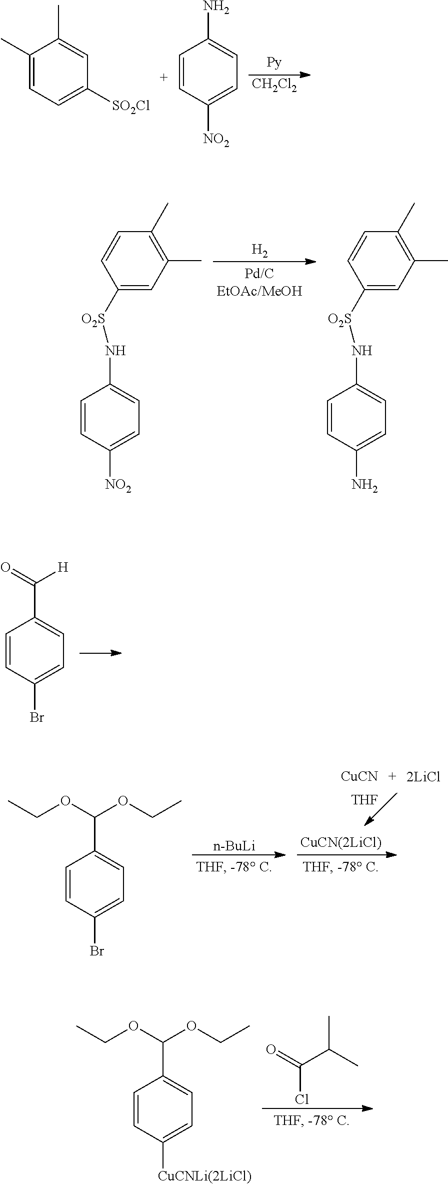

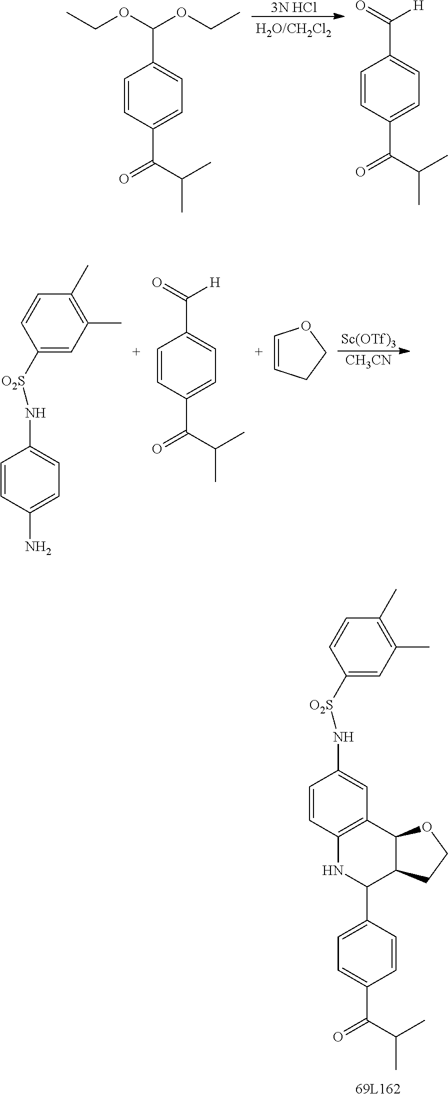

### Preparation of 3,4-dimethyl-N-(4-nitrophenyl)benzenesulfonamide

3,4-Dimethylbenzenesulfonyl chloride (7.41 g, 36.20 mmol) and 4-nitroaniline (5.00 g, 36.20 mmol) were dissolved in anhydrous methylene chloride (100 mL) at room temperature under argon. Pyridine (3.811 g, 54.30 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂) to afford product as a pale yellow solid (9.81 g, 88.5% yield). ¹H NMR (400 MHz, DMSO-d⁶): δ 11.21 (s, 1H), 8.13 (dd, J₁=7.2 Hz, J₂=2.0 Hz, 2H), 7.66 (s, 1H), 7.59 (dd, J₁=8.0 Hz, J₂=1.6 Hz, 1H), 7.35 (d, J=8.0 Hz, 1H), 7.30 (dd, J₁=7.2 Hz, J₂=2.0 Hz, 2H), 2.27 (s, 3H), 2.25 (s, 3H).

### Preparation of N-(4-aminophenyl)-3,4-dimethylbenzenesulfonamide

Compound 3,4-dimethyl-N-(4-nitrophenyl)benzenesulfonamide (5.00 g, 16.32 mmol) and Pd/C (10% Pd base, 1 g) were mixed together in MeOH (50 mL) at room temperature. Hydrogen gas was introduced via a H₂ balloon. The reaction mixture was stirred under H₂ atmosphere overnight at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (3.25 g, 72.1% yield). ¹H NMR (400 MHz, DMSO-d⁶): δ 9.38 (s, 1H), 7.43 (s, 1H), 7.33 (dd, J₁=8.0 Hz, J₂=1.6 Hz, 1H), 7.25 (d, J=8.0 Hz, 1H), 6.67 (d, J=8.4 Hz, 2H), 6.37 (d, J=8.4 Hz, 2H), 4.95 (s, 2H), 2.24 (s, 3H), 2.23 (s, 3H).

### Preparation of 4-isobutyrylbenzaldehyde

To a solution of 1-bromo-4-(diethoxymethyl)benzene (45.30 g, 174.81 mmol) in 200 mL of anhydrous tetrahydrofuran under an argon atmosphere was added dropwise n-butyllithium (76.88 mL of 2.5 N hexanes solution, 192.2 mmol) at −78° C. in a dry ice/acetone bath. After stirred at −78° C. for 2 hours, a solution of CuCN (15.66 g, 184.81 mmol) and LiCl (14.82 g, 349.62 mmol) in 200 mL anhydrous tetrahydrofuran was added dropwise with stirring at −78° C. under argon. The resulted solution was stirred at −78° C. for 30 minutes, then slowly warmed to the room temperature and stirred for another hour. The reaction was quenched by addition of 100 mL of water at the room temperature with vigorous stirring. THF solvent was removed under reduced pressure. The aqueous residue was extracted with methylene chloride (3×100 mL). The organic extracts were dried over anhydrous MgSO₄, filtered and concentrated under reduced pressure. The crude liquid was purified by column chromatography (silica gel, CH₂Cl₂) to give an oil product (28.00 g, 91.5% yield). ¹H NMR (400 MHz, DMSO-d⁶): δ 10.11 (s, 1H); 8.14 (d, J=8.0 Hz, 2H), 8.05 (d, J=8.0 Hz, 2H), 3.70 (m, 1H), 1.12 (d, J=6.8 Hz, 6H).

### Preparation of N-(4-(4-isobutyrylphenyl)-2,3,3a,4,5,9b-hexahydrofuro[3,2-c]quinolin-8-yl)-3,4-dimethylbenzenesulfonamide (69L162)

Compound N-(4-aminophenyl)-3,4-dimethylbenzenesulfonamide (0.50 g, 1.81 mmol), 4-isobutyrylbenzaldehyde (0.32 g, 1.81 mmol), Sc(OTf)₃ (0.18 g, 0.36 mmol) and 4 Å molecular sieves (1 g) were mixed together in anhydrous CH₃CN (20 mL) at room temperature under argon. The reaction mixture was stirred for one hour at room temperature. 2,3-Dihydrofuran (0.25 g, 3.62 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.55 g, 60.4% yield). cis/trans diastereomers: ¹H NMR (400 MHz, DMSO-d⁶): δ 9.59 (s, 0.6H), 9.54 (s, 0.4H), 7.97 (d, J=8.0 Hz, 2H), 7.59 (d, J=8.0 Hz, 2H), 7.48 (s, 0.6H), 7.45 (s, 0.4H), 7.41-7.37 (m, 1H), 7.29 (s, 0.6H), 7.27 (s, 0.4H), 6.91 (d, J=2.4 Hz, 0.6H), 6.83 (d, J=2.4 Hz, 0.4H), 6.77-6.71 (m, 1H), 6.57-6.54 (m, 1H), 6.23 (s, 0.6H), 5.95 (s, 0.4H), 5.01 (d, J=7.6 Hz, 0.4H), 4.64 (d, J=2.8 Hz, 0.4H), 4.30 (d, J=5.2 Hz, 0.6H), 3.86-3.82 (m, 0.6H), 3.70-3.63 (m, 2.4H), 3.57-3.52 (m, 0.6H), 3.46-3.42 (m, 0.4H), 2.65-2.62 (m, 0.4H), 2.25 (s, 3H), 2.24 (s, 3H), 1.90-1.80 (m, 1.4H), 1.52-1.49 (m, 0.6H), 1.28-1.24 (m, 0.6H), 1.10 (d, J=6.8 Hz, 6H). HRMS (ESI) calcd for C₂₉H₃₃N₂O₄S: 505.2161 [M+H]⁺, found 505.2160.

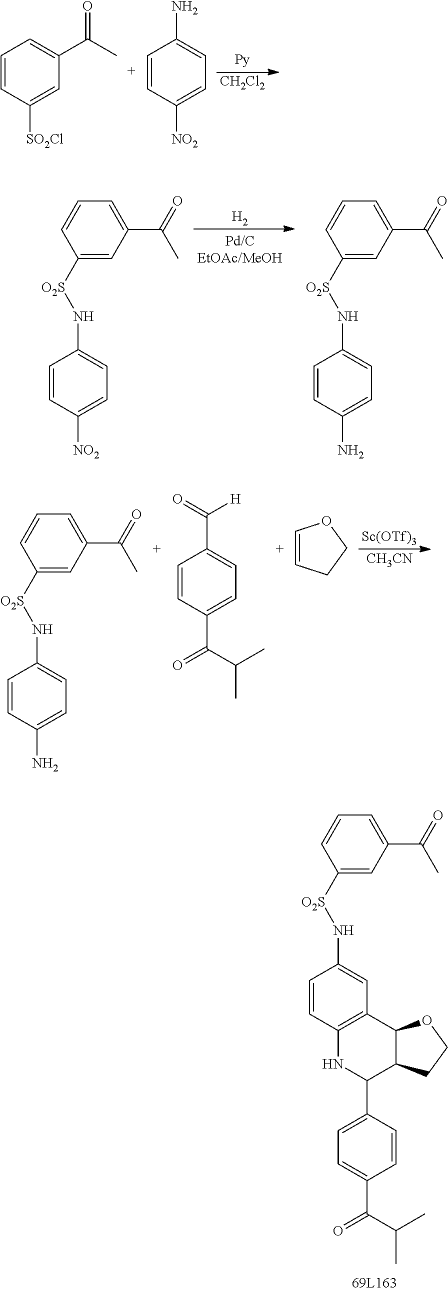

### Preparation of 3-acetyl-N-(4-nitrophenyl)benzenesulfonamide

3-Acetylbenzenesulfonyl chloride (2.5 g, 11.44 mmol) and 4-nitroaniline (1.58 g, 11.44 mmol) were dissolved in anhydrous methylene chloride (50 mL) at room temperature under argon. Pyridine (1.36 g, 17.16 mmol) was added dropwise via a syringe at room temperature. The reaction mixture was stirred at room temperature overnight under argon atmosphere. Then, the volatile was removed under reduced pressure. The yellow solid residue was subjected to flash column chromatography (silica gel, CH₂Cl₂/Acetone=9/1 v/v) to afford the desired product as a pale yellow solid (2.85 g, 77.9% yield). ¹H NMR (400 MHz, DMSO-d⁶): δ 11.42 (s, 1H); 8.36 (s, 1H), 8.24 (d, J=8.0 Hz, 1H), 8.15 (d, J=9.2 Hz, 2H), 7.10 (d, J=7.6 Hz, 1H), 7.79-7.76 (m, 1H), 7.33 (d, J=9.2 Hz, 1H), 3.37 (s, 3H).

### Preparation of 3-acetyl-N-(4-aminophenyl)benzenesulfonamide

Compound N-(3-acetylphenyl)-4-nitrobenzenesulfonamide (10.00 g, 31.22 mmol) and Pd/C (10% Pd base, 1 g) were mixed together in MeOH (100 mL) and Ethyl acetate (100 mL) at room temperature. Hydrogen gas was introduced via a H₂ balloon. The reaction mixture was stirred under H₂ atmosphere 8 hours at room temperature. The reaction mixture was filtered to remove the solid. The solution was concentrated under reduced pressure to give a pale yellow solid which was purified by column chromatography (silica gel, CH₂Cl₂/MeOH=9/1 v/v) to give a pale yellow solid product (5.60 g, 61.8% yield). ¹H NMR (400 MHz, DMSO-d⁶): δ 10.14 (s, 1H); 7.61-7.58 (m, 2H), 7.41-7.33 (m, 4H), 6.52 (d, J=8.4 Hz, 2H), 6.01 (s, 2H), 2.49 (s, 3H).

### Preparation of 3-acetyl-N-(4-(4-isobutyrylphenyl)-2,3,3a,4,5,9b-hexahydrofuro[3,2-c]quinolone-8-yl)benzenesulfonamide (69L163)

Compound N-(3-acetylphenyl)-4-aminobenzenesulfonamide (0.51 g, 1.76 mmol), 4-isobutyrylbenzaldehyde (0.31 g, 1.76 mmol), Sc(OTf)₃ (0.17 g, 0.35 mmol) and 4 Å molecular sieves (1 g) were mixed together in anhydrous CH₃CN (20 mL) at room temperature under argon. The reaction mixture was stirred for one hour at room temperature. 2,3-Dihydrofuran (0.25 g, 3.52 mmol) was added via a syringe. The resulted mixture was stirred at room temperature under argon overnight. The reaction was quenched by addition of 50 mL of water at room temperature and neutralized by adding NaHCO₃. The solution was extracted with ethyl acetate (3×50 mL). The extracts were dried over anhydrous MgSO₄, filtered and concentrated under reduced pressure to dryness. The solid residue was purified by column chromatography (silica gel, CH₂Cl₂/acetone=9/1 v/v) to give a white solid product (0.45 g, 49.5% yield). cis/trans diastereomers: ¹H NMR (400 MHz, CDCl₃): (7.98 (d, J=8.0 Hz, 2H), 7.89 (s, 0.5H), 7.82 (s, 0.5H), 7.68 (d, J=7.2 Hz, 1H), 7.61 (s, 1H), 7.51-7.45 (m, 3H), 7.41-7.36 (m, 2H), 6.89 (s, 0.5H), 6.82 (s, 0.5H), 6.58-6.55 (m, 1H), 5.19 (d, J=7.2 Hz, 0.5H), 4.86 (s, 0.5H), 4.66 (s, 0.5H), 4.52 (d, J=4.4 Hz, 0.5H), 4.37 (s, 0.5H), 4.02-3.98 (m, 0.5H), 3.92-3.84 (m, 1H), 3.72-3.69 (m, 0.5H), 3.66-3.63 (m, 0.5H), 3.59-3.52 (m, 1H), 2.78-2.74 (m, 0.5H), 2.57 (s, 3H), 2.42-2.37 (m, 0.5H), 2.06-1.97 (m, 1H), 1.72-1.69 (m, 0.5H), 1.52-1.49 (m, 0.5H), 1.23 (d, J=6.8 Hz, 6H). HRMS (ESI) calcd for C₂₉H₃₀N₂O₅S: 541.1773 [M+Na]⁺; found 541.1772.

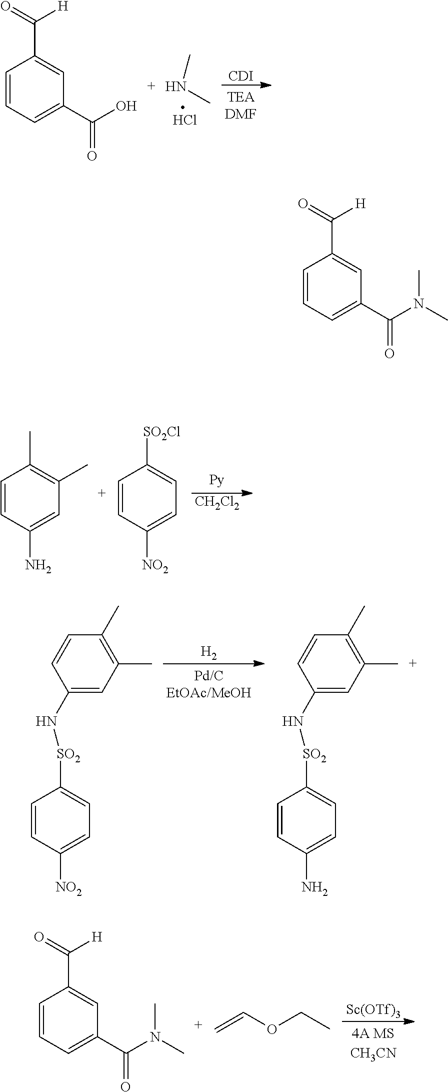

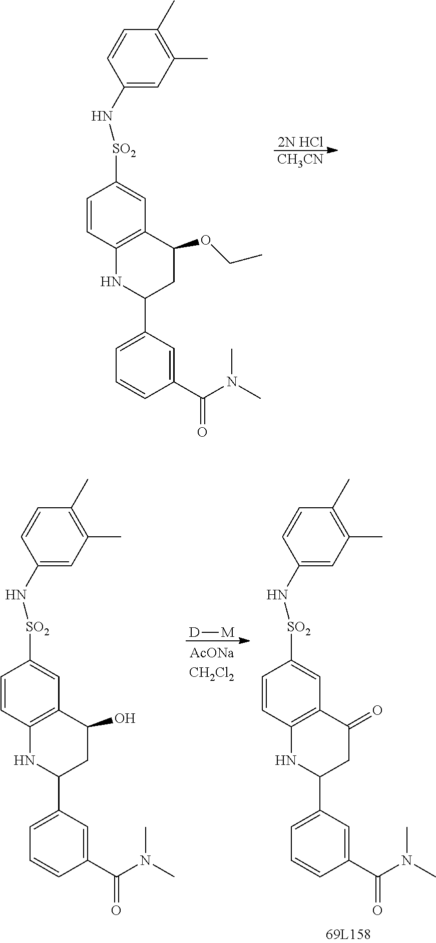

### Preparation of 3-formyl-N,N-dimethylbenzamide

¹H NMR (400 MHz, CDCl₃) δ 10.05 (s, 1H), 7.98-7.90 (m, 2H), 7.71 (dt, J₁=7.6 Hz, J₂=1.4 Hz, 1H), 7.61 (t, J=7.8 Hz, 1H), 3.15 (s, 3H), 3.01 (s, 3H).

### Preparation of 3-(6-(N-(3,4-dimethylphenylsulfamoyl)-4-oxo-1,2,3,4-tetrahydroquinolin-2-yl)-N,N-dimethylbenzamide (69L158)

Compound 3-(6-(N-(3,4-dimethylphenyl)sulfamoyl)-4-hydroxy-1,2,3,4-tetrahydroquinolin-2-yl)-N,N-dimethylbenzamide (0.12 mg, 0.25 mmol) was dissolved in anhydrous methylene chloride (10 mL) at room temperature. To this solution were added 0.11 g (0.25 mmol) of Dess-Martin reagent at room temperature. The reaction solution was stirred at room temperature for 10 minutes, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=7/3 v/v) to give a yellow solid product (45 mg, 37.8% yield). ¹H NMR (400 MHz, CDCl₃) δ 8.24 (d, J=2.2 Hz, 1H), 7.51 (dd, J₁=8.7 Hz, J₂=2.3 Hz, 1H), 7.45 (s, 1H), 7.39-7.29 (m, 3H), 6.90 (d, J=8.1 Hz, 1H), 6.82 (d, J=1.9 Hz, 1H), 6.73 (dd, J₁=8.1 Hz, J₂=2.3 Hz, 1H), 6.70 (s, 1H), 6.56 (d, J=8.7 Hz, 1H), 5.04 (s, 1H), 4.73 (dd, J₁=11.9 Hz, J₂=5.3 Hz, 1H), 3.06 (s, 3H), 2.92 (s, 3H), 2.85-2.72 (m, 2H), 2.10 (d, J=2.8 Hz, 6H),

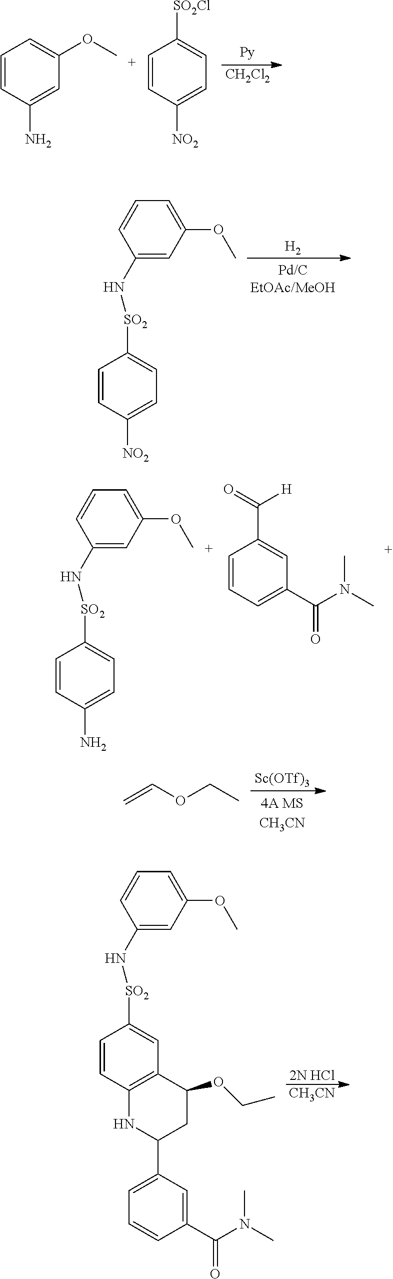

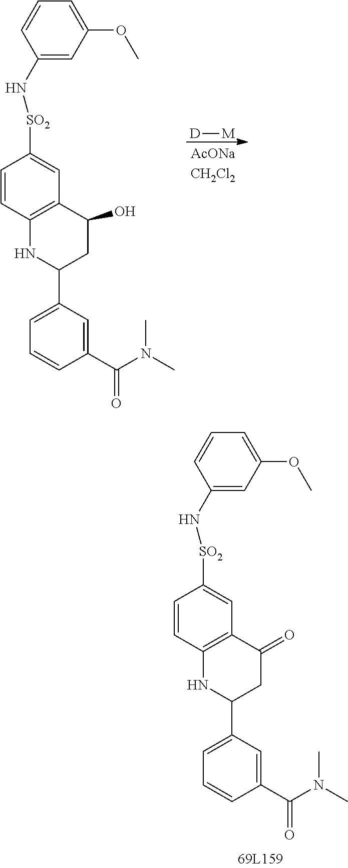

### Preparation of 3-(6-(N-(3-methoxyphenyl)sulfamoyl)-4-oxo-1,2,3,4-tetrahydroquinolin-2-yl)-N,N-dimethylbenzamide (69L159)

Compound 3-(4-hydroxy-6N-(3-methoxyphenyl)sulfamoyl)-1,2,3,4-tetrahydroquinolin-2-yl)-N,N-dimethylbenzamide (0.11 mg, 0.23 mmol) was dissolved in anhydrous methylene chloride (10 mL) at room temperature. To this solution were added 0.10 g (0.23 mmol) of Dess-Martin reagent at room temperature. The reaction solution was stirred at room temperature for one hour, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (40 mg, 36.7% yield). ¹H NMR (400 MHz, DMSO) δ 10.12 (s, 1H), 8.02-7.92. (m, 3H), 7.60 (dd, J₁=8.8 Hz, J₂=2.2 Hz, 1H), 7.50 (d, J=7.8 Hz, 1H), 7.44 (dd, J₁=9.5 Hz, J₂=4.8 Hz, 2H), 7.35 (d, J=7.5 Hz, 1H), 7.12 (t, J=8.1 Hz, 1H), 6.92 (d, J=8.9 Hz, 1H), 6.65 (d, J=6.7 Hz, 2H), 6.60-6.55 (m, 1H), 4.90 (dd, J₁=11.7 Hz, J₂=4.5 Hz, 1H), 3.66 (s, 3H), 2.95 (s, 3H), 2.84 (s, 3H), 2.72 (dd, J₁=16.2 Hz, J₂=4.4 Hz, 1H).

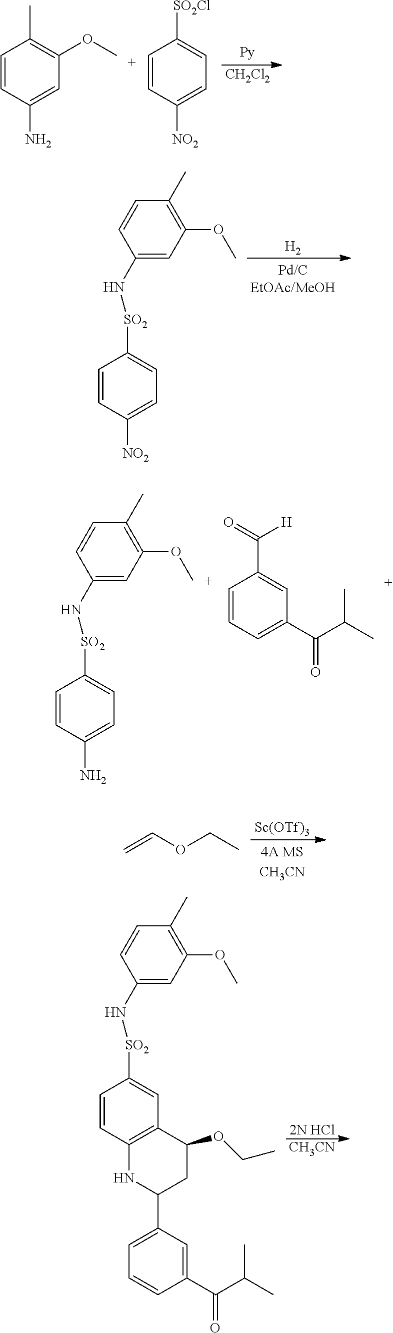

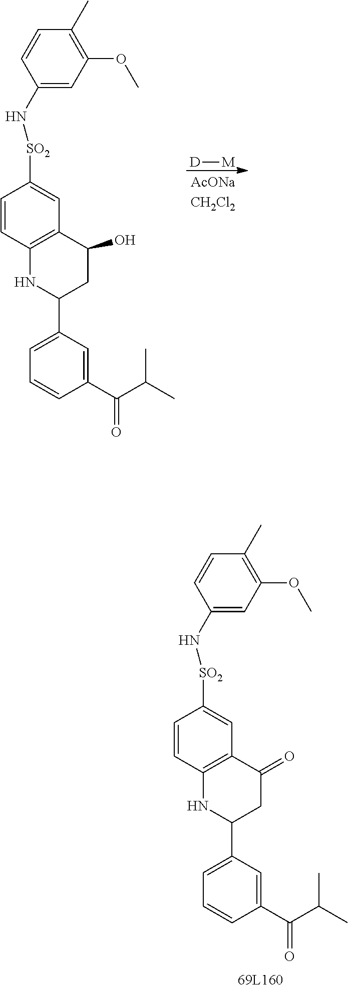

### Preparation of 2-(3-isobutyrylphenyl)-N-(3-methoxy-4-methylphenyl)-4-oxo-1,2,3,4-tetrahydroquinoline-6-sulfonamide (691160)

Compound 4-hydroxy-2-(3-isobutyrylphenyl)-N-(3-methoxy-4-methylphenyl)-1,2,3,4-tetrahydroquinoline-6-sulfonamide (0.10 mg, 0.20 mmol) was dissolved in anhydrous methylene chloride (10 mL) at room temperature. To this solution were added 86 mg (0.20 mmol) of Dess-Martin reagent at room temperature. The reaction solution was stirred at room temperature for 10 minutes, then diluted with 20 mL of water. The resulted mixture was extracted with methylene chloride (3×50 mL). The extracts were dried over anhydrous MgSO₄, filtered and concentrated under reduced pressure to dryness. The brown solid residue was purified by column chromatography (silica gel, CH₂Cl₂/Acetone=9/1 v/v) to give a yellow solid product (35 mg 35.2% yield). ¹H NMR (400 MHz, CDCl₃) δ 8.36 (d, J=2.0 Hz, 1H), 8.06 (s, 1H), 7.93 (d, J=7.7 Hz, 1H), 7.59 (dd, J₁=8.7 Hz, J₂=2.2 Hz, 2H), 7.51 (t, J=7.7 Hz, 1H), 7.12 (s, 1H), 6.93 (d, J₁=7.9 Hz, 1H), 6.74 (d, J=1.7 Hz, 1H), 6.68 (d, J=8.7 Hz, 1H) 6.50 (dd, J₁=7.9 Hz, J₂=1.9 Hz, 1H), 5.16 (s, 1H) 4.87 (dd, J₁=12.9 Hz, J₂=4.4 Hz, 1H), 3.77 (s, 3H), 3.55 (dt, J₁=13.6 Hz, J₂=6.8 Hz, 1H), 2.96-2.78 (m, 2H), 2.11 (s, 3H), 1.21 (dd, J₁=6.8 Hz, J₂=1.8 Hz, 6H).

| | Cancer cell | | | |
|---|---|---|---|---|
| Com- | (EU-1) | Mouse | | |
| pound | cytotoxicity | MTD | MDM2 | p53 |
| No. | IC50 (μM) | (mg/kg) | reduction | induction |
| MX69 | 7 | 200 | +++ | +++ |
| 69L1 | 1.5-6.0 | 300 | | |
| 69L11 | 2.3 (855 line) | | | |
| | 2.0 (855 line) | | | |
| | 1.52 (NB-1643) | | | |
| 69L28 | 8.4-11.9 | | | |
| 69L29 | 2.0-2.7 | | | |
| 69L2 | 3.5-5.3 | | | |
| 69L12 | 3.0-3.9 | | | |
| 69L13 | 6.4-9.0 | | | |
| 69L27 | 2.9-4.0 | | | |
| 69L30 | 5.9-8.2 | | | |
| 69L31 | 4.24 | | | |
| 69L32 | 7.28 | | | |
| 69L33 | >50 | | | |
| 69L34 | 3.44 | | | |
| 69L35 | 4.86 | | | |
| 69L36 | 9.20 | | | |
| 69L37 | 4.81 | | | |
| 69L38 | 0.49 (855) | | No MDM2 | |
| | 1.82 (NB-1643) | | inhibition | |
| 69L39 | 0.68 (855) | | No MDM2 | |
| | 1.61 (NB-1643) | | inhibition | |
| 69L40 | >50 | | | |
| 69L41 | >50 | | | |
| 69L42 | 19.9 | | | |
| 69L43 | 12.0 | | | |
| 69L44 | 44.9 | | | |
| 69L46 | 6.5 | | | |
| 69L47 | 2.1 | | | |
| 69L48 | 3.8 | | MDM2 | |
| | | | inhibition | |
| 69L49 | 4.6 | | | |
| 69L50 | 4.1 | | | |
| 69L51 | 4.5 | | | |
| 69L52 | 0.74 (855 cell line) | | MDM2 | p53 |
| | 0.50 (855 luc) | | inhibition | induction |
| | 0.45 (697 cell line) | | | |
| | 0.70 (NB-1643 line) | | | |
| | 3.62 (91-06 line, | | | |
| | no MDM2 and p53) | | | |
| | 5.93 (93-10 line no | | | |
| | MDM2 and p53) | | | |
| | 2.17 (DAOY line | | | |
| | with MDM2/no p53) | | | |
| | 1.1-1.4 | 0.5-0.6 | | |
| 69L52a | 1.9-2.0 | | MDM2 | p53 |
| | | | inhibition | induction |
| | | | (+) | (++) |
| 69L52b | 1.4-1.6 | | MDM2 | p53 |
| | | | inhibition | induction |
| | | | (+++) | (+++) |
| 69L52c | 2.4-2.5 | | MDM2 | p53 |
| | | | inhibition | induction |
| | | | (−) | (−) |
| 69L52d | 0.50 | | MDM2 | p53 |
| | | | inhibition | induction |
| | | | (+−) | (−) |
| 69L53 | 0.80 (855 line) | MTD | MDM2 | p53 |
| | 1.73 (NB-1643) | 300 mg/kg | inhibition | induction |
| 69L54 | 3.22 | | MDM2 | |
| | | | inhibition | |
| 69L55 | 1.64 | | No MDM2 | |
| | | | inhibition | |
| 69L56 | 10.21 | | MDM2 | |
| | | | inhibition | |
| 69L57 | 10.8 | | | |
| 69L58 | 1.61 (855) | | MDM2 | p53 |
| | 2.63 (NB-1643) | | inhibition | induction |
| 69L59 | 2.31 (855 line) | | No MDM2 | |
| | 3.09 (NB-1643) | | inhibition | |
| 69L60 | 1.81 (855 line) | | Increase | p53 |
| | 2.73 (697 line) | | MDM2 | induction |
| | 5.65 (NB-1643) | | expression | +++ |
| 69L61 | 2.43 (855 line) | | MDM2 | No p53 |
| | 3.6 (697 line) | | inhibition | induction |
| | 1.6 (NB-1643) | | ++++ | |
| | | | MYCN | |
| | | | inhibition | |
| 69L62 | 0.98 (855 line) | | No MDM2 | p53 |
| | 0.49 (697 line) | | inhibition | induction |
| | 1.3 (NB-1643) | | | (+) |
| 69L63 | 6.07 (855 line) | | MDM2 | p53 |
| | 3.94 (697 line) | | inhibition | induction |
| | | | ++ | |
| 69L64 | 1.35 (855 line) | | MDM2 | p53 |
| | 0.28 (697 line) | | inhibition | induction |
| | | | +++ | |
| 69L65 | 1.89 (855 line) | | No MDM2 | |
| | | | inhibition | |
| 69L66 | 2.56 | | No MDM2 | |
| | | | inhibition | |
| 69L67 | 5.18 | | | |
| 69L68 | 5.10 | | | |
| 69L69 | 5.81 | | | |
| 69L70 | 3.09 | | No MDM2 | |
| | | | inhibition | |
| 69L71 | 30 | | | |
| 69L72 | 2.94 | | No MDM2 | |
| | | | inhibition | |
| 69L73 | 1.56 | 69L52 | No MDM2 | |
| | | used as | inhibition | |
| | | positive | | |
| | | control: | | |
| | | 0.46 | | |
| 69L74 | 5.23 | | | |
| 69L75 | 3.21 | | No MDM2 | |
| | | | inhibition | |
| 69L76 | 5.59 | | | |
| 69L77 | 6.29 | | | |
| 69L78 | 3.67 | | No MDM2 | |
| | | | inhibition | |
| 69L79 | 2.91 | | No MDM2 | |
| | | | inhibition | |
| 69L80 | 2.66 | | No MDM2 | |
| | | | inhibition | |
| 69L81 | 3.20 | | MDM2 | p53 |
| | | | inhibition | inhibition |
| | | | XIAP | |
| | | | inhibition | |
| 69L82 | 29.7 | | | |
| 69L83 | 1.17 | | MDM2 | p53 |
| | | | inhibition | inhibition |
| 69L84 | 0.2-0.6 | | MDM2 | p53 |
| | | | inhibition | activation |
| | | | XIAP | |
| | | | inhibition | |
| 69L85 | 3.33 | | | |
| 69L86 | 3.77 | | | |
| 69L87 | 1.98 | | MDM2 | p53 |
| | | | inhibition | inhibition |
| 69L88 | 6.97 | | | |
| 69L89 | 7.2 | | | |
| 69L90 | 3.26 | | | |
| 69L91 | >10 | | | |
| 69L92 | >10 | | | |
| 69L93 | 1.95 | | MDM2 | p53 |
| | | | inhibition | induction |
| 69L94 | 3.56 | 69L84: | No MDM2 | |
| | | 0.59 | inhibition | |
| | | (as control) | | |
| 69L95 | 1.42 | | MDM2 | |
| | | | inhibition | |
| 69L96 | 2.08 | | MDM2 | |
| | | | inhibition | |
| 69L97 | 9.36 | 69L84 as | | |
| | | control | | |
| | | 0.46 | | |
| 69L98 | 1.50 | | MDM2 | P53 |
| | | | inhibition | induction |
| 69L99 | 0.23 | 69L84 as | MDM2 | |
| | | control: | inhibition | |
| | | 0.24-1.2 | | |
| 69L100 | 1.87 | | No MDM2 | |
| | | | inhibition | |
| 69L101 | 1.80 | | MDM2 | |
| | | | inhibition | |
| 69L102 | 0.23 | | MDM2 | |
| | | | inhibition | |
| | | | XIAP | |
| | | | inhibition | |
| 69L103 | 0.97 | | No MDM2 | |
| | | | inhibition | |
| 69L104 | 0.19 | | MDM2 | |
| | | | inhibition | |
| | | | XIAP | |
| | | | inhibition | |
| 69L105 | 3.12 | | No MDM2 | |
| | | | inhibition | |
| 69L106 | 0.96 | | No MDM2 | |
| | | | inhibition | |
| 69L107 | 0.93 | | MDM2 | |
| | | | inhibition | |
| 69L108 | 0.42 | | MDM2 | |
| | | | inhibition | |
| 69L109 | 0.1 | | No MDM2 | |
| | | | inhibition | |
| 69L110 | 0.27 | | | |
| 69L111 | 1.37 | | | |
| 69L112 | 0.37 | | | |
| 69L113 | <0.1 | | MDM2 | |
| | | | inhibition | |
| | | | XIAP | |
| | | | inhibition | |
| 69L114 | <0.1 | | MDM2 | |
| | | | inhibition | |
| | | | XIAP | |
| | | | inhibition | |
| 69L115 | 0.56 | | | |
| 69L116 | 0.12 | | No MDM2 | |
| | | | inhibition | |
| 69L117 | 0.30 | | No MDM2 | |
| | | | inhibition | |
| 69L118 | 0.15 | | | |
| 69L119 | 0.45 | | | |
| 69L120 | 0.35 | | | |
| 69L3 | 18.29 | | | |
| 69L4 | >50 | | | |
| 69L5 | >50 | | | |
| 69L6 | 20.5 | | | |
| 69L7 | >50 | | | |
| 69L8 | 3.61 | | | |
| 69L9 | 4.56 | | | |
| 69L10 | >50 | | | |
| 69L14 | >50 | | | |
| 69L15 | 17.9 | | | |
| 69L16 | 4.66 | | | |
| 69L17 | 8.69 | | | |
| 69L18 | 14.73 | | | |
| 69L19 | 4.06 | | | |
| 69L21 | 6.18 | | | |
| 69L22 | 19.3 | | | |
| 69L23 | 23.4 | | | |
| 69L24 | >50 | | | |
| 69L25 | >50 | | | |
| 69L26 | >50 | | | |
| 69L121 | 0.18 | | MDM2 | |
| | | | inhibition | |
| 69L122 | >10 | | | |
| 69L123 | <0.1 | | MDM3 | |
| | | | inhibition | |
| 69L124 | 0.46 | | | |
| 69L125 | 1.48 | | | |
| 69L126 | 1.14 | | | |
| 69L127 | 4.8 | | | |
| 69L128 | 1.31 | | | |
| 69L129 | 0.65 | | | |
| 69L130 | >10 | | | |
| 69L130 | >10 | | | |
| trans | | | | |
| 69L130 | >10 | | | |
| cis | | | | |
| 69L131 | 1.16 | 0.27 | | |
| 69L132 | 1.57 | | | |
| 69L133 | 1.58 | | | |
| 69L134 | 6.53 | | | |
| 69L135 | 0.15 | | | |
| 69L136 | 6.16 | | | |
| 69L137 | 5 | | | |
| 69L138 | 1.4 | | | |
| 69L139 | 0.66 | | | |
| 69L140 | 0.23 | | | |
| 69L141 | 7.5 | | | |
| 69L142 | >5 | | | |
| 69L143 | 3.96 | | | |
| 69L144 | 1.53 | | | |
| 69L145 | 3.5 | | | |
| 69L146 | 3.81 | | | |
| 69L147 | 1.16 | | | |
| 69L148 | 2.05 | | | |
| 69L149 | 0.58 | | | |
| 69L150 | 0.04 | | | |
| 69L151 | 1.9 | | | |
| 69L152 | 1.6 | | | |
| 69L153 | >5 | | | |
| 69L154 | >5 | | | |
| 69L155 | 1.45 | | | |
| 69L156 | 0.54 | | | |
| 69L157 | 0.33 | | | |
| 69L158 | 0.63 | | | |
| 69L159 | 0.65 | | | |
| 69L160 | 0.54 | | | |
| 69L161 | 0.82 | | | |
| 69L162 | >5 | | | |
| 69L163 | 2.5 | | | |
| 69L164 | 0.71 | | | |
| 69L165 | 8.9 | | | |
| 69L166 | 0.11 | | | |
| 69L167 | 3.6 | | | |
| 69L168 | 0.34 | | | |

Identification of MX69 Analogs with Increased MDM2 Binding Affinity.

Materials and Methods

Cell Lines and Cell Culture

This study used two ALL cell lines (EU-1 and EU-3) and three NB cell lines (NB-1643, SHEP1 and LA1-55N). All 5 cancer cell lines were established from pediatric ALL or NB patients and were well-characterized for their expression of MDM2 and p53-status, as reported previously. All cell lines were grown in standard culture medium (RPMI 1640 containing 10% FBS, 2 mmol/L L-glutamine, 50 U penicillin and 50 μg/mL streptomycin) at 37° C. in a humidified atmosphere containing 5% CO₂.

Immunoprecipitation and Western Blot Assay

Cells were lysed in a buffer composed of 50 mM Tris, pH 7.6, 150 mM NaCl, 1% Nonidet P-40, 10 mM sodium phosphate, 10 mM NaF, 1 mM sodium orthovanadate, 2 mM phenylmethylsulfonyl fluoride (PMSF), 10 g/mL aprotinin, 10 μg/mL leupeptin and 10 μg/mL pepstatin. After centrifugation, the clarified cell lysate was separated from the pellet of cell debris, and then incubated with 15 μL Protein G/Protein A-agarose and 1 μg of antibodies, overnight at 4° C. For the Western blot, the resulting cell lysates or immunoprecipitates were resolved by SDS-PAGE. They were then transferred to a nitrocellulose filter and probed with the specific antibodies as listed in the supplemental Materials section. Finally, proteins were visualized with a chemiluminescent detection system.

Pulse-Chase Assay

Protein turnover was assessed by a standard protein-synthesis inhibitor (CHX) assay. Briefly, cells were treated with 50 μg/mL CHX for different times before lysis, in the presence or absence of AQ-101, and then tested by Western blot analysis to reveal concurrent expression levels of MDM2, p53 and XIAP. The mRNA degradation rate was examined using a standard actinomycin D analysis: At different times after addition of 5 μg/mL of actinomycin D, in the presence or absence of AQ-101, the cells were harvested and their total RNA isolated. The MDM2 mRNA was detected by quantitative RT-PCR, as described above.

Compound-Protein Binding Assays

Isothermal titration calorimetry (ITC) assay was performed using the auto-iTC200 instrument (MicroCal, GE). MDM2 protein was loaded into a 96 DeepWell PP plate, and then compound was titrated stepwise into the protein sample cell using a syringe, for a total of 16 injections (except for the first injection, which was 0.4 μl). The equilibrium time between two adjacent injections was 210 s. The binding stoichiometry (n), binding constant (Kd), and thermodynamic parameters (ΔH and ΔS) were determined by fitting the titration curve to a one-site binding mode, using the Origin software provided by the manufacturer.

Polysome Preparation and Analysis

Cells were incubated with 100 μg/mL cycloheximide (CHX) for 15 min to arrest polyribosome migration, and then lysed (in order to isolate cytoplasmic extracts) in a buffer containing 20 mM Tris-HCl at pH 8.0, 100 mM NaCl, 5 mM MgCl₂, 0.5% Triton X-100, 500 U/mL RNAsin, and a cocktail of protease inhibitors. Fractionation was performed on a 15-45% (w/v) sucrose gradient at 39,000 rpm for 1 h (SW41Ti rotor). Fractions were collected by upward replacement in a fractionator (Isco, Lincoln, NE). The RNA from each fraction was subjected to quantitative PCR.

Clonogenic Assay

A clonogenic assay was used to determine the effect of MX69 analogs on in vitro growth of NB and normal human hematopoietic cells, respectively. For NB1 colony formation assay, cells were harvested with treatment by trypsinization, producing a single-cell suspension, and then 200 cells were seeded into a 6-well plate and cultured for approximately 2 weeks. Colonies were stained with a mixture of 6.0% glutaraldehyde and 0.5% crystal violet for 30 min. Then carefully removed the staining mixture, rinsed with tap water and counted the colonies.

For colony formation of normal human hematopoiesis assay, a bottom layer of low-melting-point, 0.5% agarose (in RPMI 1640 medium plus 10% FBS) was poured into gridded 35 mm dishes and allowed to gel. Cells were cultured in a top layer of 0.35% agarose/medium at 37° C. in a humidified atmosphere containing 5% CO₂. After 2-3 weeks, cultures were fixed with formalin and colonies scored.

Cytotoxicity Assay

The cytotoxic effect of MX 69 and MX69 analogs on cancer cells was determined using the water-soluble tetrazolium salt (WST) assay. Briefly, cells cultured in 96-well microtiter plates were treated with different concentrations of AQ-101 for a 20-h period. WST (25 μg/well) was then added and incubation continued for an additional 4 h, after which optical density (OD) was read with a microplate reader (test wavelength of 450 nm; reference wavelength of 620 nm).

Results

Both 69L52 and 69L53 have Increased MDM2 Binding Affinity

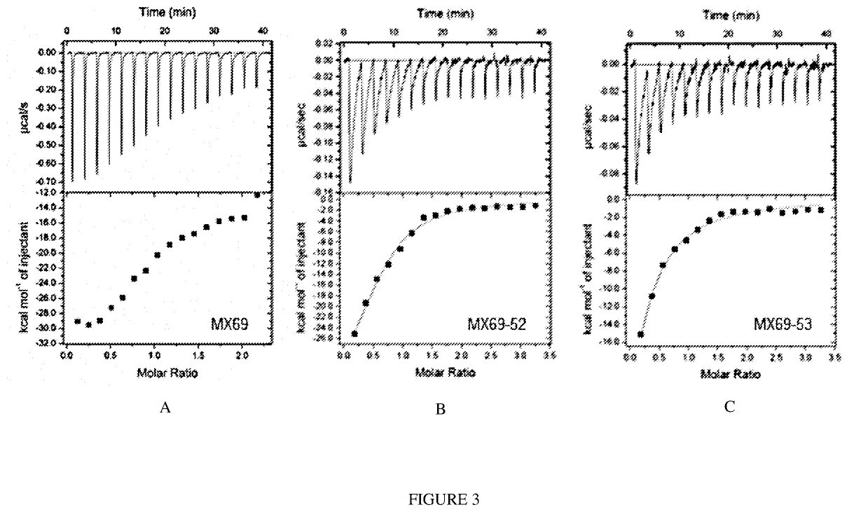

MX69 has been detected to bind to the MDM2 C-terminal RING protein. To confirm that the new analogs with improved potency maintain this mode of action, isothermal titration calorimetry (ITC) assays were performed. Both 69L52 and 53 bind to the MDM2 RING domain with increased binding affinities as compared with MX69 (FIG. 3).

Compound 69L52 Attenuates the Proliferation in Cancer Cells but Shows a Negligible Inhibitory Effect on Normal Hematopoiesis.

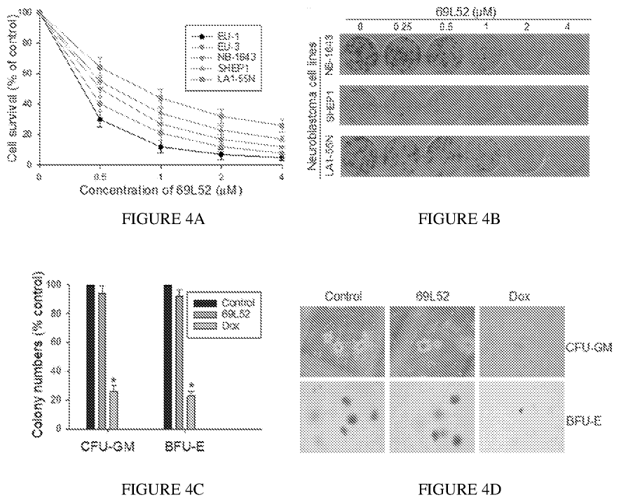

Five cancer cell lines were tested including two acute lymphoblastic (ALL) EU-1 and EU-3 and three neuroblastoma (NB) NB-1643, SHEP1 and LA1-55N in response to 69L52. WST cytotoxicity assays show that 69L52 consistently exhibited potent cytotoxicity against these tested cell lines with more sensitive of ALL (IC₅₀=0.3˜0.4 μM) than NB (IC₅₀=0.5˜1.2 μM) to 69L52 (FIG. 4A). As also seen in FIG. 4A, the much less cytotoxicity to NB line LA1-55N compared with other cell lines by compound 69L52 mostly likely because this line does not express normal p53 as characterized in our previous publication. Furthermore, results of colony formation assays showed that 69L52 potently inhibited cell growth in all 3 NB cell lines. A significant reduction in both colony number and size in 69L52-treated cells was observed as compared with controls (FIG. 4B).

To evaluate the possible inhibitory and toxic effect of 69L52 on normal hematopoiesis, clonogenic assays for CFU-GM and BFU-E were preformed using human normal bone marrow mononuclear (NBMM) cells, with doxorubicin (Dox) as a reference control. CFU-GM and BFU-E colony numbers and size in 69L52-treated samples were similar to the control, whereas both colony number and size were significantly reduced in the Dox-treated samples (FIGS. 4C and 4D).

Compound 69L52 Inhibits Expression of MDM2 Through Post-Translational Modification.

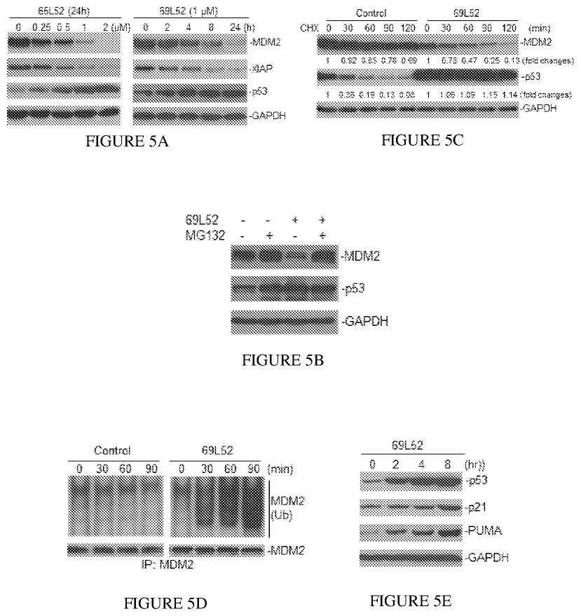

Western blot assays were performed for effects of 69L52 on MDM2 and XIAP expression. Results show that 69L52 induced a remarkable downregulation of MDM2 and XIAP in a dose-dependent manner and occurred at approximately 2-4 h after treatment, followed by steady-state suppression (FIG. 5A). Downregulation of MDM2 was accompanied by increased expression of p53. To further evaluate the mechanism by which 69L52 inhibits MDM2, MG132 (protein degradation inhibitor) treatment and cycloheximide (CHX) chase assays in EU-1 cells treated with 69L52 were performed; results show that 69L52 induced increased MDM2 protein degradation. As shown in FIG. 5B, the observed downregulation of MDM2 by 69L52 was blocked by MG132. CHX chase assay results showed that the half-life of MDM2 in untreated EU-1 cells was >120 min, whereas 69L52 treatment decreased the MDM2 half-life to <60 min (FIG. 5C). In contrast, the half-life of p53 in untreated cells was <30 min, and the time was increased to >120 min by treatment with 69L52. Since MDM2 protein stability is regulated by a self-ubiquitination mechanism, whether 69L52 induced MDM2-protein degradation is mediated through this mechanism was tested. Immunoprecipitation (IP)-Western blot assays were preformed and results show that 69L52 induced ubiquitination of endogenous MDM2 in EU-1 cells (FIG. 5D). These results show that 69L52 downregulates MDM2 through induction of MDM2 self-ubiquitination and degradation. Furthermore, activation of p53 following 69L52-mediated MDM2 ubiquitination and degradation led to activation of the p53 downstream targets p21 and PUMA (FIG. 5E).

Compound 69L52 Inhibits XIAP Translation and Activity.

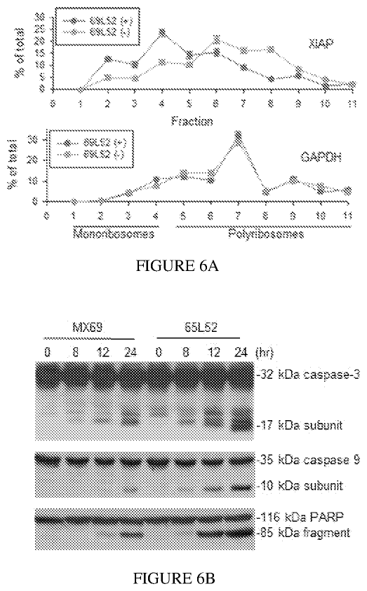

Linear sucrose-gradient fractionation was performed to assess the state of polyribosome association of XIAP mRNA in EU-1 cells subjected to 69L52 treatment. 69L52 induced a downregulation in polyribosome association. This was shown by a shift in XIAP mRNA from fractions containing enriched translating polyribosomes to fractions containing translation-inactive complexes monoribosomes (FIG. 6A). The effects of 69L52 mediated inhibition of XIAP on activation of caspases-3 and -9 were also tested, as well as cleavage of the death substrate PARP. As shown in FIG. 6B, cleavage of caspases-3, -9, and PARP can be detected 8 h after McX69-102 treatment in EU-1 cells. Simultaneously, EU-1 cells were treated with MX69 as comparison, and results show that 69L52 induced stronger cleavage of caspases-3, -9 and PARP at a much lower dose (1 μM) than MX69 (5 μM). These demonstrate that the increased potency of 69L52 than MX69 in inhibiting EU-1 cells is closely associated with the enhanced inhibition of XIAP function as well as activation of p53.

1. A compound having Formula I 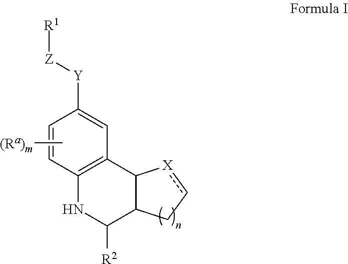
or pharmaceutically acceptable salts and prodrugs thereof wherein, [symbol]
is an optional double bond;
n is 1 or 2;
m is 0, 1, 2, or 3;
X is O, S, CH, CH₂, NR^{b}, or NH;
Y is absent, SO, SO₂, CO or NH;
Z is absent, O, S, SO₂, CO, NH, or N-alkyl;
R^{a} is independently, at each occurrence, halogen, hydroxy, nitro, cyano, or alkoxy;
R^{b} is alkyl or C(O)O-alkyl;
R¹ is absent, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵, and wherein two R¹⁵, together with the atoms to which they are attached, may form a heterocyclic ring;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

2. The compound of claim 1, wherein n is 1, X is O, Y is SO₂, Z is NH, R¹ is aryl, and R² is carbocyclyl, aryl, or heterocyclyl.

3. The compound of claim 1, wherein the compound of Formula I is a compound of Formula Ia or Formula Ib, 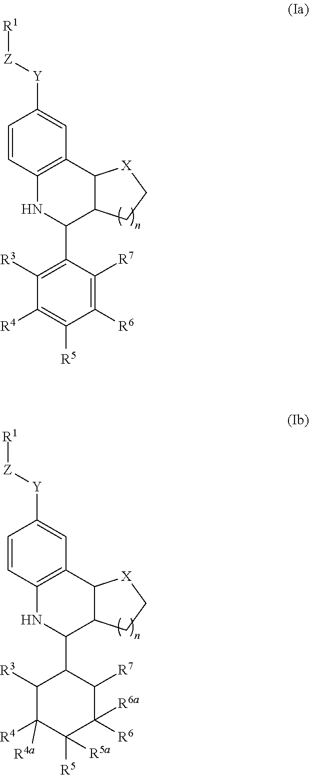
or pharmaceutically acceptable salts and prodrugs thereof wherein,
n is 1 or 2;
X is O, S, or NH;
Y is absent, SO, or SO₂;
Z is O, S, or NH;
R¹ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵;
R³ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R³ is optionally substituted with one or more, the same or different, R¹⁵;
R⁴ and R^{4a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁴ and R^{4a} are optionally substituted with one or more, the same or different, R¹⁵;
R⁵ and R^{5a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁵ and R^{5a} are optionally substituted with one or more, the same or different, R¹⁵;
R⁶ and R^{6a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁶ and R^{6a} are optionally substituted with one or more, the same or different, R¹⁵;
R⁷ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁷ is optionally substituted with one or more, the same or different, R¹⁵;
R¹⁵ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

4. The compound of claim 3, wherein n is 1, X is O, Y is SO₂, Z is NH, R¹ is aryl.

5. The compound of claim 1, wherein the compound of Formula I is a compound of Formula Ic, 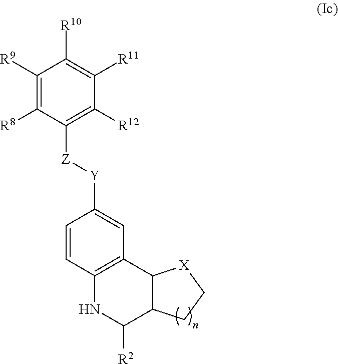
or pharmaceutically acceptable salts and prodrugs thereof wherein,
n is 1 or 2;
X is O, S, or NH;
Y is absent, SO, or SO₂;
Z is O, S, or NH;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵;
R⁸ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁸ is optionally substituted with one or more, the same or different, R¹⁵;
R⁹ is hydrogen alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁹ is optionally substituted with one or more, the same or different, R¹⁵;
R¹⁰ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹⁵;
R¹¹ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹⁵;
R¹² is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹² is optionally substituted with one or more, the same or different, R¹⁵;
R¹⁵ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is individually and independently at each occurrence alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

6. The compound of claim 5, wherein n is 1, X is O, Y is SO₂, Z is NH, R² is carbocyclyl, aryl, or heterocyclyl.

7. The compound of claim 1, wherein the compound of Formula I is a compound of Formula Id or Formula Ie, 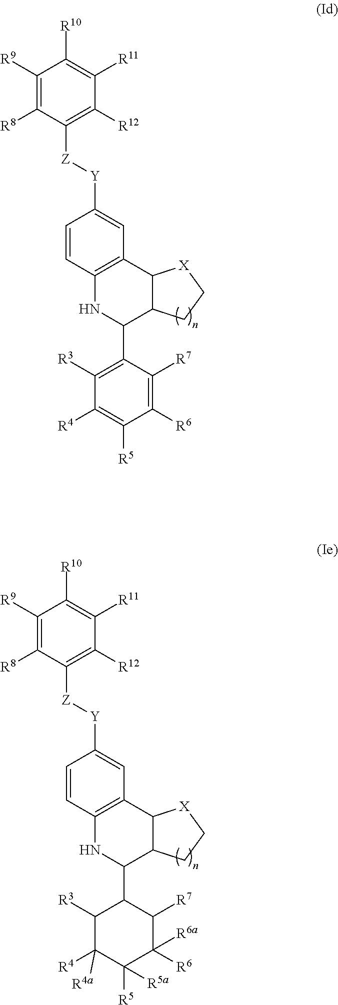
or pharmaceutically acceptable salts and prodrugs thereof wherein,
n is 1 or 2;
X is O, S, or NH;
Y is absent, SO, or SO₂;
Z is O, S, or NH;
R³ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R³ is optionally substituted with one or more, the same or different, R¹⁵;
R⁴ and R^{4a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁴ and R^{4a} are optionally substituted with one or more, the same or different, R¹⁵;
R⁵ and R^{5a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁵ and R^{5a} are optionally substituted with one or more, the same or different, R¹⁵;
R⁶ and R^{6a} are individually and independently at each occurrence hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁶ and R^{6a} are optionally substituted with one or more, the same or different, R¹⁵;
R⁷ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁷ is optionally substituted with one or more, the same or different, R¹⁵;
R⁸ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁸ is optionally substituted with one or more, the same or different, R¹⁵;
R⁹ is hydrogen alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R⁹ is optionally substituted with one or more, the same or different, R¹⁵;
R¹⁰ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹⁵;
R¹¹ is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹⁵;
R¹² is hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹² is optionally substituted with one or more, the same or different, R¹⁵;
R¹⁵ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

8. A compound of Formula IV′: 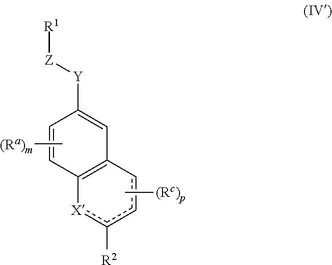
or pharmaceutically acceptable salts and prodrugs thereof;
wherein [symbol]
is an optional double bond;
m is 0, 1, 2, or 3; p is 0, 1, or 2; X′ is O or NH; Y is absent, SO, SO₂, CO or NH; Z is absent, O, S, SO₂, CO, NH, or N-alkyl; R^{a} is independently, at each occurrence, halogen, hydroxy, nitro, cyano, or C₁-C₆ alkoxy; R^{c} is independently, at each occurrence, hydroxy, alkoxy, C(O), ═O, ═S, ═NH, N(R¹⁵)₂, OR¹⁵, halogen, or aryl, wherein aryl is optionally substituted with one of more halogen; or alternatively, two R^{c}, together with the atoms to which they are attached, form a heterocyclic ring optionally fused to an aryl ring;
R¹ is absent, alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹ is optionally substituted with one or more, the same or different, R¹⁵; and wherein two R¹⁵, together with the atoms to which they are attached, may form a heterocyclic ring;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

9. The compound of claim 8, wherein the compound of Formula IV′ is a compound of Formula IV: 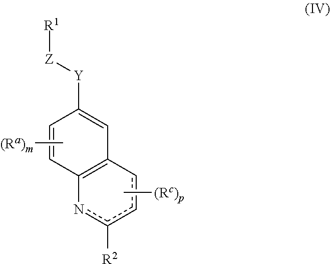
or pharmaceutically acceptable salts and prodrugs thereof; wherein [symbol] is an optional double bond; and m, p, Y, Z, R^{a}, R^{c}, R¹, R², R¹⁵, R¹⁶, and R¹⁷ are as defined in the base claim.

10. The compound of claim 9, wherein the compound of Formula IV is a compound of Formula IVa: 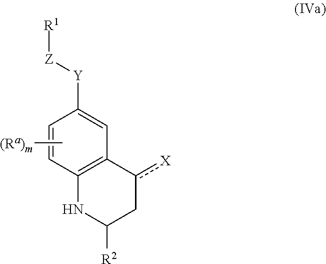
or a pharmaceutically acceptable salt thereof;
wherein [symbol]
is an optional double bond;
m is 0, 1, 2, or 3;
X is O, S, NH, N(R¹⁵)₂, OR¹⁵, or halogen;
Y is SO or SO₂;
Z is absent, O, S, or NH;
R^{a} is independently, at each occurrence, halogen, hydroxy, nitro, cyano, or C₁-C₆ alkoxy;
R² is alkyl, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R² is optionally substituted with one or more, the same or different, R¹⁵; and wherein two R¹⁵, together with the atoms to which they are attached, may form a heterocyclic ring;
R¹⁵ is alkyl, alkenyl, boronic acid, boronic ester, halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁵ is optionally substituted with one or more, the same or different, R¹⁶;
R¹⁶ is alkyl, —C(O), halogen, cyano, hydroxy, amino, mercapto, formyl, carboxy, carbamoyl, alkoxy, alkanoyl, alkylthio, alkylamino, aminoalkyl, (alkyl)₂amino, alkylsulfinyl, alkylsulfonyl, arylsulfonyl, carbocyclyl, aryl, or heterocyclyl, wherein R¹⁶ is optionally substituted with one or more, the same or different, R¹⁷; and
R¹⁷ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

11. The compound of claim 1, wherein the compound of Formula I is 4-cyclohexyl-N-(3,4-dimethylphenyl)-2,3,3a,4,5,9b-hexahydrofuro[3,2-c]quinoline-8-sulfonamide, salts or prodrugs thereof.

12. The compound of claim 1, wherein the compound of Formula I is N-(3,4-dimethylphenyl)-4-(4-isobutyrylphenyl)-2,3,3a,4,5,9b-hexahydrofuro[3,2-c]quinoline-8-sulfonamide, salts or prodrugs thereof.

13. A pharmaceutical composition comprising a therapeutically effective amount of the compound of claim 1 and a pharmaceutically acceptable carrier.

14. (canceled)

15. A method of treating cancer in a subject in need thereof comprising (i) administering to the subject a therapeutically effective amount of the compound of claim 1 wherein the cancer is leukemia selected from the group consisting of childhood acute lymphoblastic leukemia, acute lymphoblastic leukemia, acute lymphocytic leukemia, acute myeloid leukemia, adult acute lymphocytic leukemia, adult acute myeloid leukemia, and adult lymphocytic leukemia.

16. (canceled)

17. (canceled)

18. (canceled)

19. A method of reducing MDM2 protein levels in a subject in need thereof, comprising administering to the subject a therapeutically effective amount of the compound of claim 1 or the composition according to claim 13.

20. A method for treating cancer cells in a subject in need thereof, comprising administering to the subject a therapeutically effective amount of a compound or composition of claim 1, wherein the compound or composition has a cytotoxicity against ALL EU-1 cell line lower than MX69, tested under the same conditions.

21. A method of increasing expression levels of p53 in a subject in need thereof, comprising administering to the subject a therapeutically effective amount of the compound of claim 1.

22. A pharmaceutical composition comprising a therapeutically effective amount of the compound of claim 8 and a pharmaceutically acceptable carrier.

23. A method of treating cancer in a subject in need thereof comprising administering to the subject a therapeutically effective amount of the compound of claim 8, wherein the cancer is leukemia selected from the group consisting of childhood acute lymphoblastic leukemia, acute lymphoblastic leukemia, acute lymphocytic leukemia, acute myeloid leukemia, adult acute lymphocytic leukemia, adult acute myeloid leukemia, and adult lymphocytic leukemia.

24. A method of reducing MDM2 protein levels in a subject in need thereof, comprising administering to the subject a therapeutically effective amount of the compound of claim 8.
